(19) United States
(12) Patent Application Publication
(10) Pub. No.: US 2021/0388375 A1
(43) Pub. Date: Dec. 16, 2021

(54) GENES ASSOCIATED WITH RESISTANCE TO WHEAT YELLOW RUST

(71) Applicants: JOHN INNES CENTRE, Norwich, Norfolk (GB); COMMONWEALTH SCIENTIFIC AND INDUSTRIAL RESEARCH ORGANISATION, Acton (AU); THE UNIVERSITY OF SYDNEY, Sydney, New South Wales (AU)

(72) Inventors: Cristobal UAUY, Norwich (GB); Clemence MARCHAL, Norwich (GB); Evans LAGUDAH, Canberra (AU); Robert MCINTOSH, Cobbitty (AU); Jianping ZHANG, Cobbitty (AU); Peng ZHANG, Cobbitty (AU)

(73) Assignees: JOHN INNES CENTRE, Norwich, Norfolk (GB); COMMONWEALTH SCIENTIFIC AND INDUSTRIAL RESEARCH ORGANISATION, Acton (AU); THE UNIVERSITY OF SYDNEY, Sydney, New South Wales (AU)

(21) Appl. No.: 17/046,561

(22) PCT Filed: Apr. 9, 2019
(86) PCT No.: PCT/EP2019/058963

§ 371 Date: Oct. 9, 2020

(30) Foreign Application Priority Data
Apr. 9, 2018 (GB) 1805865.1

(51) Int. Cl. C12N 15/82; C12Q 1/6813

(52) U.S. Cl. CPC C12N 15/8282; C12Q 2600/13; C12Q 1/6813

(57) ABSTRACT

An isolated nucleic acid encoding a nucleotide-binding and leucine-rich repeat (NLR) polypeptide including a zinc-finger BED domain, wherein expression of the NLR polypeptide in a plant confers or enhances resistance of the plant to a fungus.

### FIELD OF THE INVENTION

The invention relates to genes associated with disease resistance in plants.

### BACKGROUND OF THE INVENTION

Crop diseases pose a threat to global food security. Genetic resistance can reduce crop losses in the field and can be selected using molecular markers. However, it often breaks down due to changes in pathogen virulence as experienced for the wheat yellow (stripe) rust fungus Puccinia striiformis f. sp. tritici (PST). This highlights the need to (i) identify genes that alone or in combination provide broad-spectrum resistance and (ii) increase our understanding of their molecular mechanisms.

NLRs are intracellular receptors which induce cell death upon pathogen recognition to prevent disease spread throughout the plant. Different modes of action for this gene family have been discovered over the past twenty years. The NB-ARC domain is the signature of the NLRs which in most cases carry additional Leucine Rich Repeats (LRR) at the C-terminus. Recent in silico analyses have identified NLRs with additional ‘integrated’ domains at different positions of the gene structure. These include zinc-finger BED domains (BED-NLRs) which are widespread across Angiosperm genomes and can confer resistance to bacterial blast in rice (Xa1).

In plant immunity, NLRs act as intracellular immune receptors that trigger a series of signalling steps ultimately leading to cell death upon pathogen recognition, preventing the disease spread throughout the plants. The NB-ARC domain is the hallmark signature of the NLRs which in most cases carry leucine-rich repeats (LRR) at the C-terminus. Recent in silico analyses have identified NLRs with additional ‘integrated’ domains, including zinc-finger BED domains (BED-NLRs). The BED domain from the DAYSLEEPER protein binds DNA in Arabidopsis, however whether BED domains from BED-NLRs conserved this function is unknown. BED-NLRs are widespread across Angiosperm genomes and this architecture provides resistance to bacterial blast in rice through Xa1.

The genetic relationship between Yr5 and Yr7 has been debated for almost 45 years. Both genes map to chromosome arm 2BL in hexaploid wheat (Triticum aestivum) and were hypothesized to be allelic, and closely linked with YrSP. While Yr5 confers resistance to almost all tested PST isolates worldwide, both Yr7 and YrSP have been overcome in the field following wide deployment (Table 1) and each display a different recognition specificity.

### SUMMARY OF THE INVENTION

According to an aspect of the invention is provided an isolated nucleic acid encoding a nucleotide-binding and leucine-rich repeat (NLR) polypeptide comprising a zinc-finger BED domain, wherein expression of the NLR polypeptide in a plant confers or enhances resistance of the plant to a fungus, for example wheat yellow (stripe) rust fungus Puccinia striiformisi f. sp. tritici.

Further aspects and embodiments are as defined in the appended claims and in the detailed description below.

### BRIEF DESCRIPTION OF THE FIGURES

FIG. 1. Yr5 and YrSP are allelic and paralogous to Yr7

(A) Left-Pictures of wild-type and selected EMS-derived susceptible mutant lines for Yr7, Yr5 and YrSP (Tables 2-3) inoculated with PST isolate 08/21 (Yr7), PST 80/11 (Yr5), PST 134 E16 A+ (YrSP). Candidate gene structures, with mutations shown with black bars, identified by RenSeq and their predicted effects on the translated protein are shown on the right. (B) Schematic representation of the physical and genetic interval of the Yr loci. Schematic representation of chromosome 2BL and the Yr loci is shown in grey with previously published SSR markers shown in black. Markers that we developed to confirm the genetic linkage between this locus and the candidate contigs are shown with black marks on the close-up underneath the chromosme. Yr loci mapping intervals are defined by the black horizontal lines. A more detailed genetic map is shown in FIG. 5.

FIG. 2: Yr7 and Yr5/YrSP encode integrated BED-domain resistance genes

(A) Schematic representation of the Yr7/Yr5/YrSP protein domain organisation. BED domains are highlighted in black, NB-ARC domains in dark grey, LRR motifs from NLR-Annotator in grey and manually annotated LRR motifs xxLxLxx in light grey. The sequence identity between YrSP and Yr5 is shown in light grey. Asterisks point the EMS-induced mutation positions. The plot shows the degree of amino acid conservation (50 AA rolling average) between Yr7 and Yr5 at the protein level based on the conservation diagram produced by Jalview (2.10.1) alignment viewer. Regions that correspond to the conserved domains have matching greyscale on the line. The amino acid changes between Yr5 and YrSP are annotated on the YrSP protein. (B) Five Yr5/YrSP haplotypes were identified in this study. Polymorphism are highlighted across the protein sequence with grey vertical bars for polymorphisms shared by at least two haplotypes and light grey vertical bars showing polymorphism that are unique to the corresponding haplotype. Matching greyscale across protein structures illustrate 100% sequence conservation.

FIG. 3: BED domains from BED-NLRs and non-NLR proteins are distinct

(A) Table representing the NLR counts in the syntenic region across genomes (see FIG. 6) showing their expansion in the Triticeae and the identification of BED-BED-NLRs. (B) WebLogo (http://weblogo.berkeley.edu/logo.cgi) diagram showing that the two BED domains from BED-BED-NLRs, BED-I and -II, are distant and only the highly conserved amino acids that define the BED domain (red bars) are conserved between the two types. (C) Gene structure most commonly observed for BED-NLRs and BED-BED-NLRs shows that BED is in most cases encoded by a single exon. (D) Neighbour-net analysis based on uncorrected P distances obtained from alignment of 153 BED domains (amino-acid sequences) extracted from the 108 BED-containing proteins (including 25 NLRs) from RefSeq v1.0. BED domains from NLRs located in the syntenic region defined in FIG. 6 and BED domains from Xal and ZBED from rice. BED_I and II clades are highlighted with the arc line, BED domains from the syntenic regions not related to either of these types are in dark grey. BED domains derived from non-NLR proteins are in black and BED domains from BED-NLRs outside the syntenic region are in light grey. For a better view, we removed the identifiers (see FIG. 8 for the detailed network). Seven BED domains from non-NLR proteins were close to BED domains from BED-NLRs.

FIG. 4: Identification of candidate contigs for the Yr loci using MutRenSeq

Annotated screen capture of RenSeq reads from the wild-type and mapping of EMS-derived mutants to the best candidate contig identified with MutantHunter for the three genes targeted in this study. From the top to the bottom: Vertical black lines represent the Yr loci, rectangles depict the motifs identified by NLR-Annotator (each motif is specific to a conserved NLR domain), while read coverage (grey histograms) is indicated on the left, e.g. [0-149], and the line from which the reads are derived on the right, e.g. CadWT for Cadenza wild-type. Vertical bars represent the position of SNP identified between the reads and reference assembly—dark grey shows C to T transitions and light grey G to A transitions. Black boxes highlight SNP for which the coverage was lower, but still superior to the 20x threshold used here.

The top screen capture shows the Yr7 allele annotated and before curation from the Cadenza genome assembly (Table 4). Light grey dashed lines illustrate the actual locus and the one that was formerly de novo assembled from Cadenza RenSeq data, lacking the 5′ region containing the BED domain and thus the Cad903 mutation. This locus was the only one for which all seven mutant lines carried a mutation. The middle screen capture illustrates the Yr5 locus annotated from the Lemhi-Yr5 de novo assembly. The results are similar to those described above for Yr7. The full locus was de novo assembled.

FIG. 5: Candidate contigs identified by MutRenSeq are genetically linked to the Yr loci mapping interval

Schematic representation of chromosome 2B from Chinese Spring (RefSeq v1.0) with the positions of published markers linked to the Yr loci and surrounding closely linked markers that were used to define their physical position (grey regions). Close-up of the physical locus indicating the positions of KASP markers that were used for the mapping (vertical bars Table 10). Light grey refers to Yr7, dark grey to Yr5 and grey to YrSP. The arrow points to the NLR cluster containing the best BLAST hits for Yr7 and Yr5/YrSP on RefSeq v1.0. Lines link the physical map to the corresponding genetic map for each targeted gene (see Methods). Values are expressed in centiMorgans.

FIG. 6: Expansion of BED-NLRs in the Triticeae and presence of BED-BED-NLRs whose BED domains are conserved across the syntenic region

Schematic representation of the physical loci containing Yr7 and Yr5/YrSP homologues on RefSeq v1.0 and its syntenic region based on gene content across RefSeq v1.0 subgenomes and selected grass genomes. Arrows represent loci. The syntenic region in other species was defined when three consecutive non-NLR genes had orthologues in the same order compared to chromosome 2BL outside the NLR cluster (see Methods). The syntenic region is bordered by conserved non-NLR genes (shown in light grey). Black arrows represent canonical NLRs and the different shades of grey arrows represent different types of BED-NLRs based on their BED domain and their relationship identified in FIG. 9. Grey lines link NLRs sharing more than 80% ID across more than 80% of their aligned sequence. Brown dashed lines represent the closest BED-NLR from the Triticeae to BED_I and II found in Brachypodium (Bd3 and Bd4, respectively).

FIG. 7: The Yr loci are phylogenetically related to surrounding NLRs on RefSeq v1.0 and their orthologs

Phylogenetic tree based on translated NB-ARC domains from the NLR-Annotator. Sequences were aligned using Muscle v3.8.13 with default parameters and the tree was built with the MPI version of the RAxML (v8.2.9) program. Node labels represent bootstrap values for 1,000 replicates. The tree was rooted at mid-point and visualized with Dendroscope v3.5.9. The greyscale pattern matches the one in FIG. 3 to highlight BED-NLRs with different BED domains. There was clear separation between NLRs belonging to the two different clusters but the sub-clades have less support. One explanation would be that conflicting phylogenetic signals due to events such as hybridization, horizontal gene transfer, recombination, or gene duplication and loss might have occured in the region. Split networks allow nodes that do not represent ancestral species and can thus represent such incompatible and ambiguous signals. We thus used this method in the following part of the analysis to analyse the relationship between the BED domains.

FIG. 8: Same Network as the one shown on FIG. 3 with the identifiers of all analysed proteins.

FIG. 9: BED-NLRs and BED-containing proteins are not differentially expressed in yellow rust-infected susceptible and resistant varieties

Heatmap representing the normalised read counts (Transcript Per Million, TPM) from the reanalysis of RNAseq data for all of the BED-containing proteins and BED-NLRs annotated on RefSeq v1.0. No expression is shown in white and expression levels increase from light grey to dark grey. Most BED-containing protein and BED-NLRs were not expressed at all in the analysed data. No striking pattern was observed for those that were expressed: difference were observed between varieties but these were independent of the presence of the yellow rust pathogen.

FIG. 10: Pedigrees of selected Thatcher-derived varieties and varieties known to carry Yr7 based on marker data.

The size of the circle is proportional to the prevalence of the variety in the tree. Greyscale illustrate the genotype with dark grey showing the absence of Yr7 and grey its presence. Varieties in light grey were not tested. Yr7 originated from Triticum durum cv. Iumillo and was introgressed into hexaploid wheat through Thatcher (top of the pedigree). All the varieties. Each variety positive for the Yr7 allele is related to a parent that was also positive for Yr7.

FIG. 11: Screen capture of the mapping of the Paragon RenSeq reads to the Cadenza NLR set showing that Paragon likely carries an identical version of Yr7

FIG. 12: Design of a allele-specific primer for Yr5. Yr5-Insertion PCR amplification products obtained from Yr5 donnor

Spelt and Yr5 Isogenic Lines AvocetS+Yr5 and Lemhi+Yr5, YrSP donor Spaldings Prolific and YrSP Isogenic Line AvocetS+YrSP, lines carrying alternate Yr5 alleles identified on FIG. 2 (Claire, Cadenza, Paragon), Negative controls AvocetS and Water. Molecular weight marker is the 2-log ladder from New England Biolab.

### DETAILED DESCRIPTION OF THE INVENTION

In a first aspect the invention relates to an isolated nucleic acid encoding a nucleotide-binding and leucine-rich repeat (NLR) polypeptide comprising a zinc-finger BED domain, wherein expression of the NLR polypeptide in a plant confers or enhances resistance of the plant to a fungus, for example wheat yellow (stripe) rust fungus Puccinia striiformisi f. sp. tritici.

The isolated nucleic acid may be isolated from a plant, for example an Angiosperm such as Aegilops tauschii, Brachypodium distachyon, Oryza sativa, Triticum turgidum or Triticum aestivum.

The BED domain may have an amino acid sequence corresponding to SEQ ID NO: 1 (BED-I sequence SVVWEHFTITEKDNGKPVKAVCRHCGNEFKCDTKTNGTSSMKKHLENEHS) or a variant thereof (see for example BED-I variants and consensus sequence shown in FIG. 3A) or a functional fragment thereof.

The NLR polypeptide may comprise a leucine-rich repeat (LRR) motif at or near the C-terminus.

The NLR polypeptide may have an amino acid sequence comprising SEQ ID NO: 2 (Yr5 protein) or SEQ ID NO: 3 (Yr7 protein), or a variant or functional fragment of either, including variants described herein. For example, the isolated nucleic acid may have a nucleotide sequence comprising SEQ ID NO: 4 (Yr5 gene nucleotide sequence), or its corresponding cDNA sequence, SEQ ID NO: 5 (Yr7 gene nucleotide sequence), or its corresponding cDNA sequence, or variants or functional fragments thereof, including other alleles described herein.

Alternatively, the NLR polypeptide may have an amino acid sequence comprising SEQ ID NO: 6 (YrSP protein) or a variant or functional fragment thereof, including variants described herein. For example, the isolated nucleic acid may have a nucleotide sequence comprising SEQ ID NO: 7 (YrSP nucleotide sequence) or its corresponding cDNA sequence, or variants or functional fragments thereof, including other alleles described herein.

The NLR polypeptide may comprise a further zinc-finger BED domain, for example having an amino acid sequence comprising SEQ ID NO: 8 (BED-II sequence KAWDNFDVIEEENGQPIKARCKYCPTEIKCGPKSGTAGMLNHNKICKD) or a variant therefore (see for example BED-II variants and consensus sequence shown in FIG. 3A) or a functional fragment thereof.

In another aspect the invention relates to a nucleotide-binding and leucine-rich repeat (NLR) polypeptide comprising a zinc-finger BED domain, wherein expression of the NLR polypeptide in a plant confers or enhances resistance of the plant to a fungus, for example wheat yellow (stripe) rust fungus Puccinia striiformisi f. sp. tritici. The BED domain may have an amino acid sequence comprising SEQ ID NO: 1 (BED-I) or a variant or functional fragment thereof

Further features of the NLR polypeptide per se of the invention may be defined as above and herein.

In another aspect the invention relates to a vector comprising an isolated nucleic acid of the invention. The vector may further comprising a regulatory sequence which directs expression of the nucleic acid, for example a regulatory sequence selected from a constitutive promotor, a strong promoter, an inducible promoter, a stress promotor or a tissue specific promoter.

In yet another aspect, the invention relates to a host cell comprising a nucleic acid, an NLR polypeptide or a vector of the invention. The host cell may be a bacterial cell, a yeast cell, plant cell or other cell type.

In another aspect, the invention relates to a method of producing a transgenic plant or plant cell comprising introducing and expressing a nucleic acid or a vector according to the invention into a plant or plant cell, wherein introducing and expressing the nucleic acid or vector confers or enhances resistance of the plant or plant cell to a fungal pathogen such as wheat yellow (stripe) rust fungus Puccinia striiformisi f. sp. tritici.

The transgenic plant or plant cell may have resistance or enhanced resistance to the fungal pathogen compared to a plant or plant cell of the same species lacking the nucleic acid or vector. The term “transgenic plant” refers to a plant comprising such a transgene. A “transgenic plant” includes a plant, plant part, a plant cell or seed whose genome has been altered by the stable integration of recombinant DNA. A transgenic plant includes a plant regenerated from an originally-transformed plant cell and progeny transgenic plants from later generations or crosses of a transformed plant. As a result of such genomic alteration, the transgenic plant is distinctly different from the related wild type plant. An example of a transgenic plant is a plant described herein as comprising one or more of the nucleic acids of the disclosure, for example encoding Yr5, YrSP or Yr7 proteins or a functional variant thereof, typically as transgenic elements. For example, the transgenic plant includes one or more nucleic acids of the present disclosure as transgene, inserted at loci different from the native locus of the corresponding Yr5, YrSP or Yr7 gene(s). Accordingly, it is herein disclosed a method for producing a transgenic plant, wherein the method comprises the steps of
(i) transforming a parent plant with no or low resistance to a fungus,
(ii) selecting a plant comprising said one or more nucleic acid(s) of the invention as transgene(s),
(iii) regenerating and
(iv) growing said transgenic plant.

In specific embodiments, said transgenic plant is an Angiosperm such as Aegilops tauschii, Brachypodium distachyon, Oryza sativa, Triticum turgidum or Triticum aestivum.

For transformation methods within a plant cell, one can cite methods of direct transfer of genes such as direct micro-injection into plant embryos, vacuum infiltration or electroporation, direct precipitation by means of PEG or the bombardment by gun of particules covered with the plasmidic DNA of interest.

It is preferred to transform the plant cell with a bacterial strain, in particular Agrobacterium, in particular Agrobacterium tumefaciens. In particular, it is possible to use the method described by Ishida et al. (Nature Biotechnology, 14, 745-750, 1996) for the transformation of monocotyledons.

Descriptions of Agrobacterium vector systems and methods for Agrobacterium-mediated gene transfer are provided by Moloney et al., Plant Cell Reports 8:238 (1989). See also, U.S. Pat. No. 5,591,616 issued Jan. 7, 1997.

Alternatively, direct gene transfer may be used. A generally applicable method of plant transformation is microprojectile-mediated transformation wherein DNA is carried on the surface of microprojectiles measuring 1 to 4 micron. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5:27 (1987), Sanford, J. C., Trends Biotech. 6:299 (1988), Klein et al., BioTechnology 6:559-563 (1988), Sanford, J. C., Physiol Plant 7:206 (1990), Klein et al., BioTechnology 10:268 (1992). Several target tissues can be bombarded with DNA-coated microprojectiles in order to produce transgenic plants, including, for example, callus (Type I or Type II), immature embryos, and meristematic tissue.

Following transformation of plant target tissues, expression of the selectable marker genes allows for preferential selection of transformed cells, tissues and/or plants, using regeneration and selection methods now well known in the art.

The foregoing methods for transformation would typically be used for producing a transgenic plant including the nucleic acids of the invention as transgenic element(s).

The transgenic plant could then be crossed, with another (non-transformed or transformed) inbred line, in order to produce a new transgenic line. Alternatively, a genetic trait which has been engineered into a particular line using the foregoing transformation techniques could be moved into another line using traditional backcrossing techniques that are well known in the plant breeding arts. For example, a backcrossing approach could be used to move an engineered trait from a public, non-elite inbred line into an elite inbred line, or from an inbred line containing a foreign gene in its genome into an inbred line or lines which do not contain that gene. As used herein, “crossing” can refer to a simple X by Y cross, or the process of backcrossing, depending on the context.

When the term transgenic plant is used in the context of the present disclosure, this also includes any plant including, as a transgenic element one or more of nucleic acids of the invention and wherein one or more desired traits have further been introduced through backcrossing methods, whether such trait is a naturally occurring one or a transgenic one. Backcrossing methods can be used with the present invention to improve or introduce one or more characteristic into the inbred. The term backcrossing as used herein refers to the repeated crossing of a hybrid progeny back to one of the parental plants. The parental plant which contributes the gene or the genes for the desired characteristic is termed the nonrecurrent or donor parent. This terminology refers to the fact that the nonrecurrent parent is used one time in the backcross protocol and therefore does not recur. The parental plant to which the gene or genes from the nonrecurrent parent are transferred is known as the recurrent parent as it is used for several rounds in the backcrossing protocol (Fehr et al, 1987).

In a typical backcross protocol, the recurrent parent is crossed to a second nonrecurrent parent that carries the gene or genes of interest to be transferred. The resulting progeny from this cross are then crossed again to the recurrent parent and the process is repeated until a plant is obtained wherein all the desired morphological and physiological characteristics of the recurrent parent are recovered in the converted plant in addition to the gene or genes transferred from the nonrecurrent parent. It should be noted that some, one, two, three or more, self-pollination and growing of a population might be included between two successive backcrosses.

In another aspect the invention relates to a method for producing a non-transgenic plant or plant cell having resistance or enhanced resistance to a fungal pathogen, the method comprising mutating or editing the genomic material of the plant or plant cell to comprise a nucleic acid of the invention.

An aspect of the present disclosure relates to a DNA fragment of the corresponding nucleic acids of the invention (either from naturally occurring coding sequence, or improved sequence, such as codon optimized sequence) combined with genome editing tools (such TALENs, CRISPR-Cas, Cpf1 or zing finger nuclease tools) to target the corresponding Yr5, YrSP or Yr7 genes within the wheat plant genome by insertion at any locus in the genome or by partial or total allele replacement at the corresponding locus.

In particular, the disclosure relates to a genetically modified (or engineered) plant, wherein the method comprises the steps of genetically modifying a parent plant to obtain in their genome one or more nucleic acids of the invention, preferably by genome-editing, selecting a plant comprising said one or more one or more nucleic acids as genetically engineered elements, regenerating and growing said wheat genetically engineered plant.

As used herein, the term “genetically engineered element” refers to a nucleic acid sequence present in the genome of a plant and that has been modified by mutagenesis or by genome-editing tools, preferentially by genome-editing tools. In specific embodiments, a genetically engineered element refers to a nucleic acid sequence that is not normally present in a given host genome in the genetic context in which the sequence is currently found but is incorporated in the genome of plant by use of genome-editing tools. In this respect, the sequence may be native to the host genome, but be rearranged with respect to other genetic sequences within the host genomic sequence. For example, the genetically engineered element is a Yr5, YrSP or Yr7 gene that is rearranged at a different locus as compared to a native gene. Alternatively, the sequence is a native coding sequence that has been placed under the control of heterologous regulatory sequences.

In specific embodiments, said genetically engineered plant is an Angiosperm such as Aegilops tauschii, Brachypodium distachyon, Oryza sativa, Triticum turgidum or Triticum aestivum.

The term “genetically engineered plant” or “genetically modified plant” refers to a plant comprising such genetically engineered element. A “genetically engineered plant” includes a plant, plant part, a plant cell or seed whose genome has been altered by the stable integration of recombinant DNA. As used herein, the term “genetically engineered plant” further includes a plant, plant part, a plant cell or seed whose genome has been altered by genome editing techniques. A genetically engineered plant includes a plant regenerated from an originally-engineered plant cell and progeny of genetically engineered plants from later generations or crosses of a genetically engineered plant. As a result of such genomic alteration, the genetically engineered plant is distinctly different from the related wild type plant. An example of a genetically engineered plant is a plant comprising mutated versions of Yr5, YrSP or Yr7 encoding genes. In another embodiment, the genetically engineered plant includes the nucleic acids as genetically engineered elements, inserted at loci different from the native locus of the corresponding Yr5, YrSP or Yr7 gene(s).

In specific embodiments, said genetically engineered plants do not include plants which could be obtained exclusively by means of an essentially biological process.

Said one or more genetically engineered element(s) enables the expression of polypeptides which restore or improve resistance to certain fungus, in particular resistance to a fungal pathogen such as wheat yellow (stripe) rust fungus Puccinia striiformisi f. sp. Tritici, as compared to the parent plant which do not comprise the genetically engineered element(s). Typically, said genetically engineered plant is a wheat plant, comprising, as the genetically engineered elements, a mutated version of Yr5, YrSP or Yr7 encoding gene, and said genetically engineered plant has an improved resistance to a fungal pathogen such as wheat yellow (stripe) rust fungus Puccinia striiformisi f. sp. Tritici.

Such genetically engineered plant with improved resistance may be screened by exposing a variety of genetically engineered plant having distinct mutated versions of Yr5, YrSP or Yr7 encoding gene, to a fungal pathogen such as wheat yellow (stripe) rust fungus Puccinia striiformisi f. sp. Tritici and selecting the plants which present improved resistance to said fungal pathogen.

In specific embodiments, a genetically engineered element includes an Yr5, YrSP or Yr7 encoding nucleic acid under the control of expression elements as promoter and/or terminator.

Another aspect of the disclosure relates to a genetically engineered wheat plant, which comprises the modification by point mutation, insertion or deletion of one or few nucleotides of an Yr5, YrSP or Yr7 encoding nucleic acid, as genetically engineered element, into the respectively Yr5, YrSP or Yr7 locus, by any of the genome editing tools including base-editing tool as described in WO2015089406 or by mutagenesis.

The present disclosure further includes methods for improving resistance to a funal pathogen in a plant by genome editing, comprising providing a genome editing tool capable of replacing partially or totally an Yr5, YrSP or Yr7 encoding nucleic acid or form in a plant by its corresponding mutated sequence as disclosed herein which confer improved resistance to said fungal pathogen when expressed in said plant.

Such genome editing tool includes without limitation targeted sequence modification provided by double-strand break technologies such as, but not limited to, meganucleases, ZFNs, TALENs (WO2011072246) or CRISPR CAS system (including CRISPR Cas9, WO2013181440), Cpfl or their next generations based on double-strand break technologies using engineered nucleases.

In another aspect, the invention relates to a plant or plant cell obtained or obtainable by a method of the invention. The plant or plant cell may be a crop plant or plant cell or a biofuel plant or plant cell, for example selected from maize, wheat, tobacco, oilseed rape, sorghum, soybean, potato, tomato, grape, barley, pea, bean, field bean, lettuce, cotton, sugar cane, sugar beet, broccoli or other vegetable brassicas or poplar.

In another aspect, the invention relates to a seed of the plant of the invention wherein the seed comprises a nucleic acid or an NLR polypeptide of the invention. The seed may be a wheat seed.

In another aspect, the invention relates to a method of limiting wheat yellow (stripe) rust in agricultural crop production, the method comprising planting a wheat seed as according to the invention and growing a wheat plant under conditions favourable for the growth and development of the wheat plant.

In another aspect, the invention relates to a method for identification or selection of an organism such as plant having resistance to a fungus such as wheat yellow (stripe) rust fungus Puccinia striiformisi f. sp. tritici, comprising the step of screening the organism for the presence or absence of: (1) a nucleic acid as defined according to the invention; and/or (2) an NLR polypeptide according to the invention, wherein presence of the nucleic acid or the NLR polypeptide indicates resistance.

Accordingly, it is disclosed herein the means for specifically detecting the nucleic acids of the present invention in a wheat plant.

Such means include for example a pair of primers for the specific amplification of a fragment nucleotide sequence specific of the nucleic acids of the invention in the plant genomic DNA.

As used herein, a primer encompasses any nucleic acid that is capable of priming the synthesis of a nascent nucleic acid in a template-dependent process, such as PCR. Typically, primers are oligonucleotides from 10 to 30 nucleotides, but longer sequences can be employed. Primers may be provided in double-stranded form though single-stranded form is preferred.

Alternatively, nucleic acid probe can be used for the specific detection of any one of the nucleic acids.

As used herein, a nucleic acid probe encompass any nucleic acid of at least 30 nucleotides and which can specifically hybridizes under standard stringent conditions with a defined nucleic acid. Standard stringent conditions as used herein refers to conditions for hybridization described for example in Sambrook et al 1989 which can comprise 1) immobilizing plant genomic DNA fragments or library DNA on a filter 2) prehybridizing the filter for 1 to 2 hours at 65° C. in 6× SSC 5× Denhardt's reagent, 0.5% SDS and 20 mg/ml denatured carrier DNA 3) adding the probe (labeled) 4) incubating for 16 to 24 hours 5) washing the filter once for 30 min at 68° C. in 6× SSC, 0.1% SDS 6) washing the filter three times (two times for 30 min in 30 ml and once for 10 min in 500 ml) at 68° C. in 2× SSC 0.1% SDS. The nucleic acid probe may further comprise labeling agent, such as fluorescent agents covalently attached to the nucleic acid part of the probe.

In certain embodiments, said nucleic acid probe is a fragment of at least 20 bp, 30 bp, 40 bp, 50 bp, 60 bp, 70 bp, 80 bp, 90 bp, 100 bp, 110 bp, 120 bp, 130 bp, 140 bp, 150 bp, 160 bp or the whole fragment of any of SEQ ID NO:4, 5 or 7.

References to “variant” include a genetic variation in the native, non-mutant or wild type sequence. Examples of such genetic variations include mutations selected from: substitutions, deletions, insertions and the like.

More generally, as used herein the term “polypeptide” refers to a polymer of amino acids. The term does not refer to a specific length of the polymer, so peptides, oligopeptides and proteins are included within the definition of polypeptide. The term “polypeptide” may include polypeptides with post-expression modifications, for example, glycosylations, acetylations, phosphorylations and the like. Included within the definition of “polypeptide” are, for example, polypeptides containing one or more analogs of an amino acid (including, for example, unnatural amino acids), polypeptides with substituted linkages, as well as other modifications known in the art both naturally occurring and non-naturally occurring.

As used herein, a “functional variant or homologue” is defined as a polypeptide or nucleotide with at least 50% sequence identity, for example at least 55% sequence identity, at least 60% sequence identity, at least 65% sequence identity, at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity with the reference sequence.

Sequence identity between nucleotide or amino acid sequences can be determined by comparing an alignment of the sequences. When an equivalent position in the compared sequences is occupied by the same base or amino acid, then the molecules are identical at that position. Scoring an alignment as a percentage of identity is a function of the number of identical amino acids or bases at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “Needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62. Default parameters for nucleotide sequence comparisons (“DNA Molecule” option) may be Gap Extend penalty: 0.5, Gap Open penalty: 10.0, Matrix: DNAfull.

In one aspect of the invention, the sequence comparison may be performed over the full length of the reference sequence.

Particular non-limiting embodiments of the present invention will now be described in detail.

### EXAMPLES

### Example 1

Introduction

Here we isolate and characterise three major yellow rust resistance genes (Yr7, Yr5, and YrSP) effective in hexaploid wheat (Triticum aestivum), each having a distinct and unique recognition specificity. We show that Yr5, which remains effective to a broad range of PST isolates worldwide, is allelic to YrSP and paralogous to Yr7, both of which have been overcome by multiple PST isolates. All three Yr genes belong to a complex gene cluster on chromosome 2B encoding nucleotide-binding and leucine-rich repeat proteins (NLRs) with a non-canonical N-terminal zinc-finger BED domain that is distinct from those found in non-NLR wheat proteins. We developed and tested diagnostic markers to accelerate haplotype analysis and marker-assisted selection for breeding, enabling stacking of the non-allelic Yr genes. Our results provide evidence that the BED-NLR gene architecture can provide effective field-based resistance to important fungal diseases such as wheat yellow rust.

Results and Discussion

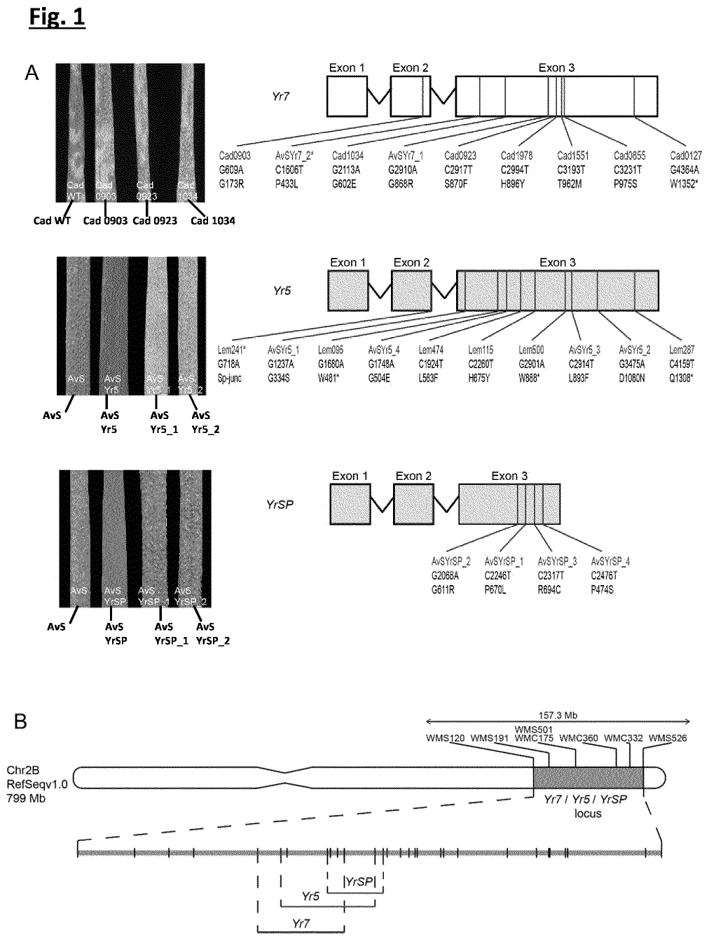

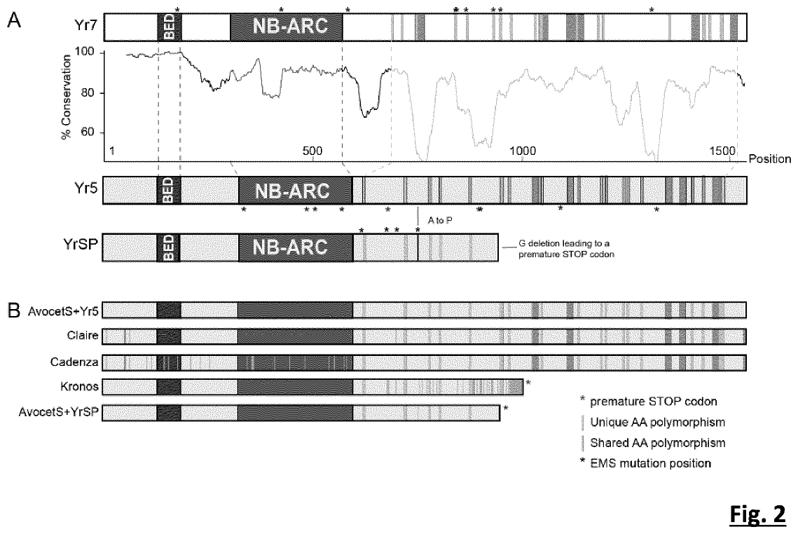

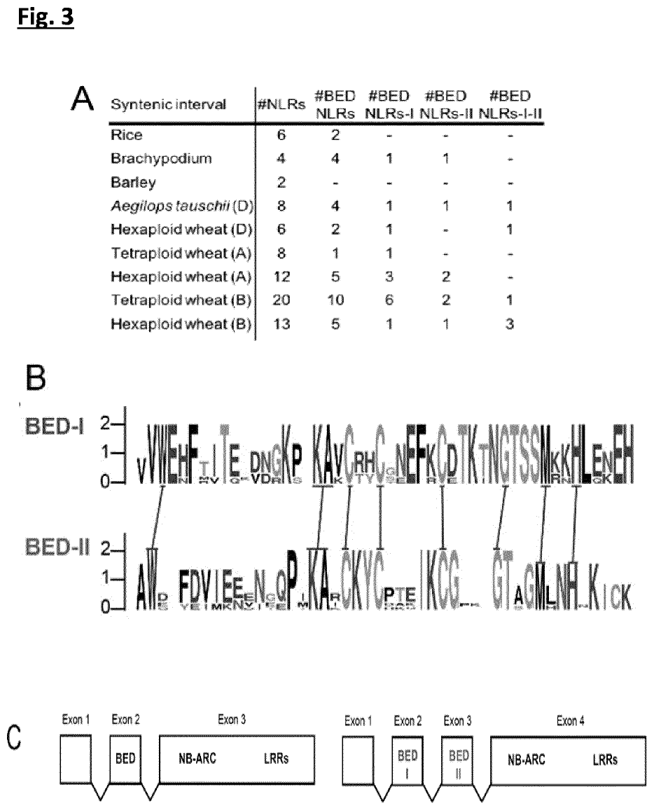

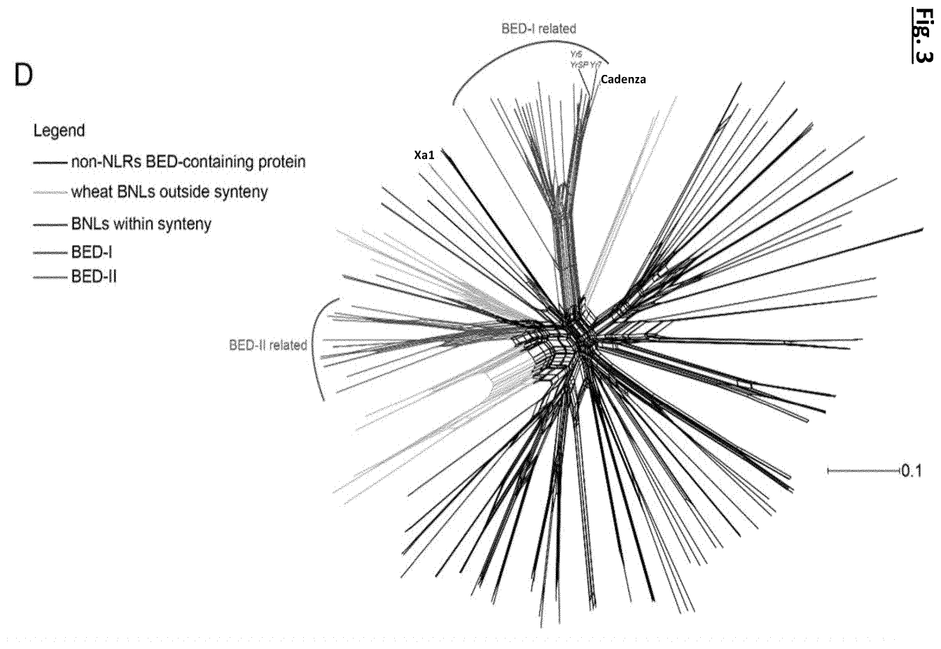

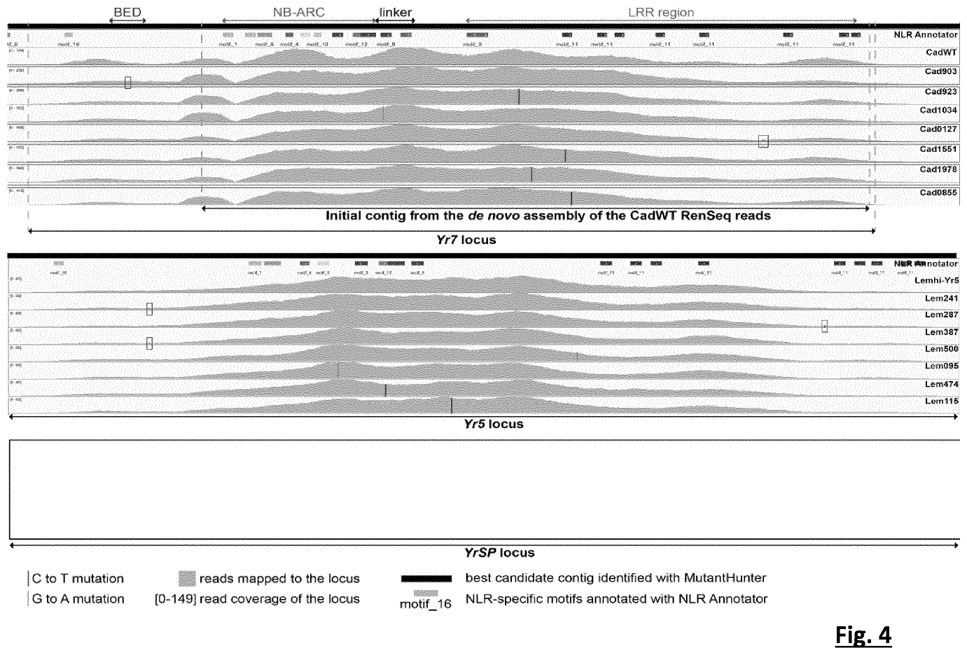

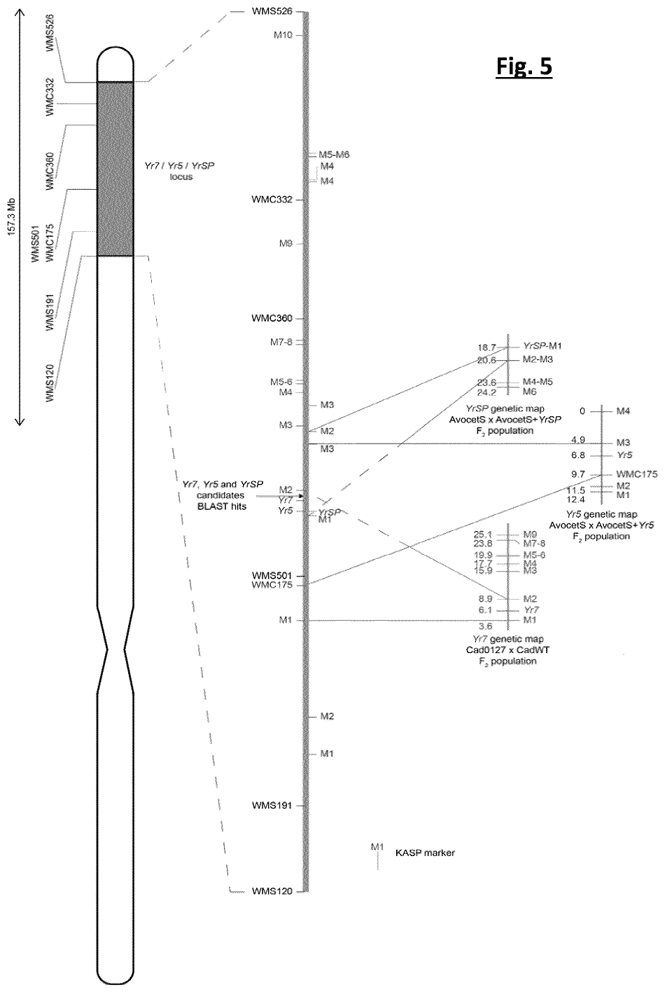

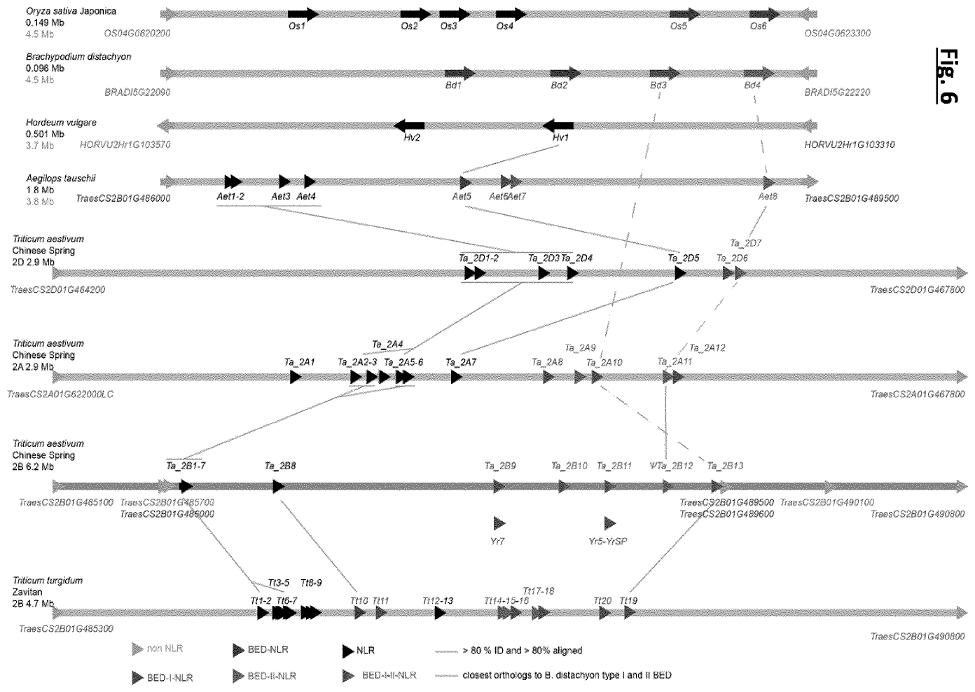

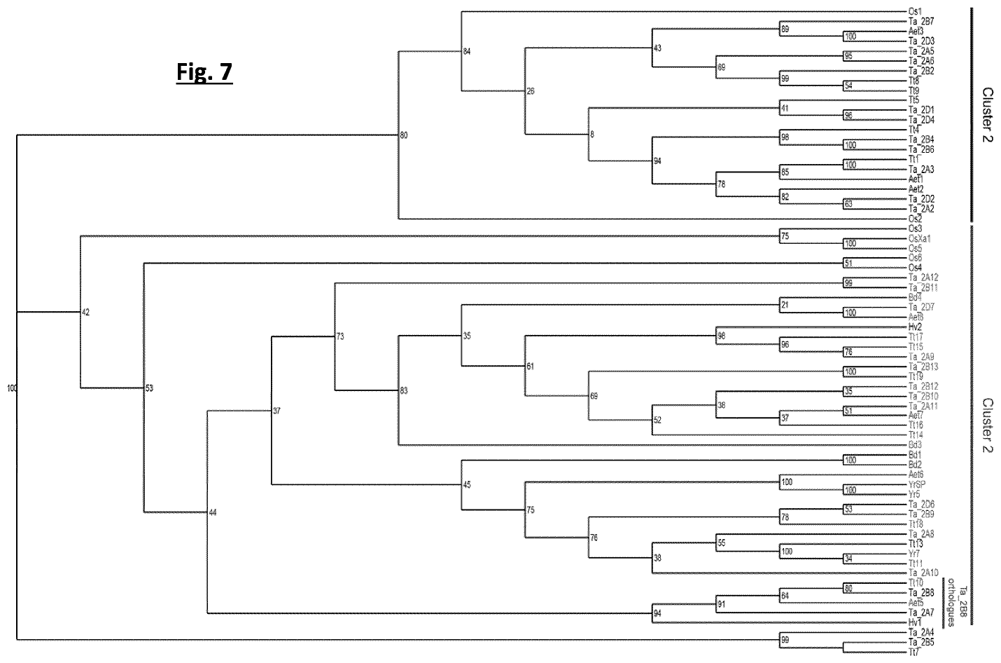

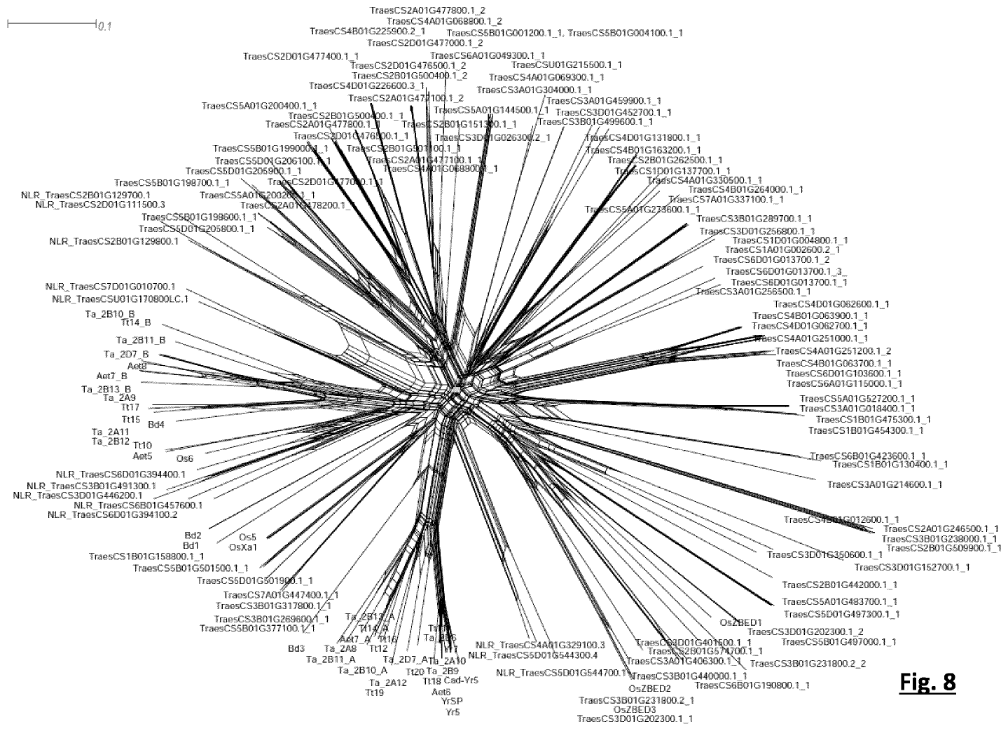

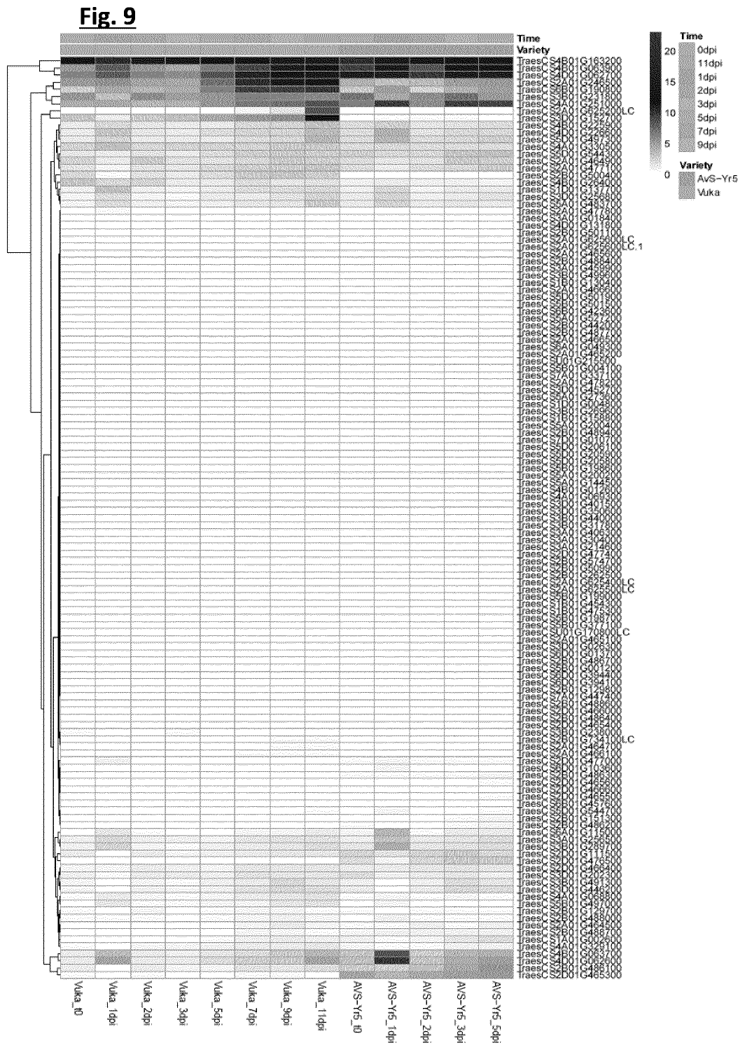

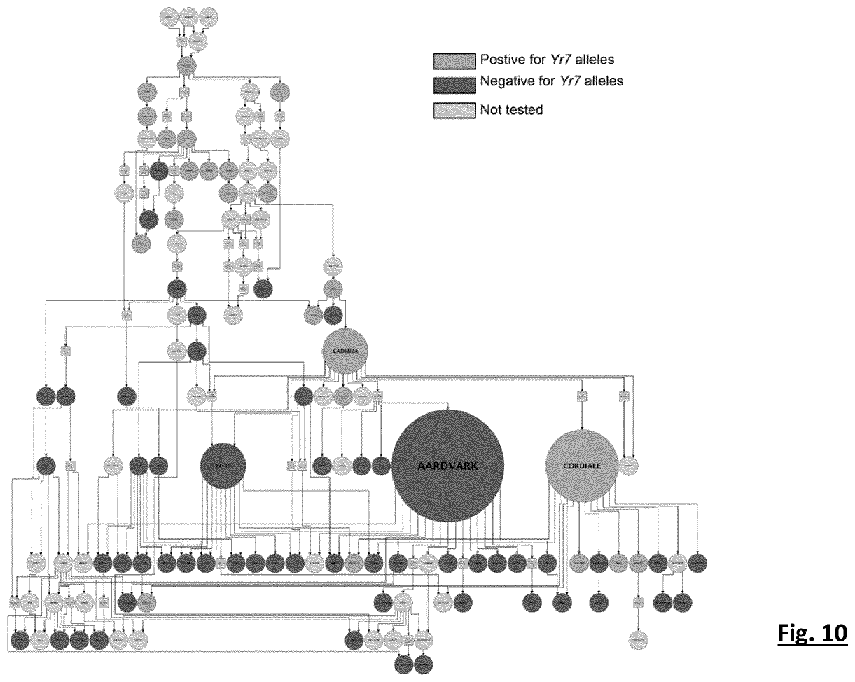

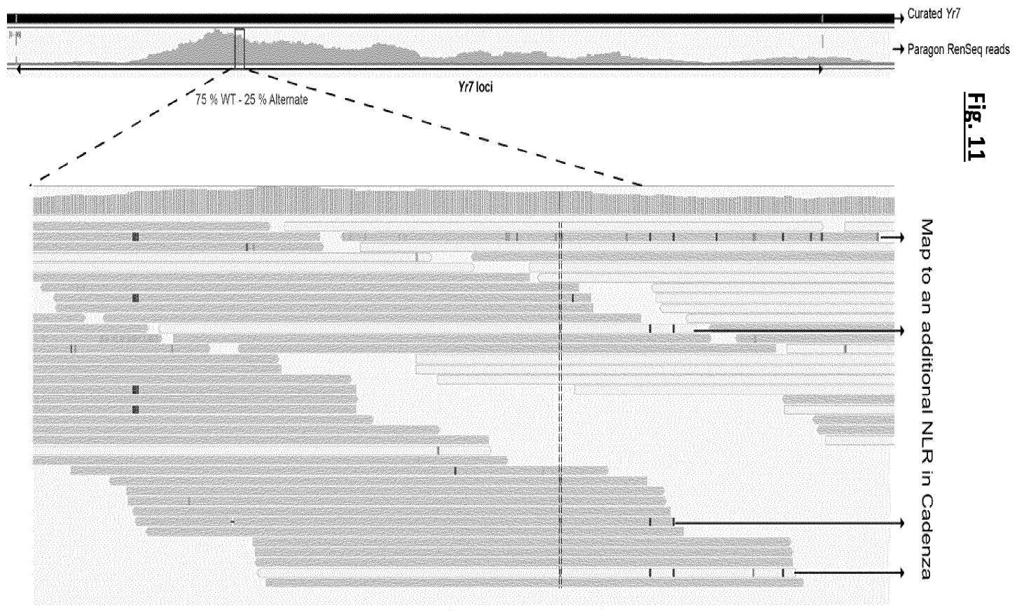

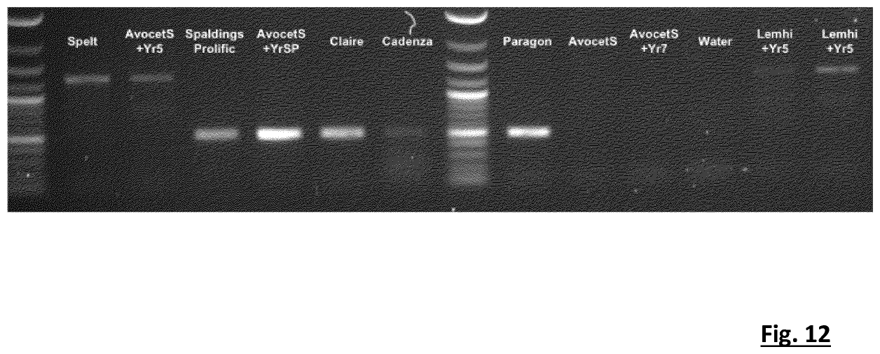

To clone the genes encoding Yr5, Yr7 and YrSp, we identified ethyl methanesulfonate-derived susceptible mutants from different genetic backgrounds carrying these genes (FIG. 1, Tables 2-3). We performed MutRenSeq (see Methods) and identified a single candidate contig for each of the three genes based on nine, ten, and four independent susceptible mutants, respectively (FIG. 1A and FIG. 4). The three candidate contigs were genetically linked to a common mapping interval previously identified for the three Yr loci. Additionally, their closest homologs in the Chinese Spring wheat genome sequence (RefSeq, https://wheat-urgi.versailles.inra.fr/Seq-Repository/Assemblies) lie between the flanking markers defining the genetic mapping interval (FIG. 1B and 5). Within each contig we predicted a single open reading frame based on RNA-Seq data. All three predicted Yr genes displayed similar exon-intron structures (FIG. 1A), although YrSP was truncated in exon 3 due to a single bp deletion that results in a premature termination codon. The DNA sequences of Yr7 and Yr5 were 77.9% identical across the complete gene, whereas YrSP was a truncated version of Yr5, sharing 99.8% identity in the common sequence. This suggests that Yr5 and YrSP are encoded by alleles of the same gene, but are paralogous to Yr7. The 23 mutations identified by MutRenSeq were confirmed by Sanger sequencing and lead to either an amino acid substitution or a truncation allele (splice junction or termination codon)(FIG. 1A, Table 3). Taken together, the mutant and genetic analyses demonstrate that these two genes encode for Yr7 and Yr5/YrSP.

The Yr7, Yr5 and YrSP proteins contain a zinc-finger BED domain at the N-terminus, followed by the canonical NB-ARC domain. Only Yr7 and Yr5 proteins encode multiple LRR motifs at the C-terminus. YrSP lost most of the LRR region due to the presence of a premature termination codon in exon 3 (FIG. 2A). However, YrSP still confers functional resistance to PST, although having a different recognition specificity to Yr5. Yr7 and Yr5/YrSP are highly conserved in the N-terminus, with a single amino-acid change in the BED domain, but this high degree of conservation is eroded after the BED domain (FIG. 2A). The BED domain is required for Yr7-mediated resistance, as a single amino acid change in the mutant line Cad0903 led to a susceptible reaction (FIG. 1A). However, recognition specificity is not solely governed by the BED domain, as the Yr5 and YrSp alleles have identical BED domain sequences and yet confer resistance to different PST isolates.

We examined the allelic variation in Yr7 and Yr5/YrSP across eight sequenced tetraploid and hexaploid wheat genomes (Table 4). Yr7 was originally derived from tetraploid durum wheat (T. turgidum ssp. durum) cultivar Iumillo and was spread globally through hexaploid cultivar Thatcher. We identified Yr7 only in Cadenza (Thatcher-derived) and Paragon, which is identical by descent to Cadenza in this interval (Table 5a and b). None of the three sequenced tetraploid accessions (Svevo, Kronos, Zavitan) carried Yr7.

For Yr5/YrSP, we identified three additional alleles in the sequenced hexaploid wheat cultivars (Table 5a and b). Claire encodes a complete NLR with only six amino-acid changes situated outside the three conserved domains (BED, NB-ARC and LRRs) and six polymorphisms in the C-terminus compared to Yr5. Robigus, Paragon and Cadenza also encode a full length NLR which shares common polymorphisms with Claire in addition to 19 amino acid substitutions across the BED and NB-ARC domains. Tetraploid Kronos and Svevo encode a fifth Yr5/YrSP protein with a truncation in the LRR region distinct from YrSP, in addition to multiple amino acid substitutions in the C-terminus. This truncated tetraploid allele is reminiscent of YrSP and is expressed in Kronos (see Methods). None of these varieties exhibit a typical Yr5 resistance response, suggesting that these amino acid changes/truncations may alter recognition specificity or protein function.

We designed diagnostic markers for Yr5 and Yr7 to facilitate their detection and use in breeding. We confirmed their presence in the donor cultvars Thatcher and Lee (Yr7), Spaldings Prolilic (YrSP), and spelt wheat cv. Album (Yr5) (Tables 10-12; FIGS. 10 and 12). To further define their specificity, we tested the markers in a collection of global landraces and European varieties released over the past one hundred years. Yr5 was only present in spelt cv. Album, AvocetS-Yr5, and Lemhi-Yr5 and was not detected in any other line (Table 19), consistent with the fact that Yr5 has not yet been deployed within European breeding programmes. Yr7 on the otherhand was more prevalent in the germplasm tested and we could track its presence across pedigrees including Cadenza derived cultivars (see Tables 11-15; FIG. 10).

We defined the Yr7/Yr5/YrSP syntenic interval across the wheat genomes and related grass species Aegilops tauschii (D genome progenitor), Hordeum vulgare (barley), Brachypodium distachyon and Oryza sativa (rice) (FIG. 6). We identified both canonical NLRs as well as integrated BED-NLRs across all genomes and species, except for barley, which contained only canonical NLRs across the syntenic region. The phylogenetic relationship based on the NB-ARC domain suggests a common evolutionary origin of these integrated domain NLR proteins before the wheat-rice divergence (50 Mya) and an expansion in the number of NLRs in the A and B genomes of polyploid wheat species (FIG. 7, FIG. 3A). Within the interval we also identified several genes in the A, B and D genomes that encode two consecutive in-frame BED domains in frame (herein named BED_I and BED_II) followed by the canonical NLR. These double BED domain genes had each BED domain fully encoded within a single exon (exons 2 and 3) and in most cases had a four-exon structure (FIG. 3B). This is consistent with the three exon structure of single BED domain genes, such as Yr7 and Yr5/YrSP (BED_I type encoded on exon 2). Very few amino acids were conserved between BED_I and II (FIG. 3B). To our knowledge this is the first report of the double BED domain NLR protein structure to date. The biological function of this molecular innovation remains to be determined, although our data show that the single BED_I structure can confer PST resistance and is required for Yr7-mediated resistance.

Among other mechanisms, integrated domains of NLRs are hypothesised to act as decoys for their intended effector targets. This would suggest that the integrated domain might be sequence-related to the host protein targeted by the effector. To identify potential host targets of AvrYr7, AvrYr5 and AvrYrSP, we retrieved all BED-domain proteins (108) from the wheat genome, including 25 BED-NLRs, and additional BED-NLRs located in the syntenic intervals (Table 6). We also retrieved the rice Xal and ZBED proteins, the latter being hypothesized to act in rice resistance against Magnaporthe. oryzae. We used the split network method implemented in Splitstree4 to represent the relationships between these BED domains (FIG. 3C, FIG. 8). We found a major split in the network, with almost all wheat non-NLR BED proteins (76 of 83) clustering together at one end and the BED-NLRs proteins of wheat and other analysed species at the other end. This clear separation is consistent with the hypothesis that integrated domains might have evolved to strengthen the interaction with the effector after integration. Among BED-NLRs, BED_I and BED_II constitute two major clades that are comprised solely of genes from within the Yr7/Yr5/YrSP syntenic region. The seven non-NLR BED domain wheat proteins that clustered with BED-NLRs are most closely related to the Brachypodium and rice proteins and were not expressed in RNA-Seq data from a Yr5-mediated resistance vs susceptible time-course (FIG. 9, Table 12). Similarly, no BED-containing protein was differentially expressed during this infection time-course. This is consistent with the prediction that effectors alter their targets' activity at the protein level. However, we cannot disprove that these closely related BED-containing proteins are involved in BED-NLRs-mediated resistance.

BED-NLRs are frequent in Triticeae and occur in other monocot and dicot tribes. However, only a single BED-NLR gene, Xa1, had been previously shown to confer resistance to plant pathogens. In the present study, we show that the distinct Yr5, YrSP, and Yr7 resistance specificities belong to a complex NLR cluster on chromosome 2B and are encoded by two BED-NLRs genes which are paralogous. We report an allelic series for the Yr5/YrSP gene with five independent alleles including three full-length BED-NLRs (including Yr5) and two truncated versions (including YrSP). This wider allelic series could be of functional significance as previously shown for the Mla and Pm3 loci that confer resistance to Blumeria graminis in barley and wheat, respectively, and the flax L locus conferring resistance to Melampsora lini. Overall, our results add strong evidence for the importance of the BED-NLR architecture in plant-pathogen interactions. The paralogous and allelic relationship of these three distinct Yr loci will inform future hypothesis-driven engineering of novel recognition specificities.

Methods

1.1. MutRenSeq

Mutant Identification

Table 2 summarises plant materials and PST isolates used for each Yr gene. We used an ethyl methanesulfonate (EMS)-mutagenised population in cultivar Cadenza to identify mutants in Yr7, whereas EMS-populations in the corresponding AvocetS-Yr near isogenic line (NIL) were used to identify Yr5 and YrSP mutants. For Yr7, we inoculated M₃ plants from the Cadenza EMS population with PST isolate 08/21 which is virulent to Yr1, Yr2, Yr3, Yr4, Yr6, Yr9, Yr17, Yr27, Yr32, YrRob, and YrSol. We hypothesised that susceptible mutants would carry mutations in Yr7. Plants were grown in 192-well trays in a confined glasshouse with no supplementary lights or heat. Inoculations were performed at the one leaf stage (Z11) with a talc-urediniospore mixture. Trays were kept in darkness at 10° C. and 100% humidity for 24 hours. Infection types (IT) were recorded 21 days post-inoculation following the Grassner and Straib scale. Identified susceptible lines were progeny tested to confirm the reliability of the phenotype and DNA from M₄ plants was used for RenSeq (see section below). Similar methods were used for AvocetS+Yr7, AvocetS+Yr5 and AvocetS+YrSp EMS-mutagenised populations with the following exceptions: PST pathotypes 108 E141 A+ (University of Sydney Plant Breeding Institute Culture no. 420),150 E16 A+(Culture no. 598) and 134 E16 A+(Culture no. 572) were used, respectively. EMS-derived susceptible mutants in Lehmi+Yr5 were previously identified and DNA from M₅ plants was used for RenSeq.

DNA Preparation and Resistance Gene Enrichment and Sequencing (RenSeq)

We extracted total genomic DNA from young leaf tissue using the large-scale DNA extraction protocol from the McCouch Rice Lab (https://ricelab.plbr.cornell.edu/dna_extraction). Total genomic DNA of all Avocet mutants and wild-types were extracted following a previously described method. We checked DNA quality and quantity on a 0.8% agarose gel and with a NanoDrop spectrophotometer (Thermo Scientific). Arbor Biosciences (Ann Arbor, Mich., USA) performed the targeted enrichment of NLRs according to the MYbaits protocol and using an improved version of the Triticeae bait library. Library construction was performed using the TruSeq RNA protocol v2 (Illumina 15026495). Libraries were pooled—one pool of samples for Cadenza mutants and one of eight samples for the Lemhi+Yr5 parent and Lemhi+Yr5 mutants. AvocetS+Yr5 and AvocetS+YrSP wild type together with their respective mutants were also processed according to the aforementioned MYbaits protocol and the same bait library were used. All enriched libraries were sequenced on a HiSeq 2500 (Illumina) in High Output mode using 250 bp paired end reads and SBS chemistry. We used Cadenza wild-type data previously generated on an Illumina MiSeq instrument.

In addition to the mutants, we also generated RenSeq data for Kronos and Paragon to confirm the presence of the Yr5 allele in Kronos and the Yr7 gene in Paragon

Details of all the lines sequenced is available in Table 3 and sequencing details are in Table 8.

1.2. MutantHunter Pipeline

We adapted the pipeline from https://github.com/steuernb/MutantHunter/to identify candidate contigs for the targeted Yr genes. First, we trimmed the RenSeq-derived reads with trimmomatic and the following parameters: ILLUMINACLIP:TruSeq2-PE.fa:2:30:10 LEADING:30 TRAILING:30 SLIDINGWINDOW:10:20 MINLEN:50 (v0.33). We made de novo assemblies of wild-type plant trimmed reads with the CLC assembly cell and default parameters apart from the word size (-w) parameter that we set to 64 (v5.0, http://www.cicbio.com/products/c1c-assembly-cell!, Table 9). We then followed the MutantHunter pipeline detailed at https://github.com/steuernb/MutantHunter/. For Cadenza mutants, we used the following MutantHunter program parameters to identify candidate contigs: -c 20-n 6-z 1000, that translates into SNPs with at least 20x coverage, six susceptible mutants must have a mutation in the contig to report it as candidate, and small deletions were filtered out by setting the number of coherent positions with zero coverage to call a deletion mutant at 1000. The -n parameter was modified accordingly in subsequent runs with the Lemhi+Yr5 (−n 6). For identifying Yr5 and YrSP contigs from Avocet mutants, we followed the aforementioned MutantHunter with all default parameters, except the use of CLC Genomics Workbench (v10) for reads QC and trimming, as well as de novo assemblies of Avocet wild-type and mapping all reads against de novo assembly of wild-type. The MutantHunter programme parameters were set all as default except for −z was set as 100. The parameter −n was set for two as the first run and then three as the second run. Regarding Yr5, two mutants were sibling lines as they carried the same mutation at identical positions (FIG. 4, Table 3).

For Yr7 we identified a single contig with six mutations, however we did not identify mutations in line Cad0903. Upon examination of the Yr7 candidate contig we predicted that the 5′ region was likely missing (FIG. 4). We thus annotated potential NLRs in the Cadenza genome assembly available from the Earlham Institute (Table 4, http://opendata.earlham.ac.uk/Triticum aestivum/EI/v1.1) with the NLR-Annotator program with standard parameters (https://github.com/steuernb/NLR-Annotator). We identified an annotated NLR in the Cadenza genome with 100% sequence identity to the Yr7 candidate contig, but that extended beyond the available sequence. We therefore replaced the previous candidate contig with the extended Cadenza sequence (100% sequence identity) and mapped the RenSeq reads from the Cadenza wild-type and mutants the same way as above. This confirmed the candidate for Yr7 as we retrieved the missing 5′ region including the BED domain, and confirmed a mutation in the outstanding mutant line Cad0903 (FIG. 4).

The Triticeae bait library does not include integrated domains in its design so they are prone to be missed, especially when located at the ends of an NLR. Sequencing technology could also have accounted for this: MiSeq was used for Cadenza wild-type whereas HiSeq was chosen for Lemhi-Yr5 and we did not observe the missing 5′ region in the latter, although coverage was lower than the regions encoding for canonical domains.

In summary, we sequenced nine, ten and four mutants for Yr7, Yr5 and YrSP and identified a single contig for each target gene which accounted for all the mutations.

1.3. Candidate Contig Confirmation and Gene Annotation

We sequenced the three candidate contigs to confirm the EMS-derived mutations using primers documented in Table 10. We first PCR-amplified the full locus from the same DNA preparations as the ones submitted for RenSeq with the Phusion® High-Fidelity DNA Polymerase (New England Biolabs) following the provider's protocol (https://www.neb.com/protocols/0001/01/01/per-protocol-m0530). We then carried out nested PCR on the obtained product to generate overlapping 600-1,000 bp amplicons that were purified using the MiniElute kit (Qiagen). The purified PCR products were sequenced by GATC following the LightRun protocol (https://www.gatc-biotech.com/shop/en/lightrun-tube-barcode.html). Resulting sequences were aligned to the wild-type contig using ClustalOmega (https://www.ebi.ac.uk/Tools/msa/clustalo/). This allowed us to curate the Yr7 locus in the Cadenza assembly that has two ‘N’ in its sequence, corresponding to a 39 bp insertion and a 129 bp deletion, and confirm the presence of the mutations in each mutant line.

We used HISATt2 (v2.1) to map RNA-Seq reads available from Cadenza and AvocetS-Yr5 onto the RenSeq de novo assemblies with curated loci to define the gene structure of the genes. We used the following parameters: —no-mixed—no-discordant to map read in pairs only. We used the—novel-splicesite-outfile to predict splicing sites which we manually checked with the genome visualisation tool IGV (v2.3.79). Predicted CDS were then translated using the ExPASy online tool (https://web.expasy.org/translate/). This allowed us to predict the effect of the mutations for each candidate gene (FIG. 1A). The long-range primers for both Yr7 and Yr5 loci were then used on the corresponding susceptible Avocet NIL mutants to determine whether the genes were present and carried mutations in that background (FIG. 1A).

1.4. Genetic Linkage Experiments

We generated a set of F₂ populations to genetically map the candidate contigs (Table 2). For Yr7 we developed an F₂ population based a cross between the susceptible mutant line Cad0127 to the Cadenza wild type control (population size 139 individuals). For Yr5 and YrSp we developed F₂ populations between AvocetS and the NILs carrying the corresponding Yr gene (94 individuals for YrSp and 376 for Yr5). We extracted DNA from leaf tissue at the seedling stage (Z11). Rqtl package was used to produce the genetic map based on a general likelihood ratio test and genetic distances were calculated from recombination frequencies (v1.41-6).

We used markers linked to Yr7, Yr5, YrSP (WMS526, WMS501 and WMC175, WMC332, respectively) in addition to closely linked markers WMS120, WMS191 and WMC360 (based on the GrainGenes database https://wheat.pw.usda.gov/GG3/) to define the physical region on RefSeq v1.0. Two different approaches were used for genetic mapping depending on the material. For Yr7, we used the public data for Cad0127 (www.wheat-tilling.com) to identify nine mutations located within the Yr7 physical interval based on BLAST analysis against RefSeq v1.0. We used KASP primers when available and manually designed additional ones including an assay targeting the Cad0127 mutation in the Yr7 candidate contig (Table 10). We genotyped the Cad0127 F₂ populations using these ten KASP assays and confirmed genetic linkage between the Cad0127 Yr7 candidate mutation and the nine mutations across the physical interval (FIG. 5).

For Yr5 and YrSP, we first aligned the candidate contigs to the best BLAST hit in an AvocetS RenSeq de novo assembly. We then designed KASP primers targeting polymorphism between these sequences and used them to genotype the corresponding F₂ population. We also used markers polymorphic between parental lines to determine the presence of Yr5/YrSP in breeding material (Table 10). For both candidate contigs we confirmed genetic linkage with the genetic intervals for these Yr genes (FIG. 5).

1.5. Yr7 Gene-Specific Markers

We aligned the Yr7 sequence with the best BLAST hits in the genomes listed on Table 2 and designed KASP primers targeting polymorphisms that were Yr7-specific. Three markers were retained after testing on a selected panel of Cadenza-derivatives and varieties that were positive for Yr7 markers in the literature, including the Yr7 reference cultivar Lee (Table 10 for the primers, Tables 11 and 12 for the results). The panel of Cadenza-derivatives was phenotyped with three PST isolates: PST 08/21 (Yr7-avirulent), PST 15/151 (Yr7-avirulent—virulent to Yr1,2,3,4,6,9,17,25,32,Rendezvous, Sp, Robigus, Solstice) and PST 14/106 (Yr7-virulent, virulent to Yr1,2,3,4,6,7,9,17,25,32, Sp, Robigus, Solstice, Warrior, Ambition, Cadenza, KWS Sterling, Apache) to determine whether Yr7-positive varieties as determined by the three KASP markers displayed a consistent specificity. Pathology assays were performed as for the screening of the Cadenza mutant population. We retrieved pedigree information for the analysed varieties from the Genetic Resources Information System for Wheat and Triticale database (GRIS, www.wheatpedigree.net) and used the Helium software (v1.17) to illustrate the breeding history of Yr7 in the UK (FIG. 10).

We used the three Yr7 KASP markers to genotype (i) varieties from the AHDB Wheat Recommended List from 2005-2018 (https://cereals.andb.org.uk/varieties/andb-recommended-lists.aspx); (ii) the Gediflux collection that gathers European bread wheat varieties released between 1920 and 2010 and (iii) the core Watkins collection, which represents a global set of wheat landraces collected in the 1930s. Results are reported in Tables 13-15.

Yr5 Gene-Specific Markers

We identified a 774 bp insertion in the Yr5 allele 29 bp upstream the STOP codon with respect to the Cadenza and Claire alleles. gDNA from YrSP confirmed that the insertion was specific to Yr5.

We used this polymorphism to design primers flanking the insertion and tested them on a subset of the collections mentioned above. We included DNA from Triticum aestivum ssp. spelta var. Album (Yr5 donor) and Spaldings Prolific (YrSP donor) to assess their amplification profiles. PCR amplification was conducted using a touchdown programme with the first 10 cycles from 67° C. to 62° C. (−0.5° C. per cycle) and the remaining 25 cycles at 62° C. This allowed to increase the specificity of the reaction. We observed three different profiles on the tested varieties (i)1,281 bp amplicon in Yr5 positive cultivars, (ii) 507 bp amplicon in the alternate Yr5 alleles carriers including YrSP, Cadenza and Claire and (iii) no amplification in other varieties. We sequenced the different amplicons and confirmed the insertion in Yr5 compared to the alternate alleles. The lack of amplicon in some varieties might respresent the absence of the loci in the tested varieties.

1.6. In Silico Allele Mining for Yr7 and Yr5

We used the Yr7 and Yr5 sequences to retrieve the best BLAST hits in the T. aestivum and T. turgdium wheat genomes listed in Table 4. The best Yr5 hits shared between 93.6 and 99.3% sequence identity, which was comparable to what was observed for alleles derived from the barley Pm3 (>97% identity) and flax L (>90% identity) genes. Yr7 was identified only in Paragon and Cadenza (Table 5a and b; see FIG. 11 for curation of the Paragon sequence).

1.7. Analysis of the Yr7 and Yr5/YrSP Cluster on RefSeq v1.0

Definition of Syntenic Regions Across Grass Genomes

We used NLR-Annotator to identify putative NLR loci on RefSeq v1.0 chromosome 2B and identified the best BLAST hits to Yr7 and Yr5 on RefSeq v1.0. Additional BED-NLRs and canonical NLRs were annotated in close physical proximity to these best BLAST hits. Therefore, to better define the NLR cluster we selected ten non-NLR genes located both distal and proximal to the region and identified orthologs in barley, Brachypodium and rice in EnsemblPlants (https://plants.ensembl.org/). We used different % ID cutoffs for each species (>92% for barley, >84% for Brachypodium and >76% for rice) and determined the syntenic region when at least three consecutive orthologues were found. A similar approach was conducted for Triticum ssp and Ae. tauschii (Table 16).

1.8. Definition of the NLR Content of the Syntenic Region

We extracted the previously defined syntenic region from the grass genomes listed in Table 4 and annotated NLR loci with NLR-Annotator. We maintained previously defined gene models where possible, but also defined new gene models which were further analysed through a BLASTx analysis to confirm the NLR domains (Tables 16-18). The presence of BED domains in these NLRs was also confirmed by CD-Search (https://www.ncbi.nlm.nih.gov/Structure/cdd/wrpsb.cgi). All NLR and BED-NLR encoding sequences were taken forward for reciprocal BLAST analyses across all genomes to identify orthologous relationships. NLRs are known to be more variable than other gene classes so we used a lower threshold to define orthologues (80% ID across 80% of the alignment for the Triticeae (brown lines on FIG. 6)).

1.9. Phylogenetic and Neighbour Network Analyses

We aligned the translated NB-ARC domains from the NLR-Annotator output with MUSCLE and standard parameters (v.3.8.31). We verified and manually curated the alignment with Jalview (v2.10.1). We built a Maximum Likelihood tree with the RAxML program and the following parameters: raxmlHPC -f a -x 12345-p 12345-N 1000-m PROTCATJTT -s <input_alignmentlasta>(MPI version v8.2.10). The best scoring tree with associated bootstrap values was visualised with Dendroscope (v3.5.9).

We used the Neighbour-net method implemented in SplitsTree4 to analyse relationships between BED domains from NLR and non-NLR proteins (v4.16). We first retrieved all BED-containing proteins from RefSeq v1.0 as follows: we used hmmer (v3.1b2, http://hmmer.org/) to identify conserved domain in protein sequences from RefSeq v1.0. We applied a cut-off of 0.01 on i-evalue to filter-off any irrelevant identified domains. We separated the set between NLR and non-NLRs based on the presence of the NB-ARC and sequence homology for single BED proteins. BED domains were extracted from the corresponding protein sequences based on the hmmer output and were verified on the CD-search database. Alignments of the BED domains were performed the same way as for NB-ARC domains and were used to generate a neighbour network in SplitsTree4 based on the uncorrected P distance matrix.

1.10. Transcriptome Analysis

Kronos Analysis

We reanalysed RNA-Seq from cultivar Kronos to determine whether the Kronos Yr5 alelle was expressed. We followed the same strategy as that described to define the Yr7 and Yr5 gene structure (candidate contig confirmation and gene annotation section). We generated a de novo assembly of the Kronos NLR repertoire from Kronos RenSeq data and used it as a reference to map read data of one replicate from the wild-type Kronos heading stage. Read depths up to 30× were present in the Yr5 allele which allowed to confirm its expression. Likewise, the RNA-Seq reads confirmed the gene structure, which is similar to YrSP, and the premature termination codon in Kronos Yr5.

Re-Analysis of RNAseq Data in Dobon et al., 2016

Briefly, two RNA-Seq time-courses were used based on samples taken from leaves at 0, 1, 2, 3, 5, 7, 9 and 11 days post-inoculation for the susceptible cultivar Vuka and 0, 1, 2, 3 and 5 days post inoculation for the resistant AvocetS-Yr5. We used normalised read counts (Transcript Per Million, TPM) from Ramirez-Gonzalez et al. (2018; under review) to produce the heatmap shown in FIG. 11 with the pheatmap R package (v1.0.8). Transcripts were clustered according to expression profile defined by a Euclidean distance matrix and hierarchical clustering. Transcripts were considered expressed if their average TPM was 0.5 TPM in at least one time point. We used the DESeq2 R package (v1.18.1) to conduct a differential expression analysis. We performed two comparisons: (1) we used a likelihood ratio test to compare the full model ˜Variety +Time +Variety:Time to the reduced model ˜Variety +Time to identify genes that were differentially expressed between the two varieties at a given time point after time 0 (workflow: https://www.bioconductor.org/help/workflows/rnaseqGene/); (2) Investigation of both time courses in Vuka and AvocetS-Yr5 independently to generate all of the comparisons between time 0 and a given time point, following the standard DESeq2 pipeline. Differentially expressed genes were considered to be those with an adjusted p-value <0.05 and a log2 fold change of 2 or higher.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

TABLE 1
Summary of the data from NIABTAG Seedstats journal (NIABTAG Network) and UK
Cereal Pathogen Virulence Survey (http://www.niab.com/pages/id/316/UKCPVS) that were used
Table 1: Cereal Weights Certified-NIAB TAG for selected Yr7 varieties from 1990 to
2016 with virYr7 prevalence among UK yellow rust isolates (UKCPVS)

| Cultivated Yr7 varieties | | 1990 | 1991 | 1992 | 1993 | 1994 | 1995 | 1996 | 1997 | 1998 | 1999 |
|---|---|---|---|---|---|---|---|---|---|---|---|
| | % virYr7_isolat [symbol] | 9 | 19 | 7 | 8 | 4 | 0 | 3 | 7 | 4 | 10 |
| CORDIALE | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| CUBANITA | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| GRAFTON | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| SKYFALL | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| RUSKIN | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| BROCK | total tons | 3666.8 | 934.4 | 389 | 127.3 | 80.7 | 0 | 0 | 0 | 0 | 0 |
| | % | 1.3 | 0.3 | 0.2 | 0.0 | 0.0 | 0.0 | 0.0 | 0.0 | 0.0 | 0.0 |
| CADENZA | total tons | 0 | 0 | 337.5 | 8011.3 | 8412.3 | 3345.3 | 1146.4 | 634.5 | 744.8 | 223.5 |
| | % | 0.0 | 0.0 | 0.1 | 3.1 | 3.4 | 1.3 | 0.4 | 0.3 | 0.3 | 0.1 |
| CAMP | total tons | 1450.35 | 462.7 | 217 | 215.9 | 81.7 | 56.8 | 31.2 | 0 | 0 | 0 |
| REMY | % | 0.5 | 0.2 | 0.1 | 0.1 | 0.0 | 0.0 | 0.0 | 0.0 | 0.0 | 0.0 |
| PROPHET | total tons | 0 | 0 | 0 | 124.2 | 29 | 0 | 0 | 0 | 0 | 0 |
| | % | 0.0 | 0.0 | 0.0 | 0.0 | 0.0 | 0.0 | 0.0 | 0.0 | 0.0 | 0.0 |
| SOLEIL | total tons | 65 | 47.7 | 152.5 | 71.5 | 60 | 15 | 0 | 0 | 0 | 0 |
| | % | 0.0 | 0.0 | 0.1 | 0.0 | 0.0 | 0.0 | 0.0 | 0.0 | 0.0 | 0.0 |
| SPARK | total tons | 0 | 0 | 2402.7 | 3734.2 | 3240.6 | 2737.9 | 2369.6 | 1627.1 | 1036.9 | 809.3 |
| | % | 0.0 | 0.0 | 1.0 | 1.5 | 1.3 | 1.0 | 0.9 | 0.7 | 0.5 | 0.4 |
| TARA | total tons | 392.3 | 3018.7 | 748 | 85.7 | 49.6 | 0 | 0 | 0 | 0 | 0 |
| | % | 0.1 | 1.1 | 0.3 | 0.0 | 0.0 | 0.0 | 0.0 | 0.0 | 0.0 | 0.0 |
| | total | 282286 | 283787 | 240546 | 255647 | 245240 | 261883 | 270400 | 247852 | 229351 | 222203 |
| | varieties | | | | | | | | | | |
| | total % | 2.0 | 1.6 | 1.8 | 4.8 | 4.9 | 2.4 | 1.3 | 0.9 | 0.8 | 0.5 |
| | Yr7 | | | | | | | | | | |
| Cultivated Yr7 varieties | | 2000 | 2001 | 2002 | 2003 | 2004 | 2005 | 2006 | 2007 | 2008 | 2009 |
| | % virYr7_isolat [symbol] | 4 | 0 | 3 | 36 | 4 | 8 | 11 | 4 | 0 | 0 |
| | total tons | 0 | 0 | 21 | 969 | 5307 | 4819 | 6466 | 8013 | 10764 | 12346 |
| | % | 0.0 | 0.0 | 0.0 | 0.5 | 2.9 | 3.1 | 4.3 | 4.3 | 5.7 | 7.1 |
| | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| | total tons | 0 | 0 | 0 | 0 | 0 | 0 | 0 | 0 | 191 | 5010 |
| | % | 0.0 | 0.0 | 0.0 | 0.0 | 0.0 | 0.0 | 0.0 | 0.0 | 0.1 | 2.9 |
| | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| | total tons | 234.8 | 132.65 | 117 | 60 | 39 | 0 | 0 | 0 | 0 | 0 |
| | % | 0.1 | 0.1 | 0.1 | 0.0 | 0.0 | 0.0 | 0.0 | 0.0 | 0.0 | 0.0 |
| | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| | total tons | 896.9 | 259.544 | 212.345 | 195 | 79 | 139 | 33 | 1 | 1 | 0 |
| | % | 0.5 | 0.1 | 0.1 | 0.1 | 0.0 | 0.1 | 0.0 | 0.0 | 0.0 | 0.0 |
| | total tons | 0 | 0 | 0 | 0 | 0 | 0 | 0 | 0 | 0 | 0 |
| | % | 0.0 | 0.0 | 0.0 | 0.0 | 0.0 | 0.0 | 0.0 | 0.0 | 0.0 | 0.0 |
| | total | 182648 | 176431 | 165486 | 186474 | 185970 | 154906 | 151525 | 184903 | 188184 | 174779 |
| | varieties | | | | | | | | | | |
| | total % | 0.6 | 0.2 | 0.2 | 0.7 | 2.9 | 3.2 | 4.3 | 4.3 | 5.8 | 9.9 |
| | Yr7 | | | | | | | | | | |
| | Cultivated Yr7 varieties | | 2010 | 2011 | 2012 | 2013 | 2014 | 2015 | 2016 | | |
| | | % virYr7_isolat [symbol] | 24 | 70 | 97 | 92 | 93 | 76 | 92 | | |
| | | total tons | 10494 | 9171 | 8389 | 6,815.20 | 6,375.10 | 4,858.90 | 3,076.30 | | |
| | | % | 5.7 | 4.7 | 4.9 | 4.0 | 3.9 | 2.8 | 1.9 | | |
| | | total tons | 0 | 0 | 0 | 65.9 | 490.9 | 197.7 | 53.9 | | |
| | | % | 0.0 | 0.0 | 0.0 | 0.0 | 0.3 | 0.1 | 0.0 | | |
| | | total tons | 10719 | 9948 | 9832 | 8,161.10 | 5,903.30 | 4,664.20 | 3,326.20 | | |
| | | % | 5.8 | 5.0 | 5.7 | 4.8 | 3.6 | 2.7 | 2.1 | | |
| | | total tons | 0 | 0 | 0 | 275 | 11,885.60 | 17,032.90 | 17,587.70 | | |
| | | % | 0.0 | 0.0 | 0.0 | 0.2 | 7.2 | 9.7 | 11.0 | | |
| | | total tons | 0 | 0 | 0 | 13.8 | 9.20 | 0 | 0 | | |
| | | % | 0.0 | 0.0 | 0.0 | 0.0 | 0.0 | 0.0 | 0.0 | | |
| | | total tons | 0 | 0 | 0 | 0 | 0 | 0 | 0 | | |
| | | % | 0.0 | 0.0 | 0.0 | 0.0 | 0.0 | 0.0 | 0.0 | | |
| | | total tons | 0 | 0 | 0 | 0 | 0 | 0 | 0 | | |
| | | % | 0.0 | 0.0 | 0.0 | 0.0 | 0.0 | 0.0 | 0.0 | | |
| | | total tons | 0 | 0 | 0 | 0 | 0 | 0 | 0 | | |
| | | % | 0.0 | 0.0 | 0.0 | 0.0 | 0.0 | 0.0 | 0.0 | | |
| | | total tons | 0 | 0 | 0 | 0 | 0 | 0 | 0 | | |
| | | % | 0.0 | 0.0 | 0.0 | 0.0 | 0.0 | 0.0 | 0.0 | | |
| | | total tons | 0 | 0 | 0 | 0 | 0 | 0 | 0 | | |
| | | % | 0.0 | 0.0 | 0.0 | 0.0 | 0.0 | 0.0 | 0.0 | | |
| | | total tons | 0 | 0 | 0 | 0 | 0 | 0 | 0 | | |
| | | % | 0.0 | 0.0 | 0.0 | 0.0 | 0.0 | 0.0 | 0.0 | | |
| | | total tons | 0 | 0 | 0 | 0 | 0 | 0 | 0 | | |
| | | % | 0.0 | 0.0 | 0.0 | 0.0 | 0.0 | 0.0 | 0.0 | | |
| | | total | 184795 | 197221 | 171034 | 170,276.70 | 164,779.00 | 174,991.40 | 159,371.70 | | |
| | | varieties | | | | | | | | | |
| | | total % | 11.5 | 9.7 | 10.7 | 9.0 | 15.0 | 15.3 | 15.1 | | |
| | | Yr7 | | | | | | | | | |

[symbol]

indicates data missing or illegible when filed

to draw the plot presented next to the table. The proportion of harvested Yr7 wheat varieties is shown in dark green and prevalence of yellow rust isolates virulent to Yr7 in orange (UK, from 1990 to 2016).

TABLE 2
Summary of the newly generated and previously published plant materials analysed for
the present study with the different PST isolates used for the pathology assays.
Table 2: Plant materials and rust isolated used in the present study

| Gene | Experiment | Plant Material | Rust isolate | Reference(s) |
|---|---|---|---|---|
| Yr7 | MutRenSeq | EMS-derived TILLING | PST 08/21 | Krasileva et al., 2017 |
| | | population in the | | |
| | | UK Cadenza cultivar | | |
| | Confirmation of the Yr7 | Avocet-Yr7 EMS mutants | | Generated for the study |
| | candidate through | | | |
| | sequencing | | | |
| | Genetic linkage | F₂ population: | | Generated for the study |
| | confirmation | Cad0127 × CadWT (139) | | |
| | Yr7 KASP primer testing | Cadenza-derived varities + | PST 08/21; PST 15/15 [symbol] | Generated for the study |
| | | Yr7 carriers | | |
| | Yr7 frequency in | UK Recommended list 2018 | | https://cereals.ahdb.org.uk/varieties/ |
| | breeding materials | | | ahdb-recommended-lists.aspx |
| | | Gediflux collecion | | Reeves et al., 2004 |
| | | Core-set of the Watkins collection | | Wingen et al., 2014 |
| Yr5 | MutRenSeq | EMS-derived Lemhi-Yr5 mutants | PST81/20 | McGrann et al., 2014 |
| | Confirmation of the Yr5 | Avocet-Yr5 EMS mutants | | Generated for the study |
| | candidate through | | | |
| | sequencing | | | |
| | Genetic linkage | F₂ population: | | Generated for the study |
| | confirmation | Avocet-S × Avocet-S-Yr5 (376) | | |
| YrSP | MutRenSeq | Avocet-YrSP EMS mutants | 134 E16A+(Culture n [symbol] | Generated for the study |
| | Genetic linkage | F₂ population: | | Generated for the study |
| | confirmation | Avocet-S × Avocet-S-Yr5 (94) | | |

[symbol]

indicates data missing or illegible when filed

TABLE 4
Summary of the available genome assemblies that we used for the in silico allele mining and synteny analysis
across rice, Brachypodium, barley and different triticeae accessions.
Table 4: Genome assemblies that were used for the present study

| Specie | Cultivar/grou [symbol] | Source | Link/ref |
|---|---|---|---|
| Triticum aestivum | Cadenza | Earlham Institute | http://opendata.earlham.ac.uk/ |
| | | | Triticum_aestivum/EI/v1.1/ |
| Triticum aestivum | Paragon | Earlham Institute | http://opendata.earlham.ac.uk/ |
| | | | Triticum_aestivum/EI/v1.1/ |
| Triticum aestivum | Claire | Earlham Institute | http://opendata.earlham.ac.uk/ |
| | | | Triticum_aestivum/EI/v1.1/ |
| Triticum aestivum | Robigus | Earlham Institute | http://opendata.earlham.ac.uk/ |
| | | | Triticum_aestivum/EI/v1.1/ |
| Triticum turgidum | Kronos | Earlham Institute | http://opendata.earlham.ac.uk/ |
| | | | Triticum_turgidum/EI/v1.1/ |
| Triticum turgidum | Svevo | The International Durum Wheat | http://d-data.interomics.eu |
| | | Genome Sequencing Consortium | |
| Triticum turgidum | Zavitan | WEWseq | Avni et al. 2017 |
| Aegilops tauschii | Tauschii | UC Davis | Luo et al. 2017 |
| Oryza sativa | Japonica | Ensembl/RAP-DB | http://plants.ensembl.org/ |
| | | | Oryza_sativa/Info/Index |
| Brachypodium distachyon | | Ensembl/Brachypodium.org | http://plants.ensembl.org/ |
| | | | Brachypodium_distachyon/Info/Index |
| Hordeum vulgare | Morex | Ensembl/IBSC | http://plants.ensembl.org/ |
| | | | Hordeum_vulgare/Info/Index |

[symbol]

indicates data missing or illegible when filed

TABLE 5a
In silica allele mining for Yr7 and Yr5/YrSP
in available genome assemblies for wheat

| Cultivar | % ID to Yr5 protein | % ID to Yr7 protein |
|---|---|---|
| Cadenza | 98.2 | 100 |
| Paragon | 98.2 | 99.8* |
| Claire | 99.3 | n.s |
| Robigus | 98.2 | n.s |
| Kronos | 93.6 | n.s |
| Svevo | 93.6 | n.s |
| Zavitan | n.s | n.s |

*due to the presence of the Ns in the Paragon sequence (see supp) haplotypes

TABLE 6
List of the identified BED-containing proteins in RefSeq v1.0 based on a hmmerscan analysis (see Methods). Several
features are added: number of identifed BED domains and the presence of other conserved domains present, the best BLAST hit
from the non-redundant database of NCBI with its description and score, and whether the BED domain was related to BED domains
from NLR proteins based on the neighbour network shown oi FIG. 10.
Table 6: List of the identified BED-containing proteins in RefSeqv1.0 based on a hmmerscan analysis

| | | | | | CD- | CD- | |
|---|---|---|---|---|---|---|---|
| | # | CD- | CD- | CD-Search/ | Search/ | Search/ | |
| | BED | Search/ [symbol] | Search | hmmer | hmmer [symbol] | hmme [symbol] | Best BLAST hit |
| TraesCS1B01G158800.1 | 1 | ZnF_BED | DUF4413 | Dimer_ | | | XP_016740977.1 |
| | | | | Tnp_hAT | | | |
| TraesCS3B01G269600.1 | 1 | ZnF_BED | DUF4413 | Dimer_ | | | XP_020177565.1 |
| | | | | Tnp_hAT | | | |
| TraesCS3B01G317800.1 | 1 | ZnF_BED | DUF4413 | Dimer_ | | | XP_020177565.1 |
| | | | | Tnp_hAT | | | |
| TraesCS5B01G377100.1 | 1 | ZnF_BED | DUF4413 | Dimer_ | | | ABA94812.1 |
| | | | | Tnp_hAT | | | |
| TraesCS5B01G501500.1 | 1 | ZnF_BED | | | | | XP_020164333.1 |
| TraesCS5D01G501900.1 | 1 | ZnF_BED | | | | | XP_020164333.1 |
| TraesCS7A01G447400.1 | 1 | ZnF_BED | DUF4413 | Dimer_ | | | XP_020177565.1 |
| | | | | Tnp_hAT | | | |
| | | | | | | | BED sequence |
| | | | | | | | related to BNLs |
| | | | | | | align- | in Neighbour |
| | | Best BLAST hit description | qlength | slentgh | % ID | ment | Network Tree |
| | TraesCS1B01G158800.1 | PREDICTED: zinc finger BED | 706 | 698 | 42.837 | 705 | Yes |
| | | domain-containing | | | | | |
| | TraesCS3B01G269600.1 | zinc finger BED domain- | 772 | 395 | 94.43 | 395 | yes |
| | | containing protein RICE [symbol] | | | | | |
| | TraesCS3B01G317800.1 | zinc finger BED domain- | 675 | 395 | 92.911 | 395 | yes |
| | | containing protein RICE [symbol] | | | | | |
| | TraesCS5B01G377100.1 | hAT family dimerisation | 728 | 709 | 58.779 | 655 | yes |
| | | domain containing prot [symbol] | | | | | |
| | TraesCS5B01G501500.1 | protein NLP4-like [Aegilops | 663 | 714 | 74.965 | 715 | yes |
| | | tauschii subsp. taus [symbol] | | | | | |
| | TraesCS5D01G501900.1 | protein NLP4-like [Aegilops | 715 | 714 | 100 | 714 | yes |
| | | tauschii subsp. taus [symbol] | | | | | |
| | TraesCS7A01G447400.1 | zinc finger BED domain- | 772 | 395 | 94.937 | 395 | yes |
| | | containing protein RICE [symbol] | | | | | |

[symbol]

indicates data missing or illegible when filed

TABLE 8
List of de novo assemblies generated from the corresponding RenSeq data
Table 8: Sequencing data details

| | | | | | | | # Read-pairs | | |
|---|---|---|---|---|---|---|---|---|---|
| | | | Enrichment | Sequence | | | mapped to | | |
| Sample | Accession | Sequencing chemistry | po [symbol] | pool | # Read-pairs | # Read-pairs | the de novo | % Read-pairs | do novo assembly |
| MW01-127_HM7MVBCXX_L1_2.fq.gz | Cad0127 | Illumina_HiSeq_2500 ( [symbol] | A | 1 | 14805176 | 14743094 | 18772686 | 64% | Cadenza-WT |
| MW01-127_HM7MVBCXX_L1_2.fq.gz | Cad0127 | Illumina_HiSeq_2500 ( [symbol] | A | 1 | 14805176 | 14743094 | | | |
| MW01-1551_HM7MVBCXX_L1_1.fq.gz | Cad1551 | Illumina_HiSeq_2500 ( [symbol] | A | 1 | 8216218 | 8184048 | 10619188 | 65% | Cadenza-WT |
| MW01-1551_HM7MVBCXX_L1_2.fq.gz | Cad1551 | Illumina_HiSeq_2500 ( [symbol] | A | 1 | 8216218 | 8184048 | | | |
| MW01-1978_HM7MVBCXX_L1_1.fq.gz | Cad1978 | Illumina_HiSeq_2500 ( [symbol] | B | 1 | 12462294 | 12409066 | 15916836 | 64% | Cadenza-WT |
| MW01-1978_HM7MVBCXX_L1_2.fq.gz | Cad1978 | Illumina_HiSeq_2500 ( [symbol] | B | 1 | 12462294 | 12409066 | | | |
| WW01-27_Cadenza_S3_L001_R1_001.fastq.gz | Cadenza-WT | Illumina_MiSeq (250b [symbol] | C | 2 | 5901019 | 5843683 | 7884202 | 67% | Cadenza-WT |
| WW01-27_Cadenza_S3_L001_R2_001.fastq.gz | Cadenza-WT | Illumina_MiSeq (250b [symbol] | C | 2 | 5901019 | 5843683 | | | |
| AvS_KD17010810-A71_HCHT7BCXY_L1_1.fq.gz | AvocetS | Illumina_HiSeq_2500 ( [symbol] | D | 3 | 12669666 | 12284950 | | | |
| AvS_KD17010810-A71_HCHT7BCXY_L1_2.fq.gz | AvocetS | Illumina_HiSeq_2500 ( [symbol] | D | 3 | 12669666 | 12284950 | | | |
| AvS_SP_KD17010810-A50_HCHT7BCXY_L1_1.fq.gz | AvocetS-YrS [symbol] | Illumina_HiSeq_2500 ( [symbol] | D | 3 | 13559810 | | | | |
| AvS_SP_KD17010810-A50_HCHT7BCXY_L1_2.fq.gz | AvocetS-YrS [symbol] | Illumina_HiSeq_2500 ( [symbol] | D | 3 | 13559810 | | | | |
| AvS_Yr5_KD17010810-A81_HCHT7BCXY_L1_1.fq.gz | AvocetS-Yr5 | Illumina_HiSeq_2500 ( [symbol] | D | 3 | 10131809 | | | | |
| AvS_Yr5_KD17010810-A81_HCHT7BCXY_L1_2.fq.gz | AvocetS-Yr5 | Illumina_HiSeq_2500 ( [symbol] | D | 3 | 10131809 | | | | |
| AvS_Yr7_KD17010810-A93_HCHT7BCXY_L1_1.fq.gz | AvocetS-Yr7 | Illumina_HiSeq_2500 ( [symbol] | D | 3 | 7698058 | | | | |
| AvS_Yr7_KD17010810-A93_HCHT7BCXY_L1_2.fq.gz | AvocetS-Yr7 | Illumina_HiSeq_2500 ( [symbol] | D | 3 | 7698058 | | | | |
| C855_KD17010810-A2_HCHT7BCXY_L1_1.fq.gz | Cad0855 | Illumina_HiSeq_2500 ( [symbol] | E | 3 | 13109055 | 12568140 | 17166458 | 68% | Cadenza-WT |
| C855_KD17010810-A2_HCHT7BCXY_L1_2.fq.gz | Cad0855 | Illumina_HiSeq_2500 ( [symbol] | E | 3 | 13109055 | 12568140 | | | |
| C903_KD17010810-A94_HCHT7BCXY_L1_1.fq.gz | Cad0903 | Illumina_HiSeq_2500 ( [symbol] | E | 3 | 9109264 | 8704600 | 11780688 | 68% | Cadenza-WT |
| C903_KD17010810-A94_HCHT7BCXY_L1_2.fq.gz | Cad0903 | Illumina_HiSeq_2500 ( [symbol] | E | 3 | 9109264 | 8704600 | | | |
| C923_KD17010810-A40_HCHT7BCXY_L1_1.fq.gz | Cad0923 | Illumina_HiSeq_2500 ( [symbol] | E | 3 | 14252713 | 13647531 | 17530654 | 64% | Cadenza-WT |
| C923_KD17010810-A40_HCHT7BCXY_L1_2.fq.gz | Cad0923 | Illumina_HiSeq_2500 ( [symbol] | E | 3 | 14252713 | 13647531 | | | |
| C1034_KD17010810-A49_HCHT7BCXY_L1_1.fq.gz | Cad1034 | Illumina_HiSeq_2500 ( [symbol] | E | 3 | 13415313 | 12889224 | 15567764 | 60% | Cadenza-WT |
| C1034_KD17010810-A49_HCHT7BCXY_L1_2.fq.gz | Cad1034 | Illumina_HiSeq_2500 ( [symbol] | E | 3 | 13415313 | 12889224 | | | |
| YSP_0_KD17071213-AK3122_HV32GBCXY_L1_l.fq.gz | AvocetS-YrS [symbol] | Illumina_HiSeq_2500 ( [symbol] | F | 4 | 20168141 | 19285244 | 25472610 | 66.04% | AvocetS-YrSP-WT |
| YSP_0_KD17071213-AK3122_HV32GBCXY_L1_2.fq.gz | AvocetS-YrS [symbol] | Illumina_HiSeq_2500 ( [symbol] | F | 4 | 20168141 | 19285244 | | | AvocetS-YrSP-WT |
| YSP_1_KD17071213-AK2489_HV32GBCXY_L1_1.fq.gz | AvocetS-YrS [symbol] | Illumina_HiSeq_2500 ( [symbol] | F | 4 | 4866592 | 4715938 | 6208114 | 65.82% | AvocetS-YrSP-WT |
| YSP_1_KD17071213-AK2489_HV32GBCXY_L1_2.fq.gz | AvocetS-YrS [symbol] | Illumina_HiSeq_2500 ( [symbol] | F | 4 | 4866592 | 4715938 | | | AvocetS-YrSP-WT |
| YSP_2_KD17071213-AK3121_HV32GBCXY_L1_1.fq.gz | AvocetS-YrS [symbol] | Illumina_HiSeq_2500 ( [symbol] | G | 4 | 22067358 | 21281452 | 28040118 | 65.88% | AvocetS-YrSP-WT |
| YSP_2_KD17071213-AK3121_HV32GBCXY_L1_2.fq.gz | AvocetS-YrS [symbol] | Illumina_HiSeq_2500 ( [symbol] | G | 4 | 22067358 | 21281452 | | | AvocetS-YrSP-WT |
| YSP_3_KD17071213-AK2464_HV32GBCXY_L1_1.fq.gz | AvocetS-YrS [symbol] | Illumina_HiSeq_2500 ( [symbol] | G | 4 | 14603831 | 14068492 | 18132636 | 64.44% | AvocetS-YrSP-WT |
| YSP_3_KD17071213-AK2464_HV32GBCXY_L1_2.fq.gz | AvocetS-YrS [symbol] | Illumina_HiSeq_2500 ( [symbol] | G | 4 | 14603831 | 14068492 | | | AvocetS-YrSP-WT |
| YSP_4_KD17071213-AK2483_HV32GBCXY_L1_1.fq.gz | AvocetS-YrS [symbol] | Illumina_HiSeq_2500 ( [symbol] | H | 4 | 16757582 | 15993630 | 20438956 | 63.90% | AvocetS-YrSP-WT |
| YSP_4_KD17071213-AK2483_HV32GBCXY_L1_2.fq.gz | AvocetS-YrS [symbol] | Illumina_HiSeq_2500 ( [symbol] | H | 4 | 16757582 | 15993630 | | | AvocetS-YrSP-WT |
| Y5_0_KD17071213-AK2488_HV32GBCXY_L1_1.fq.gz | AvocetS-Yr5- | Illumina_HiSeq_2500 ( [symbol] | H | 4 | 18106714 | 17329780 | 23756414 | 68.54% | AvocetS-Yr5-WT |
| Y5_0_KD17071213-AK2488_HV32GBCXY_L1_2.fq.gz | AvocetS-Yr5- | Illumina_HiSeq_2500 ( [symbol] | H | 4 | 18106714 | 17329780 | | | AvocetS-Yr5-WT |
| Y5_1_KD17071213-AK2485_HV32GBCXY_L1_1.fq.gz | AvocetS-Yr5- | Illumina_HiSeq_2500 ( [symbol] | I | 4 | 12149902 | 11617256 | 14917602 | 64.20% | AvocetS-Yr5-WT |
| Y5_1_KD17071213-AK2485_HV32GBCXY_L1_2.fq.gz | AvocetS-Yr5- | Illumina_HiSeq_2500 ( [symbol] | I | 4 | 12149902 | 11617256 | | | AvocetS-Yr5-WT |
| Y5_2_KD17071213-AK2486_HV32GBCXY_L1_1.fq.gz | AvocetS-Yr5- | Illumina_HiSeq_2500 ( [symbol] | I | 4 | 18064931 | 16987606 | 23153166 | 68.15% | AvocetS-Yr5-WT |
| Y5_2_KD17071213-AK2486_HV32GBCXY_L1_2.fq.gz | AvocetS-Yr5- | Illumina_HiSeq_2500 ( [symbol] | I | 4 | 18064931 | 16987606 | | | AvocetS-Yr5-WT |
| Y5_3_KD17071213-AK2487_HV32GBCXY_L1_1.fq.gz | AvocetS-Yr5- | Illumina_HiSeq_2500 ( [symbol] | J | 4 | 15563606 | 14814817 | 19915922 | 67.22% | AvocetS-Yr5-WT |
| Y5_3_KD17071213-AK2487_HV32GBCXY_L1_2.fq.gz | AvocetS-Yr5- | Illumina_HiSeq_2500 ( [symbol] | J | 4 | 15563606 | 14814817 | | | AvocetS-Yr5-WT |

[symbol]

indicates data missing or illegible when filed

TABLE 9
Sequencing details of RenSeq data generated in this study.
Table 9: de novo assemblies from RenSeq data statistics

| de novo assembly | assembler | #contigs | #NLR-contigs | #complete_NLR |
|---|---|---|---|---|
| Cadenza-WT | CLC assembly cell | 29706 | 5572 | 431 |
| AvocetS | CLC assembly cell | 400158 | | |
| AvocetS + YrSP | CLC assembly cell | 530695 | | |
| AvocetS + Yr7 | CLC assembly cell | 278126 | | |
| AvocetS + Yr5 | CLC assembly cell | 362856 | | |
| Paragon | | | | |
| Kronos | | | | |
| AvocetS + YrSP_AU | CLC Genomics Wo [symbol] | 268235 | 5361 | 791 |
| AvocetS + Yr5_AU | CLC Genomics Wo [symbol] | 109608 | 5180 | 782 |

[symbol]

indicates data missing or illegible when filed

TABLE 10
Summary of primers designed for the present study. (Part 1/2)

| | | | | KASP_R- | | | | |
|---|---|---|---|---|---|---|---|---|
| Primer_Name | Gene | Primer_Type | chromosome | gene_allele | KASP_alternate_allele | common | product_size | Comment |
| Yr7 | | | | | | | | |
| detection | | | | | | | | |
| Yr7-A | Yr7 | KASP | 2BL | TTAGTCCTGCC | TTAGTCCAGCCCATAAGCc | CAGTGTT | 41 | |
| | | | | CCATAAGCg | | AAAACCA | | |
| | | | | | | GGGAGGA | | |
| Yr7-B | Yr7 | KASP | 2BL | TGGAGGTATCA | TGGAGGTATCATCGGGTGAa | CATCAAA | 70 | Dominant |
| | | | | TCTGGTGAg | | ATCATCG | | marker: |
| | | | | | | CCTATGT | | alternate |
| | | | | | | | | allele is |
| | | | | | | | | actually |
| | | | | | | | | not |
| | | | | | | | | amplified |
| Yr7-C | Yr7 | KASP | 2BL | CACATGAGTCG | CACACGACCTAATACTGAGa | ACTGCAA | 48 | Dominant |
| | | | | ATACTGAGg | | TGCCTTC | | marker: |
| | | | | | | CCATA | | alternate |
| | | | | | | | | allele is |
| | | | | | | | | actually |
| | | | | | | | | not |
| | | | | | | | | amplified |
| Yr7-D | Yr7 | KASP | 2BL | GCTGGAAAGGC | GCTGGAAAGGCTTGAGATCg | AATGGCG | 48 | |
| | | | | TTGACATCa | | TGGTAAG | | |
| | | | | | | GACAGA | | |

| Primer_Name | Forward | Reverse | Product size Y | Product size YrSP | Alternate profile |
|---|---|---|---|---|---|
| Yr5 detection | | | | | |
| Yr5-Insertion | CTCACGCATT | TATTGCATAA [symbol] | 1281 | 507 | no amplification |

| Primer_Name | Gene | Primer_Type | chromosome | KASP_WT_allele | KASP_mutant_allele | common | product_size |
|---|---|---|---|---|---|---|---|
| Yr7 mapping | | | | | | | |
| Cad0127 | Yr7 | KASP | 2BL | AAGTGATGTCGGGA | AAGTGATGTCGGGAGGAGt | TGGAGAATG | 83 |
| | | | | GGAGc | | GAAGTTCTT | |
| | | | | | | TTGTGT | |
| Cad1551 | Yr7 | KASP | 2BL | CACAATCATCAAGA | CACAATCATCAAGATGAA | CCAACAATA | 51 |
| | | | | TGAAGCg | GCa | TCTCAGTTA | |
| | | | | | | CCTCATTG | |
| Cad1978 | Yr7 | KASP | 2BL | TGCATCCTTCCAGG | TGCATCCTTCCAGGACAA | AACCAGGGA | 79 |
| | | | | ACAAATg | ATa | GGACGCTTA | |
| | | | | | | TG | |
| Cad0127_M1 | Yr7 mapping | KASP | 2BL | ACATTTACGTGGAG | ACATATTCGTGGAGGCCGa | TGGTGAACT | 94 |
| | | | | GCCGg | | CTGATAGGA | |
| | | | | | | ACTTC | |
| Cad0127_M2 | Yr7 mapping | KASP | 2BL | TTCTCCTGCGCCTC | TTCTCCTGCGCCTCTCTGa | GGAGGGTCT | 59 |
| | | | | TCTGg | | GGCCTCTGT | |
| Cad0127_M3 | Yr7 mapping | KASP | 2BL | CGGAACCAATCACC | CGGAACCAATCACCTCGGa | ATGTTGTCC | 78 |
| | | | | TCGGg | | ACGGCGATT | |
| | | | | | | AA | |
| Cat0127_M4 | Yr7 mapping | KASP | 2BL | GAAAGCAGCAGCCA | GAAAGCAGCAGCCACAGt | TTGGTCGGC | 55 |
| | | | | CAGc | | TCTTGAACT | |
| | | | | | | TT | |
| Cad0127_M5 | Yr7 mapping | KASP | 2BL | CATCATCCATTTTC | CATCATCCATTTTCCCTC | AGCTTCTTT | 51 |
| | | | | CCTCTCGc | TCGt | AGAACATGC | |
| | | | | | | CAAC | |
| Cac0127_M6 | Yr7 mapping | KASP | 2BL | ACTGCTCGCAACAC | ACTGCTCGCAACACATAC | CCCAATTAT | 67 |
| | | | | ATACAc | At | TTGCAGTGC | |
| | | | | | | TTGAG | |
| Cad0127_M7 | Yr7 mapping | KASP | 2BL | GCTTCAGTGAACAA | GCTTCAGTGAACAAGGTG | GAGAGGAGA | 36 |
| | | | | GGTGATGc | ATGt | AATGACATC | |
| | | | | | | CTAGAT | |
| Cad0127_M8 | Yr7 mapping | KASP | 2BL | AGAACCAGAGAATT | AGAACCAGAGAATTTGTT | CGACTATGG | 103 |
| | | | | TGTTGTTGTAg | GTTGTAa | AGAACCTTG | |
| | | | | | | AGAGA | |
| Cad0127_M9 | Yr7 mapping | KASP | 2BL | GCCTTTCTTCATCT | GCCTTTCTTCATCTGGCC | TGTGGTACG | 78 |
| | | | | GGCCTTTAGc | TTTAGt | AGTTGGCAT | |
| | | | | | | ACC | |

| Primer_Name | Gene/Name | Primer_Type | chromosome | KASP_Target | KASP_Alt | common | product_size |
|---|---|---|---|---|---|---|---|
| Yr5 mapping | | | | | | | |
| Yr5_candidate | Yr5 | KASP | 2BL | CAGGAGATCTTG | CAGGAGATCT | AAACTCTTTGACT | 44 |
| | | | | AAGGACAT | TAAAGGAATA | GGTACTCG | |
| Yr5_M1 | W90K_Kukri_ [symbol] | KASP | 2BL | | | | ask SEB |
| Yr5_M2 | W90K_RAC87 | KASP | 2BL | | | | |
| Yr5_M3 | W90K_Tduru [symbol] | KASP | 2BL | | | | |
| WMC175 | | KASP | 2BL | | | | |
| Yr5_M4 | W901_Ra__c6 [symbol] | KASP | 2BL | | | | |
| Yr5_M5 | W90K_GENE- [symbol] | KASP | 2BL | | | | |
| Yr5_M6 | W90Kt_wsnp_ [symbol] | KASP | 2BL | | | | |
| YrSP mapping | | | | | | | |
| Yr5_candidate | YrSP | KASP | 2BL | CAGGAGATCTTG | CAGGAGATCTT | AAACTCTTTGACT | 44 |
| | | | | AAGGACAT | AAAGGAATA | GGTACTCG | |
| YrSP_M1 | W90K_JD_c2 [symbol] | KASP | 2BL | | | | |
| YrSP_M2 | RAC875_rep_ [symbol] | KASP | 2BL | | | | |
| Yr5P_M3 | BobWhite_c3 [symbol] | KASP | 2BL | | | | |

[symbol]

indicates data missing or illegible when filed

TABLE 10
Summary of primers designed for the present study. (Part 2/2)

| Primer name | Forward | Reverse | product size (bp) |
|---|---|---|---|
| Yr7 cloning | | | |
| Yr7_locus | AGCCAGCAGAAGTCTTAGAAACAG | CTACGAGATATATGTTGAGCAGCTTG | 6.6 kb |
| A | TCTTAGAAACAGCCACGTC | ACGTCGATCAAACAGAGG | 704 |
| B | TTGTACTTCGGCATCCTC | ACACTTCGCTTTCACTGG | 709 |
| C | TCAATCTTTGGGTTGTGC | TGTGCCGAAAAGAAACAT | 791 |
| D | CTGAGGTCGAGAGAGTCG | TTTCCGTTGGACGAACTA | 746 |
| E | CTGATAACCAACCCACCA | CGCGAAGTTGTTAATTCC | 702 |
| F | GATCCAGCGCTACTTCAA | AACGGATTGCCCTTTAAC | 829 |
| G | TTGTCTGTTGCACAAAGGT | AGGAATGTTCCCCTTCAG | 728 |
| H | AAGAATTGGATGGGGAAG | ATAAGCGTCCTCCCTGGT | 784 |
| I | CTACCCAATGGCTTGTTG | GCCATGATCCCTGAATG | 768 |
| J | AGGTGAAGTTGAGCAGCA | CATCAGCGATAGCCACTT | 713 |
| K | CAGATGTGACGGCAGAGT | GTTGCGTGCCCTCTAGTA | 734 |
| L | AGAAACGCTGCAAGTCTG | CTGAAACGCTCATTCTGG | 792 |
| Yr5 cloning | | | |
| Yr5_locus | CGCTTAATTCCCCTTCCTTC | CACGTCAGACTGGATCAAAGCTCTA | 4.9 kb |
| A | Yr5_locus_F | TGGCTCCTTATTCGTTCTCTTTC | 813 |
| B | GGGAACACTTCACGATCA | AATTCCTTCATGCCTTCC | 901 |
| C | CTTGCTCCAAGGAAAGTG | CCCTGTGACATCCAGAAA | 890 |
| D | AGGGAAACCCACTAGCAG | TGGTTGCAATGGAAGAGT | 900 |
| E | GTGTGCTGCAAATGTCTG | ATGACCTCTGCCCAGTTT | 819 |
| F | GAGAAACCTGCCCAAAGT | ATGGTATGCGCAACAGTC | 884 |
| G | GGTTGCCGGAATCTAAGT | GATGGGTCTTGGATGTGA | 890 |
| H | GCAACCCTGCTTTCCTAGC | Yr5_locus_R | 671 |

TABLE 18
Corresponding gene models

| NLR Annotator | Longest overlap in Ensembl | BLASTx best hit | comments | |
|---|---|---|---|---|
| Os1 | LOC_Os04g52970.1.1 | | | |
| Os2 | Os04t0621500-00_LOC_Os04g53030.1.1 | | | |
| Os3 | Transcript: LOC_Os04g53040.1.1 | | | |
| Os4 | Transcript: LOC_Os04g53050.1.1 && | | | |
| | Transcript: LOC_Os04g53060.1.1 | | | |
| Os5 | Transcript: LOC_Os04g53120.1.1 | | | |
| Os6 | Transcript: LOC_Os04g53160.1.1 | | | |
| Bd1 | BRADI_5g22145v3 | | Phytozome: Bradi5g22146.1 | |
| Bd2 | BRADI_5G22160.1 && | | truncated genes so kept Annotator | |
| | BRADI_5G22160.1 | | locus | |
| Bd3 | BRADI_5g22179v3 | | | |
| Bd4 | BRADI5G22187 | | | |
| Hv1 | HORVU2Hr1G103460.1 | XP_020186889.1 | Traces of BED but not annotated | |
| | | | as such by CD search | |
| Hv2 | HORVU2Hr1G103440.1 | | truncated gene so kept Annotator | |
| | | | locus | |
| Aet1 | EMT18301 | | | |
| Aet2 | X | EMS51583.1 | kept Annotator locus | |
| Aet3 | EMT06562 | | | |
| Aet4 | EMT29760 | | | |
| Aet5 | EMT12526 | | | |
| Aet6 | EMT02111 | | | |
| Aet7 | EMT18676 | | | |
| Aet8 | EMT12939 | | | |
| Tt1 | TRIDC2BG071010.1 | EMS62808.1 | | |
| Tt2 | TRIDC2BG071030.1 | EMS62808.1 | no conserved domain in gene model | |
| Tt3 | X | | kept Annotator locus | |
| Tt4 | TRIDC2BG071040.1 | | | |
| Tt5 | X | EMS51583.1 | kept Annotator locus | |
| Tt6 | TRIDC2BG071050.1 | EMS51583.1 | | |
| Tt7 | X | | kept Annotator locus | |
| Tt8 | TRIDC2BG071070.1 | CAD45026.1 | | |
| Tt9 | TRIDC2BG071070.18 | EMS62808.1 | kept Annotator locus | |
| Tt10 | TRIDC2BG071180.3 | XP_020186889 | | |
| Tt11 | X | | kept Annotator locus | |
| Tt12 | TRIDC2BG071220.1 | XP_020186937.1 | no conserved domain in gene model | |
| Tt13 | X | XP_003579311 | | |
| Tt14 | TRIDC2BG071240.1 | XP_020186937.1 | | |
| Tt15 | X | XP_003579311.1 | kept Annotator locus | |
| Tt16 | X | XP_014751374.1 | kept Annotator locus | |
| Tt17 | X | XP_003579311.1 | kept Annotator locus | |
| Tt18 | X | BAJ98893.1 | kept Annotator locus | |
| Tt19 | X | KQJ84588.2 | kept Annotator locus | |
| Tt20 | TRIDC2BG071280.1 | XP_003579311.1 | | |
| Ta_2A1 | TraesCS2A01G464500 | | | |
| Ta_2A2 | TraesCS2A01G464700 | | | |
| Ta_2A3 | TraesCS2A01G464900 | | | |
| Ta_2A4 | X | | partial NLR | kept Annotator locus |
| Ta_2A5 | TraesCS2A01G465100 | | | |
| Ta_2A6 | TraesCS2A01G465200 | | | |
| Ta_2A7 | TraesCS2A01G465600 | | | |
| Ta_2A8 | TraesCS2A01G466100 | | | |
| Ta_2A9 | X | XP_020186937.1 | | kept Annotator locus |
| Ta_2A10 | TraesCS2A01G625200LC | | partial gene model | kept Annotator locus |
| Ta_2A11 | TraesCS2A01G625400LC- | | | kept Annotator locus |
| | TraesCS2A01G625500LC- | | | |
| | TraesCS2A01G625600LC | | | |
| Ta_2A12 | TraesCS2A01G466500- | | | kept Annotator locus |
| | TraesCS2A01G625600LC- | | | |
| | TraesCS2A01G466600 | | | |
| Ta_2D1 | TraesCS2D01G465300 | | | |
| Ta_2D2 | TraesCS2D01G465400 | | | |
| Ta_2D3 | TraesCS2D01G465500 | | | |
| Ta_2D4 | TraesCS2D01G465600 | | | |
| Ta_2D5 | TraesCS2D01G466000 | | | |
| Ta_2D6 | TraesCS2D01G466400 | | | |
| Ta_2D7 | TraesCS2D01G466600 | | Modified gene model rescued one | |
| | | | additional BED domain | |
| Ta_2B1 | TraesCS2B01G486100 | | | |
| Ta_2B2 | TraesCS2B01G485200 | | | |
| Ta_2B3 | X | | partial NLR | kept Annotator locus |
| Ta_2B4 | TraesCS2B01G486300 | | | |
| Ta_2B5 | X | | partial NLR | kept Annotator locus |
| Ta_2B6 | TraesCS2B01G486400 | | | |
| Ta_2B7 | TraesCS2B01G486700 | | | |
| Ta_2B8 | TraesCS2B01G487700 | | | |
| Ta_2B9 | TraesCS2B01G488000 | | | |
| Ta_2B10 | TraesCS2B01G488400 | | | |
| Ta_2B11 | TraesCS2B01G488600- | | | |
| | TraesCS2B01G488700 | | | |
| Ta_2B12 | TraesCS2B01G734100LC | | | |
| Ta_2B13 | TraesCS2B01G489400 | | | |

SELECTED SEQUENCE INFORMATION

>Yr7_locus
(SEQ ID NO: 5)

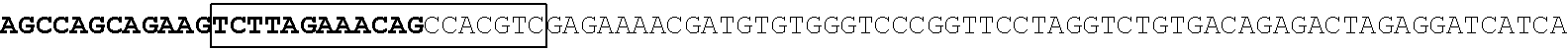

TCGGTTCTCGGTTCTCGGTTTTCGGGTTTGTGAAGCCTCTGACCCTGGCATTTGCTCGGGTTCGGTTCTGCTCTAGGTGCCTACTGGCTA
CGGCCAACGCGCCTCCTGTCGGGGCGGTTTTCCACGCAACTTAGCATCCGGCAACTTATATATAACAAACCTGCGTTCCTTCTTCTCGCT
CCACCGGTTTCCAAGCTCAGAGCTTCAAGCCAAACCCATTTCCAGTGAAGCAGTCGATGGAGCTCCTCACCTTCCTCTTCAGAATGGTGG
CCCTGATCCCCGGCGCATTACGCAACGCGGAGAAGCTGCCCGGTGCTCTCATCTCGTGCGGCGTCGTCCAAGCCGCGGCGGCGCTCTTCC

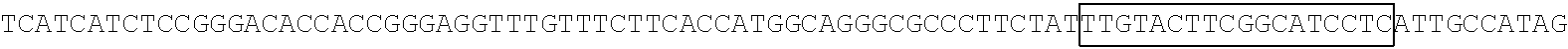

TGGTATTCGGGCTTGTGGAGGCGTCCGCCGGATTTTATGTGTCCGGCGATGTGGCCGGACGCCGTGCTGCCGGGAAGACCATCCTGTGGG

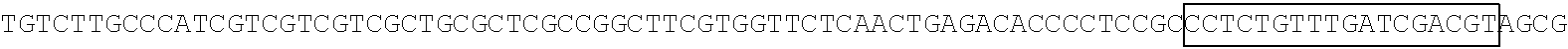

CCCGTTCATGTTGTATAGAATATAATGAGTGTATGTGTGTGTGTGTGTGTGTGTGTGTGTGTGTGTGTGCTGGGGGGCCATTTTGGTCAG
TGTGTGCTTTGGGGACGGGGGAATCAGTAGTAGGTTGTACCAGCACGAGTGTTTTAGACTTCATATACTTTCATTCTTTTTTTCACTTGA

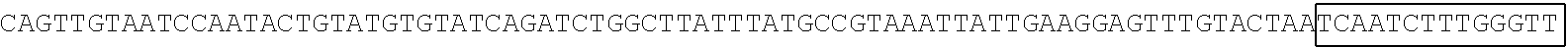

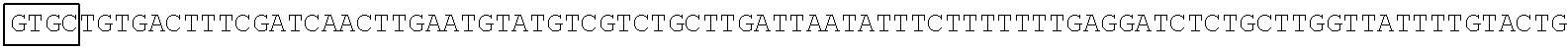

CTTCTCATGCCGTGTTCGGGCCGTATTCTCGAGCATAAAGTTCGGCCCACTAAGTGTCGAAAGAAAGCTGCTTCTAATTGACCTTCTGCT

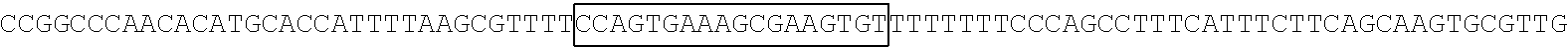

TGTGTTGTGGCTGGTGTTCTTCCCCGCTCGTCTCGTCTGCTCCCCATTCCACACGCTTAATTCCCCTTCCTTCATTGACTCGAGCTCGAG
ACCTGCTCCTGCCGGATCTGATAATGGAGCCGGCGGGAGACTCTTCCCTGGAGGCCGCGATTGCATGGCTGGTGCAGACCATCCTTGCAA

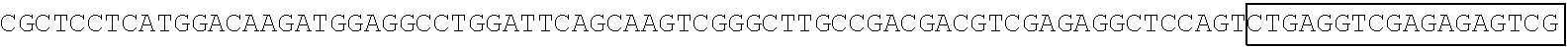

ACACGGTGGTGGCTGCTGTGAAGGGGAGGGCAGCCGGGAACATGCCTCTGTCCCGGTCTCTCGCTCGTGTCAAGGAGCTTCTCTATGACG
CCGACGACGTGATCGACGAGCTAGACTACTACAGGCTCCAACACCAAGTCGAAGGAGGTAGTAAGCATAATCCCATTATATATCGAATCT

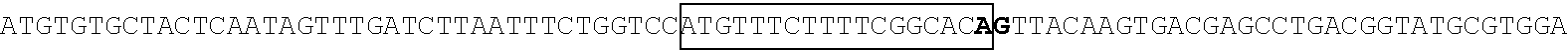

GCTGAAAGAGTGGATGAAATATCAAGGGGCCATGTCGATACACTGAATGTCAGTGTTGGCAAATTACGGTCCCCGGTATGGGAACACTTC
ACCATCACAGAAACAACTATCGACGGGAAGCGTTCAAAAGCCAAATGTAAGTACTGTGGAAATGATTTTAATTGCGAAACGAAGACAAAC
GGGACTTCATCTATGAAAAAACATTTGGAGAAGGAGCATTCCGTGACTTGCACGAATAAATCTGCAGTGCACCCCCCAAACACTTCAAGG
TACCAGCAGGAATTTATACCTTGCTTCAACGAATTTGTTGTAATTGTTTATATACGTCTGCTTGAGAGCCCATTGTTGTTCTGAATTTCT

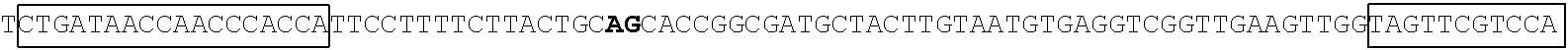

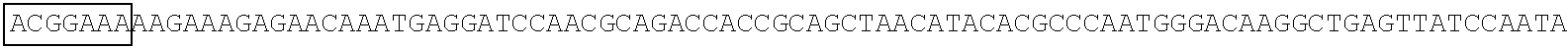

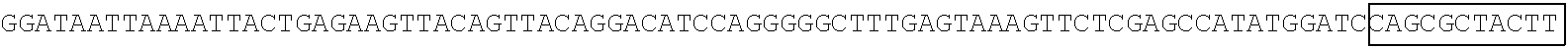

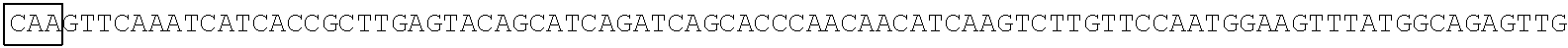

CAGAAAAGAATAAGATCAAAAAGTCAATAACTGAAAACCAATCTGGTGGTGTAAATGTTCTGCCTATTGTAGGCATTGCAGGTGTTGGAA
AGACAACTCTTGCTCAATTTGTGTATAATGATCCAGACGTGAAAAGTCAATTTCACCACAGGATATGGGTTTGTGTGTCCTGCAAATTTG

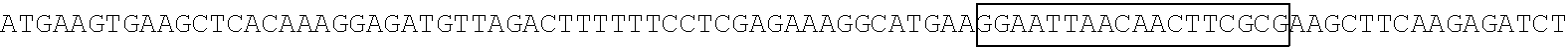

TGAAAGAACATGTCGAGTACCAAGCAAAGAGTTTTCTGCTCATTTTAGATGATGTCTCGGACAGTATGGATTATCATAAATGGAACAAAT

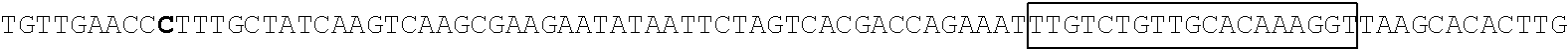

AACCGATCAAGTTAGGTGCTTTAGAAAACGATGATATGTGGTTATTGCTCAAGTCATGTGCATTTGGTTTTGGGAACTATGAAGGTACGG

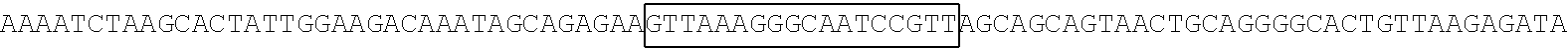

ATCTTAGCATTGATCATTGGAGTAACATTCTCAAGAATGAGAAGTGGAAATCGCTGGGACTCAGTGGGGGCATCATGCCTGCTTTGAAGC
TTAGTTATGATGAGTTGACGTACCGTTTACAACAATGTTTCTCGTATTGCTCTATATTTCCTGACAAATATAGGTTTCTCGGGAAGGATT
TGGTCTATATTTGGATTTCTCAGGGATTTGTGAATTGCACCCAAAATAAGAGATTGGAGGAGACGGGATGGGAATATCTGAATCAATTGG

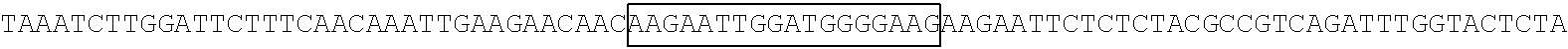

TGTGTGATCTCATGCATGATTTCGCAAGGATGATTTCAAGGACTGAATGTGCGACTATAGATGGTCTACAGTGCAATAAAATATTCCCAA

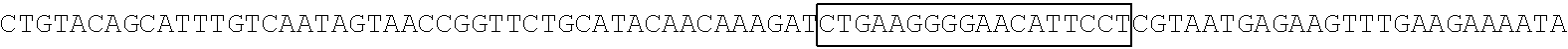

TGAGAAATTCAGTTACATCAGTTACCAAATTGAGAACATTGGTTGTGCTTGGGAACTTTGACTCTTTCTTTGTACGGTTGTTCCAAGATA
TATTCCAGAAGGCACAAAATTTACGCCTGCTGCTAGTATCTCTAGCATCCACTTATCTGTCTCAAGTGCCTGCTGCATTCAATGATTTTA
ATTCCTTCCTGTGCAATTTGGCAAATCCTTTGCATCTTCGTTACCTAAAACTTGAGTTGGATGGGATTGTGCCACAAGTTTTGAGTACGT

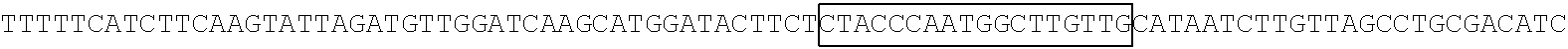

TTGTTGCACACAAGAGAGTCCATTCTTCCATTACTAGCATTGGTAACATGACATCTATCCAGGAGCTACATGATTTTGAAGTTCGAATTT

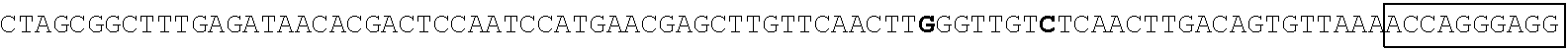

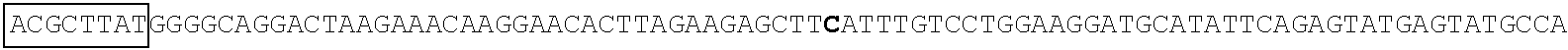

GTGACACTGAATTTGAATCTTCTGCAAACATGGCAAGAGAAGTGATTGAGGGTCTTGAACCACACATGGATTTAAAACATCTACAAATAT
CTCAGTATAATGGTACCACTTCACCAGCTTGGCTTGCCAACAATATCTCAGTTACCTCATTGCAGACGCTTCATCTTGATGATTGTGGAG

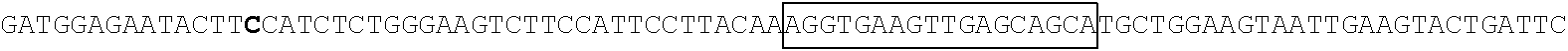

CTTCACTGGAGGAGCTAGTTCTAATTAAAATGCCGAAGTTAGTGAGATGCTCAAGCACTTCTGCCGAGGGTCTGAGCTCTAGCTTAAGGG

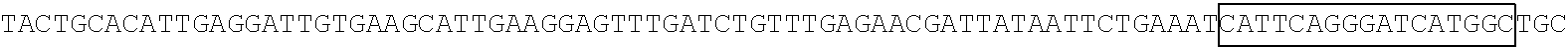

CTGGTCTTAGGAATTTGATTCTATATTGTTGCCCTCATTTGAAAGTGTTGAAGCCTCTTCCACCTTCAACTACCTTTTCTAAGGTACTCA
TCAGAGAAATTTCAAGATTTCCGTCTATGGAGGTATCATCTGGTGAGAAGTTACAAATTGGGAATATTGATGTGTACATAGGCGATGATT
TTGATGAGTCTTCTGATGAGTTGAGCATACTGGATGACAAAACTTTGGCGTTCCATAATCTTAGAAACCTGAAATCGATGGAGATATATG
GTTGCAGAAATCTAAGGTCTTTTTCGTTCGAAGGTTTCAGTCATCTTGTCTCTTTAACAAGTTTGAAAATAGTAAGCTGTGAACAACTTT

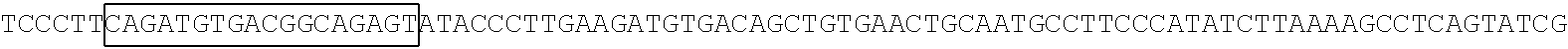

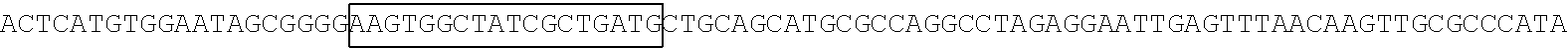

TAACAAGAGTAGTGTTACCGATGGAAGAGGAAGAAAACAATCTATTAACAACAGTACTGTCATCAGGAAATCAAGATGAGGCATTGACAT
GGTTAGTTCGTGACGGACTCTTGCACATTCCATCAAATCTCGTCTCCTCTCTCAAGAATATGAGTATTACTCAGTGCCCTCGCCTAAAGT
TTAACTCAGGCAAGGACTGCTTCTCTGGATTTACCTCGCTTGAGAAGCTTGAAATTTGGGGATCGTTGGTGGATGATGACGGAAGTGATG
ACCTGGAGAATGGAAGTTCTTTTGTGTTCGGAGAGGAGGATCAACCCCTGGGGGCGAACGGAAGATGGCTCCTCCCGACATCACTTCAGG

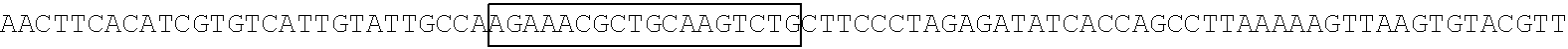

CCGGCCAAGGTTTGCAATCTCTACAGCTGTACTCATGCACGGCACTGGAAGAATTGGCAATTTCCGGCTCTGGATCGGTCACCGTCACTG

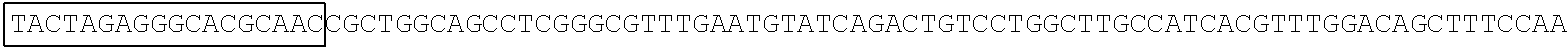

GGTTGTGCCCTCGGCTGGAAAGGCTTGACATCAATGACCCATCTGTCCTTACCACGCCATTCTGCAAGCACCTCACCTCCCTGCAACGCC
TAAAACTTGGCTTCTTGAAAGTGACGAGACTAACAGATGAGCAAGAACGAGCGCTTGTGCTCCTCAAGTCACTGAAAGAGCTCGAGATTT
TTTATTGTACTCATCTCATAGATCTTCCTGCGGGGCTGCAGACCCTTCCTTCCCTCAAGAGTTTGAAGATAGAAGAGGGTCGAGGCATCT
CAAGGCTGCCGGAAGCAGGCCTCCCACATTCGCTGGAAGAACTGGAAATCAAAATTTGCAGCAAGCTAGAAGATGAATGCAGGCGGCTAG
CAACATGCGAAGGCAAGCTAAAAGTCAAAATTGATGGTCGATATGTGAATTAATTATGTTTCTGGCCTCATGTGCAAAGTGTACCGCTTG

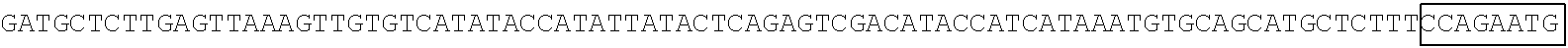

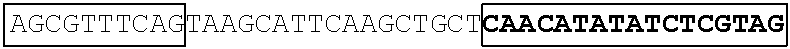

>Yr7_CDS
ATGGAGCCGGCGGGAGACTCTTCCCTGGAGGCCGCGATTGCATGGCTGGTGCAGACCATCCTTGCAACGCTCCTCATGGACAAGATGGAG
GCCTGGATTCAGCAAGTCGGGCTTGCCGACGACGTCGAGAGGCTCCAGTCTGAGGTCGAGAGAGTCGACACGGTGGTGGCTGCTGTGAAG
GGGAGGGCAGCCGGGAACATGCCTCTGTCCCGGTCTCTCGCTCGTGTCAAGGAGCTTCTCTATGACGCCGACGACGTGATCGACGAGCTA
GACTACTACAGGCTCCAACACCAAGTCGAAGGAGTTACAAGTGACGAGCCTGACGGTATGCGTGGAGCTGAAAGAGTGGATGAAATATCA
AGGGGCCATGTCGATACACTGAATGTCAGTGTTGGCAAATTACGGTCCCCGGTATGGGAACACTTCACCATCACAGAAACAACTATCGAC
GGGAAGCGTTCAAAAGCCAAATGTAAGTACTGTGGAAATGATTTTAATTGCGAAACGAAGACAAACGGGACTTCATCTATGAAAAAACAT
TTGGAGAAGGAGCATTCCGTGACTTGCACGAATAAATCTGCAGTGCACCCCCCAAACACTTCAAGCACCGGCGATGCTACTTGTAATGTG
AGGTCGGTTGAAGTTGGTAGTTCGTCCAACGGAAAAAGAAAGAGAACAAATGAGGATCCAACGCAGACCACCGCAGCTAACATACACGCC
CAATGGGACAAGGCTGAGTTATCCAATAGGATAATTAAAATTACTGAGAAGTTACAGTTACAGGACATCCAGGGGGCTTTGAGTAAAGTT
CTCGAGCCATATGGATCCAGCGCTACTTCAAGTTCAAATCATCACCGCTTGAGTACAGCATCGAATCAGCACCCAACAACATCAAGTCTT
GTTCCAATGGAAGTTTATGGCAGAGTTGCAGAAAAGAATAAGATCAAAAAGTCAATAACTGAAAACCAATCTGGTGGTGTAAATGTTCTG
CCTATTGTAGGCATTGCAGGTGTTGGAAAGACAACTCTTGCTCAATTTGTGTATAATGATCCAGACGTGAAAAGTCAATTTCACCACAGG
ATATGGGTTTGTGTGTCCTGCAAATTTGATGAAGTGAAGCTCACAAAGGAGATGTTAGACTTTTTTCCTCGAGAAAGGCATGAAGGAATT
AACAACTTCGCGAAGCTTCAAGAGATCTTGAAAGAACATGTCGAGTACCAAGCAAAGAGTTTTCTGCTCATTTTAGATGATGTCTCGGAC
AGTATGGATTATCATAAATGGAACAAATTGTTGAACCCTTTGCTATCAAGTCAAGCGAAGAATATAATTCTAGTCACGACCAGAAATTTG
TCTGTTGCACAAAGGTTAAGCACACTTGAACCGATCAAGTTAGGTGCTTTAGAAAACGATGATATGTGGTTATTGCTCAAGTCATGTGCA
TTTGGTTTTGGGAACTATGAAGGTACGGAAAATCTAAGCACTATTGGAAGACAAATAGCAGAGAAGTTAAAGGGCAATCCGTTAGCAGCA
GTAACTGCAGGGGCACTGTTAAGAGATAATCTTAGCATTGATCATTGGAGTAACATTCTCAAGAATGAGAAGTGGAAATCGCTGGGACTC
AGTGGGGGCATCATGCCTGCTTTGAAGCTTAGTTATGATGAGTTGACGTACCGTTTACAACAATGTTTCTCGTATTGCTCTATATTTCCT
GACAAATATAGGTTTCTCGGGAAGGATTTGGTCTATATTTGGATTTCTCAGGGATTTGTGAATTGCACCCAAAATAAGAGATTGGAGGAG
ACGGGATGGGAATATCTGAATCAATTGGTAAATCTTGGATTCTTTCAACAAATTGAAGAACAACAAGAATTGGATGGGGAAGAAGAATTC
TCTCTACGCCGTCAGATTTGGTACTCTATGTGTGATCTCATGCATGATTTCGCAAGGATGATTTCAAGGACTGAATGTGCGACTATAGAT
GGTCTACAGTGCAATAAAATATTCCCAACTGTACAGCATTTGTCAATAGTAACCGGTTCTGCATACAACAAAGATCTGAAGGGGAACATT
CCTCGTAATGAGAAGTTTGAAGAAAATATGAGAAATTCAGTTACATCAGTTACCAAATTGAGAACATTGGTTGTGCTTGGGAACTTTGAC
TCTTTCTTTGTACGGTTGTTCCAAGATATATTCCAGAAGGCACAAAATTTACGCCTGCTGCTAGTATCTCTAGCATCCACTTATCTGTCT
CAAGTGCCTGCTGCATTCAATGATTTTAATTCCTTCCTGTGCAATTTGGCAAATCCTTTGCATCTTCGTTACCTAAAACTTGAGTTGGAT
GGGATTGTGCCACAAGTTTTGAGTACGTTTTTTCATCTTCAAGTATTAGATGTTGGATCAAGCATGGATACTTCTCTACCCAATGGCTTG
TTGCATAATCTTGTTAGCCTGCGACATCTTGTTGCACACAAGAGAGTCCATTCTTCCATTACTAGCATTGGTAACATGACATCTATCCAG
GAGCTACATGATTTTGAAGTTCGAATTTCTAGCGGCTTTGAGATAACACGACTCCAATCCATGAACGAGCTTGTTCAACTTGGGTTGTCT
CAACTTGACAGTGTTAAAACCAGGGAGGACGCTTATGGGGCAGGACTAAGAAACAAGGAACACTTAGAAGAGCTTCATTTGTCCTGGAAG
GATGCATATTCAGAGTATGAGTATGCCAGTGACACTGAATTTGAATCTTCTGCAAACATGGCAAGAGAAGTGATTGAGGGTCTTGAACCA
CACATGGATTTAAAACATCTACAAATATCTCAGTATAATGGTACCACTTCACCAGCTTGGCTTGCCAACAATATCTCAGTTACCTCATTG
CAGACGCTTCATCTTGATGATTGTGGAGGATGGAGAATACTTCCATCTCTGGGAAGTCTTCCATTCCTTACAAAGGTGAAGTTGAGCAGC
ATGCTGGAAGTAATTGAAGTACTGATTCCTTCACTGGAGGAGCTAGTTCTAATTAAAATGCCGAAGTTAGTGAGATGCTCAAGCACTTCT
GCCGAGGGTCTGAGCTCTAGCTTAAGGGTACTGCACATTGAGGATTGTGAAGCATTGAAGGAGTTTGATCTGTTTGAGAACGATTATAAT
TCTGAAATCATTCAGGGATCATGGCTGCCTGGTCTTAGGAATTTGATTCTATATTGTTGCCCTCATTTGAAAGTGTTGAAGCCTCTTCCA
CCTTCAACTACCTTTTCTAAGGTACTCATCAGAGAAATTTCAAGATTTCCGTCTATGGAGGTATCATCTGGTGAGAAGTTACAAATTGGG
AATATTGATGTGTACATAGGCGATGATTTTGATGAGTCTTCTGATGAGTTGAGCATACTGGATGACAAAACTTTGGCGTTCCATAATCTT
AGAAACCTGAAATCGATGGAGATATATGGTTGCAGAAATCTAAGGTCTTTTTCGTTCGAAGGTTTCAGTCATCTTGTCTCTTTAACAAGT
TTGAAAATAGTAAGCTGTGAACAACTTTTCCCTTCAGATGTGACGGCAGAGTATACCCTTGAAGATGTGACAGCTGTGAACTGCAATGCC
TTCCCATATCTTAAAAGCCTCAGTATCGACTCATGTGGAATAGCGGGGAAGTGGCTATCGCTGATGCTGCAGCATGCGCCAGGCCTAGAG
GAATTGAGTTTAACAAGTTGCGCCCATATAACAAGAGTAGTGTTACCGATGGAAGAGGAAGAAAACAATCTATTAACAACAGTACTGTCA
TCAGGAAATCAAGATGAGGCATTGACATGGTTAGTTCGTGACGGACTCTTGCACATTCCATCAAATCTCGTCTCCTCTCTCAAGAATATG
AGTATTACTCAGTGCCCTCGCCTAAAGTTTAACTCAGGCAAGGACTGCTTCTCTGGATTTACCTCGCTTGAGAAGCTTGAAATTTGGGGA
TCGTTGGTGGATGATGACGGAAGTGATGACCTGGAGAATGGAAGTTCTTTTGTGTTCGGAGAGGAGGATCAACCCCTGGGGGCGAACGGA
AGATGGCTCCTCCCGACATCACTTCAGGAACTTCACATCGTGTCATTGTATTGCCAAGAAACGCTGCAAGTCTGCTTCCCTAGAGATATC
ACCAGCCTTAAAAAGTTAAGTGTACGTTCCGGCCAAGGTTTGCAATCTCTACAGCTGTACTCATGCACGGCACTGGAAGAATTGGCAATT
TCCGGCTCTGGATCGGTCACCGTCACTGTACTAGAGGGCACGCAACCCGCTGGCAGCCTCGGGCGTTTGAATGTATCAGACTGTCCTGGC
TTGCCATCACGTTTGGACAGCTTTCCAAGGTTGTGCCCTCGGCTGGAAAGGCTTGACATCAATGACCCATCTGTCCTTACCACGCCATTC
TGCAAGCACCTCACCTCCCTGCAACGCCTAAAACTTGGCTTCTTGAAAGTGACGAGACTAACAGATGAGCAAGAACGAGCGCTTGTGCTC
CTCAAGTCACTGAAAGAGCTCGAGATTTTTTATTGTACTCATCTCATAGATCTTCCTGCGGGGCTGCAGACCCTTCCTTCCCTCAAGAGT
TTGAAGATAGAAGAGGGTCGAGGCATCTCAAGGCTGCCGGAAGCAGGCCTCCCACATTCGCTGGAAGAACTGGAAATCAAAATTTGCAGC
AAGCTAGAAGATGAATGCAGGCGGCTAGCAACATGCGAAGGCAAGCTAAAAGTCAAAATTGATGGTCGATATGTGAATTAA
>Yr7_protein
(SEQ ID NO: 3)
MEPAGDSSLEAAIAWLVQTILATLLMDKMEAWIQQVGLADDVERLQSEVERVDTVVAAVKGRAAGNMPLSRSLARVKELLYDADDVIDEL
DYYRLQHQVEGVTSDEPDGMRGAERVDEISRGHVDTLNVSVGKLRSPVWEHFTITETTIDGKRSKAKCKYCGNDFNCETKTNGTSSMKKH
LEKEHSVICTNKSAVHETNTSSTGDATCNVRSVEVGSSSNGKRKRTNEDDTQTTAANIHAQWDKADLSNRIIKITEKLQLQDIQGALSKV
LEPYGSSATSSSNHHRLSTASDQHPTTSSLVPMEVYGRVAEKNKIKKSITENQSGGVNVLPIVGIAGVGKTTLAQFVYNDPDVKSQFHHR
IWVCVSCKFDEVELTKEMLDFFPRERHEGINNFAKLQEILKEEVEYQAKSFLLILDDVSDSMDYEKWNKLLNPLLSSQAKNIILVTTRNL
SVAQRLSTLEPIKLGALENDDMWLLLKSCAFGFGNYEGTENLSTIGRQIAEKLKGNPLAAVTAGALLEDNLSIDEWSNILKNEKWKSLGL
SGGIMPALKLSYDELTYRLQQCFSYCSIFPDKYRFLGKDLVYIWISQGFVNCTQNKRLEETGWEYLNQLVNLGPPQQIEEQQELDGEEEP
SLRRQIWYSMCDLMHDFARMISRTECATIDGLQCNKIFETVQHLSIVTGSAYNKPLKGNIPRNEKKEDNMRNSVISVTKLRTLVVLGNED

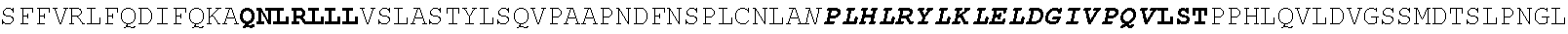

LHNLVSLRHLVAHKRVHSSITSIGNMTSIQELHDPEVRISSGFEITRLQSMNELVQLGLSQLDSVKTREDAYGAGLRNKEHLEELHLSWK
DAYSEYEYASDTEFESSANMAREVIEGLEPHMDLKHLQISQYNGTTSPAWLANNISVTSLQTLHLDDCGGWRILPSLGSLPFLTKVKLSS

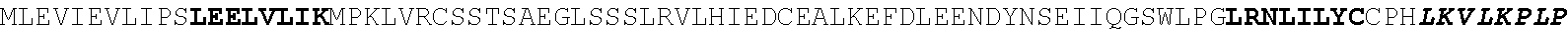

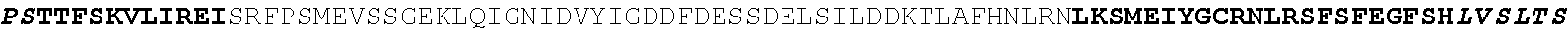

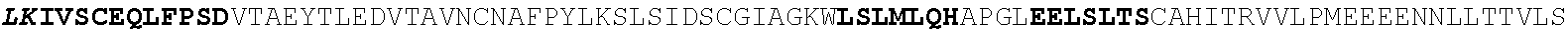

SGNQDEALTWLVADGLLHIPSNLVSSLKNMSITQCPRLKFNSGKDCFSGFTSLEKLEIWGSLVDDDGSDDLENGSSFVFGEEDQPLGANG
RWLLPTSLQELHIVSLYCQETLQVCFPRDITSLKKLSVRSGQGLQSLQLYSCTALEELAISGSGSVIVTVLEGTQPAGSLGRLNVSDCPG
LPSRLDSFPRLCPRLERLDINDPSVLTTPFCKHLTSLQRLKLGFLKVTRLTDEQERALVLLKSLKELEIPYCTHLIDLPAGLQTLPSLKS
LKIEEGRGISRLPEAGLPHSLEELEIKICSKLEDECRRLATCEGKLKVKIDGRYVN-
>Yr5_locus
(SEQ ID NO: 4)
ATGGAGCCGGCGGGAGACTCTTCCGTGGAGGCCGCGATTGCATGGCTGGTGCAGACCATCCTTGCAACGCTCCTCATGGACAAGATGGAG
GAGTGGATTCGGCAAGTCGGGCTTGCCGACGACGTCGAGAGGCTCCAGTCTGAGGTCGAGAGAGTCGACACGGTGGTGGCTGCTGTGAAG
GGGAGGGCAGCCGGGAACAGGCCTCTGTCCCGGGCTCTCGCTCGTGTCAAGGAGCTTCTCTACGACGCCGACGACTTGATCGACGAGCTA
GACTACTACAGGCTCCAACAACAAGTCGAAGGAGGTAGTAAGCATAATCCCATTATATATCGAATATATGTAAGCTCAAGATATTTATTT
TGGGATGGAGGGAGTAGTTTGATCTTAATTTCTGGTCCATATTTTTTTCGGCACAGTTACGAGTGACGACCCTGACGGTATGCGTGGAGC
TGAAAGAGTGGATGAAATATCAAGGGGCCATGTCGATACACTGAATTGCAGTGTTGGCAAATTACGATCCCCGGTATGGGAACACTTCAC
GATCACAGAAACAACTATCGACGGGAAGCGTTCAAAAGCCAAATGTAACTACTGTGGAAATGATTTTAATTGCGAAACGAAGACAAACGG
GACTTCATCTATGAAAAAACATTTGGAGAAAGAGCATTCCGTGACTTGTACGAAGAAACCTGGAGCCCATCCACCAAACCCTTCAAGGTA
CCCAAAGGAAATTATATGTTGCATCAGCGCATTTATATTCGTTTATATATATCTGCTTGAGAGCCCATTGTTGTTCTACATTTCTTCTGA
TAACTGACCCACCATTTTCTCTCTTAATGCAGCACCGGCTATGCAACTGAAAATGTGACGCTTGTTGAAGTTGGTAGTTCATCCAACAGA
AAAAGAAAGAGAACGAATAAGGAGCCAGCACAAACCACCGCAGATAACACCCGTTGGGACAAGGCTGAGTTATCCGATACAATAAAAAAG
ATTACTAGCCAGTTACAGTTACAGTTACAGGGTATCCTATGGGCTTTCAGTAAAGTTCTCGAGCCACATGGGTCTAGCTCTGCGTCGAGT
TCAAATCATCACCAACCGAGTACAACCTCAGATCAGCACGCAAAAACATCAAGTCTTGCTCCAAGGAAAGTGTATGGCAGAGTAGCAGAA
ATGAACTCCATCAGAAATTTAATAGCAGAAAAGAAATGTGATGCTCTAACTGTTCTGCCTATTGTGgGCATTGCTGGTGTTGGAAAGACA
ACTCTCGCTCAATCTGTATACAATGATCCAGATATAAAAAGTCAATTTCACCACAAGATATGGGTTTGCGTGTCCCGCAAATTTGATGAA
GTGATGCTCACAAGGGAGATGTTAGACTTTGAAAGACACGAGGGATCTCCTCATGAAAATGGAAGGCATGAAGGAATTAGTAGCCTTGCT
AAGCTTCAGGAGATCTTGAAGGACATTATCGAGTACCAGTCAAAGAGTTTTCTGCTTATTTTAGATGATGTATGGGACAGTATGGATGAT
CATCAATGGAGAAAACTGGTGTGTCCTTTTGTATCAAGTCAAGCAAAGGGTAATTTAATTCTAGTCACAACCAGAAATTTGTCAGTTGCA
CACATGTTAGGAACACGTGAGCCGATAAAGTTGGGTGCTTTGGAAAATGATGTTATGTGGTTGCTGCTCAAGTCATGTGCATTTCGTGAT
GTGAATTATGAAGGGAACCAAAGTCTAAGCATTGTCGgGAGGCAAATATCAGAGAAGTTAAAGGGAAACCCACTAGCAGCAGAAACAGCG
GGGGCACTATTAAGGAAGAAATTTAGCATTGATTATTGGAAAATCATTTTAAAGAATGAAGACTGGAAATCCATGGAGCTCGGTAATGGA
ATCATGGCTGCTCTAAAGCTTAGCTATGATCAACTTCCCTACCATTTACAACAATGTTTCTCATATTGCTCCATATTCCCCGACGGTTAT
CAGTTTCTTGGTGAGGAGTTGGTCGGTTTCTGGATGTCACAGGGATTTGTAAAGTGCAACAACTCTAGTCAGAGATTGGAGCAGATAGGA
CAGTGCTATCTGATTGATTTGGTTAACTTAGGCTTCTTTGAAGAAGTTAAAAGAGAAGAACCATATCTGGGCTGTCGAGTTATGTATGGC
ATATGTGGTCTCATGCATGATTTTGTGATTATGGTGTCAAGGACTGACTGTGCAAGTATAGATGGTCTGCAGCGCAACAAAATGCCTCAA
ACTCTACGACATTTGTCAATAGTAACTGGATCCGCGTACAAGAAAAATCAGCACGGAAACATTCCTCGTAATAATAGGTTTGAAGAAAAT
CTGAGAAATACAATTACATCAGTTAGCGAGTTGAGGACATTGGTGTTACTTGGGCATTATGACTTTTCCTTCTTACTATTATTCCAAGAT
ATATTTCAAAAGGCACATAACTTACGTGTGCTGCAAATGTCTGCAGCACCTGCTGATTTTCTCAAACATAGGTTTGAGGAGGTGGATGGG
TCTTTCCCTCAAATTTTGAGCAAATTGTACCATCTCCAAGTATTAGACGTCGGTGCATACACTGATCGTACTATGCCTGGTTGTATTGAT
AATCTTGTTAGCCTGCGGCATCTTGTTGTACACAAGGGAGTGTACTCTTCCATTGCAACCATTGATAATATGCTATCATTTCAGGAACAA
CATGGTTTCAAGTTTCATATTTCTAGTGGCTTTGAGATAACACGACTCCAATCCACTGAACATTGGATGCATGTTGATACTCTGGAAGAT
GTTTATGAGGCAGGACTGGTAAACAATGAACTCTCAGAAAAGTTGCACCTGTCCTGGAAGGATTCTCCTGAGGACATAGGCATGGAGGTT
GAGGATTGGGAACCACATTGGGACTTAAGGGTTcTCGAGATATCTGGGTATAATTTTGGTTCGCCAATTGTGGTTGACATCATTATCTTG
GTTACATCCTCCCAGACGGTTGAGATATCCAATTGTAGTGAATGGAAAATACTTCCATCTTTGGAAAGATTTCAGTTTTTGACAAATCTG
GAGTTGAGAAACCTGCCCAAAGTAATAGAAATACTGGTTCCTTCACTGGAGGAGCTAGCATTAGTTACAATGCCAAAGTTGAAGAAATGT
TCATGCACTCCCGTGGAAGGTATGAGCTCTAGACTAAGAGCACTGCGGATCGAGGATTGTCAATCACTGAAGGAGTTTGATCTGTTTGAG
AACAATGATAAATTCGAAACTGGGCAGAGGTCATGGGCTCCTAGTCTTAGGGAACTAAGTCTGGAGAATTGCCCCCATTTGAAAGTGTTG
AAGCCTCTTCCACTCTCACTCATGTGTTCTGAGTTACTCATAAGTGGAGTTTCAACACTTCCGTACATGAAGGGGTCATCTGATAGAAAG
TTATGTATTGGGTATGATGATAAGTATGACTACTATGGTTTTGACGAATCTTCCgATGAGTTGAAGATACTGGATGACAAAATTTTTATG
TTCCATAATCTGAAAAACCTCAAATCAATGGTGATATATGGTTGCCGGAATCTAAGTTCCATTTCGTTAAAAGGTTTTAGTTACCTCATC
TCTTTAACGAGCTTGGAAATAAGAGACTGTGAAAAACTTTTTGCTTCAGATGAGATGCCAGAGCATACCCTTGAAGATGTGACACCTGCG
AATTGCAAGGCTTTCCCATCTCTTGAATGTCTCAGTATTGATTCATGTGGTATAGTGGGGAAGTGGCTATCTCTGATGCTGCAACATGCG
CCATGCCTAGAGGAGTTGTATTTGTCTTCCCGAGAGGAAGAAAATTCAGAAGAAGAAAATTCAGAAGAGGAAGAAAACAGTATATCAAAT
CTTAGCTCAACCAGGGAGGGCACATCATCCGGAAATCCAGATGACGGATTAGCTCTAGACCGACTGTTGCGCATACCATTAAATCTCATC
TCCATTCTAAAGAGTATAACTATTGAGAGATGCCCTCATCTAACATTTAACTGGGGCAAGGAAGGCGTCTCGGGATTTACCTCCCTTGAG
AAGCTAATCGTTTTGGACCGCCCCGACATGGTGCTTACAAACGGAAGATGGCTCCTCCCAAACTCACTTGGCGAACTTGAAAGCAATGAC
TATTCCCGAGGAACGCTGCAACCCTGCTTTCCTAGCGATATCACTAGCCTTAAAAAGTTAAAGGTACGTCGCAGCCCAGGTTTGCAATCT
CTACAGCTGCACTCATGCATGGCACTGGAAGAATTGGATATTCAAGATTGTCGAAGGCTCGCTGCACTGCAGGGTCTGCAATTCCTTGGC

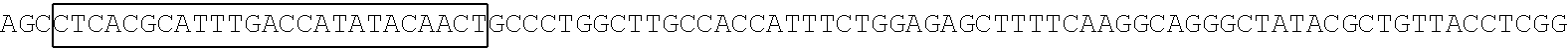

CTGAAAAGGCTTCACATCCAAGACCCATCTGTCCTTACCACGTCATTCTGCAGGCACCTTACCTCCCTGCAACACCTAAAACTTACTTGG
TTGGAAGAAGTGAGACTAACAGATGAGCAAGAGCAAGCGCTTGTGCTCCTCAAGTCCCTGCAAGAGCTCCAATTTCATTATTGTTCCAAT
CTCGTAGATCTTCCTGCGGTGCTGCACAACCTTCCTTCCCTGAAGACTTTGAAGGTAGATGGGTGTAGGGGCATCTCAAGGCTGCCAGAA
ACAGGCCTCCCATTTTCGCTGGAAGAACTGGAAATCGAGTGGTGCAGCAAGGAGCTCGCTGATCAATGCAGGCTGCTAGCATCAAACAAG

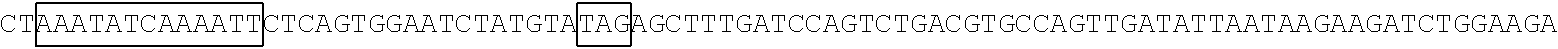

TGAAGATACCTCTTAAGAATAAAATCTTTGCATGGTATCTTCGTCGCGGAGTCATTCTTACTAAAGATAACCTTATTAAGAGAAATTGGC
ATGGAAGTACGCAATGTGTATTTTGTCCGCATGATGAGACAATAAAACATTTGTTCTTCCAATGTAAATTGGCTCGTTCTATATGGTCAG
TCATCCAAATAGCTTCTGGCTTGTACCCTCCTTGTAGTGTTGCTAATATATTTGGCAATTGGTTACATGGGATTGATCACAAGTTCAGAA
GTCTACTTAGGGTGGGAGCGCTTGCCGTGATTTGGTCGCTTTGGCTATGTAGAAATGATAAGATTTTTAACGATAAAAGTACTTCGCTTA
TGCAGGTTATCTACAGATGTACTGGGACGCTTCGTTTATGGTCCTCTCTACAACGAGTGGAGAATCGAGACCTGTTTACGGAGGTGTGTA
CACGATTGGAGGTTACGGCGAGGGATACTTTTATCCAACATGGGTGGCGGCATGATCTTAGGATTGGGCCACCGACGGTTTAGGCGCTAT
ACAAATATACTTTCTTTGTATTTCGCCTTCCTTTTTTATTTTTATTTTTCGCTTGTTGTGAGGATATTGTTGGCTGTGTGCATCTCAGTT

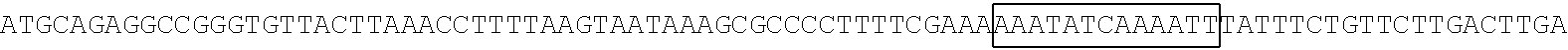

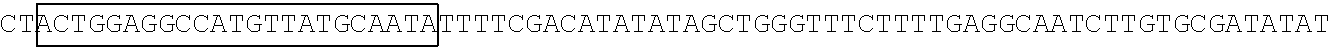

>YrSP_locus
(SEQ ID NO 7)
ATGGAGCCGGCGGGAGACTCTTCCGTGGAGGCCGCGATTGCATGGCTGGTGCAGACCATCCTTGCAACGCTCCTCATGGACAAGATGGAG
GAGTGGATTCGGCAAGTCGGGCTTGCCGACGACGTCGAGAGGCTCCAGTCTGAGGTCGAGAGAGTCGACACGGTGGTGGCTGCTGTGAAG
GGGAGGGCAGCCGGGAACAGGCCTCTGTCCCGGGCTCTCGCTCGTGTCAAGGAGCTTCTCTACGACGCCGACGACTTGATCGACGAGCTA
GACTACTACAGGCTCCAACAACAAGTCGAAGGAGGTAGTAAGCATAATCCCATTATATATCGAATATATGTAAGCTCAAGATATTTATTT
TGGGATGGAGGGAGTAGTTTGATCTTAATTTCTGGTCCATATTTTTTTCGGCACAGTTACGAGTGACGACCCTGACGGTATGCGTGGAGC
TGAAAGAGTGGATGAAATATCAAGGGGCCATGTCGATACACTGAATTGCAGTGTTGGCAAATTACGATCCCCGGTATGGGAACACTTCAC
GATCACAGAAACAACTATCGACGGGAAGCGTTCAAAAGCCAAATGTAACTACTGTGGAAATGATTTTAATTGCGAAACGAAGACAAACGG
GACTTCATCTATGAAAAAACATTTGGAGAAAGAGCATTCCGTGACTTGTACGAAGAAACCTGGAGCCCATCCACCAAACCCTTCAAGGTA
CCCAAAGGAAATTATATGTTGCATCAGCGCATTTATATTCGTTTATATATATCTGCTTGAGAGCCCATTGTTGTTCTACATTTCTTCTGA
TAACTGACCCACCATTTTCTCTCTTAATGCAGCACCGGCTATGCAACTGAAAATGTGACGCTTGTTGAAGTTGGTAGTTCATCCAACAGA
AAAAGAAAGAGAACGAATAAGGAGCCAGCACAAACCACCGCAGATAACACCCGTTGGGACAAGGCTGAGTTATCCGATACAATAAAAAAG
ATTACTAGCCAGTTACAGTTACAGTTACAGGGTATCCTATGGGCTTTCAGTAAAGTTCTCGAGCCACATGGGTCTAGCTCTGCGTCGAGT
TCAAATCATCACCAACCGAGTACAACCTCAGATCAGCACGCAAAAACATCAAGTCTTGCTCCAAGGAAAGTGTATGGCAGAGTAGCAGAA
ATGAACTCCATCAGAAATTTAATAGCAGAAAAGAAATGTGATGCTCTAACTGTTCTGCCTATTGTGGGCATTGCTGGTGTTGGAAAGACA
ACTCTCGCTCAATCTGTATACAATGATCCAGATATAAAAAGTCAATTTCACCACAAGATATGGGTTTGCGTGTCCCGCAAATTTGATGAA
GTGATGCTCACAAGGGAGATGTTAGACTTTGAAAGACACGAGGGATCTCCTCATGAAAATGGAAGGCATGAAGGAATTAGTAGCCTTGCT
AAGCTTCAGGAGATCTTGAAGGACATTATCGAGTACCAGTCAAAGAGTTTTCTGCTTATTTTAGATGATGTATGGGACAGTATGGATGAT
CATCAATGGAGAAAACTGGTGTGTCCTTTTGTATCAAGTCAAGCAAAGGGTAATTTAATTCTAGTCACAACCAGAAATTTGTCAGTTGCA
CACATGTTAGGAACACGTGAGCCGATAAAGTTGGGTGCTTTGGAAAATGATGTTATGTGGTTGCTGCTCAAGTCATGTGCATTTCGTGAT
GTGAATTATGAAGGGAACCAAAGTCTAAGCATTGTCGGGAGGCAAATATCAGAGAAGTTAAAGGGAAACCCACTAGCAGCAGAAACAGCG
GGGGCACTATTAAGGAAGAAATTTAGCATTGATTATTGGAAAATCATTTTAAAGAATGAAGAGTGGAAATCCATGGAGCTCGGTAATGGA
ATCATGGCTGCTCTAAAGCTTAGCTATGATCAACTTCCCTACCATTTACAACAATGTTTCTCATATTGCTCCATATTCCCCGACGGTTAT
CAGTTTCTTGGTGAGGAGTTGGTCGGTTTCTGGATGTCACAGGGATTTGTAAAGTGCAACAACTCTAGTCAGAGATTGGAGCAGATAGGA
CAGTGCTATCTGATTGATTTGGTTAACTTAGGCTTCTTTGAAGAAGTTAAAAGAGAAGAACCATATCTGGGCTGTCGAGTTATGTATGGC
ATATGTGGTCTCATGCATGATTTTGTGATTATGGTGTCAAGGACTGACTGTGCAAGTATAGATGGTCTGCAGCGCAACAAAATGCCTCAA
ACTCTACGACATTTGTCAATAGTAACTGGATCCGCGTACAAGAAAAATCAGCACGGAAACATTCCTCGTAATAATAGGTTTGAAGAAAAT
CTGAGAAATACAATTACATCAGTTAGCGAGTTGAGGACATTGGTGTTACTTGGGCATTATGACTTTTCCTTCTTACTATTATTCCAAGAT
ATATTTCAAAAGGCACATAACTTACGTGTGCTGCAAATGTCTGCACCACCTGCTGATTTTCTCAAACATAGGTTTGAGGAGGTGGATGGG
TCTTTCCCTCAAATTTTGAGCAAATTGTACCATCTCCAAGTATTAGACGTCGGTGCATACACTGATCGTACTATGCCTGGTTGTATTGAT
AATCTTGTTAGCCTGCGGCATCTTGTTGTACACAAGGGAGTGTACTCTTCCATTGCAACCATTGATAATATGCTATCATTTCAGGAACAA
CATGGTTTCAAGTTTCATATTTCTAGTGGCTTTGAGATAACACGACTCCAATCCACTGAACATTGGATGCATGTTGATACTCTGGAAGAT
GTTTATGAGGCAGGACTGGTAAACAATGAACTCTCAGAAAAGTTGCACCTGTCCTGGAAGATTCTCCTGAGGACATAGGCATGGAGGTTG
AGGATTGGGAACCACATTGGGACTTAAGGGTTCTCGAGATATCTGGGTATAATTTTGGTTCGCCAATTGTGGTTGACATCATTATCTTGG
TTACATCCTCCCAGACGGTTGAGATATCCAATTGTAGTGAATGGAAAATACTTCCATCTTTGGAAAGATTTCAGTTTTTGACAAATCTGG
AGTTGAGAAACCTGCCCAAAGTAATAGAAATACTGGTTCCTTCACTGGAGGAGCTAGCATTAGTTACAATGCCAAAGTTGAAGAAATGTT
CATGCACTCCCGTGGAAGGTATGAGCTCTAGACTAAGAGCACTGCGGATCGAGGATTGTCAATCACTGAAGGAGTTTGATCTGTTTGAGA
ACAATGATAAATTCGAAACTGGGCAGAGGTCATGGGCTCCTAGTCTTAGGGAACTAAGTCTGGAGAATTGCCCCCATTTGAAAGTGTTGA
AGCCTCTTCCACTCTCACTCATGTGTTCTGAGTTACTCATAAGTGGAGTTTCAACACTTCCGTACATGAAGGGGTCATCTGATAGAAAGT
TATGTATTGGGTATGATGATAAGTATGACTACTATGGTTTTGACGAATCTTCCGATGAGTTGAAGATACTGGATGACAAAATTTTTATGT
TCCATAATCTGAAAAACCTCAAATCAATGGTGATATATGGTTGCCGGAATCTAAGTTCCATTTCGTTAAAAGGTTTTAGTTACCTCATCT
CTTTAACGAGCTTGGAAATAAGAGACTGTGAAAAACTTTTTGCTTCAGATGAGATGCCAGAGCATACCCTTGAAGATGTGACACCTGCGA
ATTGCAAGGCTTTCCCATCTCTTGAATGTCTCAGTATTGATTCATGTGGTATAGTGGGGAAGTGGCTATCTCTGATGCTGCAACATGCGC
CATGCCTAGAGGAGTTGTATTTGTCTTCCCGAGAGGAAGAAAATTCAGAAGAAGAAAATTCAGAAGAGGAAGAAAACAGTATATCAAATC
TTAGCTCAACCAGGGAGGGCACATCATCCGGAAATCCAGATGACGGATTAGCTCTAGACCGACTGTTGCGCATACCATTAAATCTCATCT
CCATTCTAAAGAGTATAACTATTGAGAGATGCCCTCATCTAACATTTAACTGGGGCAAGGAAGGCGTCTCGGGATTTACCTCCCTTGAGA
AGCTAATCGTTTTGGACCGCCCCGACATGGTGCTTACAAACGGAAGATGGCTCCTCCCAAACTCACTTGGCGAACTTGAAAGCAATGACT
ATTCCCGAGGAACGCTGCAACCCTGCTTTCCTAGCGATATCACTAGCCTTAAAAAGTTAAAGGTACGTCGCAGCCCAGGTTTGCAATCTC
TACAGCTGCACTCATGCATGGCACTGGAAGAATTGGATATTCAAGATTGTCGAAGGCTCGCTGCACTGCAGGGTCTGCAATTCCTTGGCA

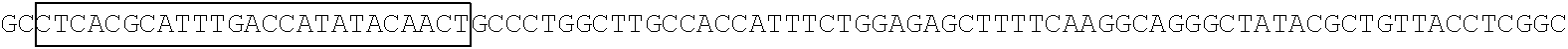

TGAAAAGGCTTCACATCCAAGACCCATCTGTCCTTACCACGTCATTCTGCAGGCACCTTACCTCCCTGCAACACCTAAAACTTACTTGGT
TGGAAGAAGTGAGACTAACAGATGAGCAAGAGCAAGCGCTTGTGCTCCTCAAGTCCCTGCAAGAGCTCCAATTTCATTATTGTTCCAATC
TCGTAGATCTTCCTGCGGTGCTGCACAACCTTCCTTCCCTGAAGACTTTGAAGGTAGATGGGTGTAGGGGCATCTCAAGGCTGCCAGAAA
CAGGCCTCCCATTTTCGCTGGAAGAACTGGAAATCGAGTGGTGCAGCAAGGAGCTCGCTGATCAATGCAGGCTGCTAGCATCAAACAAGC

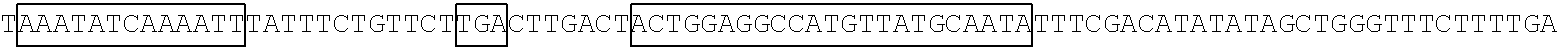

GGCAATCTTGTGCG
>Yr.5_CDS
ATGGAGCCGGCGGGAGACTCTTCCGTGGAGGCCGCGATTGCATGGCTGGTGCAGACCATCCTTGCAACGCTCCTCATGGACAAGATGGAG
GAGTGGATTCGGCAAGTCGGGCTTGCCGACGACGTCGAGAGGCTCCAGTCTGAGGTCGAGAGAGTCGACACGGTGGTGGCTGCTGTGAAG
GGGAGGGCAGCCGGGAACAGGCCTCTGTCCCGGGCTCTCGCTCGTGTCAAGGAGCTTCTCTACGACGCCGACGACTTGATCGACGAGCTA
GACTACTACAGGCTCCAACAACAAGTCGAAGGAGTTACGAGTGACGACCCTGACGGTATGCGTGGAGCTGAAAGAGTGGATGAAATATCA
AGGGGCCATGTCGATACACTGAATTGCAGTGTTGGCAAATTACGATCCCCGGTATGGGAACACTTCACGATCACAGAAACAACTATCGAC
GGGAAGCGTTCAAAAGCCAAATGTAACTACTGTGGAAATGATTTTAATTGCGAAACGAAGACAAACGGGACTTCATCTATGAAAAAACAT
TTGGAGAAAGAGCATTCCGTGACTTGTACGAAGAAACCTGGAGCCCATCCACCAAACCCTTCAAGCACCGGCTATGCAACTGAAAATGTG
ACGCTTGTTGAAGTTGGTAGTTCATCCAACAGAAAAAGAAAGAGAACGAATAAGGAGCCAGCACAAACCACCGCAGATAACACCCGTTGG
GACAAGGCTGAGTTATCCGATACAATAAAAAAGATTACTAGCCAGTTACAGTTACAGTTACAGGGTATCCTATGGGCTTTCAGTAAAGTT
CTCGAGCCACATGGGTCTAGCTCTGCGTCGAGTTCAAATCATCACCAACCGAGTACAACCTCAGATCAGCACGCAAAAACATCAAGTCTT
GCTCCAAGGAAAGTGTATGGCAGAGTAGCAGAAATGAACTCCATCAGAAATTTAATAGCAGAAAAGAAATGTGATGCTCTAACTGTTCTG
CCTATTGTGGGCATTGCTGGTGTTGGAAAGACAACTCTCGCTCAATCTGTATACAATGATCCAGATATAAAAAGTCAATTTCACCACAAG
ATATGGGTTTGCGTGTCCCGCAAATTTGATGAAGTGATGCTCACAAGGGAGATGTTAGACTTTGAAAGACACGAGGGATCTCCTCATGAA
AATGGAAGGCATGAAGGAATTAGTAGCCTTGCTAAGCTTCAGGAGATCTTGAAGGACATTATCGAGTACCAGTCAAAGAGTTTTCTGCTT
ATTTTAGATGATGTATGGGACAGTATGGATGATCATCAATGGAGAAAACTGGTGTGTCCTTTTGTATCAAGTCAAGCAAAGGGTAATTTA
ATTCTAGTCACAACCAGAAATTTGTCAGTTGCACACATGTTAGGAACACGTGAGCCGATAAAGTTGGGTGCTTTGGAAAATGATGTTATG
TGGTTGCTGCTCAAGTCATGTGCATTTCGTGATGTGAATTATGAAGGGAACCAAAGTCTAAGCATTGTCGGGAGGCAAATATCAGAGAAG
TTAAAGGGAAACCCACTAGCAGCAGAAACAGCGGGGGCACTATTAAGGAAGAAATTTAGCATTGATTATTGGAAAATCATTTTAAAGAAT
GAAGACTGGAAATCCATGGAGCTCGGTAATGGAATCATGGCTGCTCTAAAGCTTAGCTATGATCAACTTCCCTACCATTTACAACAATGT
TTCTCATATTGCTCCATATTCCCCGACGGTTATCAGTTTCTTGGTGAGGAGTTGGTCGGTTTCTGGATGTCACAGGGATTTGTAAAGTGC
AACAACTCTAGTCAGAGATTGGAGCAGATAGGACAGTGCTATCTGATTGATTTGGTTAACTTAGGCTTCTTTGAAGAAGTTAAAAGAGAA
GAACCATATCTGGGCTGTCGAGTTATGTATGGCATATGTGGTCTCATGCATGATTTTGTGATTATGGTGTCAAGGACTGACTGTGCAAGT
ATAGATGGTCTGCAGCGCAACAAAATGCCTCAAACTCTACGACATTTGTCAATAGTAACTGGATCCGCGTACAAGAAAAATCAGCACGGA
AACATTCCTCGTAATAATAGGTTTGAAGAAAATCTGAGAAATACAATTACATCAGTTAGCGAGTTGAGGACATTGGTGTTACTTGGGCAT
TATGACTTTTCCTTCTTACTATTATTCCAAGATATATTTCAAAAGGCACATAACTTACGTGTGCTGCAAATGTCTGCAGCACCTGCTGAT
TTTCTCAAACATAGGTTTGAGGAGGTGGATGGGTCTTTCCCTCAAATTTTGAGCAAATTGTACCATCTCCAAGTATTAGACGTCGGTGCA
TACACTGATCGTACTATGCCTGGTTGTATTGATAATCTTGTTAGCCTGCGGCATCTTGTTGTACACAAGGGAGTGTACTCTTCCATTGCA
ACCATTGATAATATGCTATCATTTCAGGAACAACATGGTTTCAAGTTTCATATTTCTAGTGGCTTTGAGATAACACGACTCCAATCCACT
GAACATTGGATGCATGTTGATACTCTGGAAGATGTTTATGAGGCAGGACTGGTAAACAATGAACTCTCAGAAAAGTTGCACCTGTCCTGG
AAGGATTCTCCTGAGGACATAGGCATGGAG
GTTGAGGATTGGGAACCACATTGGGACTTAAGGGTTCTCGAGATATCTGGGTATAATTTTGGTTCGCCAATTGTGGTTGACATCATTATC
TTGGTTACATCCTCCCAGACGGTTGAGATATCCAATTGTAGTGAATGGAAAATACTTCCATCTTTGGAAAGATTTCAGTTTTTGACAAAT
CTGGAGTTGAGAAACCTGCCCAAAGTAATAGAAATACTGGTTCCTTCACTGGAGGAGCTAGCATTAGTTACAATGCCAAAGTTGAAGAAA
TGTTCATGCACTCCCGTGGAAGGTATGAGCTCTAGACTAAGAGCACTGCGGATCGAGGATTGTCAATCACTGAAGGAGTTTGATCTGTTT
GAGAACAATGATAAATTCGAAACTGGGCAGAGGTCATGGGCTCCTAGTCTTAGGGAACTAAGTCTGGAGAATTGCCCCCATTTGAAAGTG
TTGAAGCCTCTTCCACTCTCACTCATGTGTTCTGAGTTACTCATAAGTGGAGTTTCAACACTTCCGTACATGAAGGGGTCATCTGATAGA
AAGTTATGTATTGGGTATGATGATAAGTATGACTACTATGGTTTTGACGAATCTTCCGATGAGTTGAAGATACTGGATGACAAAATTTTT
ATGTTCCATAATCTGAAAAACCTCAAATCAATGGTGATATATGGTTGCCGGAATCTAAGTTCCArrrCGTTAAAAGGrrTTAGTTACCTC
ATCTCTTTAACGAGCTTGGAAATAAGAGACTGTGAAAAACTTTTTGCTTCAGATGAGATGCCAGAGCATACCCTTGAAGATGTGACACCT
GCGAATTGCAAGGCTTTCCCATCTCTTGAATGTCTCAGTATTGATTCATGTGGTATAGTGGGGAAGTGGCTATCTCTGATGCTGCAACAT
GCGCCATGCCTAGAGGAGTTGTATTTGTCTTCCCGAGAGGAAGAAAATTCAGAAGAAGAAAATTCAGAAGAGGAAGAAAACAGTATATCA
AATCTTAGCTCAACCAGGGAGGGCACATCATCCGGAAATCCAGATGACGGATTAGCTCTAGACCGACTGTTGCGCATACCATTAAATCTC
ATCTCCATTCTAAAGAGTATAACTATTGAGAGATGCCCTCATCTAACATTTAACTGGGGCAAGGAAGGCGTCTCGGGATTTACCTCCCTT
GAGAAGCTAATCGTTTTGGACCGCCCCGACATGGTGCTTACAAACGGAAGATGGCTCCTCCCAAACTCACTTGGCGAACTTGAAAGCAAT
GACTATTCCCGAGGAACGCTGCAACCCTGCTTTCCTAGCGATATCACTAGCCTTAAAAAGTTAAAGGTACGTCGCAGCCCAGGTTTGCAA
TCTCTACAGCTGCACTCATGCATGGCACTGGAAGAATTGGATATTCAAGATTGTCGAAGGCTCGCTGCACTGCAGGGTCTGCAATTCCTT
GGCAGCCTCACGCATTTGACCATATACAACTGCCCTGGCTTGCCACCATTTCTGGAGAGCTTTTCAAGGCAGGGCTATACGCTGTTACCT
CGGCTGAAAAGGCTTCACATCCAAGACCCATCTGTCCTTACCACGTCATTCTGCAGGCACCTTACCTCCCTGCAACACCTAAAACTTACT
TGGTTGGAAGAAGTGAGACTAACAGATGAGCAAGAGCAAGCGCTTGTGCTCCTCAAGTCCCTGCAAGAGCTCCAATTTCATTATTGTTCC
AATCTCGTAGATCTTCCTGCGGTGCTGCACAACCTTCCTTCCCTGAAGACTTTGAAGGTAGATGGGTGTAGGGGCATCTCAAGGCTGCCA
GAAACAGGCCTCCCATTTTCGCTGGAAGAACTGGAAATCGAGTGGTGCAGCAAGGAGCTCGCTGATCAATGCAGGCTGCTAGCATCAAAC
AAGCTAAATATCAAAATTCTCAGTGGAATCTATGTATAG
>YrSP_CDS
ATGGAGCCGGCGGGAGACTCTTCCGTGGAGGCCGCGATTGCATGGCTGGTGCAGACCATCCTTGCAACGCTCCTCATGGACAAGATGGAG
GAGTGGATTCGGCAAGTCGGGCTTGCCGACGACGTCGAGAGGCTCCAGTCTGAGGTCGAGAGAGTCGACACGGTGGTGGCTGCTGTGAAG
GGGAGGGCAGCCGGGAACAGGCCTCTGTCCCGGGCTCTCGCTCGTGTCAAGGAGCTTCTCTACGACGCCGACGACTTGATCGACGAGCTA
GACTACTACAGGCTCCAACAACAAGTCGAAGGAGTTACGAGTGACGACCCTGACGGTATGCGTGGAGCTGAAAGAGTGGATGAAATATCA
AGGGGCCATGTCGATACACTGAATTGCAGTGTTGGCAAATTACGATCCCCGGTATGGGAACACTTCACGATCACAGAAACAACTATCGAC
GGGAAGCGTTCAAAAGCCAAATGTAACTACTGTGGAAATGATTTTAATTGCGAAACGAAGACAAACGGGACTTCATCTATGAAAAAACAT
TTGGAGAAAGAGCATTCCGTGACTTGTACGAAGAAACCTGGAGCCCATCCACCAAACCCTTCAAGCACCGGCTATGCAACTGAAAATGTG
ACGCTTGTTGAAGTTGGTAGTTCATCCAACAGAAAAAGAAAGAGAACGAATAAGGAGCCAGCACAAACCACCGCAGATAACACCCGTTGG
GACAAGGCTGAGTTATCCGATACAATAAAAAAGATTACTAGCCAGTTACAGTTACAGTTACAGGGTATCCTATGGGCTTTCAGTAAAGTT
CTCGAGCCACATGGGTCTAGCTCTGCGTCGAGTTCAAATCATCACCAACCGAGTACAACCTCAGATCAGCACGCAAAAACATCAAGTCTT
GCTCCAAGGAAAGTGTATGGCAGAGTAGCAGAAATGAACTCCATCAGAAATTTAATAGCAGAAAAGAAATGTGATGCTCTAACTGTTCTG
CCTATTGTGGGCATTGCTGGTGTTGGAAAGACAACTCTCGCTCAATCTGTATACAATGATCCAGATATAAAAAGTCAATTTCACCACAAG
ATATGGGTTTGCGTGTCCCGCAAATTTGATGAAGTGATGCTCACAAGGGAGATGTTAGACTTTGAAAGACACGAGGGATCTCCTCATGAA
AATGGAAGGCATGAAGGAATTAGTAGCCTTGCTAAGCTTCAGGAGATCTTGAAGGACATTATCGAGTACCAGTCAAAGAGTTTTCTGCTT
ATTTTAGATGATGTATGGGACAGTATGGATGATCATCAATGGAGAAAACTGGTGTGTCCTTTTGTATCAAGTCAAGCAAAGGGTAATTTA
ATTCTAGTCACAACCAGAAATTTGTCAGTTGCACACATGTTAGGAACACGTGAGCCGATAAAGTTGGGTGCTTTGGAAAATGATGTTATG
TGGTTGCTGCTCAAGTCATGTGCATTTCGTGATGTGAATTATGAAGGGAACCAAAGTCTAAGCATTGTCGGGAGGCAAATATCAGAGAAG
TTAAAGGGAAACCCACTAGCAGCAGAAACAGCGGGGGCACTATTAAGGAAGAAATTTAGCATTGATTATTGGAAAATCATTTTAAAGAAT
GAAGACTGGAAATCCATGGAGCTCGGTAATGGAATCATGGCTGCTCTAAAGCTTAGCTATGATCAACTTCCCTACCATTTACAACAATGT
TTCTCATATTGCTCCATATTCCCCGACGGTTATCAGTTTCTTGGTGAGGAGTTGGTCGGTTTCTGGATGTCACAGGGATTTGTAAAGTGC
AACAACTCTAGTCAGAGATTGGAGCAGATAGGACAGTGCTATCTGATTGATTTGGTTAACTTAGGCTTCTTTGAAGAAGTTAAAAGAGAA
GAACCATATCTGGGCTGTCGAGTTATGTATGGCATATGTGGTCTCATGCATGATTTTGTGATTATGGTGTCAAGGACTGACTGTGCAAGT
ATAGATGGTCTGCAGCGCAACAAAATGCCTCAAACTCTACGACATTTGTCAATAGTAACTGGATCCGCGTACAAGAAAAATCAGCACGGA
AACATTCCTCGTAATAATAGGTTTGAAGAAAATCTGAGAAATACAATTACATCAGTTAGCGAGTTGAGGACATTGGTGTTACTTGGGCAT
TATGACTTTTCCTTCTTACTATTATTCCAAGATATATTTCAAAAGGCACATAACTTACGTGTGCTGCAAATGTCTGCACCACCTGCTGAT
TTTCTCAAACATAGGTTTGAGGAGGTGGATGGGTCTTTCCCTCAAATTTTGAGCAAATTGTACCATCTCCAAGTATTAGACGTCGGTGCA
TACACTGATCGTACTATGCCTGGTTGTATTGATAATCTTGTTAGCCTGCGGCATCTTGTTGTACACAAGGGAGTGTACTCTTCCATTGCA
ACCATTGATAATATGCTATCATTTCAGGAACAACATGGTTTCAAGTTTCATATTTCTAGTGGCTTTGAGATAACACGACTCCAATCCACT
GAACATTGGATGCATGTTGATACTCTGGAAGATGTTTATGAGGCAGGACTGGTAAACAATGAACTCTCAGAAAAGTTGCACCTGTCCTGG
AAGATTCTCCTGAGGACATAG
>Yr5_protein
(SEQ ID NO: 2)
MEPAGDSSVEAAIAWLVQTILATLLMDKMEEWIRQVGLADDVERLQSEVERVDTVVAAVKGRAAGNRPLSRALARVKELLYDADDLIDEL
DYYRLQQQVEGVTSDDPDGMRGAERVDEISRGHVDTLNCSVGKLRSPVWEHFTITETTIDGKRSKAKCNYCGNDFNCETKTNGTSSMKKH
LEKEHSVTCTKKPGAHPPNPSSTGYATENVTLVEVGSSSNRKRKRTNKEPAQTTADNTRWDKAELSDTIKKITSQLQLQLQGILWAFSKV
LEPHGSSSASSSNHHQPSTTSDQHAKTSSLAPRKVYGRVAEMNSIRNLIAEKKCDALTVLPIVGIAGVGKTTLAQSVYNDPDIKSQFHHK
IWVCVSRKFDEVMLTREMLDFERHEGSPHENGRHEGISSLAKLQEILKDIIEYQSKSFLLILDDVWDSMDDHQWRKLVCPFVSSQAKGNL
ILVTTRNLSVAHMLGTREPIKLGALENDVMWLLLKSCAFRDVNYEGNQSLSIVGRQISEKLKGNPLAAETAGALLRKKFSIDYWKIILKN
EDWKSMELGNGIMAALKLSYDQLPYHLQQCFSYCSIFPDGYQFLGEELVGFWMSQGFVKCNNSSQRLEQIGQCYLIDLVNLGFFEEVKRE
EPYLGCRVMYGICGLMHDFVIMVSRTDCASIDGLQRNKMPQTLRHLSIVTGSAYKKNQHGNIPRNNRFEENLRNTITSVSELRTLVLLGH
YDFSFLLLFQDIFQKAHNLRVLQMSAAPADFLKHRFEEVDGSFPQILSKLYHLQVLDVGAYTDRTMPGCIDNLVSLRHLVVHKGVYSSIA
TIDNMLSFQEQHGFKFHISSGFEITRLQSTEHWMHVDTLEDVYEAGLVNNELSEKLHLSWKDSPEDIGMEVEDWEPHWDLRVLEISGYNF
GSPIVVDIIILVTSSQTVEISNCSEWKILPSLERFQFLTNLELRNLPKVIEILVPSLEELALVTMPKLKKCSCTPVEGMSSRLRALRIED
CQSLKEFDLFENNDKFETGQRSWAPSLRELSLENCPHLKVLKPLPLSLMCSELLISGVSTLPYMKGSSDRKLCIGYDDKYDYYGFDESSD
ELKILDDKIFMFHNLKNLKSMVTYGCRNLSSISLKGFSYLISLTSLEIRDCEKLFASDEMPEHTLEDVTPANCKAFPSLECLSIDSCGIV
GKWLSLMLQHAPCLEELYLSSREEENSEEENSEEEENSISNLSSTREGTSSGNPDDGLALDRLLRIPLNLISILKSITIERCPHLTFNWG
KEGVSGFTSLEKLIVLDRPDMVLTNGRWLLPNSLGELESNDYSRGTLQPCFPSDITSLKKLKVRRSPGLQSLQLHSCMALEELDIQDCRR
LAALQGLQFLGSLTHLTIYNCPGLPPFLESFSRQGYTLLPRLKRLHIQDPSVLTTSFCRHLTSLQHLKLTWLEEVRLTDEQEQALVLLKS
LQELQFHYCSNLVDLPAVLHNLPSLKTLKVDGCRGISRLPETGLPFSLEELEIEWCSKELADQCRLLASNKLNIKILSGIYV-
>YrSP_protein
(SEQ ID NO: 6)
MEPAGDSSVEAAIAWLVQTILATLLMDKMEEWIRQVGLADDVERLQSEVERVDTVVAAVKGRAAGNRPLSRALARVKELLYDADDLIDEL
DYYRLQQQVEGVTSDDPDGMRGAERVDEISRGHVDTLNCSVGKLRSPVWEHFTITETTIDGKRSKAKCNYCGNDFNCETKTNGTSSMKKH
LEKEHSVTCTKKPGAHPPNPSSTGYATENVTLVEVGSSSNRKRKRTNKEPAQTTADNTRWDKAELSDTIKKITSQLQLQLQGILWAFSKV
LEPHGSSSASSSNHHQPSTTSDQHAKTSSLAPRKVYGRVAEMNSIRNLIAEKKCDALTVLPIVGIAGVGKTTLAQSVYNDPDIKSQFHHK
IWVCVSRKFDEVMLTREMLDFERHEGSPHENGRHEGISSLAKLQEILKDIIEYQSKSFLLILDDVWDSMDDHQWRKLVCPFVSSQAKGNL
ILVTTRNLSVAHMLGTREPIKLGALENDVMWLLLKSCAFRDVNYEGNQSLSIVGRQISEKLKGNPLAAETAGALLRKKFSIDYWKIILKN
EDWKSMELGNGIMAALKLSYDQLPYHLQQCFSYCSIFPDGYQFLGEELVGFWMSQGFVKCNNSSQRLEQrGQCYLIDLVNLGFFEEVKRE
EPYLGCRVMYGICGLMHDFVIMVSRTDCASIDGLQRNKMPQTLRHLSIVTGSAYKKNQHGNIPRNNRFEENLRNTITSVSELRTLVLLGH
YDFSFLLLFQDIFQKAHNLRVLQMSAPPADFLKHRFEEVDGSFPQILSKLYHLQVLDVGAYTDRTMPGCIDNLVSLRHLVVHKGVYSSIA
TIDNMLSFQEQHGFKFHISSGFEITRLQSTEHWMHVDTLEDVYEAGLVNNELSEKLHLSWKILLRT-
>Yr7_with_Ns
ATGGAGCCGGCGGGAGACTCTTCCCTGGAGGCCGCGATTGCATGGCTGGTGCAGACCATCCTTGCAACGCTCCTCATGGACAAGATGGAG
GCCTGGATTCAGCAAGTCGGGCTTGCCGACGACGTCGAGAGGCTCCAGTCTGAGGTCGAGAGAGTCGACACGGTGGTGGCTGCTGTGAAG
GGGAGGGCAGCCGGGAACATGCCTCTGTCCCGGTCTCTCGCTCGTGTCAAGGAGCTTCTCTATGACGCCGACGACGTGATCGACGAGCTA
GACTACTACAGGCTCCAACACCAAGTCGAAGGAGGTAGTAAGCATAATCCCATTATATATCGAATCTATGTGTGCTACTCAATAGTTTGA
TCTTAATTTCTGGTCCATGTTTCTTTTCGGCACAGTTACAAGTGACGAGCCTGACGGTATGCGTGGAGCTGAAAGAGTGGATGAAATATC
AAGGGGCCATGTCGATACACTGAATGTCAGTGTTGGCAAATTACGGTCCCCGGTATGGGAACACTTCACCATCACAGAAACAACTATCGA
CGGGAAGCGTTCAAAAGCCAAATGTAAGTACTGTGGAAATGATTTTAATTGCGAAACGAAGACAAACGGGACTTCATCTATGAAAAAACA
TTTGGAGAAGGAGCATTCCGTGACTTGCACGAATAAATCTGCAGTGCACCCCCCAAACACTTCAAGGTACCAGCAGGAATTTATACCTTG
CTTCAACGAATTTGTTGTAATTGTTTATATACGTCTGCTTGAGAGCCCATTGTTGTTCTGAATTTCTTCTGATAACCAACCCACCATCCT
TTTCTTACTGCAGCACCGGCGATGCTACTTGTAATGTGAGGTCGGTTGAAGTTGGTAGTTCGTCCAACGGAAAAAGAAAGAGAACAAATG
AGGATCCN
AAGGCTGAGTTATCCAATAGGATAATTAAAATTACTGAGAAGTTACAGTTACAGGACATCCAGGGGGCTTTGAGTAAAGTTCTCGAGCCA
TATGGATCCAGCGCTACTTCAAGTTCAAATCATCACCGCTTGAGTACAGCATCAGATCAGCACCCAACAACATCAAGTCTTGTTCCAATG
GAAGTTTATGGCAGAGTTGCAGAAAAGAATAAGATCAAAAAGTCAATAACTGAAAACCAATCTGGTGGTGTAAATGTTCTGCCTATTGTA
GGCATTGCAGGTGTTGGAAAGACAACTCTTGCTCAATTTGTGTATAATGATCCAGACN
CAGAAAAGAATAAGATCAAAAAGTCAATAACTGAAAACCAATCTGGTGGTGTAAATGTTCTGCCTATTGTAGGCATTGCAGGTGTTGGAA
AGACAACTCTTGCTCAATTTGTGTATAATGATCCAGACGTGAAAAGTCAATTTCACCACAGGATATGGGTTTGTGTGTCCTGCAAATTTG
ATGAAGTGAAGCTCACAAAGGAGATGTTAGACTTTTTTCCTCGAGAAAGGCATGAAGGAATTAACAACTTCGCGAAGCTTCAAGAGATCT
TGAAAGAACATGTCGAGTACCAAGCAAAGAGTTTTCTGCTCATTTTAGATGATGTCTCGGACAGTATGGATTATCATAAATGGAACAAAT
TGTTGAACCCTTTGCTATCAAGTCAAGCGAAGAATATAATTCTAGTCACGACCAGAAATTTGTCTGTTGCACAAAGGTTAAGCACACTTG
AACCGATCAAGTTAGGTGCTTTAGAAAACGATGATATGTGGTTATTGCTCAAGTCATGTGCATTTGGTTTTGGGAACTATGAAGGTACGG
AAAATCTAAGCACTATTGGAAGACAAATAGCAGAGAAGTTAAAGGGCAATCCGTTAGCAGCAGTAACTGCAGGGGCACTGTTAAGAGATA
ATCTTAGCATTGATCATTGGAGTAACATTCTCAAGAATGAGAAGTGGAAATCGCTGGGACTCAGTGGGGGCATCATGCCTGCTTTGAAGC
TTAGTTATGATGAGTTGACGTACCGTTTACAACAATGTTTCTCGTATTGCTCTATATTTCCTGACAAATATAGGTTTCTCGGGAAGGATT
TGGTCTATATTTGGATTTCTCAGGGATTTGTGAATTGCACCCAAAATAAGAGATTGGAGGAGACGGGATGGGAATATCTGAATCAATTGG
TAAATCTTGGATTCTTTCAACAAATTGAAGAACAACAAGAATTGGATGGGGAAGAAGAATTCTCTCTACGCCGTCAGATTTGGTACTCTA
TGTGTGATCTCATGCATGATTTCGCAAGGATGATTTCAAGGACTGAATGTGCGACTATAGATGGTCTACAGTGCAATAAAATATTCCCAA
CTGTACAGCATTTGTCAATAGTAACCGGTTCTGCATACAACAAAGATCTGAAGGGGAACATTCCTCGTAATGAGAAGTTTGAAGAAAATA
TGAGAAATTCAGTTACATCAGTTACCAAATTGAGAACATTGGTTGTGCTTGGGAACTTTGACTCTTTCTTTGTACGGTTGTTCCAAGATA
TATTCCAGAAGGCACAAAATTTACGCCTGCTGCTAGTATCTCTAGCATCCACTTATCTGTCTCAAGTGCCTGCTGCATTCAATGATTTTA
ATTCCTTCCTGTGCAATTTGGCAAATCCTTTGCATCTTCGTTACCTAAAACTTGAGTTGGATGGGATTGTGCCACAAGTTTTGAGTACGT
TTTTTCATCTTCAAGTATTAGATGTTGGATCAAGCATGGATACTTCTCTACCCAATGGCTTGTTGCATAATCTTGTTAGCCTGCGACATC
TTGTTGCACACAAGAGAGTCCATTCTTCCATTACTAGCATTGGTAACATGACATCTATCCAGGAGCTACATGATTTTGAAGTTCGAATTT
CTAGCGGCTTTGAGATAACACGACTCCAATCCATGAACGAGCTTGTTCAACTTGGGTTGTCTCAACTTGACAGTGTTAAAACCAGGGAGG
ACGCTTATGGGGCAGGACTAAGAAACAAGGAACACTTAGAAGAGCTTCATTTGTCCTGGAAGGATGCATATTCAGAGTATGAGTATGCCA
GTGACACTGAATTTGAATCTTCTGCAAACATGGCAAGAGAAGTGATTGAGGGTCTTGAACCACACATGGATTTAAAACATCTACAAATAT
CTCAGTATAATGGTACCACTTCACCAGCTTGGCTTGCCAACAATATCTCAGTTACCTCATTGCAGACGCTTCATCTTGATGATTGTGGAG
GATGGAGAATACTTCCATCTCTGGGAAGTCTTCCATTCCTTACAAAGGTGAAGTTGAGCAGCATGCTGGAAGTAATTGAAGTACTGATTC
CTTCACTGGAGGAGCTAGTTCTAATTAAAATGCCGAAGTTAGTGAGATGCTCAAGCACTTCTGCCGAGGGTCTGAGCTCTAGCTTAAGGG
TACTGCACATTGAGGATTGTGAAGCATTGAAGGAGTTTGATCTGTTTGAGAACGATTATAATTCTGAAATCATTCAGGGATCATGGCTGC
CTGGTCTTAGGAATTTGATTCTATATTGTTGCCCTCATTTGAAAGTGTTGAAGCCTCTTCCACCTTCAACTACCTTTTCTAAGGTACTCA
TCAGAGAAATTTCAAGATTTCCGTCTATGGAGGTATCATCTGGTGAGAAGTTACAAATTGGGAATATTGATGTGTACATAGGCGATGATT
TTGATGAGTCTTCTGATGAGTTGAGCATACTGGATGACAAAACTTTGGCGTTCCATAATCTTAGAAACCTGAAATCGATGGAGATATATG
GTTGCAGAAATCTAAGGTCTTTTTCGTTCGAAGGTTTCAGTCATCTTGTCTCTTTAACAAGTTTGAAAATAGTAAGCTGTGAACAACTTT
TCCCTTCAGATGTGACGGCAGAGTATACCCTTGAAGATGTGACAGCTGTGAACTGCAATGCCTTCCCATATCTTAAAAGCCTCAGTATCG
ACTCATGTGGAATAGCGGGGAAGTGGCTATCGCTGATGCTGCAGCATGCGCCAGGCCTAGAGGAATTGAGTTTAACAAGTTGCGCCCATA
TAACAAGAGTAGTGTTACCGATGGAAGAGGAAGAAAACAATCTATTAACAACAGTACTGTCATCAGGAAATCAAGATGAGGCATTGACAT
GGTTAGTTCGTGACGGACTCTTGCACATTCCATCAAATCTCGTCTCCTCTCTCAAGAATATGAGTATTACTCAGTGCCCTCGCCTAAAGT
TTAACTCAGGCAAGGACTGCTTCTCTGGATTTACCTCGCTTGAGAAGCTTGAAATTTGGGGATCGTTGGTGGATGATGACGGAAGTGATG
ACCTGGAGAATGGAAGTTCTTTTGTGTTCGGAGAGGAGGATCAACCCCTGGGGGCGAACGGAAGATGGCTCCTCCCGACATCACTTCAGG
AACTTCACATCGTGTCATTGTATTGCCAAGAAACGCTGCAAGTCTGCTTCCCTAGAGATATCACCAGCCTTAAAAAGTTAAGTGTACGTT
CCGGCCAAGGTTTGCAATCTCTACAGCTGTACTCATGCACGGCACTGGAAGAATTGGCAATTTCCGGCTCTGGATCGGTCACCGTCACTG
TACTAGAGGGCACGCAACCCGCTGGCAGCCTCGGGCGTTTGAATGTATCAGACTGTCCTGGCTTGCCATCACGTTTGGACAGCTTTCCAA
GGTTGTGCCCTCGGCTGGAAAGGCTTGACATCAATGACCCATCTGTCCTTACCACGCCATTCTGCAAGCACCTCACCTCCCTGCAACGCC
TAAAACTTGGCTTCTTGAAAGTGACGAGACTAACAGATGAGCAAGAACGAGCGCTTGTGCTCCTCAAGTCACTGAAAGAGCTCGAGATTT
TTTATTGTACTCATCTCATAGATCTTCCTGCGGGGCTGCAGACCCTTCCTTCCCTCAAGAGTTTGAAGATAGAAGAGGGTCGAGGCATCT
CAAGGCTGCCGGAAGCAGGCCTCCCACATTCGCTGGAAGAACTGGAAATCAAAATTTGCAGCAAGCTAGAAGATGAATGCAGGCGGCTAG
CAACATGCGAAGGCAAGCTAAAAGTCAAAATTGATGGTCGATATGTGAATTAA
>curated_Yr7
ATGGAGCCGGCGGGAGACTCTTCCCTGGAGGCCGCGATTGCATGGCTGGTGCAGACCATCCTTGCAACGCTCCTCATGGACAAGATGGAG
GCCTGGATTCAGCAAGTCGGGCTTGCCGACGACGTCGAGAGGCTCCAGTCTGAGGTCGAGAGAGTCGACACGGTGGTGGCTGCTGTGAAG
GGGAGGGCAGCCGGGAACATGCCTCTGTCCCGGTCTCTCGCTCGTGTCAAGGAGCTTCTCTATGACGCCGACGACGTGATCGACGAGCTA
GACTACTACAGGCTCCAACACCAAGTCGAAGGAGGTAGTAAGCATAATCCCATTATATATCGAATCTATGTGTGCTACTCAATAGTTTGA
TCTTAATTTCTGGTCCATGTTTCTTTTCGGCACAGTTACAAGTGACGAGCCTGACGGTATGCGTGGAGCTGAAAGAGTGGATGAAATATC
AAGGGGCCATGTCGATACACTGAATGTCAGTGTTGGCAAATTACGGTCCCCGGTATGGGAACACTTCACCATCACAGAAACAACTATCGA
CGGGAAGCGTTCAAAAGCCAAATGTAAGTACTGTGGAAATGATTTTAATTGCGAAACGAAGACAAACGGGACTTCATCTATGAAAAAACA
TTTGGAGAAGGAGCATTCCGTGACTTGCACGAATAAATCTGCAGTGCACCCCCCAAACACTTCAAGGTACCAGCAGGAATTTATACCTTG
CTTCAACGAATTTGTTGTAATTGTTTATATACGTCTGCTTGAGAGCCCATTGTTGTTCTGAATTTCTTCTGATAACCAACCCACCATCCT
TTTCTTACTGCAGCACCGGCGATGCTACTTGTAATGTGAGGTCGGTTGAAGTTGGTAGTTCGTCCAACGGAAAAAGAAAGAGAACAAATG
AGGATCCAACGCAGACCACCGCAGCTAACATACACGCCCAATGGGACAAGGCTGAGTTATCCAATAGGATAATTAAAATTACTGAGAAGT
TACAGTTACAGGACATCCAGGGGGCTTTGAGTAAAGTTCTCGAGCCATATGGATCCAGCGCTACTTCAAGTTCAAATCATCACCGCTTGA
GTACAGCATCAGATCAGCACCCAACAACATCAAGTCTTGTTCCAATGGAAGTTTATGGCAGAGTTGCAGAAAAGAATAAGATCAAAAAGT
CAATAACTGAAAACCAATCTGGTGGTGTAAATGTTCTGCCTATTGTAGGCATTGCAGGTGTTGGAAAGACAACTCTTGCTCAATTTGTGT
ATAATGATCCAGAC
GTGAAAAGTCAATTTCACCACAGGATATGGGTTTGTGTGTCCTGCAAATTTGATGAAGTGAAGCTCACAAAGGAGATGTTAGACTTTTTT
CCTCGAGAAAGGCATGAAGGAATTAACAACTTCGCGAAGCTTCAAGAGATCTTGAAAGAACATGTCGAGTACCAAGCAAAGAGTTTTCTG
CTCATTTTAGATGATGTCTCGGACAGTATGGATTATCATAAATGGAACAAATTGTTGAACCCTTTGCTATCAAGTCAAGCGAAGAATATA
ATTCTAGTCACGACCAGAAATTTGTCTGTTGCACAAAGGTTAAGCACACTTGAACCGATCAAGTTAGGTGCTTTAGAAAACGATGATATG
TGGTTATTGCTCAAGTCATGTGCATTTGGTTTTGGGAACTATGAAGGTACGGAAAATCTAAGCACTATTGGAAGACAAATAGCAGAGAAG
TTAAAGGGCAATCCGTTAGCAGCAGTAACTGCAGGGGCACTGTTAAGAGATAATCTTAGCATTGATCATTGGAGTAACATTCTCAAGAAT
GAGAAGTGGAAATCGCTGGGACTCAGTGGGGGCATCATGCCTGCTTTGAAGCTTAGTTATGATGAGTTGACGTACCGTTTACAACAATGT
TTCTCGTATTGCTCTATATTTCCTGACAAATATAGGTTTCTCGGGAAGGATTTGGTCTATATTTGGATTTCTCAGGGATTTGTGAATTGC
ACCCAAAATAAGAGATTGGAGGAGACGGGATGGGAATATCTGAATCAATTGGTAAATCTTGGATTCTTTCAACAAATTGAAGAACAACAA
GAATTGGATGGGGAAGAAGAATTCTCTCTACGCCGTCAGATTTGGTACTCTATGTGTGATCTCATGCATGATTTCGCAAGGATGATTTCA
AGGACTGAATGTGCGACTATAGATGGTCTACAGTGCAATAAAATATTCCCAACTGTACAGCATTTGTCAATAGTAACCGGTTCTGCATAC
AACAAAGATCTGAAGGGGAACATTCCTCGTAATGAGAAGTTTGAAGAAAATATGAGAAATTCAGTTACATCAGTTACCAAATTGAGAACA
TTGGTTGTGCTTGGGAACTTTGACTCTTTCTTTGTACGGTTGTTCCAAGATATATTCCAGAAGGCACAAAATTTACGCCTGCTGCTAGTA
TCTCTAGCATCCACTTATCTGTCTCAAGTGCCTGCTGCATTCAATGATTTTAATTCCTTCCTGTGCAATTTGGCAAATCCTTTGCATCTT
CGTTACCTAAAACTTGAGTTGGATGGGATTGTGCCACAAGTTTTGAGTACGTTTTTTCATCTTCAAGTATTAGATGTTGGATCAAGCATG
GATACTTCTCTACCCAATGGCTTGTTGCATAATCTTGTTAGCCTGCGACATCTTGTTGCACACAAGAGAGTCCATTCTTCCATTACTAGC
ATTGGTAACATGACATCTATCCAGGAGCTACATGATTTTGAAGTTCGAATTTCTAGCGGCTTTGAGATAACACGACTCCAATCCATGAAC
GAGCTTGTTCAACTTGGGTTGTCTCAACTTGACAGTGTTAAAACCAGGGAGGACGCTTATGGGGCAGGACTAAGAAACAAGGAACACTTA
GAAGAGCTTCATTTGTCCTGGAAGGATGCATATTCAGAGTATGAGTATGCCAGTGACACTGAATTTGAATCTTCTGCAAACATGGCAAGA
GAAGTGATTGAGGGTCTTGAACCACACATGGATTTAAAACATCTACAAATATCTCAGTATAATGGTACCACTTCACCAGCTTGGCTTGCC
AACAATATCTCAGTTACCTCATTGCAGACGCTTCATCTTGATGATTGTGGAGGATGGAGAATACTTCCATCTCTGGGAAGTCTTCCATTC
CTTACAAAGGTGAAGTTGAGCAGCATGCTGGAAGTAATTGAAGTACTGATTCCTTCACTGGAGGAGCTAGTTCTAATTAAAATGCCGAAG
TTAGTGAGATGCTCAAGCACTTCTGCCGAGGGTCTGAGCTCTAGCTTAAGGGTACTGCACATTGAGGATTGTGAAGCATTGAAGGAGTTT
GATCTGTTTGAGAACGATTATAATTCTGAAATCATTCAGGGATCATGGCTGCCTGGTCTTAGGAATTTGATTCTATATTGTTGCCCTCAT
TTGAAAGTGTTGAAGCCTCTTCCACCTTCAACTACCTTTTCTAAGGTACTCATCAGAGAAATTTCAAGATTTCCGTCTATGGAGGTATCA
TCTGGTGAGAAGTTACAAATTGGGAATATTGATGTGTACATAGGCGATGATTTTGATGAGTCTTCTGATGAGTTGAGCATACTGGATGAC
AAAACTTTGGCGTTCCATAATCTTAGAAACCTGAAATCGATGGAGATATATGGTTGCAGAAATCTAAGGTCTTTTTCGTTCGAAGGTTTC
AGTCATCTTGTCTCTTTAACAAGTTTGAAAATAGTAAGCTGTGAACAACTTTTCCCTTCAGATGTGACGGCAGAGTATACCCTTGAAGAT
GTGACAGCTGTGAACTGCAATGCCTTCCCATATCTTAAAAGCCTCAGTATCGACTCATGTGGAATAGCGGGGAAGTGGCTATCGCTGATG
CTGCAGCATGCGCCAGGCCTAGAGGAATTGAGTTTAACAAGTTGCGCCCATATAACAAGAGTAGTGTTACCGATGGAAGAGGAAGAAAAC
AATCTATTAACAACAGTACTGTCATCAGGAAATCAAGATGAGGCATTGACATGGTTAGTTCGTGACGGACTCTTGCACATTCCATCAAAT
CTCGTCTCCTCTCTCAAGAATATGAGTATTACTCAGTGCCCTCGCCTAAAGTTTAACTCAGGCAAGGACTGCTTCTCTGGATTTACCTCG
CTTGAGAAGCTTGAAATTTGGGGATCGTTGGTGGATGATGACGGAAGTGATGACCTGGAGAATGGAAGTTCTTTTGTGTTCGGAGAGGAG
GATCAACCCCTGGGGGCGAACGGAAGATGGCTCCTCCCGACATCACTTCAGGAACTTCACATCGTGTCATTGTATTGCCAAGAAACGCTG
CAAGTCTGCTTCCCTAGAGATATCACCAGCCTTAAAAAGTTAAGTGTACGTTCCGGCCAAGGTTTGCAATCTCTACAGCTGTACTCATGC
ACGGCACTGGAAGAATTGGCAATTTCCGGCTCTGGATCGGTCACCGTCACTGTACTAGAGGGCACGCAACCCGCTGGCAGCCTCGGGCGT
TTGAATGTATCAGACTGTCCTGGCTTGCCATCACGTTTGGACAGCTTTCCAAGGTTGTGCCCTCGGCTGGAAAGGCTTGACATCAATGAC
CCATCTGTCCTTACCACGCCATTCTGCAAGCACCTCACCTCCCTGCAACGCCTAAAACTTGGCTTCTTGAAAGTGACGAGACTAACAGAT
GAGCAAGAACGAGCGCTTGTGCTCCTCAAGTCACTGAAAGAGCTCGAGATTTTTTATTGTACTCATCTCATAGATCTTCCTGCGGGGCTG
CAGACCCTTCCTTCCCTCAAGAGTTTGAAGATAGAAGAGGGTCGAGGCATCTCAAGGCTGCCGGAAGCAGGCCTCCCACATTCGCTGGAA
GAACTGGAAATCAAAATTTGCAGCAAGCTAGAAGATGAATGCAGGCGGCTAGCAACATGCGAAGGCAAGCTAAAAGTCAAAATTGATGGT
CGATATGTGAATTAA
>Yr7_Paragon_with_Ns
ATGGAGCCGGCGGGAGACTCTTCCCTGGAGGCCGCGATTGCATGGCTGGTGCAGACCATCCTTGCAACGCTCCTCATGGACAAGATGGAG
GCCTGGATTCAGCAAGTCGGGCTTGCCGACGACGTCGAGAGGCTCCAGTCTGAGGTCGAGAGAGTCGACACGGTGGTGGCTGCTGTGAAG
GGGAGGGCAGCCGGGAACATGCCTCTGTCCCGGTCTCTCGCTCGTGTCAAGGAGCTTCTCTATGACGCCGACGACGTGATCGACGAGCTA
GACTACTACAGGCTCCAACACCAAGTCGAAGGAGGTAGTAAGCATAATCCCATTATATATCGAATCTATGTGTGCTACTCAATAGTTTGA
TCTTAATTTCTGGTCCATGTTTCTTTTCGGCACAGTTACAAGTGACGAGCCTGACGGTATGCGTGGAGCTGAAAGAGTGGATGAAATATC
AAGGGGCCATGTCGATACACTGAATGTCAGTGTTGGCAAATTACGGTCCCCGGTATGGGAACACTTCACCATCACAGAAACAACTATCGA
CGGGAAGCGTTCAAAAGCCAAATGTAAGTACTGTGGAAATGATTTTAATTGCGAAACGAAGACAAACGGGACTTCATCTATGAAAAAACA
TTTGGAGAAGGAGCATTCCGTGACTTGCACGAATAAATCTGCAGTGCACCCCCCAAACACTTCAAGGTACCAGCAGGAATTTATACCTTG
CTTCAACGAATTTGTTGTAATTGTTTATATACGTCTGCTTGAGAGCCCATTGTTGTTCTGAATTTCTTCTGATAACCAACCCACCATCCT
TTTCTTACTGCAGCACCGGCGATGCTNA
CGGAAAAAGAAAGAGAACAAATGAGGATCCAACGCAGACCACCGCAGCTAACATACACGCCCAATGGGACAAGGCTGAGTTATCCAATAG
GATAATTAAAATTACTGAGAAGTTACAGTTACAGGACATCCAGGGGGCTTTGAGTAAAGTTCTCGAGCCATATGGATCCAGCGCTACTTC
AAGTTCAAATCATCACCGCTTGAGTACAGCATCAGATCAGCACCCAACAACATCAAGTCTTGTTCCAATGGAAGTTTATGGCAGAGTTGC
AGAAAAGAATAAGATCAAAAAGTCAATAACTGAAAACCAATCTGGTGGTGTAAATGN
CTTGAGTACAGCATCAGATCAGCACCCAACAACATCAAGTCTTGTTCCAATGGAAGTTTATGGCAGAGTTGCAGAAAAGAATAAGATCAA
AAAGTCAATAACTGAAAACCAATCTGGTGGTGTAAATGTTCTGCCTATTGTAGGCATTGCAGGTGTTGGAAAGACAACTCTTGCTCAATT
TGTGTATAATGATCCAGACGTGAAAAGTCAATTTCACCACAGGATATGGGTTTGTGTGTCCTGCAAATTTGATGAAGTGAAGCTCACAAA
GGAGATGTTAGACTTTTTTCCTCGAGAAAGGCATGAAGGAATTAACAACTTCGCGAAGCTTCAAGAGATCTTGAAAGAACATGTCGAGTA
CCAAGCAAAGAGTTTTCTGCTCATTTTAGATGATGTCTCGGACAGTATGGATTATCATAAATGGAACAAATTGTTGAACCCTTTGCTATC
AAGTCAAGCGAAGAATATAATTCTAGTCACGACCAGAAATTTGTCTGTTGCACAAAGGTTAAGCACACTTGAACCGATCAAGTTAGGTGC
TTTAGAAAACGATGATATGTGGTTATTGCTCAAGTCATGTGCATTTGGTTTTGGGAACTATGAAGGTACGGAAAATCTAAGCACTATTGG
AAGACAAATAGCAGAGAAGTTAAAGGGCAATCCGTTAGCAGCAGTAACTGCAGGGGCACTGTTAAGAGATAATCTTAGCATTGATCATTG
GAGTAACATTCTCAAGAATGAGAAGTGGAAATCGCTGGGACTCAGTGGGGGCATCATGCCTGCTTTGAAGCTTAGTTATGATGAGTTGAC
GTACCGTTTACAACAATGTTTCTCGTATTGCTCTATATTTCCTGACAAATATAGGTTTCTCGGGAAGGATTTGGTCTATATTTGGATTTC
TCAGGGATTTGTGAATTGCACCCAAAATAAGAGATTGGAGGAGACGGGATGGGAATATCTGAATCAATTGGTAAATCTTGGATTCTTTCA
ACAAATTGAAGAACAACAAGAATTGGATGGGGAAGAAGAATTCTCTCTACGCCGTCAGATTTGGTACTCTATGTGTGATCTCATGCATGA
TTTCGCAAGGATGATTTCAAGGACTGAATGTGCGACTATAGATGGTCTACAGTGCAATAAAATATTCCCAACTGTACAGCATTTGTCAAT
AGTAACCGGTTCTGCATACAACAAAGATCTGAAGGGGAACATTCCTCGTAATGAGAAGTTTGAAGAAAATATGAGAAATTCAGTTACATC
AGTTACCAAATTGAGAACATTGGTTGTGCTTGGGAACTTTGACTCTTTCTTTGTACGGTTGTTCCAAGATATATTCCAGAAGGCACAAAA
TTTACGCCTGCTGCTAGTATCTCTAGCATCCACTTATCTGTCTCAAGTGCCTGCTGCATTCAATGATTTTAATTCCTTCCTGTGCAATTT
GGCAAATCCTTTGCATCTTCGTTACCTAAAACTTGAGTTGGATGGGATTGTGCCACAAGTTTTGAGTACGTTTTTTCATCTTCAAGTATT
AGATGTTGGATCAAGCATGGATACTTCTCTACCCAATGGCTTGTTGCATAATCTTGTTAGCCTGCGACATCTTGTTGCACACAAGAGAGT
CCATTCTTCCATTACTAGCATTGGTAACATGACATCTATCCAGGAGCTACATGATTTTGAAGTTCGAATTTCTAGCGGCTTTGAGATAAC
ACGACTCCAATCCATGAACGAGCTTGTTCAACTTGGGTTGTCTCAACTTGACAGTGTTAAAACCAGGGAGGACGCTTATGGGGCAGGACT
AAGAAACAAGGAACACTTAGAAGAGCTTCATTTGTCCTGGAAGGATGCATATTCAGAGTATGAGTATGCCAGTGACACTGAATTTGAATC
TTCTGCAAACATGGCAAGAGAAGTGATTGAGGGTCTTGAACCACACATGGATTTAAAACATCTACAAATATCTCAGTATAATGGGACCAC
TTCACCAGCTTGGCTTGCCAACAATATCTCAGTTACCTCATTGCAGACGCTTCATCTTGATGATTGTGGAGGATGGAGAATACTTCCATC
TCTGGGAAGTCTTCCATTCCTTACAAAGGTGAAGTTGAGCAGCATGCTGGAAGTAATTGAAGTACTGATTCCTTCACTGGAGGAGCTAGT
TCTAATTAAAATGCCGAAGTTAGTGAGATGCTCAAGCACTTCTGCCGAGGGTCTGAGCTCTAGCTTAAGGGTACTGCACATTGAGGATTG
TGAAGCATTGAAGGAGTTTGATCTGTTTGAGAACGATTATAATTCTGAAATCATTCAGGGATCATGGCTGCCTGGTCTTAGGAATTTGAT
TCTATATTGTTGCCCTCATTTGAAAGTGTTGAAGCCTCTTCCACCTTCAACTACCTTTTCTAAGGTACTCATCAGAGAAATTTCAAGATT
TCCGTCTATGGAGGTATCATCTGGTGAGAAGTTACAAATTGGGAATATTGATGTGTACATAGGCGATGATTTTGATGAGTCTTCTGATGA
GTTGAGCATACTGGATGACAAAACTTTGGCGTTCCATAATCTTAGAAACCTGAAATCGATGGAGATATATGGTTGCAGAAATCTAAGGTC
TTTTTCGTTCGAAGGTTTCAGTCATCTTGTCTCTTTAACAAGTTTGAAAATAGTAAGCTGTGAACAACTTTTCCCTTCAGATGTGACGGC
AGAGTATACCCTTGAAGATGTGACAGCTGTGAACTGCAATGCCTTCCCATATCTTAAAAGCCTCAGTATCGACTCATGTGGAATAGCGGG
GAAGTGGCTATCGCTGATGCTGCAGCATGCGCCAGGCCTAGAGGAATTGAGTTTAACAAGTTGCGCCCATATAACAAGAGTAGTGTTACC
GATGGAAGAGGAAGAAAACAATCTATTAACAACAGTACTGTCATCAGGAAATCAAGATGAGGCATTGACATGGTTAGTTCGTGACGGACT
CTTGCACATTCCATCAAATCTCGTCTCCTCTCTCAAGAATATGAGTATTACTCAGTGCCCTCGCCTAAAGTTTAACTCAGGCAAGGACTG
CTTCTCTGGATTTACCTCGCTTGAGAAGCTTGAAATTTGGGGATCGTTGGTGGATGATGACGGAAGTGATGACCTGGAGAATGGAAGTTC
TTTTGTGTTCGGAGAGGAGGATCAACCCCTGGGGGCGAACGGAAGATGGCTCCTCCCGACATCACTTCAGGAACTTCACATCGTGTCATT
GTATTGCCAAGAAACGCTGCAAGTCTGCTTCCCTAGAGATATCACCAGCCTTAAAAAGTTAAGTGTACGTTCCGGCCAAGGTTTGCAATC
TCTACAGCTGTACTCATGCACGGCACTGGAAGAATTGGCAATTTCCGGCTCTGGATCGGTCACCGTCACTGTACTAGAGGGCACGCAACC
CGCTGGCAGCCTCGGGCGTTTGAATGTATCAGACTGTCCTGGCTTGCCATCACGTTTGGACAGCTTTCCAAGGTTGTGCCCTCGGCTGGA
AAGGCTTGACATCAATGACCCATCTGTCCTTACCACGCCATTCTGCAAGCACCTCACCTCCCTGCAACGCCTAAAACTTGGCTTCTTGAA
AGTGACGAGACTAACAGATGAGCAAGAACGAGCGCTTGTGCTCCTCAAGTCACTGAAAGAGCTCGAGATTTTTTATTGTACTCATCTCAT
AGATCTTCCTGCGGGGCTGCAGACCCTTCCTTCCCTCAAGAGTTTGAAGATAGAAGAGGGTCGAGGCATCTCAAGGCTGCCGGAAGCAGG
CCTCCCACATTCGCTGGAAGAACTGGAAATCAAAATTTGCAGCAAGCTAGAAGATGAATGCAGGCGGCTAGCAACATGCGAAGGCAAGCT
AAAAGTCAAAATTGATGGTCGATATGTGAATTAA
>curated_TraesCS2B01G48800_Ta_2B09
ATGATGGAGCCGGCGGGAGACTCTTTTGTGGAGGCCGCGATTGCATGGCTGGTGCAGACCATCCTTGCAACGCTCCTGATGGACAAGATG
GAGGAGTGGATTCGGCAAGTCGGTCTTGCCGACGACGTCGAGAGGCTCCAGCGCGAGGTCGAGAGAGTCGACATGGTGGTGGCTGCTGTG
AAGGGGAGGGCAGCCGGGAACAGGCCTCTGTCCCGGGCTCTCGCTCGTGTCAAGGAGCTTCTCTACGACGCCGACGACGTGGTCGACGAA
CTGGACTACTACAGGCTCCAACAGCAAGTCGAAGGAGGTAGTAAGCATAATCCCATTATATCGAAACTATTATGATACTTAATACTCCCT
CTGTTTCTAAATATAAGTATTTTTAGAAATTTCCGTATGTAGTCCATATTGAAATCTCTAAAAGGAATTATATTTAGTAACGGAGGGCGT
AGTTTGATCTTAATTTCTGGTCCATATTTCTTTTCGGCACAGTTACGAGTGACAAGCCTGACGATATGCGTGGAGCTGAAAGAGTGGATG
AAATATCAAGGGGCCATGTCGATACACTGAATGTCAGTGTTGGGAAATTACGGTCCTCGGTATGGGAACACTTTACCATCACAGAAACTG
TCGACCGGAAGCGTTCAAAAGCCAAATGTAAGTACTGTAGAAAGGATTTTAATTGCGAAACGAAGACAAACGGGACTTCATCTATGAAAA
AACATTTGGAGAAAGAGCATTCCGTAACTTGTACGAAGAAACGTGGAGCCCATCCACCAAACCCTTCAAGGTACCCAAAGGAAATTGTAT
GTTGCACCAGTGCATTTGTATTACAAGTTTATATATATCTGCTTGAGAGCCCATTGTTGCTCTACATTTCTTCTGATAACTGACCCACCA
TCCGTTTCTTGTTGCAGCACCGGTGATGCGACTTGTAATGTGAGGTCGGTTGAAGTTGGTAGTTCGTCCAACGGAAAAAGAAAGAGAACA
AATGAGGATCCAACACAAACCACCGCAGCTAACACACACACCCAATGGGACAAGGCTGAGTTTTCCAATAGGATAATTAAAATTACAGGC
CAGTTACAGTCACAGGACATCCAAGGGGCTTTGAGTAAAGTTCTTGGGCCATATGGACCTAGCGCTACTTCAAGTTCAAGTCATCACCGC
CCGAGTACAACCTCAGCTCAGCACCCAACAACATCAAGTCTTGTTCCACTGGAAGTTTATGGCAGAGTTGCAGAAAAGAACAAGATCAAA
AAGTCAATAACTGAAAACCAATCTGGTGGTGTAAATGTTCTACCTATTGTAGGCATTGCAGGTGTTGGAAAGACAACTCTCGCTCAATTT
GTGTATAATGATCCAGACGTGAAAAGTCAATTTCACCACAGGATATGGGTTTGTGTGTCCCGTAAATTTGATGAAGTGAAGCTCACAAAG
GAGATGTTAGACTTTTTTCCTCGAGAAAGGTATGAAGGAATTAGCAATTTTGCGAAGCTTCAAGAGATCTTGAAAGAACATATCGAGTAC
CAGTCGAAGAGCTTTCTGCTTGTATTAGACGATGTCTCGGACAATGTTGATTATCATAAATGGAACAAATTGTTGTACCCTTTGATGTCA
AGTCAAGCAAAGGGTAATATAATTCTAGTCACAACCAAAAATTTGTCTGTTGCACAAAGGTTAAGAACACTTGAACCGATCAAGTTAGGT
GCTTTAGAAAATGATGATATGTGGTTATTGCTCAAGTCATGTGCATTTGGTTTTGGGGACTACAAAGGTCCGGGAAATCTAAGAGCTATT
GGAATGCAAATAGCAGAGAAGTTAAAGGGCAACCCGTTAGCAGCAGTAACTGCAGGGGCACTGTTAAGAGATCATCTTAGCGTTGATCAT
TGGAGTAACATTCTCAAGAAAGAGAAGTGGAAATCGTTGGGACTCCATGGGGGCATCATGCCTGCTTTGAAGCTTAGCTATGATGAGCTA
CCGTACCATTTACAACAATGTTTCTCGTATTGTTCTATATTTTCTGAAAAATATAGGTTTCTTCGGAAGGAACTGGTCTATATTTGGATT
TCTCAAGGATTTTTGAATCACACTAAGAGATTGGAGGAGATAGGATGGGAATGTCTGAATAATTTGGTGAACCTGGGATTCTTTCAGCAG
ATTGGAGAGCAACAGGAAGGGGATGAAGATGAGGAAGAAGATTTTTTTCTAGGCAGTAAAATTTGGTATTGTATGTCTGGTCTCATGCAC
GATTTTGCAAGGATGGTTTCAAGGACTGAGTGTGCAACCATGGATGGTCTTCAGTGTAATAATATGTTACCAACTATACGTCACTTGTCA
ATTGTGACCAATTCTGCATATAGCAAAGAACAGCATGGAACCATACCTCGCAATATCAAGTTTGAAGAGAACCTGAGAAATGCATTTGCA
TCAGTGAGGAAATTGAGGACATTAGTTTTATTTGGGCACTACGACTCTTTCTTCTTCAAATTGTTCCTTGATATATTCCAGAAGGACCAG
AACTTGCGTCTGCTGCAAATGTCTGCAACATGTGCTGATTTTGATTCCTTCATGTGTAGTTTGGTAAATCCTGCACATCTTCGCTATCTA
AAACGTGAACCTGATGAGGTGAATGGTGCTTCCCCTCAAATTTTGAGCAAGTTGTACCATCTTCAAATATTAGATGTTGGCTCATACACT
GATCCTATACCTGATGGTAATAATAATCTAGTTAGCCTGCGGCATCTTATTCCAGAAAATGGAGTATACTCTTCCATTGCTAGCATTGGT
AGAATGACATCACTTAAAGAGCTACATCATTTTAAGGTTCGGTTTTGTTCTAGAGGATTTGAGATATCACAACTCCAATGCATGAACGAG
CTTGTACAACTTGGGGTGTCTCGAGTTGATAGTGTTAAAACTCGGGAGGAGGCTTATGGAGCAGGACTGAGAAGCAAAGAATACTTGAAA
AATCTGCACTTGTCCTGGAAGGATACCTTGTCACAGAAGGAATGTGACACTAGCTCTGAATATTCTGCAGACGAAAACGAGGAGCTCTCA
CAAATGGATACAGCAAGAGAGGTGCTCGAGGGACTTGAACCTCACATGAACTTAAAGCATCTACATATATCTGGGTATAATGGTACTACT
TCACCAACTTGGCTTGCCAACAATCTCTCAGTTACCTCCTTGCAGACGCTTCACCTTGATGGTTGTCGAAGATGGAGAATACTTCCATCT
CTTGAAAGTCTTCCATTTCTTACAAAGCTGAAGTTGAGCAGCATGCTGGAAGTAATAGAAGTATTGGTTCCTTCACTGGAGGAGCTAGTT
TTGATGGACATGCCTAAGTTAGTGAGATGCTCAAGCATTTCTGTGGGGGCTCTGAACTCTAGCTTACGAGCACTACGGATCGAGGATTGT
GAAGCACTAAAGGAGTTTGATCTGTTTGAGAACGATGATAATTCTGAAATCATTCAGGGGTCATGGCTGCCTGGTCTTAGGAATTTGATT
GTGAAATGTTGCCCTCATTTGAAAGTGTTGAAGCCTCTTTCACCTTCAACTACCTTTTCTAAGGTAGTCATCAGAGAAGTTCCAAGATTT
CCGTATATGGAGGTATCATCTGGTGAAAAGTTAGAAATTGGGAAATTTGATGAGGACGGAGATGATTTTGATGAATCTTGTGATGAGTTG
AGGATACTGGATGACAAAATTTTGGCATTCCACAATCTTAGAAACCTCAAATCGATGGAGATATATGGTTGCAGAAATCTAAGGTCTTTT
CTGTTCGAAGGTTTCAGTCATCTTGTCTCTTTATTAAGTTTGGATATAACAAAGTGTGAACAACTTTTCTCTTCGGATATGTCGCCAGAG
TATACCCTTGAAGATGTGAGAGCTGTGAACTTCAATGCCTTCCCATTTCTCAAAAATCTCAGTATTGACTCATGCGGAATAGCGGGGAAG
TGGCTATCGCTGATGCTGCAGCATGCGCCAGGCCTAGAGGAATTGCGTTTAAGATATTGCGCACATATAACAAGAGTAGTGTTACCGATG
GAAGAGGAAGAAAACAGTCTCTTAACAACAGTAGTGTCATCAGGAAATCAAGATGAGGCATTGACCTGGTTAGTTCGTGACGGACTCTTG
CACATTCCATCAAATCTCGTCTCCTCTCTCAAGAAGATGACTATTGGTCAGTGCCCTCGCCTAAAGTTTAACTCGGGCAAGGACTGCTTC
TCTGGATTTACCTCGCTTGAGAAGCTTGAAATTTGGGGATCATTGGTGGATGATGACGGAAGTGATGACCTGGAGAATGGAAGTCCTTTT
GTGTTCGGAGAGGAGGATCAACCCCTGGGAGCGAATGGAAGATGGCTCCTCCCGACATCACTTCAGGAGCTTAACATCGGGTGGTTCTGT
TACCAAGAAACGCTGCAACCCTGCTTTCCTAGAGATATCACCAGCCTTAAAGAGTTAAGTGTACGTTCAATCCAAGGTTTGCAATCTCTA
CAGCTGCACTCATGCACGGCACTGGAAGGATTGGAGATTAGAGGCTGTGAATCGCTCACCGTCACTGTACTAGAGGGCATGCAACCCATT
GGCAGCCTCGTGCGTTTGAATGTATCAGACAGTACTGGCTTGCCACCATGTTTGGAGAGCTTTTCAACGCTGTGCCCTCGGCTTGAAAGG
CTTTGCACCGATGACCCATCTGTCCTTACCACGTCATTCTGCAAGCACCTCACCTCCCTACAAAGACTAGAACTTAGTTTCTTGAAAGTG
ACGAGACTAACAGATGAGCAAGAGCAAGCGCTTGTGCTGCTCAAATCCCTGCAAAAGCTCGAATTCATTTGGTGTTCTGCTCTAGTAGTT
CTTCCTGAGGGGCTGCACACCCTTCCTTCCCTCAAGAGATTGGAGATAAACCAGTGTGGACGCATCACAAGGCTGCCAGAAGCAGGCCTC
CCACATTCGCTGGAAGAACTCGAAATCCGGTCTTGCAGCCAGGAGCTAGATGATGAATGCAGGCGGCTAGCAACAAGCAAACTGAAAGTC
AAGATTGATTGGACGTATGTGAATTAA
>curated_TraesCS2901G48800_Ta_2B09
MMEPAGDSFVEAAIAWLVQTILATLLMDKMEEWIRQVGLADDVERLQREVERVDMVVAAVKGRAAGNRPLSRALARVKELLYDADDVVDE
LDYYRLQQQVEGVTSDKPDDMRGAERVDEISRGHVDTLNVSVGKLRSSVWEHFTITETVDRKRSKAKCKYCRKDFNCETKTNGTSSMKKH
LEKEHSVTCTKKRGAHPPNPSSTGDATCNVRSVEVGSSSNGKRKRTNEDPTQTTAANTHTQWDKAEFSNRIIKITGQLQSQDIQGALSKV
LGPYGPSATSSSSHHRPSTTSAQHPTTSSLVPLEVYGRVAEKNKIKKSITENQSGGVNVLPIVGIAGVGKTTLAQFVYNDPDVKSQFHHR
IWVCVSRKFDEVKLTKEMLDFFPRERYEGISNFAKLQEILKEHIEYQSKSFLLVLDDVSDNVDYHKWNKLLYPLMSSQAKGNIILVTTKN
LSVAQRLRTLEPrKLGALENDDMWLLLKSCAFGFGDYKGPGNLRAIGMQIAEKLKGNPLAAVTAGALLRDHLSVDHWSNILKKEKWKSLG
LHGGIMPALKLSYDELPYHLQQCFSYCSIFSEKYRFLRKELVYIWISQGFLNHTKRLEEIGWECLNNLVNLGFFQQIGEQQEGDEDEEED
FFLGSKIWYCMSGLMHDFARMVSRTECATMDGLQCNNMLPTIRHLSIVTNSAYSKEQHGTIPRNIKFEENLRNAFASVRKLRTLVLFGHY
DSFFFKLFLDIFQKDQNLRLLQMSATCADFDSFMCSLVNPAHLRYLKREPDEVNGASPQILSKLYHLQILDVGSYTDPIPDGNNNLVSLR
HLIPENGVYSSIASIGRMTSLKELHHFKVRFCSRGFEISQLQCMNELVQLGVSRVDSVKTREEAYGAGLRSKEYLKNLHLSWKDTLSQKE
CDTSSEYSADENEELSQMDTAREVLEGLEPHMNLKHLHISGYNGTTSPTWLANNLSVTSLQTLHLDGCRRWRILPSLESLPFLTKLKLSS
MLEVIEVLVPSLEELVLMDMPKLVRCSSISVGALNSSLRALRIEDCEALKEFDLFENDDNSEIIQGSWLPGLRNLIVKCCPHLKVLKPLS
PSTTFSKVVIREVPRFPYMEVSSGEKLEIGKFDEDGDDFDESCDELRILDDKILAFHNLRNLKSMEIYGCRNLRSFLFEGFSHLVSLLSL
DITKCEQLFSSDMSPEYTLEDVRAVNFNAFPFLKNLSIDSCGIAGKWLSLMLQHAPGLEELRLRYCAHITRVVLPMEEEENSLLTTVVSS
GNQDEALTWLVRDGLLHIPSNLVSSLKKMTIGQCPRLKFNSGKDCFSGFTSLEKLEIWGSLVDDDGSDDLENGSPFVFGEEDQPLGANGR
WLLPTSLQELNIGWFCYQETLQPCFPRDITSLKELSVRSIQGLQSLQLHSCTALEGLEIRGCESLTVTVLEGMQPIGSLVRLNVSDSTGL
PPCLESFSTLCPRLERLCTDDPSVLTTSFCKHLTSLQRLELSFLKVTRLTDEQEQALVLLKSLQKLEFIWCSALVVLPEGLHTLPSLKRL
EINQCGRITRLPEAGLPHSLEELEIRSCSQELDDECRRLATSKLKVKIDWTYVN-
>curated_TraesCS2B01G488400_Ta_2B10
ATGGCGGCCGCGATTgGGTGGCTGGTTGAGACCATCTCTGCGACCCTCCAAATCGACAAGCTCGACGCCTGGATTCGGCAAGTCGGTCTT
GCCGATGACATCGAGAAGCTCAAGTCGGAGATCCGGAGAGTCAACATAGTGGTCACTGCTGCCAAGGGCAGGGGGGTAGGGAGCGAGCTG
CTGGATGGACCTTTCGCTCTTCTGGAGGAGCGGCTCTATGAAGCCGACGACGTGGTCGACGAGCTCGACTACTACAGGCTCCAACACCAA
GTCCAAGGTCTGCCGGCACCTGCAGATCCAAGCGAGCCAGTCCCACTCCCAGTCCCAGGAGGTAAGCGTAAATCTGTCTAGACCCAAGTA
ATCCAAGTCTGCTAATTATTAGTTTGATCTTATGTTGCTCCAAAAATGTAAATTGGTCGTATCTGATCAAGGACGACCGTTCTTTAATTT
CTGGTCCACGATTTCTTTTGGCACAGTTACAAGGGGTGAGCCCGAAGGCGTGCTTGTAGCTGAGCAATTCAATGAGATATCGAGGGGCGG
TGGTGATGTACCACAGAGCAATGTTGGCAAATTACGGTCCGTGGTATGGGAACACTTTATGATCACAGAAAGAGATAACGGAAAACCCAA
CAAGGCAGTATGCCGACACTGTAGCAATGAGTTTAAGTGTGACACCAAGACGAACGGTACATCATCTATGAAAAAGCATTTGGAGAATGA
GCATTCTGTGACTTGTACAAAGAAACCTCCTGGAGCACATCTACCAAACCCTTCAAGGTACTTAAAAGAGAATTGGGTATAGAGAGTAGA
GTATTCTTTCTAATCTTAAGTGTACATTTTTAAAAAGTTGTTTATATACATATGCTTGAGGCGATTGTGGTCCTGATTAATAAGCACATC
CCCCGCAAAATAAATAAATACGCACCTCTTTTTTTCTCACCACAGCACCGGTGAGCCTACTATAATTGCCAGCTCATCCAGCAAAAAACG
AAAGAGACGACGGTCCAAGGCATGGGAATTTTTTGATGTCATAGAAGAAGTAAACGAACAGCCTATGAAAGCAAGATGTAAATACTGTCC
CGCAGAGATCAAGTGCGGCCCAACAAGTGGGACAGCAGGTATGCTCAACCATAACAAGATTTGTAAGAACAAACCTGGACCAAATGACCA
GTTGCCAAACCTGTCAAGGTAACTAAAGAATCTATATGTTGCGTCGAAAAACAATTAGAAGTCATTAAGTTAAGAGTCTCATTGTGGTTC
TAATAGTCAATTAACGTTCTTTTTTCTTATTGTAGCACCGGTGATGCTAATGCGGATGTGACGCCAATTCTAATAGGTAACTCGTCCACC
AGAAAAGGGAGAATGGATGATTCCATACAAATTGATGTGACTAACACAGTCACCCCTTGGGACATGGCCGAATTATCCAGCAGGATACGA
AAAATAGCTAGTCAGTTGCAATACATCCAAGAGGAAACGACTGAAATTCTCAAGCTACATGGATCGGACTCTACTTCAAGTTCAGATCAT
CACCAGAGTACAACATCATATCAGCACCTCAGAACATCAAGTCTTGTTCCAAGGAATGTGTATGGAAGAGTTAAAGAAAAGGAACACATC
ATGAAATTGATGATGACAGAAGGCAGATCTGACAAAGTAATTGTTGTGCCTATTGTAGGCATTGCAGGTATTGGAAAGACAACTCTCACT
CAACTTGTGTACAACGATCCAGAAGTGGAAAGGCAATTTGAACATAGGATATGGGTTTGGGTGTCTCGCAACTTTGATGAAATGAGGCTC
ACAAGGGATATGCTGAGCTTTGTTTCTCAAGAAAGTCATGAAGGAATAGGCTGCTTTGGGAAGCTTCAGGAGATCCTGAGAAGTCATGTC
AAATCAAAGAGGGTTTTACTTATTTTAGATGATGTATGGTATGACAAGAAAGATGCCCGATGGAACCAACTATTGGCTCCCTTTAAGCCT
CATAGTGCCAATGGCAATGTGATTCTTGTGACAACTAGAAAAATGACCGTTGCAAAAATGATTGGAACAGTGGTGCCAATTAAGTTAGCT
ACTATTGAAAATGATGACTTTTGGTTATTATTCAAATCATGTGCTTTTGTTGATGGAAACTATGAATGTCTTGGAAATCTTAGCACTATT
GGACGGCAAATAGCAGAAAAGTTAAAGGGTAACCCGTTAGCAGCAGTGACTACAGGGGCACTATTAAGGAACCAACTTACCGTTGATCAT
TGGAGTAAAATTCTCAAGGAAGAAAATTGGAAATCATTAGGACTTAGTGGAGGCATCATGCCTGCTTTGAAGCTTAGTTATGATGAGTTG
ACATACCGTTTACAACAATGTTTCTTGTATTGTTCTATATTTCCTGACAAATATAGGTTTCTTGGTAAGGATTTGGTATATATGTGGATT
TCTCAGGGATTTGTGAATTGCACCCAAAATAAGAGATTGGAGGAGATAGGATTGGAATATCTGAATCATTTGGTAAACCTGGGATTCTTT
CAGCAAATTGAAGAACAGCAAGAATTGGATGAGGAAAAAGAATTCTCTCTACGCGGTCAGATTTGGTATTCTATGTGTGATCTCATGCAT
GATTTTGCGAGGATGGTTTCGGTGACTGAATATGCGAGGATAGATGGTCTGCAGTGTAAGAAAATCTTACCGACTATACACTATTTGTCA
ATAGTAACTGGTTCTGCATACAACAGAGATCTGCATGGGAATATTCCTCGCAATGAGAAGTTTGAAGAAAATCTGAGAAATTCTGTTACA
TCAGTTACCAAATTGAGAACACTGGTTGTACTTGGGAGCTTTGACTATTTCTTTGTACAGTTGTTCCAAGATATATTTCAAAAGGCCCAA
AATTTACGCCTGCTGCGAGTATCTCCAGAATCCACTTATCTGTTTCAAGTGCCTGCAGCATCCACTGATTTTAATTCCTTCCTGTGCAGT
TTGGCAAATCCTTTGCATCTTCGTTATCTAAAACTTGATTTAGACGGGATTGTGCCACAAGTTCTCAGTACTTTTCTTCTTCTTCAAGTA
TTAGATGTTGGCTCAAACAGGGATACTTCTCTACCCAATAGCTTGCATAATCTTGTTAGCCTGCGACATCTTGTTGCACACAAGAGAGTC
CATTCTTCCATTGCTAGCATTGGCAACATGACATCTATCCAGGAGCTACATGATTTTGAGGTTCGAATTTCTAGCGGCTTTGAGATTACA
CAACTCAAATCCATGAACAAGCTTGTTCAACTTGGAGTGTCTCAACTTGACAGTGTTAAAACCCGGGAGGAGGCTTATGGGGCAGGACTA
AGAAACAAGGAACACTTAGAAGAGCTTCACTTGTGTTGGAAGCATGCATTTTCAGTGGATAAGGATGTCAGTGACACTAGATTTGAATCT
TCTGCAGACATGGCCAGAGAAGTGATTGAGGGTCTTGAACCACACATGGATCTAAAACATCTACAAATATCTCGGTATAATGGTACCACT
TCACCGACTTGGCTTGCCAATAATATCTCAGTTACCTCACTGCAGACGCTTCATCTTGATGATTGTGGAGGATGGAGAATACTTCCATCT
CTGGGAAGTCTTCCATTTCTTACAAAGTTGAAGTTGAGCAACATGTGGGAAGTAACAGAAGTATTGGTTCCTTCACTGGAGGAGCTAATT
TTACTCAACATGCCCAAGTTAGTGAGATGCTCAAGTACTTCTGTGGGGGCTCTGAACTTTAGTTTACGAGCACTGCGGATCGAGGATTGT
GAAGCACTGAAGGAGTTAGATCTGTTTGAGAACGATGATAATTCTGAAATCATTCAGGGGTCATGGCTGCCTGGTCTTAGGAATTTGATT
GTGAAATATTGCCCTCATTTGAAAGTGTTGAAGCCACTTCCACCTTCAGCTACCTTTTCTAAGGTACTCATCAAAGTGGTTTCAAGATTT
CCGTCTATGAAGGTATCATCGGGTGAAAAGTTAGAAATTTGGGATGCTAATTACCGCAGAGGCGATCGATCTTGTGATGAGTTGATCATA
CTGGATGACAAAATTTTGGTGTTCCATAATCTTAGAAACCTCAAATCGATGGAGATATTTGGTTGCAGAAATCTAAGGTCTTTCTCGTTT
GAAGGTTTCAGTCATCTCGTCTCTTTAACGAGCTTGAAAATAAGAGGCTGTGAAAAACTTTTCTCTTCACATGAGATGCCAGCCATTGAA
CATGTGACAGCTGTGAACTGCGATTCTTTCCCATCTCTTAAAAGTCTCAGTATTAAGTCATGTGGAATAGCGGGGAAGTGGCTATCGTTG
ATGCTGCAGCATGCGCCAGGCCTAGAGAAATTGAGTTTAAGATATTGCGCACATATAACAACAGTACTGTTACCGATGGAAGAGGAAGAA
AACAATCTATTAACAACAGTACTGTCATCAGGAAATCAAGATGAGGCATTGACCTGGTTAGCTCGAGAGGGACTCTTGCACATTCCATCA
AATCTCGTCTCCTCTCTCAAGAATATGAGTATTAGTGAGTGCCCTCGTCTAAAATTTAACTGGGGCACGGACTGCTTCTCTGGATTTATC
TCGCTTGAGAAGCTTGAAATCTGGGGATCGTTGGTGGATGATGACGGAAGTTATGACCCCGAGAATGGAAGTTCTTTTGTGTTCGAAGAG
GAGGATCAACCCCTGGGGGCGAACGGAAGATGGCTCCTCCCGACATCACTTCAGGAACTTAACATCAGGTTCTTGTGTTACCAAGAAACG
CTGCAACCCTGCTTTACTAGAGATATCACCAGCCTTAAAAAGTTATATGTAAGCTTCAGCCCAGGTTTGCAATCTCTACAGCTGCACTCA
TGCACGGCACTGGAAGAATTGGCAATTGTCGGCTGTGGATCAGTCACCGTCACTGTACTAGAAGACTCTCCTGGCTTGCTGCCATGTTTG
GAAAGGCTTTGCATCAATGACCCATCTGTCCTTACCACGTCATTCTGCAAGCACCTCACCTCCCTGCAACGCCTACGACTTGGTTTCTTG
AAAGTGAGGAGACTAACAGATGAGCAAGAGCAAGCGCTTGTGCTGCTCAAATCCCTGAAAGAGTTCCAATTCTATTTGTGTAATGATCTC
GTAAATCTTCCTGCTGGGCTGCACACCCTTCCTTCCCTCAAGAGGTTGGAGATAGAACGGTGTGGACGCATCTCAAGGCTGCCAGAAGCA
GGCCTCCCACATTCGCTGGAAGAACTGAAAATCGAGTCTTGCAGCCAGGAGCTATATGATGAATGCAGGCAGCTAGCAACAAGCAAACTG
AAAGTCAAAATTGGTGGGAGATATGAGAATTAA
>curated_TraesCS2B01G488400_Ta_2B10
MAAAIGWLVETISATLQIDKLDAWIRQVGLADDIEKLKSEIRRVNIVVTAAKGRGVGSELLDGPFALLEERLYEADDVVDELDYYRLQHQ
VQGLPAPADPSEPVPLPVPGVTRGEPEGVLVAEQFNEISRGGGDVPQSNVGKLRSVVWEHFMITERDNGKPNKAVCRHCSNEFKCDTKTN
GTSSMKKHLENEHSVTCTKKPPGAHLPNPSSTGEPTIIASSSSKKRKRRRSKAWEFFDVIEEVNEQPMKARCKYCPAEIKCGPTSGTAGM
LNHNKICKNKPGPNDQLPNLSSTGDANADVTPILIGNSSTRKGRMDDSIQIDVTNTVTPWDMAELSSRIRKIASQLQYIQEETTEILKLH
GSDSTSSSDHHQSTTSYQHLRTSSLVPRNVYGRVKEKEHIMKLMMTEGRSDKVIVVPIVGIAGIGKTTLTQLVYNDPEVERQFEHRIWVW
VSRNFDEMRLTRDMLSFVSQESHEGIGCFGKLQEILRSHVKSKRVLLILDDVWYDKKDARWNQLLAPFKPHSANGNVILVTTRKMTVAKM
IGTVVPIKLATIENDDFWLLFKSCAFVDGNYECLGNLSTIGRQIAEKLKGNPLAAVTTGALLRNQLTVDHWSKILKEENWKSLGLSGGIM
PALKLSYDELTYRLQQCFLYCSIFPDKYRFLGKDLVYMWISQGFVNCTQNKRLEEIGLEYLNHLVNLGFFQQIEEQQELDEEKEFSLRGQ
IWYSMCDLMHDFARMVSVTEYARIDGLQCKKILPTIHYLSIVTGSAYNRDLHGNIPRNEKFEENLRNSVTSVTKLRTLVVLGSFDYFFVQ
LFQDIFQKAQNLRLLRVSPESTYLFQVPAASTDFNSFLCSLANPLHLRYLKLDLDGIVPQVLSTFLLLQVLDVGSNRDTSLPNSLHNLVS
LRHLVAHKRVHSSIASIGNMTSIQELHDFEVRISSGFEITQLKSMNKLVQLGVSQLDSVKTREEAYGAGLRNKEHLEELHLCWKHAFSVD
KDVSDTRFESSADMAREVIEGLEPHMDLKHLQISRYNGTTSPTWLANNISVTSLQTLHLDDCGGWRILPSLGSLPFLTKLKLSNMWEVTE
VLVPSLEELILLNMPKLVRCSSTSVGALNFSLRALRIEDCEALKELDLFENDDNSEIIQGSWLPGLRNLIVKYCPHLKVLKPLPPSATFS
KVLIKVVSRFPSMKVSSGEKLEIWDANYRRGDRSCDELIILDDKILVFHNLRNLKSMEIFGCRNLRSFSFEGFSHLVSLTSLKIRGCEKL
FSSHEMPAIEHVTAVNCDSFPSLKSLSIKSCGIAGKWLSLMLQHAPGLEKLSLRYCAHITTVLLPMEEEENNLLTTVLSSGNQDEALTWL
AREGLLHIPSNLVSSLKNMSISECPRLKFNWGTDCFSGFISLEKLEIWGSLVDDDGSYDPENGSSFVFEEEDQPLGANGRWLLPTSLQEL
NIRFLCYQETLQPCFTRDITSLKKLYVSFSPGLQSLQLHSCTALEELAIVGCGSVTVTVLEDSPGLLPCLERLCINDPSVLTTSFCKHLT
SLQRLRLGFLKVRRLTDEQEQALVLLKSLKEFQFYLCNDLVNLPAGLHTLPSLKRLEIERCGRISRLPEAGLPHSLEELKIESCSQELYD
ECRQLATSKLKVKIGGRYEN-
>curated_TraesCS2B01G488600_TraesCS2B01G488700_Ta_2B11
ATGGAGGCCGCGATTGCATGGCTGGTGCAGACCATCCTTGCAACCCTCCTGATCGATAAGCTCGATGCGTGGATTCGGCAAGTCGGGCTT
GCCGATGACGTTGAAAAGCTCAAGTCAGAGATCAGGAGAGTCAAGATGGTGGTCTCGGCTGTGAAGGAGAGAGGGATCAGGAACGAGTCG
CTGGATGAATCTCTCGCTCTTCTCGTGGAGCGACTCTACGAAGCCGACGACGTGGTCGACGAGCTGGATTACTACAGGCTCCAAGAGCTG
GTTGAAGGTGCCCGGCCCCGGCTGCCTGCAGATCCAACCGTGCTGGTTCCTTCCAACCTGCCCATCCAAGGAGAAGGAGGTACGCATACT
TCTTCCTGTAGATCCAACACAAAGTTCTTTCATAGGCCGAGTATCGAAGTGTGACAAACTACTAGTAATTGTTAGTCTGATGATCCTATC
TTACTTAGGACAAATTAATGAAATTTATATTATCTGATCAAGGACGACCATGCTTTTCTGGTCCATTTTTCTGTTGGCACAGCTACAAGA
AACGAGCCCGAAGGTAACAGTGCTGGCAAATCACGGTCCGTGGTCTGGGAAAACTTTACAGTCACAGAAACTGTTGACAGAAAGTCCGCC
AAAGCAGTATGTAGACACTGTGGCAATGAGTTCAAGTGTGATACGAAGATCAACGGTACATCATCTATGAAGAAACATTTAGAGAAGGAG
CATCCCGATAAGATGAAACCTCCTGGAGCGCATCCACCAAACCCTTCAAGGTACCTAAAGAAGAATTGAGCATGAGCCCATTTAATTAGA
AATCGTTTATATACCTCTTTCTTTTTTCTTGAATGGTTATATACATCTTCTTGACAGCGCACTAATTTTGGTCCTAATAGCCAACCCACC
ACTTTTTTCTTACTGCAGCACTGCTGAGCCTATTGCCATTGCCAGCTCATCCAGGGGAAAAGGAAAGAAACAGCGGTCCAAGGCATGGGA
TAATTTTGATGTTATAGAAAATGACATTGGACAGCCAACCAAAGCAATATGTAAATACTGCCACACAGAGATCAAGTGCGGAATGAAGAC
CGGGACAGCGGGTATGCTTAACCATAACAAGATTTGCAAGAAGAAACCTGAACCAAATGACCAGCCACCAAACCTGTCGAGGTAGCTACC
TTGCATCAGCAAATTTTTGGATGTTGTTTTATAAACAATCCCCACCATGGTTCTAATAGCCGTTTGTTCATGATCTTTTTCTTACTGCAA
CATTGGTGATGCTACTGCAAATGCGACATATATTGTGGTTTATGACGATTCAGCTACAAGAAAAAGAAGGAGAGTGGATGAGGAGTCAGC
AGAAATCACTGCAGCTAATACACACACCTGTTGGGACAAGGCTACATTATCCAATATGATACGAAAAATTATTAGTCAGTTACAAGAGAT
CCAAGGGCAAGTGAGGGAGGTTATCGAGTTACATGGATCAGACTTATCTTCCAGTTCAAATCACCATCAAAATACAACCTTATATCAGCG
CCTACGGACATCAAGTCTTGGTCCAAGAAAAGTGTATGGAAGAGTTGCAGAAAAGAACTCCATTGTAAGGATGATAACAGGAGAAAAGTC
TGGTGGTTTAGTTGTTCTGCCTATTGTAGGCATTGCAGGTGTTGGCAAAACAACTCTTGCTCAACTTGTATACAATGATCCATATTTGGA
TGATCATTTTGACCAAAGGATATGGGTTTGGGTGTCTCGCAATTTTGATGAAGTGAGACTAACAAGGGAGATTTTGAACTCTGTTTATCA
AGAAAGGCATGAAGATATAAAATGTTTTGCGAAGCTTCAGGAGATCTTGAAGCATCAGGCCGACTCACAGCGACTTTTAATCATTTTAGA
TGATGTCTGGGATGACATGAACGATAATATCCAACACCATAAAATGTTGGCTCCTCTGGTATCAAGTCATGTGAAGGGTAATGTGATTCT
AGTCACAACCAGAAGTATGTCTGTTGCACAAAGCTTAGGCACCCTCAAGCCAGTCAAGTTAGGTGCTCTGGCAAATGATGACTTTTGGTT
ATTGTTCAAATCACACGCATTTGGTTACGAGAACTGTCAGGAGCATCAAAGTTTAAGTATCATCGGGCGGCAAATAGCCGAGAAGTTAAA
GGGCAACCCATTAGCAGTTGTATCTACAGCAGAACTATTACGGAAGAAACTTAACACCGATTATTGGAGAATCGTTCTAAAGAACGAAGA
GTGGAAATACATGCATCACAATAGAGGGATCATGGCTGCTCTGAAGCTTAGCTATGATCAACTTCCGTACCATTTACAACGGTGTTTCTC
ATATTGCTCCATATTCCCTGACAGTTATCAGTTTCTTAGTGAGGAGTTGGTCGGTTTCTGGATATCACAGGGATTTGTAAAGTGCAACGG
CTCTAGTCAGAGATTGGAGGATATAGGGCGGGGATATCTGATTGATTTGGTTAACCTGGGCTTCTTTGAAGAAGCTAAAAGAGAAGAACC
ATATCTAGGCAGTCAAGTTATGTATGCCATATGCGGTCTCATGCATGATTTTGCGATGATGGTTTCAAGGACTGACAGTGCAAGTATAGA
TGGTCGACCCTACAAAAAAATGCCTCGAACTCTACGACATTTGTCAATAGTAAATGGATCCGCATACCAGAAAGATCAGCATGGGAACAT
TTATCATGATGAGAAGTTTGAAGAAAATCTGAAAAATGCAATTACATCAGTTAGTGAACTGAGGACATTAGTGTTACTTGGGCACTATGA
CTTTTCCTTCTTACTATTATTCCAATATATATTCCAAAAGGCACATAACTTACGTGTGCTACAAATGTCTGCAGCATCTGCTGATTTTCT
CAAACATGGGATTGAGGAGGTGGATGGGTCTTTCCCTCAAATTTTGAGCAAATTGTACCATCTCCAAGTATTAGTCGGTTCATACAATGA
TCGTACTATGCCTGGTTGTATTGATAATCTTGTTAGCCTGCGGCATCTTGTTGTACACAAGGGAGTGTACTCTTCCATTGCAACCATTGA
TAATATGCTATCATTTCAGGAACGACATGGTTTCAAGTTTCATATTTCTAGTGGCTTTGAGATAACACAACTCCAATCCACTGAACATTG
GATGCATGTTAATACTCTGGAAGATGTTTATGAGGCAGGACTGGTAAACAATGAACTCTCAGAAAAGTTGCACTTGTCCTGGAAGGATTC
TCCTGCGGACATGGTCATGGAGGTTGAGGGTTGGGAACCACATTGSGACTTAAGGGTTCTCGAGATATCTGGGTATAATTTTGCTTGGAC
AATTATGGTTGACAACATTATCTTGGTTACCTCCTCCCAGACGGTTCACATATGCGATTGCATTGAATGGAAAATACTTCCATCTTTGGA
AAGGTTTCGGTTTTTGACAAAGCTGGAGTTGAGAAACCTGCCTAAAGTAATACAAATACTGGTTCCTTCACTGGAGGAGCTAGCTTTAGT
TAAAATGCCAAAGTTGGAGAAATGTACATGCACTTCCGTGGAAGGTATGAGCTCTAGACTAAGAGCACTGCAGATCAAGGATTGTCAATC
ACTGAAGGAGTTTGATCTGTTTGAGAACAACGATAAATTCGAAACTGGGCAGAGGTCATAGGCTCCTAGTCTTAGGGAACTAAGTCTGGA
GAATTGCCCCCATTTGAAAGTGTTGAAGCCTCTTCCACGCTCAAGCATGTGTTCTGAGTTACTCATCTGTGACGTTTCAACACTTCCGTA
CATGAAGGGATCATCTGATGAAGAGTTATGTATTGGGTATGATGGTGAGTATGGCTATGGTTTTGACGAATCTTCCGATGAGTTGAAGAT
ACTGGATGACAAAATTTTGCTGTTCCATAATCTGAAAAACCTCAAATCGATGGTGATACATGGTTGCCGGAATCTAAGTTCCATTTCATT
AAAAGGTTTTAGTTACCTCGTCTCTTTAACGAGCTTGAAAATAAGAAATTGTGAAAAACTTTTTGCTTCAAATGAGATGCCAGAGCATAC
CCTCGAAGATGTGACACTTGTGAATTGCAAGGCTTTCCCATCTCTGGAATGTCTCAGTATTGATTCATGTGGTATAGTGGGGAAGTGGCT
ATCTTTGATGCTGCAACATGCGCCATGCCTAGAGGAATTGTATTTGTCTTCCCAAGAGGAAGAAAAATCAGAAGAGGAAGAAAACAGTAT
ATCAAATCTTAGCTCAACCAGGGAGGGCACATCATCCGGAAATCCAGATGACGGATTAGCTCTAGACCGACTGTTGTGCATCCCATTAAA
TCTCATCTCCATTCTAAAGAGGATAACTATTGAGAGGTGCCCTCATCTAACATTTAACTGGGGCAAGGAAGGCGTCTCGGGATTTACCTC
CCTTGAGAAGCTAGTCATTTTAGACCGCCCTGACCTGCTCTCGTCGTTGGTGCATACAGACGGAGGATGGCTACTCCCGAACTCACTTGG
CCAACTTGAAATCGATGGCCATTCCCAAGTAA
>curated_TraesCS2B01G488600_TraesCS2B01G488700_Ta_2B11
MEAAIAWLVQTILATLLIDKLDAWIRQVGLADDVEKLKSEIRRVKMVVSAVKERGIRNESLDESLALLVERLYEADDVVDELDYYRLQEL
VEGARPRLPADPTVLVPSNLPIQGEGATRNEPEGNSAGKSRSVVWENFTVTETVDRKSAKAVCRHCGNEFKCDTKINGTSSMKKHLEKEH
PDKMKPPGAHPPNPSSTAEPIAIASSSRGKGKKQRSKAWDNFDVTENDIGQPTKAICKYCHTEIKCGMKTGTAATRKRRRVDEESAEITA
ANTHTCWDKATLSNMIRKIISQLQEIQGQVREVIELHGSDLSSSSNHHQNTTLYQRLRTSSLGPRKVYGRVAEKNSIVRMITGEKSGGLV
VLPIVGIAGVGKTTLAQLVYNDPYLDDHFDQRIWVWVSRNFDEVRLTREILNSVYQERHEDIKCFAKLQEILKHQADSQRLLIILDDVWD
DMNDNIQHHKMLAPLVSSHVKGNVILVTTRSMSVAQSLGTLKPVKLGALANDDFWLLFKSHAFGYENCQEHQSLSIIGRQIAEKLKGNPL
AVVSTAELLRKKLNTDYWRIVLKNEEWKYMHHNRGIMAALKLSYDQLPYHLQRCFSYCSIFPDSYQFLSEELVGFWISQGFVKCNGSSQR
LEDIGRGYLIDLVNLGFFEEAKREEPYLGSQVMYAICGLMHDFAMMVSRTDSASIDGRPYKKMPRTLRHLSIVNGSAYQKDQHGNIYHDE
KFEENLKNAITSVSELRTLVLLGHYDFSFLLLFQYIFQKAHNLRVLQMSAASADFLKHGIEEVDGSFPQILSKLYHLQVLVGSYNDRTMP
GCIDNLVSLRHLVVHKGVYSSIATIDNMLSFQERHGFKFHISSGFEITQLQSTEHWMHVNTLEDVYEAGLTEDGYSRTHLANLKSMAIPK
-
>curated_TraesCS2B01G734100LC_Ta_2912
GTATATTGTTTCTGCTCTGCTCGCGTGCTCCCCACCCTCGAGCCTCGACTCCCCCCACACTCTCCACTGACAAGAAACCATCTCCAGCGA
ACATCTTCTGCCGGATCTGATGGCGGCCTCGATTGGGTGGCTGGTTGAGACCATCTCTGCAACCCTCAAGATCGATAAGCTCGATGCCTG
GATTCGGCAAGTCGGACTTGCCGATGACATCCAGAAGATCAAGTCGGAGATCTGGAAAGTCCAGACAGTGGTCACTACTCTACTGCCAAG
AGTACGGGGGTCGCAAACGAGCTTCTGGATGAAGCTTTCGCTCTTGTCGAAGAGCGGCTCTATGAAGCCGACGATCTTGTCGACGAGCTC
GACTACTACAGGCTCCAACACCAAGTCCAAGGTCTGCCTGCCCCTGCAGATCCAAGCGAGCTACTCCGAAGAGGTAAGCGTAAATCTCTC
TACACCCAATTAATCCAAGTCAGCTAATTATTAGTTTGATCTTATATTGCGCCAAAAATTTAAATTGGTCGTATCTGATCAAGGACGCCA
TTGCTTTTCTGCTCCACGATTTCTTTTGGCACAGTTACAAGGGGTGAGCCCGAAGGTGTGCTTGTAGCTGAGCGACTCAATGAGATACCG
AGGGGTGATGGTGATATAGCACAGAGACAGAGCAATGTTGGCAAATTACGGTCCGTGGTATGGGAACACTTCACGATCACACAAAGAGAT
AATGGAAAACCTGTCAAAGCAGTATGTGTACACTGTAGAAATGAGTTTAAGTGCGATACGAAGACGAACGGTACATCATCTATGAAAAAG
CATTTGGAGAATGAGCATTCTGTGACTTGTGCAAAGAAACCTCCTGGAGAACATCCAGCAAACCCTTCAAGGTACTTAAAAGAGAATTGG
GTATAGAGTAGAGTATTCTTTCAAGCTCAGATGTACATACACCCCTTACCTTGTACTCCCTCCGTTCCATATTAATCGTCGCTGATTAGT
ACAACTAATATGGAACGGAGGGAGTATGAGGGAGGCTATGAGCACATTTAAGAAAAAAGTGTTCATATACATCTGCTTGAGGCCATTATA
TGTTCCTAATAACCCCATCTTTTTATTACTGCAGCACCGGTGAGCCTACTGTAATTGGCAGCTCATCCAGCAGAAAAGGAAAGAGACGAC
GGTCCAAGGCATGGGAACTTTTTGATGTCATACAAGAAGTAAACGAACAGCCTATGAAAGCAAGATGTAAATACTGTCCCACAGAGATCA
AGTGCGGACCAACGAGTGGGACAGCAGGTATGCTCAACCATAGCAAGATTTGTATACCTGGACTAAACAACCAGCCGCCAAACCCGTCAA
GGTAACTAAAGAATCTATACATTGCACCGAAAAATATTAGAAGTCATTAAGTTAAGAGTCTCACTGTGGTTCTAATAGCCAATTCACGGT
CTTTTTCCTATTGCAGCACTAGTGATGCTAATGCAAATGTGACGCCAATTACTGCGGCTAACACGGTCACCCCTTGGGACATGGCTGAAT
TGTCCAACAAGATTAAAAAAATAGCTGGTCAGTTGCAATACATCGGAAGGGAAGTGGGTGAGATTCTAAAGCTACATGGATCCGACTGTA
CTTCAAGTTCAGATCAGCACCTCAGAACACCAAGTCTTGTTCCAAGGAATGTGTATGGAAGAGTTAAGGAAAAGGAACACATCATGAAAT
TGATGATGACAGAAGGCAGATCTGACAAATTAATTGTTGTGCCTATTGTAGGCATTGCAGGTGTTGGAAAGACAACTCTCACTCAACTTG
TATACAATGATGTAGAAGTGGAAAGGCAATTTCACCATAGAATATGGGTTTGGGTGTCTCGCAACTTTGATGAAATGAGGCTCACAAGAG
AGATGTTGAGCTTTGTTTCTCAAGAAAGACATGAAGGAATAGACTGCTTTGTGAAGCTTCAGGAGATCTTGAAAAGTTATGTTAAATCAA
AGAGGATTTTACTTATTTTAGATGATGTTTGGGATGACAAGAACAATTACCAGTGGAACCAACTATTGGCTCCTTTTCGGCACGACAATG
CTATTGGTAATGTGATTCTTGTGACAACTAGAAAATTGTCTGTTGCAAAAATGATTGGAACAACAAGACCAATTAAGTTAGGTGCATTGG
AAAATGATGACTTCGAGTTATTGTTCAAATCATGTGCATTAGGTGATGGAAACTATGAATTTCCTGGAAATTTTAGCACAATTGGGCAGC
ACATAATAGAGAAGTTAAAGGGCAACCCCTTAGCAGCAATAACTACTGGGTCGCTATTAAGGGATCATCTTACCGCTGATCATTGGAGTA
ACATTCTCAAGAAAGAAAGTTGGAAGTCACTGGGAGTCAGTGGAGGCATCATGCCTGCTTTGAAGCTTAGTTATGATGAGCTACCATACC
GTTTACAACAATGTTTCTCTTACTGTTCTATATTTCCTAACAAATATAGGTTTCTTGGTAAGGATTTAGTCTATATTTGGATTTCTCAGG
GATTTGTGAATTGCACCCAAAATAAGAGATTGGAGGATACAGGGTGGGAATATCTGAATCAATTGGTAAACCTGGGATTCTTTCAACAAA
TTGAAGAACAACAAGAATTGGATGAGGAAGAAGAATTCTCTCTATGCCGTCAGATTTGGTACTCTATGTGTGATCTCATGCATGATTTTG
CGAGGATGGTTTCAAGGACCAAATGTGCGACTATAGATGGTCCACAGTGCAATAAAATATTGCCAACTGTACAGCATTTGTCAATAGTAA
CCGGTTCTGCATACAACAAAGATCTGCACGGGAACATTCCTCGTAATGAGAAGTTTGAAGAACATCTGAGAAATTCAGTTACATCAGTTA
CCAAGTTGAGAACATTGGTTGTACTTGGAAAATTTGACTCTTCCTTTGTACAGTTGTTCCAAGATATATTCCAAAAGGCACAAAATTTAC
GCCTGCTACGAGTATCTTATCCACTTATCTGTTTCAAGTGCCTGAAGCATCCACCGGTTTTAATTCCTTCCTGTGCAGTTTGGCAAATCC
TTTGCATCTTCGTTACCTAAAACTTGAGTTGGATGGGATTGTGCCACAAGTTTTGAGTACGTTTTTGCATCTTCAAGTATTAGATGTTGG
ATCAAGCATGGATACTTCTCTACCCAATGGCTTGTTGCATAATCTTGTTAGCCTGCGACATCTAGTTGCACACAAGAGAGTCCATTCTTC
CATTACTAGCATTGGTAACATGACATCTATCCAGGAGCTACATGATTTTAAGGTTCGAATTTCTGGTGGCTTTGAGATAACACAACTCAA
ATACATGAACGAGCTTGTTCAACTTGGGGTGTCTCAGCTTGACAGTGTTAAAACCCGGGAGGAGGCTTATGGAGCAGGATTAAGAAACAA
GGAACACTTAGAAGAGCTTCACTTGTCCTGGAAGGATGCATATTCAGAGTATGAGTTTGTCAGTGACACTAGATTTGAATCTTCTGCAAA
CATGGCAAGAGAAGTGATTGAGGGTCTTGAACCATACATGGATTTAAAACATCTACAAATATCTTGGTATAATGGTACCACTTCACCAGC
TTGGCTTGCCAACAATATCTCAGTTACCTCATTGCAGTCGCTTCATCTTAATTATTGTGGAACATGGAGAACACTTCCATCTCTGGGAAG
TCTTCCATTTCTTACAAAGCTGAAGTTGAGCAACATGTGGGAAGTAAAAGAAGTATTGATTCCTTCACTGGAGGAGCTAGTTTTGATCGA
CATGCCTAAGTTAGTGAGATGCTCAAGCACTTCTGTCGAGGGTCTGTGCTCCAGCTTAAGGGTACTGCAGATCAAATATTGTAAAGCATT
GAAGGAGTTTGATCTGTTTGATAACGATGATAATTCTGGAATCACTCAGGGATCATGGCTGCCCGGTCTTAGGAATTTGATTCTGGATTA
TTACCCTCATTTGGAAGTGTTGAAGCCTCTTCCACCTTCAACTACGTGTTGTAAGGTACTCATCAGAGAAGTTCCAAGATTTCCGTATAT
GGAGGTATCATCTGGAGAAAAGTTAGAAATTGGGAATACTTATGGGTACAGAGGCGATGGTTTTGATGAATCTTCTGATGAATTGAGGAT
ACTGGATGACAAAACTTTGGCATTCCATAACCTTGGAAACCTCAAATTGATGGAGATATATGGTTGCAGAAATCTAAGGTCTTTTTCGTT
CGAAGGTTTTAGTCATCTTGTCTCTTTAGCAAGTTTGACAATAGTAGACTGCGAACAACTTTTCCCTTCAGATGTGTCGCCAGAGTATAC
CCTTGAGGATGTGACAGCTATGAACTGCAATGCCTTCCCATCTCTTAAAAGTCTCAGTATTCAGTCATGTGGAATAGCGGGGAAGTGGCT
ATCGTTGATGCTGCAACATGCGCCAGGCCTAGAGAAATTGGCTTTAGCAAATTGCGCCCATATAACAACAGTACTATTAACAACAGTATT
GTCCGATGGAAGAGGAAGAAAACAGACTATTAACAACAGTACTGTCATCAGGAAATCCAGATGAGGCATTGACCTGGTTAGCTCGAGACT
GACTCTTGCACGTTCAGTCACTCAAGATGATTGATATTTGGGACTGCCCCCGCCTAACATTTAACGGGGCCAAGGAATGCTTCTCTGGAT
TTACCTCCCTTGAGAAGCTAGTCATTCGAGGCTGCCCCGACCTGTTCTCGTCATTGGTACATAAAGACGTAACAGATGACCAGGCAAGCG
GAAGATGGCTCCTCCCGAAATCACTTCAGGAACTTGAGATCGTTGAATATTCCCAAGAAAAGCTGCAGCTCTGCTTCCCTAGAGATATCA
CAAGCCTTAAAAAGTTAAATGTATATCACAGCCCAGGTTTGCAATCTCTACGGCTGCACTCATGCACGGCACTGGAAGAATTGGAGATTA
GATGCTGTGGATCGCTCACCGTCACTGAACTAGAAGGCATACAACGCCTTGGCAGCCTCGGGCGTTTGAATGTATCAGACTGTCCTGGCT
TGCCACCATGTTTGGAGAGCTTTTCAACGCTGTGCCCTCGGCTGGAAAGGCTTGAGATCGATGACCCATCTGTCCTTACCACGTCATTCT
GCAAGCACCTCACCTCCCTGCAAAGACTACATCTTGGTCCCATGAAAATGACGAGACTCACAGATGAGCAAGAGCGGGCGCTTGTGCTGC
TGAAGTCCCTGCAAGAGCTCGAATTCAATCGGTGTCGTGATCTCGTAGATCTTCCTGGGGGCCTGCACAACCTTCCTTCCCTCAAGAGGT
TAAAGATATGGGATTGTCTGGGCATCTCAAGGCTGCCGGAAGCAGGTCTCCCATTTTCACTGGAAGAACTGGAAATCAATCATTGCAGCA
AGGAACTAGCTGACCAATGCAGTCTGCTAGAAACAAGCAAGCGAAAAGTGAAAATTACTTTATGTACTCCAATTGATTACTGGCTGCTAT
GTTAAGCACATGTTTCTAAGCTGTCTCTGCTTTTGAGGAAATCTTCCGCCGTATACCCTCAGAGTTGACAGACCCTCATAAATGTGCAGT
GTGCTCATTCCAGAATGAGCTGTCTCTGCAGGCATTCAATTAGGCTGCTCAACATATACTATCATGCAACAGGTAAACCGGCATGTTTCG
CTGTTTGCTATTCATCTTGTCTTGTCAACTGAAAAATATAATTAATTTTCATTTCCTTGACTGCACAGAGAACTACTCCCTCCGTTCCTA
AATATAAGTCTTTGTAGAGATTCCACTATAGACTACATACGGAGCAAAATGAGTGAATCTACGCTTAAAATGCATTTATATACATTCGTA
TGTGGTTCATACTAATATCTCTACAAAGACTTATATTTAGGAACGGAGGGAGTACACGAGATAAACCTGCAGATGTTTTATGTTGTTTGT
TGCACAAGTTGTGTCCGAAATTTCCGCCATTCAGATATGCTCTGCAGCTACAACAATGCACCTTTTCAAGGAAAAAAAAGCTAAAACAAA
GCACTTCAGAGACAGGAATAGTAGCTCTCGTCTGACACGAGAAGGAGGATATGTGGGGTTACTCTTAACTAAATTCATGTGTTGATCAGC
CAGACTCAGAAGTCAGGATGGCCTCGGCAGACGCCTAATGTGTGCAAGAATGATTAAAGTTGGATATGCAAGCCTGTAACCTGGTGTGCC
GTCGCCGATTACTAGTTTCCTGTTGTGATATCAGCGACGCAGTGTGTGTGTAGTATACTACTATGCTATCTTGGTACATCCTAATGAGCT
CATCTCTTCCCATTTTCCTTTATCTTTGTGATGCTTCAAACTATCTTTGTGATGCAGTGTGTCTGTACTATCCTATCTTGGATCTTCACA
GAATTTTGCTACTGGTCTGGACTCATTCTGTCAGTGGTTGTTTGCTTTGTGGACTTGTGCTCGTGGTCTCTGTTTTTTCAAGCTGATCCT
GAAGCTTGCTGGAGCCTGTGAGGCACGATAAAAATTCTCATCAAAGTGAGGCACAATAAAGCTCCTCGTTTCTTGTTGACTGTACGAGCT
CCTTTCTCCAGTGTGTAACTGAAAATGGGACGAGAATGCCGAAGGTTTGCTCATAAGGTCATATCACCATGCGAAACCCCAACAGTAACG
TCGGGGAAACAGAGTTGATATGGCCTCCTGTAAGAAAAAAGAGCTGGTACGGCCCGCTCCAGTTTCATCATTTCATTGCCATCCCTCGCA
TGTGTAGCGCTGTATCGGAGGAGCTCTCCTCTTTTGCGTGATATATTGCGTTATCAATAAGAAAACTATTCATGTCTTTGCTTCGGATAT
TTTTATGTATCTGAATTTTCTTGATCAGAAGAAAACTCTTTTTACTCTGTTTGTGATGCTGGACAAGTCATGCTGTCTTCGAACTGTGCA
TGAATAATTTTGCTCCTGATCTGGAGCACTTACATCGAGTGGTAGCTTACTTTGATGTGTGCACTAACAAAAGATTAGAAAATGTACATT
ATACCTGATGGCGTAATCAATCTTTTCTGTTGTGCTCAAGTTGTTGTCGATCATGCTTATCGTTTTCAGACTTCCTGAGCTGGCCGGCCT
GTGAATGTGGTAAGCAAACAAATTTTCTAGTCAATGATATATAGGCACAAGTAAAGAACAGGACAAGTTAACTGAATCCAAGGCAACCTG
CACATCTCAGAAACAAGTACTCACTCAAATCATACTGTTCAAGTAAGACGCTACAGGAAGTTAAGCTGCCCATCGTCTTAAACCAGCATA
GGATGCTCCCTTAACTCAAAATAAAGCTGTTAAAACAAGCTCCTCTGCAATGCAAGAACTTCATCAGTTCATGGAGAATAAACAGGGAGC
TCGACAGTACCGCAGGATGACGAGGAGCCACTGCCCACCAGAGATTGGTAAGTTGCGGTTGGATCTGGCCACAGCGCCTCCGCATCGGCG
CCCAGAGGTTGGTCGGATGGGGGATGTTGGCGAGCTCGCCTGCGAGGCGTTCCCTGAGCGCACTGCCATCACGGCGGGCCAGCCCCCGCT
TGCAGGAACGTCGGGCATCCCGGGCGGCGGCGTCTTGCAACTATCGGCGCGTGGCGTGGGAGGGCAAGCCTGAAGAAGACAAACTAGCTA
AATGGGCCGGACATTGGCACAGGCCATTGGCGCATATATTTTTATATTTTCCCAAAAAGTATACATATTAAAAATATATTCAGTAATCAC
TTTATATTTCTCAAAAAAATAATCAATTTA
>curated_TraesCS2B01G734100LC_Ta_2B12
MAASIGWLVETISATLKIDKLDAWIRQVGLADDIQKIKSEIWKVQTVVTTLLPRVRGSQTSFWMKLSLFSKSGSMKPTILSTSSTTTGSN
TKSKVCLPLQIQASYSEEDAIAFLLHDFFWHSYKGTGEPTVIGSSSSRKGKRRRSKAWELFDVIQEVWEQPMKARCKYCPTEIKCGPTSG
TAGMLNHSKICIPGLNNQPPNPSSTSDANANVTPITAANTVTPWDMAELSNKIKKIAGQLQYIGREVGEILKLHGSDCTSSSDQHLRTPS
LVPRNVYGRVKEKEHIMKLMMTEGRSDKLIVVPIVGIAGVGKTTLTQLVYNDVEVERQFHHRIWVWVSRNFDEMRLTREMLSFVSQERHE
GIDCFVKLQEILKSYVKSKRILLILDDVWDDKNNYQWNQLLAPFRHDNAIGNVILVTTRKLSVAKMIGTTRPIKLGALENDDFELLFKSC
ALGDGNYEFPGNFSTIGQHIIEKLKGNPLAAITTGSLLRDHLTADHWSNILKKESWKSLGVSGGIMPALKLSYDELPYRLQQCFSYCSIF
PNKYRFLVLDVGSSMDTSLPNGLLHNLVSLRHLVAHKRVHSSITSIGNMTSIQELHDFKVRISGGFEITQLKYMNELVQLGVSQLDSVKT
REEAYGAGLRNKEHLEELHLSWKDAYSEYEFVSDTRFESSANMAREVIEGLEPYMDLKHLQISWYNGTTSPAWLANNISVTSLQSLHLNY
CGTWRTLPSLGSLPFLTKLKLSNMWEVKEVLIPSLEELVLIDMPKLVRCSSTSVEGLCSSLRVLQIKYCKALKEFDLFDNDDNSGITQGS
WLPGLRNLILDYYPHLEVLKPLPPSTTCCKVLIREVPRFPYMEVSSGEKLEIGNTYGYRGDGFDESSDELRILDDKTLAFHNLGNLKLME
IYGCRNLRSFSFEGFSHLVSLASLTIVDCEQLFPSDVSPEYTLEDVTAMNCNAFPSLKSLSIQSCGIAGKWLSLMLQHAPGLEKLALANC
AHITTSLKMIDIWDCPRLTFNGAKECFSGFTSLEKLVIRGCPDLFSSLVHKDVTDDQASGRWLLPKSLQELEIVEYSQEKLQLCFPRDIT
SLKKLNVYHSPGLQSLRLHSCTALEELEIRCCGSLTVTELEGIQPLGSLGRLNVSDCPGLPPCLESFSTLCPRLERLEIDDPSVLTTSFC
KHLTSLQRLHLGPMKMTRLTDEQERALVLLKSLQELEFNRCRDLVDLPGGLHNLPSLKRLKIWDCLGISRLPEAGLPFSLEELEINHCSK
ELADQCSLLETSKRKCAHSRMSCLCRHSIRLLNIYYHATARLRSQDGLGRRLIVSVLSYLGSSQNFATGLDSFCQWLFALWTCARGLCFF
KLILKLAGAYFLSWPACECAVKTSSSAMQELHQFMENKQGARQYRRMTRSHCPPEIGKLRLDLATAPPHRRPEVGRMGDVGELACEAFPE
RTAITAGQPPLAGTSGIPGGGVLQLSARGVGGQA-
>curated_TraesCS2B01G489400_Ta_2913
ATGTTGCTCGGAATCTTCGAAACAGCTGAGCAGGCCGCGAGAACCTACGATGCGGCGGCGCTGCGCTTCAAGGGCGCCAAGGCCAAGCTC
AACTACCCCGAGGGTTTCCAGGGACGCACCGACCTCGGCTTCAAAGTCACCCGCAGCATACCGGACGGATTACAACAACATCGCCACTAC
CCCTCCACCATGGAGGCGCCAGCAACGCAGCCGTCGCCGCAACAGCAGCCGACCGTCCCAGTCCTCATGCGGCACGAACTGCCGCCTCAG
GGCGCCGGCAGCTCCAGGGGCGCTGTCAACCTGCCCTTCGGCGCCATGTCGGCCCCGTCCACGTCGTCCACCTCATCGCCGCACATGCTC
GTCCCTCCGCTTGCGTCCGAGGACCATACAATGAGAAGAACTGTAAGTGTAGAAGAGGAAGCTAACGACACACATGACGGAGTGACGGCG
CGCACACAATCTAGCAAGTTTGTGAACAGTTTTTACGGTTTTGCAAGTGCGTGTGCATTCTTTACTTTATCTGACTCTGGTCAAAGGACG
ACCCTTTTTCTTTTTCTTTTGGCAGTTGCAAGGAACAACGCCGAATGTATGCACGGTGCAGACAGGGTCGATGAGATATCAAGGGGCGAT
GCTGACACACCGAGTAACATTGTTGGCAAATTGCGGTCCGTCGTATGGGAACACTTTACGATCACAGAAAAAGATAATGGAAAACCGCTC
AAAGCAGTATGTAGACACTGTGGCAATGAGTTTAAGTGTGATACAAAGACCAACGGTACATCGTCTATGAAAAAACATTTGGAGAACGAG
CATGCCGTGACCTTTACCAAGAAACCTCCTAGAGGGCGTCCACCAAACCCTTCAAGGTACCCTCCCAAAAGAGAATTGGGCATATACCTT
GCATGAGCATATTTTTAGAAACTCGTTAATACACATCTGCTTCGGGAGCCCGATAATTGTGGTCCTAATAGCCAACCTAATGTCTCATTT
TCTTACTGCAGCACTAGTGAGCCTATCTTAATCGGCAACTCGTCCAGGACAAAAGGAAAGAGACGATGGTCCAAGGCATGGCAACTTTTT
GATATCATAGAAGAAGAAAACGGAGAGCCTATCAAAGCAATATGTAAATATTGTCCAACAAAGATCAAGTGTGGACCAATGTGTGGGACA
GCTGGTATGCTCAACCATAACAAGATTTGTAAGAACAAACCTGGACCATATGACCAGTCACCAAACCCATCAAGGTAGCTAATGAATCTA
TACCTTGCATCGACACATTTTTACAAGTCATTTAATTAAGAGGTCTCACCGTGGTTCTAGTAGCCAATTCACGGTTTCTTACATTAATTG
CTGCAGCACGGGTGATGCTACTGCACATGTGAAGCCTTCATCTAGCAGAAAAAGGAGGAGACCCGAATCAACACAAATGACCGCGCCTAA
CACCGCGACTGGTTGGGACAAGGTCGAGATATCCAATAGGATACAAAACATAACTAGTGAGCTACAAGGCATCCAACTGGAAGTGCCTAA
GGCTTTCTATCCATGTGGATCAAGCTTATCTTCAAATTCAGATCACCACCAGAGTACAATCTCAGATCAGCGCCTAAAGACATCAAGTCT
TGTTCAAAAGAAAGTGTATGGGAGAGATGTAGAAAAGAACTCCATCGTGAAGTTGGTGAGGGCAAAAAACAAATCTCACGGTGTAACTAT
TTTGCCTATTGTAGGGATTGCGGGCGTTGGAAAGACAACTCTCGCTCAACTTGTATACAATGATCCATATAGTGAAAGTCAATTTGATCA
CAAGATATGGGTTTGGGTGTCTCACAACTTTGATGGCATGAGGCTCACAAGAGAAATGTTGACCTCTGTTTCTCAACAAAGGCATGAAGG
AATAGACTGCTTTGTGAAGCTTCAGGAGATCTTAAAAAGTCATATCAAATCAAAGAGGGTTTTACTAATTTTAGATGACGTCTGGGATGA
CAAGGATGATTGCCGCTTGAACCAACTAATGGCTCCTTTTAAGAATGATAGTGATAATGGCAATGTGATTCTTGTGACAACTAGAAAACT
TTCTGCTGCAAAAATGATTGGAACAACGGAGCCAATTAAGTTAGGTGCTTTAGAAAAGGATGACCTCTGGTTATTGTTCAAATCATGTGC
ATTTGGTGATGAAAACTATGACTGTCTTGGAAATATTAGCACAATTGGACGACAAATAGCAGAGAAGTTAGAAGGCAACCCGTTGGTAGC
AGTAACTACAGGGGCACTATTAAGAGGTCATCTTACCGTTGATCATTGGAGTAACATTCTCAAGAAAGAAAGTTGGAAATCACTGGGACT
CAATGGAGGCATCATGCCTGCTTTGAAGCTTAGTTATGATGAGTTGCCACACCATTTACAACAATGTCTCTCACATTGTTCTATATTTCC
CAAAAAATATAGGTTTCTTGGTAAGGATTTAGTCTATATTTGGATTTCTCAGGGATTCGTGGATCGCACCCATTTAAGTGAGAGATTGGA
GGAGGCAGGATTGGAATATTTGAATGATTTGATGAGCCTGGGATTCTTTCAGCAAGTTGAAGACCAGCAGGATGAAGATGGGGATGAGGA
TGAGGAAGAAGAATCCTCTCTAGGCAGTCAAATTCGGTACTCTATGTGTGGTCTCATGCATGATTTTGCCAAGATGGTTTCAAGGACTGA
ATGTGCAACTATAGATGGTCTACACTGCAAAATGCTGCCAAATATACGTCATTTGGCGATAGTAACTGATTCTGCATACAACAAAGATTG
GTATGGGAACATTCCTCGTAATGAGAATTTTGAAGAAAATCTGAGAAACACGGTTACATCGGTCAGCAAATTGAGGACGCTGGTTTTAGT
TGGGCACTATGACTCTTTCTTCATAGAATTGTTCCAAACTATATTCCGAAAGGCACATAATTTACGCCTGCTGCAAGTGTCTGCAACATC
CACTGGTTTTAACTCCTTTTGTTGTGTTTTGGCAAATCCTTTGCATCTACGTTATCTAAAACTTGAGTTGCACGGGGTTGTGCCACAAGT
TTTGAGTAAGTCCTTTCATCTTCAAGTATTAGATGTTGGCTCAGACATGAATACTTCTGTACCCAATGGCATGCATAATCTTGTCAGCCT
GCGCCATCTTATTGCACGCAACAGAGTGCGCTCTTCAATTGCTAGCATTGGCATCATGGCATCTCTTCAGGAGCTACATGATTTTGAGGT
TCGAAATGCTAGCGGCTTTGAGATAACACAACTCCAATCCATGAACGAGCTTGTACAACTTGGGGTGTCTCAACTTGATAATGTTAAAAC
TCGGGATGACGCTTATAGGGCAGGACTAAGAAACAAAGAACACTTAGAAGAGCTTCATTTGTCCTGGAAGTATGCACTGTTAGAAAATGA
ATATAGCAGTGAAAAGGCAAGAGAAGTTCTTGAGGGTCTTGAACCACATATGGGTTTAAAGCATCTACAAATATCTAAGTATAATGGTAC
TACTTCACCAACTTGGCTTGCCAACAAAATCTCGGTTACCTCCTTGCAGACACTTCATCTTGATGATTGTCGTGGATGGAGAATACTTCC
ATCTCTGGGAAGTCTTCCATTTCTTACAAAGCTGAAGTTGAGCACCATGTGTGAAGTAATAGAAGTATTACTTCCTTCACTAGAGGACTT
GGTACTAATTAACATGCCAAAGTTAGAGAGATGCTCAAGCACTTCTGTGGAGGGTTTGAGCTCTAACTTGAGGGTGCTGCAGATCGAGCA
TTGCAAAGCACTAACGTCATTTGATCTGCTTGAGAATAATGATAAATTCAAAATCGAGCAGAGCTCGTGCTTGGCTGGTCTTAGGAAATT
AATTTTGTATGATTGCCCTCGTTTGAAAGTGTTGAACCCTCTTCCACCTTCAACAACATGTTCCGAGTTACTCATCAGTGGAGTTTCAAT
ACTTCCGAGTATGAAGGGATCATCAAGTGATAATTTACGTATTGGGCTCATTAATGAGTCTATAATCTATGGCAGTATTGATGGATACGC
TGATGAGTCGAGGATAATGGATGACAAAATTTTTGCGTTCCATAATCTTAGAAACCTCAAATCGATGGTGATATTTGGTTGCCAAAATTT
AAGGTCATTTTCATTTGAAGATTTTAGTCATCTCAGCTCTTTAAAGAATTTGGAAATATCAATGTGCAAGGAACTTTTCTCTTCAGATGT
GATGCCAGAGCATACCCTTCAAAATGTGGCAACCACGAAATGCAGGGCCTTCCCATCTCTTGAAAGTCTCAGTATTAGGTCATGTGGAAT
AACAGGGAAGTGGGTATCTTTGATGCTCCAACATGCGTGGATCCTTGAGGAATTGAGTTTGGAAGATTGCCTACACACAACAATAATACA
ATTGCCGACGGAAGAGGAAGAAAACAGTCTATCAGATCTTATCTCAGCCAGGGAGGACTCATCATCAGGAGATCAAGACACATTGACCTG
GTTAGCTCGAGATAGACTCTTGCACATTCCATCAAATATCACCTCCTCTCTCAAGTGGTTAACCATTTGGAAGTGCCGTGGTGTAACATT
TAATGGGAGTGAAAAAGGTTTCTCCAGATTTACCTCCCTTAAGGAGCTACAAATTAGGGGATGCCCCGAGCTAGTCTTGCATTTGGTGGA
TAAAGATGGAACTTATTACTGCACGAACGGAAGATGGTTCCTCCCATCATCACTTGAGGTACTGGGCATCGACAACTATTTCCAAGAAAA
GCTTCAACCCTGCTTTCTGAATGATCTCACCAGCCTTAAAAGGTTATCCGTCTCGTCCAGGCCATGGTTGAAATCTCTACAGCTGCACTC
ATGCACAGCACTAGAAGAGTTGAAAGTCATTCAGTGTGAATCGCTCACGACACTAGAGGGCTTGCAATTCCTTGGCACCCTCAGGCATTT
GACAGTATACGACTGCCCTGGCATGTCTACCTGTTTGAAGAGCCTTTCATGGCGCTACGGGCTATGCTCTCGGCTGGAAACGCTCGGAAT
TGGTGATCCATCAGTCCTTACCACATCATTCTGCAAGCTCCTCACATCGCTGCAATGCCTAAAATTATATCATTTTGGGTGGGAAGTAAC
GAGGCTAACCGATAACCAAGAGATAGCCCTTGTGTTCCTCAAGTCCCTGCAAGAGCTCCACTTTTTGTGCTGTTATGATCTAGTAGATCT
TCCTGCGGGGCTGCACAACCTTCCTTCCCTCAAGAAGTTGAAAATAGACACTTGTCCGCGCGTCTCAAGGCTGCCGAAAACAGGTCTCCC
ACTTCCGCTGGAAGAACTGGAAATCGAGTTTTGCAGCAAGAAGCTGGCTGATCAATGCAGGCTGCTAGAAACAAGCAAGCTAAAAGTCAA
AATTAGTCTATGCTCTTGA
>curated_TraesCS2B01G489400
MLLGIFETAEQAARTYDAAALRFKGAKAKLNYPEGFQGRTDLGFKVTRSIPDGLQQHRHYPSTMEAPATQPSPQQQPTVPVLMRHELPPQ
GAGSSRGAVNLPFGAMSAPSTSSTSSPHMLVPPLASEDHTMRRTVSVEEEANDTHDGVTARTQSSKFVNSFYGFASACAFFTLSDSGQRT
TLFLFLLAVARNNAECMHGADRVDEISRGDADTPSNIVGKLRSVVWEHFTITEKDNGKPLKAVCRHCGNEFKCDTKTNGTSSMKKHLENE
HAVTFTKKPPRGRPPNPSSTSEPILIGNSSRTKGKRRWSKAWQLFDIIEEENGEPIKAICKYCPTKIKCGPMCGTAGMLNHNKICKNKPG
PYDQSPNPSSTGDATAHVKPSSSRKRRRPESTQMTAPNTATGWDKVEISNRIQNITSELQGIQLEVPKAFYPCGSSLSSNSDHHQSTISD
QRLKTSSLVQKKVYGRDVEKNSIVKLVRAKNKSHGVTILPIVGIAGVGKTTLAQLVYNDPYSESQFDHKIWVWVSHNFDGMRLTREMLTS
VSQQRHEGIDCFVKLQEILKSHIKSKRVLLILDDVWDDKDDCRLNQLMAPFKNDSDNGNVILVTTRKLSAAKMIGTTEPIKLGALEKDDL
WLLFKSCAFGDENYDCLGNISTIGRQIAEKLEGNPLVAVTTGALLRGHLTVDHWSNILKKESWKSLGLNGGIMPALKLSYDELPHHLQQC
LSHCSIFPKKYRFLGKDLVYIWISQGFVDRTHLSERLEEAGLEYLNDLMSLGFFQQVEDQQDEDGDEDEEEESSLGSQIRYSMCGLMHDF
AKMVSRTECATIDGLHCKMLPNIRHLAIVTDSAYNKDWYGNIPRNENFEENLRNTVTSVSKLRTLVLVGHYDSFFIELFQTIFRKAHNLR
LLQVSATSTGFNSFCCVLANPLHLRYLKLELHGVVPQVLSKSFHLQVLDVGSDMNTSVPNGMHNLVSLRHLIARNRVRSSIASIGIMASL
QELHDFEVRNASGFEITQLQSMNELVQLGVSQLDNVKTRDDAYRAGLRNKEHLEELHLSWKYALLENEYSSEKAREVLEGLEPHMGLKHL
QISKYNGTTSPTWLANKISVTSLQTLHLDDCRGWRILPSLGSLPFLTKLKLSTMCEVIEVLLPSLEDLVLINMPKLERCSSTSVEGLSSN
LRVLQIEHCKALTSFDLLENNDKFRIEQSSCLAGLRKLILYDCPRLKVLNPLPPSTTCSELLISGVSILPSMKGSSSDNLRIGLINESII
YGSIDGYADESRIMDDKIFAFHNLRNLKSMVIFGCQNLRSFSFEDFSHLSSLKNLEISMCKELFSSDVMPEHTLQNVATTKCRAFPSLES
LSIRSCGITGKWVSLMLQHAWILEELSLEDCLHTTIIQLPTEEEENSLSDLISAREDSSSGDQDTLTWLARDRLLHIPSNITSSLKWLTI
WKCRGVTFNGSEKGFSRFTSLKELQIRGCPELVLHLVDKDGTYYCTNGRWFLPSSLEVLGIDNYFQEKLQPCFLNDLTSLKRLSVSSRPW
LKSLQLHSCTALEELKVIQCESLTTLEGLQFLGTLRHLTVYDCPGMSTCLKSLSWRYGLCSRLETLGIGDPSVLTTSFCKLLTSLQCLKL
YHFGWEVTRLTDNQEIALVFLKSLQELHFLCCYDLVDLPAGLHNLPSLKKLKIDTCPRVSRLPKTGLPLPLEELEIEFCSKKLADQCRLL
ETSKLKVKISLCS-
>curated_TraesCS2D01G466600
TACTGTTGTACAGTTGTACTTTCCCCCCATTTGATGGAGGCCGCGATCGCGTGGCTGGTGGAGACCATCCTTGCAACACTCCTGATCGAC
AAGCTTGATGCTTGGATTCGCCAAGCCGGGCTTGCCGATGACATCGAGAAGCTCAAGTCGGAGATCAGGAGAATCAAGATGGTGATCTCT
GCTCTCAAGGGCAGAGGGATCCGGAAAGAGGCACTGGCTGAATCTCTCGCCCTTCTGGAGGATCACCTCTACGTACGACGCCGGCGACGT
GGTGGACGAGCTCGACTACTACAGGCTCCAACAGCAGGTCCGGGGACAAGGGGGCACTCCCACTGCCTGGCCGCCTGCAGATCCAAGCGT
GCATGGTACGCGTACTAGTGCTCGTAGATCCAAATCAAAGTGTACTAATTATTACTAGTTCGGTCTAATATATCTTGCTTCAAAAGACAA
ATTGATCTTATCTTATCAAGAATATGCATTTCTTTCCTGGGCATGTGTTTTTGGGCACAGTTGCAAGCGACGAGCGGCAAGGTGTGGATG
GAGCCGAGCGAGTCAATGAGATACCGAGGGGCGATGCTGCTACACGTAATAGCAGTGTTGGCAAATTACGGTCGCTCGTATGGGAGCACT
TCACGATCACACAAAAGGATGACGGAAAGCCTGTGAAAGCAAAATGTACATACTGTACAGAAGAGTTCAGATGCGAAACAAAGACGAATG
GCACGTCATCTATGAGGAACCATTTGGAGAAAGAGCATTCCGTGATTTGTACGAAGAGACCTGGAGCGCATCCACCAAATCTTTCAAGGT
ACCTTCAAAAGGACTTTTGTTTTTCGAAAATGAGGTTGAATCTTCTGTCTCTGCATTAAGCCATGCACACGGCCATTTTATTATATTATT
CAAAAATGCCTTATACAAGATACTAAAACTTTGATCCTTCAGAATCCATCTTCTAGACGATAAAAGTCGCACCACCTACAAGCTTGAGGA
TAATGGTGGTCATGATCAGGGCCACATGCCCTGACCTCACCCCTACACAAATCATCCAAAACCGGAACGCCGGTCCAGCGGACCCTTAGC
GCATCACATGCGTACACTCCGAAAGTCGCCACCGCCGCCTTTTGCGAACCCATCTTCGATGTAGGGATCAATGAAAAGACCTTGTCAGGT
ATGCCGTTGACGCCACCGCGAAGCCAGACCGCGTCACCGCCCTGCACGCGTCCATCATCGAGAGTCCGCCGCCGAGACTTGTCGTCTTCG
ACTCGTAAGACCACACAACTCCACCTCAGGATCCCTTCGGCCAGCACATGCTCCAGAAAAACGATGCCTCGGGAGGGTAAACGGCTCCGC
GCGCCGCTATCATCCGATCCGGGAGACCCGGATCTAGGGTTTCTCCCAGTGCGGCCTGGGCGGGAAGACAACAACTACATCAATGATGCC
TCTAACAAGAAAATGACGCCGTCATCGTCCGCCATGACGGAAGTCGGCGCATTTTTACGGGTAGCCTCACCTCCTCGAACCCATGGCTGG
CTTCCGATCCACAAATCCCGGAGGGTTGCGGATCTCCCACATCAAGCGTCGTAGACGCCGGAGAAAACTCCGGCCGCCACACGCCTCCAG
CAACGAACTCGGGTATATGATCCCTTGATCCACCGCCCCCGACACAGCCACGTGAAGCTGTCTCCTGGCCCGTCATCCCCGCCAGAGGGG
CCGCTGCCGCCGCCGTGTCCGGAGCCACCGCTCCAGGGCCCCTGCGCCGTAGATTGCTCACTAGAATTAATTGCATTGTGAGATTTTTGT
TAGTATACTTTGTGTTGTTGTTTGATCGCGATTCTTCTGCTCTGTGTTCTCATCTTTGCTAGTAGTATACACATACAAGGAATTGATTTT
TGCGAGAACTATAAAGTGCAGGTTCCGAAAGCGTTTTCATTGGGATCGATCTAACCACACTGGTAACAATGATTGACCACAGACTGCTCG
GGCTTCATGCCGGGCCTTGGGCTTCGGGCTTTCATGCCGGGCCAGACTCGGGCTTGCATTTAGACAAAATGTCAGGCTTCATGGTCAGGC
TCGGGCTTGAGATATGACGGTCGGGCTTTTTAAAGCTGAGCCCAAAACCCGGCCCGGCCCGGCCCAAGGTATGCCCAGGTTTGCCGCCCA
GTCTCAGTGTATAGTTGTAAAAAAGAGCCTGAATCAGATGTAACAGCATGGTCTGTAGTAGTGATATATCTTCCAGGGGCCCTTTTACAA
CACAAAAATTGTGTGTGCTGCCTTTAAATGCCCACTACTTGGGATCGTGCATATAGCTCTGCTTACCACACTCATTGCGTATAATATGTT
AGCTCTTGTGTGCCACAAATAGATGAATCGACCTACAGGCTACAGGACGCTAGTATGGATCTCCTGATCCAGTGTGGTGTTGATAGCTCT
CTCTATCAACAGGATCTCCTGATTTATCACAACTACAGATTTTGCTCTACTGAAACTGAAACAACCCGACACCCAAGCATATGGTCTTGC
TGAGGGGTCAAATGCATACCCTCATCGAGAGAGAACTGAACCTTTGGGAGATCTTGGAATCTTAATGCCACCAAAAAAATACTTGAGTTG
ACCCAAATTCTTAACCTCAAATCTGTTGCTAAACCTCACCTTCAGGCGACTTACCTCCACATTTACATCTCCCATGATAATAATATTGTC
CACATTAATAACAAGGATGTTAATTTGTTTCTTAATGCTGACATAATATCGTATGATCTCCATTTCATTGTTTGTGGCTCACCGAAACCT
GTCAAACCTCGCTCTTTGTAACTGCTTGTGACCTCCCGCAAAAAAAAAACTGCTTGTGACCTCCCGCAAAAAAAAAACTGCTTGTGACCA
TACAAAGACTTCTTCAATTTGCACACCTTTCCATTGGTTCTGGGGTACTAAAACTAGACGGGGTCTCCAAATAACGCTCCATGCAGATAT
GCATTCTTGACATCCAAGTATCCAACTGATCCAATGGCCAACCAAAGTTAGCGGTGCAAGAAATAAGTGATCT
TTTTTGCGAGAAAATTTTCAATCTATTCATTTTCAATCATGCAGTACAACGAATACCAGAAATAATAGAAATTACATCCAGATCTGTAGA
CCACCTAGTGACGACTACCAACACTGACGCGAGCTGAAGGCGCGCCGCTGTCATCGCCCCTCCATTGGCGGAGTTGGGCACAACTTGTTG
TAGTAGACAGCCGGGAAGTCGTCGTGCTAAGACCCCGTAGGACCAGCGCACCAGAACAGCAGTCGCCGCAGCTGAAGAATAACGTAGACC
AGAAGGATCCAATCCGAAGACACACGAACGTAGACGAACAACGACGAGATCCGAGCAAATCCACCAAAGATAGATCCGCCGGAGACACAC
CTCCACACGCCCACCAACGGTGCTAGACGCACTGCCGGAAGGGGGCTAGGCGGGGAGACCTTTATTCCATCTTCAGGAAGCCGATGCCGT
CTCGTCTTCCTTAGCAGGAACAAACCCTAGCAAAACTGAAAGAAACGACTAAAAACGGATCCCTCCCGCCGGCCCTTGCCGAGATCCACC
GCGCCCCTAGGGCCATCGGAGAGGAGGCGGACCTGCGGCGGCGTCGGCGCGAGGCAGAAACCCCAACTTTTTTGTGGAGGAGGAGGAGGC
GGCTAGAAAGGCTTCCGTGTCCGTAATAGTCAATCCCATAGATTTATGGACTTGGAATGTGTTTGGTTGACATCTTTGTTTTTGAGCATT
TTGCATACTTTTCCCAGTTGAGCCTGTTTGAGCTAATGCATGCAAAAAACCAACATCTGCATGTAGTTTGGTTGCCTACATTTAGGCTAC
CTGCATCAGGGAAGCAATTTTTACCATGGTATTTGGTTGCTTGCATCGCAGTTGTTAGACAAACTACATGCTGTTAATTTGGTTGCAAAT
GGCATAAGGTCTGATCACTTCTCACTAGTGATGACCTTGCCACACACGGGTTGAACATTGCCTCGGTCCTAACTTGGAAAGATATGGCAA
TTTATCCTAGCTACTAACAAATAGCATACAAATTAAGAGCCATATGCCTGAATAAGGGAAAGTTCATCGATGCTAAATAGGGTGAAGTCC
ATCCTCATCCTTTGTTCTTCCAGGCTTCGCTGTCAAATGCCTCCACACCATGACTGGAGCTGACAACATCATCAGGCTTCACATCTTTCT
CCTCCAGCACAAGTTCATCACAACCTCATTGTAGGATCCAGTTATGAAGGATGCAACATGCAAGAACAAGTTTACCCTGGGTAGGGTAAG
GGTGAAATGACTTTTGATCCAGGATCTTAAACATATTCTTCATAGCTCTAAATGCCCTCTCAACCATAACTCTAAGGCTGGAGTATCAGA
GATTAAAAAGTTTATGTGGAGTCGTAGGATAGTTTCTACCAGAGAACTCGTTCAGATGGTACCTGGTTTTCCTGAGAGGTGGAAGAGCAC
CCGGCCGACATGCATAGCCAACATCTCCTAGGTAGAACTTGCCATCGGGGATATTGATGCCATCAGGTCTACTCATGTTGTCACTTAGAA
TGTTAGCATCAGTGCTGATCCTTCCCAACCAGCTAGCACATATGTGAACTTCAGATCGAAGTCAACAGCACCAAGAACATTCTGGCTTAT
AGAAGAAATTAGTGGTGTTGGTATTACCCTTATAGAAGAAAGAAAATGAAACAACAATTAAAAACAAATGATGAAAAACTTGCACACAGT
TTGTACTGAAATTGCATATTTTTATGAATGCAAAAATAGGCAGATAATAATGCAATTTTGCACTACAGTATAATTTATACACATTGTATA
ATACTTTTGTATATATTTACACACGCACACCTAATATTTACACATACGCATAAAGAAAAAGAAAAACTGACTAGAAATACTTGATAAACA
ATAATAAATACTAAAACTAGTACGAAGCTAAAAGACAAAAACTGAATTTTCCCTAAGGTAGAATGAATTAGGTGCATTGGTTTCCCCTCT
AAAAAAGAAATAAAGAAAACTTGAAACAGACGACAATAGAAAATTTTGCACATGAAATGCGCGGTTGCACAATATGCAAAAACAAGTATA
CCGTAATTTTCAGATAACAAAGACACATGCATGTGCATACATGCACATGGCTGCAATGCACGAAGAGCATACACAAAGTCACTCACAACA
CCAGCACCAGCACATGCAGGTCCCTTGCAAGCAGGCAAGACACACACATGCACGCACACAAAATCTGACACATAAGAAAAGAAAAAAACA
GACAAAATATTTAGTAGAAGAAAAGAGTGACTGACCCAAAAGTAAATTTCAGAAGACTTAAATGTAGCAAAACTGATATACATCAGCTTG
AGAGCCCATGGTTTTCCTAATAGCCAGCCCACCATCTTTTTCTGACTGCAGCACCGGCGAGCCTATTGTAATTGGCAGCTCATCCAAGGG
AAAAGGAAAGAAACGACGGTCCAAGGCATGGGATTCTTTTGATGTCATAAAAGAAGTAAACGGACAGCCTATCAAAGCAAGATGTAAATA
CTGTCCCACAGAGATCAAGTGCGGAACCGGGAACGGGACAGCAGGTATGCTCAACCATAACAAGATTTGTAAGAAGAAACCTGGACTAGA
TGACCAGCCACCAAACTCGTCAAGGTAGCTGATGAATCTTTGCACCGTGACATTTTTAGGGGGTTGTTTAAATAAGAGCCCCATTGTGGT
TCTATTTTCCAATTGACGGTCTCTTCCTTACTGCAGCACCAATGATACTACCGCAAATGATGCTACCACAAATGCAAGGCCTAATCTAAT
TGGTGATTCATCTAGCAGAAAAAGAAGGAGAGTTGATGAGGAATCCGCACAAAATATCGCAGCTAACACAAGTACCCCTTGGAACAAGGC
TGAATTATCAAACAGAATACAACAAATAATTAGTCGGTTACAGGACATCCGAGGGGAAGTGAGTGAGGTTTTCAAGCTACATGAATCAGA
CTCTGCTTCAAGTTTAGATCACAACCGGAGTACAACCTCGGATCAGCATCTGAGAACATCAAGTCTTATTTCAAGGCAATTGTATGGGAG
AGTTGCAGAAAAGAAATCCATCTTGAAGTTGATGATGTCAGATGACACATCTAATAGCATAATTGTTCTGCCTATTGTAGGCGTTGCAGG
TGTTGGAAAGACAGCTCTCACTCAACTTGTATACAATGAACCAAACGTGGAGAGTCGATTTCAGCACAGGGTATGGATTTGGGTGTCTCG
AAACTTTGATGAAGTGAGGATAACAAGGGAGATGTTAAACTTTGTTTCTAGAGAAAAACATGAAGAAATAAACTGCTTTGTGAAGCTTCA
GGAGATCTTGAAAATTCATGTAAAATCAAAGAGGGTTTTAATAATTTTAGATGATGTCTGGGATGACATGAACGACTGCCGATGGAACCA
ATTGTTGGCTCCTTTTAAGTTTAATAGTGCTAATGGCAATGTGATTCTTGTGACAACAAGAAAACTATCTGTTGCAAAAATGGTTGGAAC
AACTGAGCCAATTAAGATAGGTGCTTTGGAAGAGGACGATTTCTGGTTATTGTTTAAATCATGTGCACTTGGTGATAGAGCCTCTGAAAA
TCCTGGAAATCTATGCACTATTGGACGACAAATAGCAGGCAAGTTAAAGGGCAATCCGTTAGCAGCAGTAACTGCAGGGGCACTATTACG
AGATCATCTTACTGTTGATCATTGGAGTAACATTCTCAAGAAAGAAGACTGGAAATCGTTGGGTCTCAGCGGAGGCATCATGCCTGCTTT
GAAGCTTAGCTATGATGAACTGCCATACCATTTACAAAGATGCCTATCATATTGTTCTATATTTCCTAACAAGCATAAGTTCTCGGGTAA
GGATTTGGTTTATATATGGATTTCCCAAGGATTTGTGAGTTGCGCCAATTTAAGTAAGAGCTTGGAGGAGATAGGATGGCAATATTTAAT
TGATATGACGAACATGGGCTTATTTCAGCAAGTCAGAGGAGAAGAGTCGTCTTCATTCTTTCACTCAAATTGCCAAACATGGTATGTTAT
GTGTGGTCTTATGCATGATTTTGCAAGGATGATCTCAAGAACTGAGTGTGCAACTATAGATGGTTTACAGTGCAATGGGATGATGTCAAC
TGTGCGACATTTATCAATAGTAACTGACTCTGCATACAAGAAAGATCAGCATGGGAATATTCTTCGTAATGAGAAGTTCGAAGAATATCT
AAGGAGTACAGTTACATCAGTTGGTAAATTAAGGACGTTGATTTTACTTGGGCACTATGACTCTTTCTTCTCACAGTTGTTCAAAGATAT
TTTCAAAGAGGCACATAATTTACACCTGCTGCAGATGTCTGCAACATCTGCTGATTTTAGTTCCTTCCTATGTGGTTTGGCAAGCGCGGT
GCATCTTCGTTATCTAAAACTTGAGTCAGATGGGTTGGAGGGGGATTTTCCACAAGTTTTGGTCAATCTTTTTCATCTTCAGGTATTAGA
TGTTGGCTCAAACACCGATCCTATTTTACCTAATGGCATGCATAATCTTGTGAACCTGCGGTATCTTGTTGCAGAAAAGGGAGTATACTC
TTCCATTGCTAGCATTGGTAGCATGACATCACTTCAACAACTTCATAATATTAAGGTTCAATTTTCTTGTATCGGCTTTGAGATAACACA
ACTCCAGTCTATGAACGAGCTTGTACAACTTGGTGTGTCTGAACTTGAAAATGTCAAAACTAGATATGAGGCTAATGGAGCAAAACTGAG
AGACAAAAGACACTTAGAAGAGTTGCGCTTGTTGTGGACGCATACTCCGTCACGAGATGAATATGCCACTGACACGAGCTTTCAACATCC
AGTGGACAATGTAGAAAGAGATGTAGAGCTCTTGCCAATGGTTGAAAGAGGGCCAAGTTCCGAGCCTTGTCTGGACAGAGCAAGAGAGGT
GCTAGAGGGTCTTGAACCACATCAAGACTTAAAACATCTTCAGATATCTGGGTACTATGGTGCTACATCCCCAACTTGGCTTGCCAACAA
TATCTCAGTTACCTCCCTGCGAACCCTTCATCTAGACAGTTGTGGAGAATGGGAAATACTTCCGTTTATGGAAAGGTTTCCACTTCTGAT
AAAACTGAAGTTGACCAACCTGCGGAAAGTAATCGAAGTATTGGTTCCTTCACTGGAGGAGCTAGTTTTAGTTGAAATGCCAAAGTTGCA
AAGATGTTTGTGCATTTCCGTGGGGGGTCTGAGCTCTAGCTTAAGGGCATTGCACATCGATAAGTGTCAAGCACTAAAGACGTTTGATCT
GTTTATGAACGATCATAAAATCAAACTAGAGCAGAGGCCATGGTTGTCTGGTCTTAGGAAATTAATTATGCGTGATTGCCCTCATTTAAA
AGTATTGAACCCTCTTCCACCTTCAGCCACCTTTTCTGAGTTACTCATCAGTGGAGTTTCAACACTTCCAAGTATGAAGGGGTCATCTAG
TGAAACGTTACATATTGGATCTTTCAATTGGTTTATTGATCACTCTTCTGGTGAGTTGACGGTACTGGATGATAAAATATTGGCATTCCA
CAACCTGAGGAGAATCAAATTGATGAGAATATATGGTTGCCGGAATCTAACTTCTATTTCATTCGAAGGTTTTAGTCATCTCGTCTCTTT
AGAGAGGTTGGAAATACACTGGTGCGAAAAATTGTTCTCTTCACATGTTTTTCCAGAGCATATCCTTGAAGATGTGCCGACTGCAAATTG
CAAGGCCTTCCCTTCTCTTGAAAGTCTCACTATTGAGTTCTGTGGAATAGCAGGGAAGTGGCTATCTCTGATGCTGCAACATGCGCCAAA
CCTAGAAGAATTGATTTTAGAGAATTGCCCCCGTATAACAACGCTGTTATCGACAGAAGAGGAAGAAAACAGTCCATCAAATCTTATCAT
GGACAGGGGGTACTCGTCATCAGGAAATCTAGATGACGCATTGGCAGGGTTAGCTCAAGACGAACTCTTGCACGTTCCATCAAATCCCGT
CTCCTCTCTTAGGAAGATAACTATTCAGGGCTGCCCTTGTCTGACATTTAATGGGAGCAAGAACGGCTTCTCTAGATTTACCTCCCTTGA
GGAGATAACGATCTACAACTGCCCCGAGCTGTTCTCGCCTTTGGTGCATAAAGCCGGAAATGATGACCGCACAAACGGAAGATGGCTATT
CCCAACATCACTTGGGGAACTTGACATCGACGGCTATTCCCAAGAGACGCTGCAGCCGTGTTTTCCAAGTCCTCTCACCAGCCTTAAAAA
GTTGGAGGTACTGAGCAGCCCAGGTTTGGAATCTCTGCAGCTTCAGTCATGCACGGCACTTGAAGAGCTGATAATTGGAGGCTGTGGATC
ACTCACCGCACTAGAGGGCTTGCAATCCATTGGCAACCTCAGGCATTTGAAAGTATCTGATTGCCCTGGCCTGCCTCCATATTTAGAGAG
CTTGTCAAGGCAGGGCTATGAGATCTGCCCTCGACTGGAAGGACTTCACATCGATGACCCATCTGTCCTTAGCAAGTCATTCTGCAAGCA
TCTCACCTCCCTCCAACGCCTAGAACTGGGTCATTTGAGCATGGAAGCGACAACACTGACTGATGAGCAAGAGAGAGCGCTTCTGCTGCT
TAAGTCCCTGCAAGAGCTCGACATTTGTGGTTGTTATCATCTCGTAGATCTTCCTGCGAGGCTGGACACCCTTACTTCCCTCAATAGGTT
CAAGATACATTCCTGCTCCATCATCTCAAGGCTCCCACTAGCATTTTAGCAGTACACATGTATTCCTGATGTTTTGTAATCAATAATTTG
CCACAGACCTGCATGCACTAGGCTGCCCAGATTCTGTGACCACTGTCCCTCTGCTCTCCTAAACTTGGGCCATACATTATGTTATATTCA
GAATTGATATACCCTCATAAATGTGCACTATGCTCAATGTAAAAAAGACCGTCTCTCTGCATATGATTCGGTCTTCAGACAATTTTCCTA
AAGCCCTTCTATCAGTTGTAGCATGCTTTGCCGTATGCGTTAACAAAAGATTAACAAATGTACATGATAGCTGATGGTCTAATCAATCTT
TCTATTGTGATCAGGATGT
>curated_TraesCS2D01G466600
MEAAIAWLVETILATLLIDKLDAWIRQAGLADDIEKLKSEIRRIKMVISALKGRGIRKEALAESLALLEDHLYVRRRRRGGRARLLQAPT
AGPGTRGHSHCLAACRSKLASDERQGVDGAERVNEIPRGDAATRNSSVGKLRSLVWEHFTITQKDDGKPVKAKCTYCTEEFRCETKTNGT
SSMRNHLEKEHSVICTKRPGAHPPNLSSTGEPIVIGSSSKGKGKKRRSKAWDSFDVTKEVNGQPIKARCKYCPTEIKCGTGNGTAGMLNH
NKICKKKPGLDDQPPNSSSTNDTTANDATTNARPNLIGDSSSRKRRRVDEESAQNIAANTSTPWNKAELSNRIQQIISRLQDIRGEVSEV
FKLHESDSASSLDHNRSTTSDQHLRTSSLISRQLYGRVAEKKSILKLMMSDDTSNSIIVLPIVGVAGVGKTALTQLVYNEPNVESRFQHR
VWIWVSRNFDEVRITREMLNFVSREKHEEINCFVKLQEILKIHVKSKRVLIILDDVWDDMNDCRWNQLLAPFKFNSANGNVILVTTRKLS
VAKMVGTTEPIKIGALEEDDFWLLFKSCALGDRASENPGNLCTIGRQIAGKLKGNPLAAVTAGALLRDHLTVDHWSNILKKEDWKSLGLS
GGIMPALKLSYDELPYHLQRCLSYCSIFPNKHKFSGKDLVYIWISQGFVSCANLSKSLEEIGWQYLIDMTNMGLFQQVRGEESSSFFHSN
CQTWYVMCGLMHDFARMISRTECATIDGLQCNGMMSTVRHLSIVTDSAYKKDQHGNILRNEKFEEYLRSTVTSVGKLRTLILLGHYDSFF
SQLFKDIFKEAHNLHLLQMSATSADFSSFLCGLASAVHLRYLKLESDGLEGDFPQVLVNLFHLQVLDVGSNTDPILPNGMHNLVNLRYLV
AEKGVYSSIASIGSMTSLQQLHNIKVQFSCIGFEITQLQSMNELVQLGVSELENVKTRYEANGAKLRDKRHLEELRLLWTHTPSRDEYAT
DTSFQHPVDNVERDVELLPMVERGPSSEPCLDRAREVLEGLEPHQDLKHLQISGYYGATSPTWLANNISVTSLRTLHLDSCGEWEILPFM
ERFPLLIKLKLTNLRKVIEVLVPSLEELVLVEMPKLQRCLCISVGGLSSSLRALHIDKCQALKTFDLFMNDHKIKLEQRPWLSGLRKLIM
RDCPHLKVLNPLPPSATFSELLISGVSTLPSMKGSSSETLHIGSFNWFIDHSSGELTVLDDKILAFHNLRRIKLMRIYGCRNLTSISFEG
FSHLVSLERLEIHWCEKLFSSHVFPEHILEDVPTANCKAFPSLESLTIEFCGIAGKWLSLMLQHAPNLEELILENCPRITTLLSTEEEEN
SPSNLIMDRGYSSSGNLDDALAGLAQDELLHVPSNPVSSLRKITIQGCPCLTFNGSKNGFSRFTSLEEITIYNCPELFSPLVHKAGNDDR
TNGRWLFPTSLGELDIDGYSQETLQPCFPSPLTSLKKLEVLSSPGLESLQLQSCTALEELIIGGCGSLTALEGLQSIGNLRHLKVSDCPG
LPPYLESLSRQGYEICPRLEGLHIDDPSVLSKSFCKHLTSLQRLELGHLSMEATTLTDEQERALLLLKSLQELDICGCYHLVDLPARLDT
LTSLNRFKIHSCSIISRLPLAF-

1. An isolated nucleic acid encoding a nucleotide-binding and leucine-rich repeat (NLR) polypeptide comprising a zinc-finger BED domain, wherein expression of the NLR polypeptide in a plant confers or enhances resistance of the plant to a fungus.

2. The isolated nucleic acid according to claim 1, wherein the nucleic acid is isolated from a plant.

3. The isolated nucleic acid according to claim 1, wherein the BED domain has an amino acid sequence corresponding to SEQ ID NO: 1 or a variant or functional fragment thereof.

4. The isolated nucleic acid according to claim 1, wherein the NLR polypeptide comprises a leucine-rich repeat (LRR) motif at or near the C-terminus.

5. The isolated nucleic acid according to claim 1, wherein the NLR polypeptide has an amino acid sequence comprising SEQ ID NO: 2 or SEQ ID NO: 3, or a variant or functional fragment of either.

6. The isolated nucleic acid according to claim 5, having a nucleotide sequence comprising SEQ ID NO: 4 or SEQ ID NO: 5.

7. The isolated nucleic acid taccording to claim 1, wherein the NLR polypeptide has an amino acid sequence comprising SEQ ID NO: 6 or a variant or functional fragment thereof.

8. The isolated nucleic acid according to claim 7, having a nucleotide sequence comprising SEQ ID NO: 7.

9. The isolated nucleic acid according to claim 1, wherein the NLR polypeptide comprises a further zinc-finger BED domain.

10. A nucleotide-binding and leucine-rich repeat (NLR) polypeptide comprising a zinc-finger BED domain, wherein expression of the NLR polypeptide in a plant confers or enhances resistance of the plant to a fungus.

11. The NLR polypeptide according to claim 10, wherein the BED domain has an amino acid sequence comprising SEQ ID NO: 1 or a variant or functional fragment thereof.

12. The NLR polypeptide according to claim 10, comprising a leucine-rich repeat (LRR) motif at or near the C-terminus.

13. The NLR polypeptide according to claim 10, having an amino acid sequence comprising SEQ ID NO: 2 or SEQ ID NO: 3, or a variant or functional fragment of either.

14. The NLR polypeptide according to claim 10, having an amino acid sequence comprising SEQ ID NO: 6 or a variant or functional fragment thereof.

15. A vector comprising an isolated nucleic acid as defined in claim 1.

16. The vector according to claim 15, further comprising a regulatory sequence which directs expression of the nucleic acid.

17. A host cell comprising a nucleic acid as defined in claim 1, an NLR polypeptide or a vector.

18. The host cell according to claim 17, which is a bacterial cell, a yeast cell or a plant cell.

19. A method of producing a transgenic plant or plant cell comprising introducing and expressing a nucleic acid according to claim 1 or a vector into a plant or plant cell, wherein introducing and expressing the nucleic acid or vector confers or enhances resistance of the plant or plant cell to a fungal pathogen such as wheat yellow (stripe) rust fungus Puccinia striiformisi f. sp. tritici.

20. The method of claim 19, wherein the transgenic plant or plant cell has resistance or enhanced resistance to the fungal pathogen compared to a plant or plant cell of the same species lacking the nucleic acid or vector.

21. A method for producing a non-transgenic plant or plant cell having resistance or enhanced resistance to a fungal pathogen, the method comprising mutating or editing the genomic material of the plant or plant cell to comprise a nucleic acid as defined in claim 1.

22. A plant or plant cell obtained or obtainable by the method as defined in claim 19.

23. The plant or plant cell of claim 22, wherein the plant or plant cell is a crop plant or plant cell or a biofuel plant or plant cell.

24. A seed of the plant of claim 22, wherein the seed comprises a nucleic acid or an NLR polypeptide

25. The seed according to claim 24, which is a wheat seed.

26. A method of limiting wheat yellow (stripe) rust in agricultural crop production, the method comprising planting a wheat seed as defined in claim 25 and growing a wheat plant under conditions favourable for the growth and development of the wheat plant.

27. A method for identification or selection of an organism such as plant having resistance to a fungus such as wheat yellow (stripe) rust fungus Puccinia striiformisi f. sp. tritici, comprising the step of screening the organism for the presence or absence of:
(1) a nucleic acid as defined in claim 1; and/or
(2) an NLR polypeptide, wherein presence of the nucleic acid or the NLR polypeptide indicates resistance.


SEQUENCE LISTING (26 sequences)

SEQ ID NO: 1 - protein, 50 aa, artificial sequence
  BED-I sequence
SVVWEHFTITEKDNGKPVKAVCRHCGNEFKCDTKTNGTSSMKKHLENEHS

SEQ ID NO: 2 - protein, 1522 aa, artificial sequence
  Yr5_protein
MEPAGDSSVEAAIAWLVQTILATLLMDKMEEWIRQVGLADDVERLQSEVERVDTVVAAVKGRAAGNRPLSRALARVKELLYDADDLIDELDYYRLQQQVEGVTSDDPDGMRGAERVDEISRGHVDTLNCSVGKLRSPVWEHFTITETTIDGKRSKAKCNYCGNDFNCETKTNGTSSMKKHLEKEHSVTCTKKPGAHPPNPSSTGYATENVTLVEVGSSSNRKRKRTNKEPAQTTADNTRWDKAELSDTIKKITSQLQLQLQGILWAFSKVLEPHGSSSASSSNHHQPSTTSDQHAKTSSLAPRKVYGRVAEMNSIRNLIAEKKCDALTVLPIVGIAGVGKTTLAQSVYNDPDIKSQFHHKIWVCVSRKFDEVMLTREMLDFERHEGSPHENGRHEGISSLAKLQEILKDIIEYQSKSFLLILDDVWDSMDDHQWRKLVCPFVSSQAKGNLILVTTRNLSVAHMLGTREPIKLGALENDVMWLLLKSCAFRDVNYEGNQSLSIVGRQISEKLKGNPLAAETAGALLRKKFSIDYWKIILKNEDWKSMELGNGIMAALKLSYDQLPYHLQQCFSYCSIFPDGYQFLGEELVGFWMSQGFVKCNNSSQRLEQIGQCYLIDLVNLGFFEEVKREEPYLGCRVMYGICGLMHDFVIMVSRTDCASIDGLQRNKMPQTLRHLSIVTGSAYKKNQHGNIPRNNRFEENLRNTITSVSELRTLVLLGHYDFSFLLLFQDIFQKAHNLRVLQMSAAPADFLKHRFEEVDGSFPQILSKLYHLQVLDVGAYTDRTMPGCIDNLVSLRHLVVHKGVYSSIATIDNMLSFQEQHGFKFHISSGFEITRLQSTEHWMHVDTLEDVYEAGLVNNELSEKLHLSWKDSPEDIGMEVEDWEPHWDLRVLEISGYNFGSPIVVDIIILVTSSQTVEISNCSEWKILPSLERFQFLTNLELRNLPKVIEILVPSLEELALVTMPKLKKCSCTPVEGMSSRLRALRIEDCQSLKEFDLFENNDKFETGQRSWAPSLRELSLENCPHLKVLKPLPLSLMCSELLISGVSTLPYMKGSSDRKLCIGYDDKYDYYGFDESSDELKILDDKIFMFHNLKNLKSMVIYGCRNLSSISLKGFSYLISLTSLEIRDCEKLFASDEMPEHTLEDVTPANCKAFPSLECLSIDSCGIVGKWLSLMLQHAPCLEELYLSSREEENSEEENSEEEENSISNLSSTREGTSSGNPDDGLALDRLLRIPLNLISILKSITIERCPHLTFNWGKEGVSGFTSLEKLIVLDRPDMVLTNGRWLLPNSLGELESNDYSRGTLQPCFPSDITSLKKLKVRRSPGLQSLQLHSCMALEELDIQDCRRLAALQGLQFLGSLTHLTIYNCPGLPPFLESFSRQGYTLLPRLKRLHIQDPSVLTTSFCRHLTSLQHLKLTWLEEVRLTDEQEQALVLLKSLQELQFHYCSNLVDLPAVLHNLPSLKTLKVDGCRGISRLPETGLPFSLEELEIEWCSKELADQCRLLASNKLNIKILSGIYV

SEQ ID NO: 3 - protein, 1586 aa, artificial sequence
  Yr7_protein
MEPAGDSSLEAAIAWLVQTILATLLMDKMEAWIQQVGLADDVERLQSEVERVDTVVAAVKGRAAGNMPLSRSLARVKELLYDADDVIDELDYYRLQHQVEGVTSDEPDGMRGAERVDEISRGHVDTLNVSVGKLRSPVWEHFTITETTIDGKRSKAKCKYCGNDFNCETKTNGTSSMKKHLEKEHSVTCTNKSAVHPPNTSSTGDATCNVRSVEVGSSSNGKRKRTNEDPTQTTAANIHAQWDKAELSNRIIKITEKLQLQDIQGALSKVLEPYGSSATSSSNHHRLSTASDQHPTTSSLVPMEVYGRVAEKNKIKKSITENQSGGVNVLPIVGIAGVGKTTLAQFVYNDPDVKSQFHHRIWVCVSCKFDEVKLTKEMLDFFPRERHEGINNFAKLQEILKEHVEYQAKSFLLILDDVSDSMDYHKWNKLLNPLLSSQAKNIILVTTRNLSVAQRLSTLEPIKLGALENDDMWLLLKSCAFGFGNYEGTENLSTIGRQIAEKLKGNPLAAVTAGALLRDNLSIDHWSNILKNEKWKSLGLSGGIMPALKLSYDELTYRLQQCFSYCSIFPDKYRFLGKDLVYIWISQGFVNCTQNKRLEETGWEYLNQLVNLGFFQQIEEQQELDGEEEFSLRRQIWYSMCDLMHDFARMISRTECATIDGLQCNKIFPTVQHLSIVTGSAYNKDLKGNIPRNEKFEENMRNSVTSVTKLRTLVVLGNFDSFFVRLFQDIFQKAQNLRLLLVSLASTYLSQVPAAFNDFNSFLCNLANPLHLRYLKLELDGIVPQVLSTFFHLQVLDVGSSMDTSLPNGLLHNLVSLRHLVAHKRVHSSITSIGNMTSIQELHDFEVRISSGFEITRLQSMNELVQLGLSQLDSVKTREDAYGAGLRNKEHLEELHLSWKDAYSEYEYASDTEFESSANMAREVIEGLEPHMDLKHLQISQYNGTTSPAWLANNISVTSLQTLHLDDCGGWRILPSLGSLPFLTKVKLSSMLEVIEVLIPSLEELVLIKMPKLVRCSSTSAEGLSSSLRVLHIEDCEALKEFDLFENDYNSEIIQGSWLPGLRNLILYCCPHLKVLKPLPPSTTFSKVLIREISRFPSMEVSSGEKLQIGNIDVYIGDDFDESSDELSILDDKTLAFHNLRNLKSMEIYGCRNLRSFSFEGFSHLVSLTSLKIVSCEQLFPSDVTAEYTLEDVTAVNCNAFPYLKSLSIDSCGIAGKWLSLMLQHAPGLEELSLTSCAHITRVVLPMEEEENNLLTTVLSSGNQDEALTWLVRDGLLHIPSNLVSSLKNMSITQCPRLKFNSGKDCFSGFTSLEKLEIWGSLVDDDGSDDLENGSSFVFGEEDQPLGANGRWLLPTSLQELHIVSLYCQETLQVCFPRDITSLKKLSVRSGQGLQSLQLYSCTALEELAISGSGSVTVTVLEGTQPAGSLGRLNVSDCPGLPSRLDSFPRLCPRLERLDINDPSVLTTPFCKHLTSLQRLKLGFLKVTRLTDEQERALVLLKSLKELEIFYCTHLIDLPAGLQTLPSLKSLKIEEGRGISRLPEAGLPHSLEELEIKICSKLEDECRRLATCEGKLKVKIDGRYVN

SEQ ID NO: 4 - DNA, 5654 bp, Triticum aestivum
atggagccggcgggagactcttccgtggaggccgcgattgcatggctggtgcagaccatccttgcaacgctcctcatggacaagatggaggagtggattcggcaagtcgggcttgccgacgacgtcgagaggctccagtctgaggtcgagagagtcgacacggtggtggctgctgtgaaggggagggcagccgggaacaggcctctgtcccgggctctcgctcgtgtcaaggagcttctctacgacgccgacgacttgatcgacgagctagactactacaggctccaacaacaagtcgaaggaggtagtaagcataatcccattatatatcgaatatatgtaagctcaagatatttattttgggatggagggagtagtttgatcttaatttctggtccatatttttttcggcacagttacgagtgacgaccctgacggtatgcgtggagctgaaagagtggatgaaatatcaaggggccatgtcgatacactgaattgcagtgttggcaaattacgatccccggtatgggaacacttcacgatcacagaaacaactatcgacgggaagcgttcaaaagccaaatgtaactactgtggaaatgattttaattgcgaaacgaagacaaacgggacttcatctatgaaaaaacatttggagaaagagcattccgtgacttgtacgaagaaacctggagcccatccaccaaacccttcaaggtacccaaaggaaattatatgttgcatcagcgcatttatattcgtttatatatatctgcttgagagcccattgttgttctacatttcttctgataactgacccaccattttctctcttaatgcagcaccggctatgcaactgaaaatgtgacgcttgttgaagttggtagttcatccaacagaaaaagaaagagaacgaataaggagccagcacaaaccaccgcagataacacccgttgggacaaggctgagttatccgatacaataaaaaagattactagccagttacagttacagttacagggtatcctatgggctttcagtaaagttctcgagccacatgggtctagctctgcgtcgagttcaaatcatcaccaaccgagtacaacctcagatcagcacgcaaaaacatcaagtcttgctccaaggaaagtgtatggcagagtagcagaaatgaactccatcagaaatttaatagcagaaaagaaatgtgatgctctaactgttctgcctattgtgggcattgctggtgttggaaagacaactctcgctcaatctgtatacaatgatccagatataaaaagtcaatttcaccacaagatatgggtttgcgtgtcccgcaaatttgatgaagtgatgctcacaagggagatgttagactttgaaagacacgagggatctcctcatgaaaatggaaggcatgaaggaattagtagccttgctaagcttcaggagatcttgaaggacattatcgagtaccagtcaaagagttttctgcttattttagatgatgtatgggacagtatggatgatcatcaatggagaaaactggtgtgtccttttgtatcaagtcaagcaaagggtaatttaattctagtcacaaccagaaatttgtcagttgcacacatgttaggaacacgtgagccgataaagttgggtgctttggaaaatgatgttatgtggttgctgctcaagtcatgtgcatttcgtgatgtgaattatgaagggaaccaaagtctaagcattgtcgggaggcaaatatcagagaagttaaagggaaacccactagcagcagaaacagcgggggcactattaaggaagaaatttagcattgattattggaaaatcattttaaagaatgaagactggaaatccatggagctcggtaatggaatcatggctgctctaaagcttagctatgatcaacttccctaccatttacaacaatgtttctcatattgctccatattccccgacggttatcagtttcttggtgaggagttggtcggtttctggatgtcacagggatttgtaaagtgcaacaactctagtcagagattggagcagataggacagtgctatctgattgatttggttaacttaggcttctttgaagaagttaaaagagaagaaccatatctgggctgtcgagttatgtatggcatatgtggtctcatgcatgattttgtgattatggtgtcaaggactgactgtgcaagtatagatggtctgcagcgcaacaaaatgcctcaaactctacgacatttgtcaatagtaactggatccgcgtacaagaaaaatcagcacggaaacattcctcgtaataataggtttgaagaaaatctgagaaatacaattacatcagttagcgagttgaggacattggtgttacttgggcattatgacttttccttcttactattattccaagatatatttcaaaaggcacataacttacgtgtgctgcaaatgtctgcagcacctgctgattttctcaaacataggtttgaggaggtggatgggtctttccctcaaattttgagcaaattgtaccatctccaagtattagacgtcggtgcatacactgatcgtactatgcctggttgtattgataatcttgttagcctgcggcatcttgttgtacacaagggagtgtactcttccattgcaaccattgataatatgctatcatttcaggaacaacatggtttcaagtttcatatttctagtggctttgagataacacgactccaatccactgaacattggatgcatgttgatactctggaagatgtttatgaggcaggactggtaaacaatgaactctcagaaaagttgcacctgtcctggaaggattctcctgaggacataggcatggaggttgaggattgggaaccacattgggacttaagggttctcgagatatctgggtataattttggttcgccaattgtggttgacatcattatcttggttacatcctcccagacggttgagatatccaattgtagtgaatggaaaatacttccatctttggaaagatttcagtttttgacaaatctggagttgagaaacctgcccaaagtaatagaaatactggttccttcactggaggagctagcattagttacaatgccaaagttgaagaaatgttcatgcactcccgtggaaggtatgagctctagactaagagcactgcggatcgaggattgtcaatcactgaaggagtttgatctgtttgagaacaatgataaattcgaaactgggcagaggtcatgggctcctagtcttagggaactaagtctggagaattgcccccatttgaaagtgttgaagcctcttccactctcactcatgtgttctgagttactcataagtggagtttcaacacttccgtacatgaaggggtcatctgatagaaagttatgtattgggtatgatgataagtatgactactatggttttgacgaatcttccgatgagttgaagatactggatgacaaaatttttatgttccataatctgaaaaacctcaaatcaatggtgatatatggttgccggaatctaagttccatttcgttaaaaggttttagttacctcatctctttaacgagcttggaaataagagactgtgaaaaactttttgcttcagatgagatgccagagcatacccttgaagatgtgacacctgcgaattgcaaggctttcccatctcttgaatgtctcagtattgattcatgtggtatagtggggaagtggctatctctgatgctgcaacatgcgccatgcctagaggagttgtatttgtcttcccgagaggaagaaaattcagaagaagaaaattcagaagaggaagaaaacagtatatcaaatcttagctcaaccagggagggcacatcatccggaaatccagatgacggattagctctagaccgactgttgcgcataccattaaatctcatctccattctaaagagtataactattgagagatgccctcatctaacatttaactggggcaaggaaggcgtctcgggatttacctcccttgagaagctaatcgttttggaccgccccgacatggtgcttacaaacggaagatggctcctcccaaactcacttggcgaacttgaaagcaatgactattcccgaggaacgctgcaaccctgctttcctagcgatatcactagccttaaaaagttaaaggtacgtcgcagcccaggtttgcaatctctacagctgcactcatgcatggcactggaagaattggatattcaagattgtcgaaggctcgctgcactgcagggtctgcaattccttggcagcctcacgcatttgaccatatacaactgccctggcttgccaccatttctggagagcttttcaaggcagggctatacgctgttacctcggctgaaaaggcttcacatccaagacccatctgtccttaccacgtcattctgcaggcaccttacctccctgcaacacctaaaacttacttggttggaagaagtgagactaacagatgagcaagagcaagcgcttgtgctcctcaagtccctgcaagagctccaatttcattattgttccaatctcgtagatcttcctgcggtgctgcacaaccttccttccctgaagactttgaaggtagatgggtgtaggggcatctcaaggctgccagaaacaggcctcccattttcgctggaagaactggaaatcgagtggtgcagcaaggagctcgctgatcaatgcaggctgctagcatcaaacaagctaaatatcaaaattctcagtggaatctatgtatagagctttgatccagtctgacgtgccagttgatattaataagaagatctggaagatgaagatacctcttaagaataaaatctttgcatggtatcttcgtcgcggagtcattcttactaaagataaccttattaagagaaattggcatggaagtacgcaatgtgtattttgtccgcatgatgagacaataaaacatttgttcttccaatgtaaattggctcgttctatatggtcagtcatccaaatagcttctggcttgtaccctccttgtagtgttgctaatatatttggcaattggttacatgggattgatcacaagttcagaagtctacttagggtgggagcgcttgccgtgatttggtcgctttggctatgtagaaatgataagatttttaacgataaaagtacttcgcttatgcaggttatctacagatgtactgggacgcttcgtttatggtcctctctacaacgagtggagaatcgagacctgtttacggaggtgtgtacacgattggaggttacggcgagggatacttttatccaacatgggtggcggcatgatcttaggattgggccaccgacggtttaggcgctatacaaatatactttctttgtatttcgccttccttttttatttttatttttcgcttgttgtgaggatattgttggctgtgtgcatctcagttatgcagaggccgggtgttacttaaaccttttaagtaataaagcgccccttttcgaaaaaatatcaaaatttatttctgttcttgacttgactactggaggccatgttatgcaatatttcgacatatatagctgggtttcttttgaggcaatcttgtgcgatatat

SEQ ID NO: 5 - DNA, 6523 bp, Triticum aestivum
agccagcagaagtcttagaaacagccacgtcgagaaaacgatgtgtgggtcccggttcctaggtctgtgacagagactagaggatcatcatcggttctcggttctcggttttcgggtttgtgaagcctctgaccctggcatttgctcgggttcggttctgctctaggtgcctactggctacggccaacgcgcctcctgtcggggcggttttccacgcaacttagcatccggcaacttatatataacaaacctgcgttccttcttctcgctccaccggtttccaagctcagagcttcaagccaaacccatttccagtgaagcagtcgatggagctcctcaccttcctcttcagaatggtggccctgatccccggcgcattacgcaacgcggagaagctgcccggtgctctcatctcgtgcggcgtcgtccaagccgcggcggcgctcttcctcatcatctccgggacaccaccgggaggtttgtttcttcaccatggcagggcgcccttctatttgtacttcggcatcctcattgccatagtggtattcgggcttgtggaggcgtccgccggattttatgtgtccggcgatgtggccggacgccgtgctgccgggaagaccatcctgtgggtgtcttgcccatcgtcgtcgtcgctgcgctcgccggcttcgtggttctcaactgagacacccctccgccctctgtttgatcgacgtagcgcccgttcatgttgtatagaatataatgagtgtatgtgtgtgtgtgtgtgtgtgtgtgtgtgtgtgtgtgctggggggccattttggtcagtgtgtgctttggggacgggggaatcagtagtaggttgtaccagcacgagtgttttagacttcatatactttcattctttttttcacttgacagttgtaatccaatactgtatgtgtatcagatctggcttatttatgccgtaaattattgaaggagtttgtactaatcaatctttgggttgtgctgtgactttcgatcaacttgaatgtatgtcgtctgcttgattaatatttctttttttgaggatctctgcttggttattttgtactgcttctcatgccgtgttcgggccgtattctcgagcataaagttcggcccactaagtgtcgaaagaaagctgcttctaattgaccttctgctccggcccaacacatgcaccattttaagcgttttccagtgaaagcgaagtgttttttttcccagcctttcatttcttcagcaagtgcgttgtgtgttgtggctggtgttcttccccgctcgtctcgtctgctccccattccacacgcttaattccccttccttcattgactcgagctcgagacctgctcctgccggatctgataatggagccggcgggagactcttccctggaggccgcgattgcatggctggtgcagaccatccttgcaacgctcctcatggacaagatggaggcctggattcagcaagtcgggcttgccgacgacgtcgagaggctccagtctgaggtcgagagagtcgacacggtggtggctgctgtgaaggggagggcagccgggaacatgcctctgtcccggtctctcgctcgtgtcaaggagcttctctatgacgccgacgacgtgatcgacgagctagactactacaggctccaacaccaagtcgaaggaggtagtaagcataatcccattatatatcgaatctatgtgtgctactcaatagtttgatcttaatttctggtccatgtttcttttcggcacagttacaagtgacgagcctgacggtatgcgtggagctgaaagagtggatgaaatatcaaggggccatgtcgatacactgaatgtcagtgttggcaaattacggtccccggtatgggaacacttcaccatcacagaaacaactatcgacgggaagcgttcaaaagccaaatgtaagtactgtggaaatgattttaattgcgaaacgaagacaaacgggacttcatctatgaaaaaacatttggagaaggagcattccgtgacttgcacgaataaatctgcagtgcaccccccaaacacttcaaggtaccagcaggaatttataccttgcttcaacgaatttgttgtaattgtttatatacgtctgcttgagagcccattgttgttctgaatttcttctgataaccaacccaccatccttttcttactgcagcaccggcgatgctacttgtaatgtgaggtcggttgaagttggtagttcgtccaacggaaaaagaaagagaacaaatgaggatccaacgcagaccaccgcagctaacatacacgcccaatgggacaaggctgagttatccaataggataattaaaattactgagaagttacagttacaggacatccagggggctttgagtaaagttctcgagccatatggatccagcgctacttcaagttcaaatcatcaccgcttgagtacagcatcagatcagcacccaacaacatcaagtcttgttccaatggaagtttatggcagagttgcagaaaagaataagatcaaaaagtcaataactgaaaaccaatctggtggtgtaaatgttctgcctattgtaggcattgcaggtgttggaaagacaactcttgctcaatttgtgtataatgatccagacgtgaaaagtcaatttcaccacaggatatgggtttgtgtgtcctgcaaatttgatgaagtgaagctcacaaaggagatgttagacttttttcctcgagaaaggcatgaaggaattaacaacttcgcgaagcttcaagagatcttgaaagaacatgtcgagtaccaagcaaagagttttctgctcattttagatgatgtctcggacagtatggattatcataaatggaacaaattgttgaaccctttgctatcaagtcaagcgaagaatataattctagtcacgaccagaaatttgtctgttgcacaaaggttaagcacacttgaaccgatcaagttaggtgctttagaaaacgatgatatgtggttattgctcaagtcatgtgcatttggttttgggaactatgaaggtacggaaaatctaagcactattggaagacaaatagcagagaagttaaagggcaatccgttagcagcagtaactgcaggggcactgttaagagataatcttagcattgatcattggagtaacattctcaagaatgagaagtggaaatcgctgggactcagtgggggcatcatgcctgctttgaagcttagttatgatgagttgacgtaccgtttacaacaatgtttctcgtattgctctatatttcctgacaaatataggtttctcgggaaggatttggtctatatttggatttctcagggatttgtgaattgcacccaaaataagagattggaggagacgggatgggaatatctgaatcaattggtaaatcttggattctttcaacaaattgaagaacaacaagaattggatggggaagaagaattctctctacgccgtcagatttggtactctatgtgtgatctcatgcatgatttcgcaaggatgatttcaaggactgaatgtgcgactatagatggtctacagtgcaataaaatattcccaactgtacagcatttgtcaatagtaaccggttctgcatacaacaaagatctgaaggggaacattcctcgtaatgagaagtttgaagaaaatatgagaaattcagttacatcagttaccaaattgagaacattggttgtgcttgggaactttgactctttctttgtacggttgttccaagatatattccagaaggcacaaaatttacgcctgctgctagtatctctagcatccacttatctgtctcaagtgcctgctgcattcaatgattttaattccttcctgtgcaatttggcaaatcctttgcatcttcgttacctaaaacttgagttggatgggattgtgccacaagttttgagtacgttttttcatcttcaagtattagatgttggatcaagcatggatacttctctacccaatggcttgttgcataatcttgttagcctgcgacatcttgttgcacacaagagagtccattcttccattactagcattggtaacatgacatctatccaggagctacatgattttgaagttcgaatttctagcggctttgagataacacgactccaatccatgaacgagcttgttcaacttgggttgtctcaacttgacagtgttaaaaccagggaggacgcttatggggcaggactaagaaacaaggaacacttagaagagcttcatttgtcctggaaggatgcatattcagagtatgagtatgccagtgacactgaatttgaatcttctgcaaacatggcaagagaagtgattgagggtcttgaaccacacatggatttaaaacatctacaaatatctcagtataatggtaccacttcaccagcttggcttgccaacaatatctcagttacctcattgcagacgcttcatcttgatgattgtggaggatggagaatacttccatctctgggaagtcttccattccttacaaaggtgaagttgagcagcatgctggaagtaattgaagtactgattccttcactggaggagctagttctaattaaaatgccgaagttagtgagatgctcaagcacttctgccgagggtctgagctctagcttaagggtactgcacattgaggattgtgaagcattgaaggagtttgatctgtttgagaacgattataattctgaaatcattcagggatcatggctgcctggtcttaggaatttgattctatattgttgccctcatttgaaagtgttgaagcctcttccaccttcaactaccttttctaaggtactcatcagagaaatttcaagatttccgtctatggaggtatcatctggtgagaagttacaaattgggaatattgatgtgtacataggcgatgattttgatgagtcttctgatgagttgagcatactggatgacaaaactttggcgttccataatcttagaaacctgaaatcgatggagatatatggttgcagaaatctaaggtctttttcgttcgaaggtttcagtcatcttgtctctttaacaagtttgaaaatagtaagctgtgaacaacttttcccttcagatgtgacggcagagtatacccttgaagatgtgacagctgtgaactgcaatgccttcccatatcttaaaagcctcagtatcgactcatgtggaatagcggggaagtggctatcgctgatgctgcagcatgcgccaggcctagaggaattgagtttaacaagttgcgcccatataacaagagtagtgttaccgatggaagaggaagaaaacaatctattaacaacagtactgtcatcaggaaatcaagatgaggcattgacatggttagttcgtgacggactcttgcacattccatcaaatctcgtctcctctctcaagaatatgagtattactcagtgccctcgcctaaagtttaactcaggcaaggactgcttctctggatttacctcgcttgagaagcttgaaatttggggatcgttggtggatgatgacggaagtgatgacctggagaatggaagttcttttgtgttcggagaggaggatcaacccctgggggcgaacggaagatggctcctcccgacatcacttcaggaacttcacatcgtgtcattgtattgccaagaaacgctgcaagtctgcttccctagagatatcaccagccttaaaaagttaagtgtacgttccggccaaggtttgcaatctctacagctgtactcatgcacggcactggaagaattggcaatttccggctctggatcggtcaccgtcactgtactagagggcacgcaacccgctggcagcctcgggcgtttgaatgtatcagactgtcctggcttgccatcacgtttggacagctttccaaggttgtgccctcggctggaaaggcttgacatcaatgacccatctgtccttaccacgccattctgcaagcacctcacctccctgcaacgcctaaaacttggcttcttgaaagtgacgagactaacagatgagcaagaacgagcgcttgtgctcctcaagtcactgaaagagctcgagattttttattgtactcatctcatagatcttcctgcggggctgcagacccttccttccctcaagagtttgaagatagaagagggtcgaggcatctcaaggctgccggaagcaggcctcccacattcgctggaagaactggaaatcaaaatttgcagcaagctagaagatgaatgcaggcggctagcaacatgcgaaggcaagctaaaagtcaaaattgatggtcgatatgtgaattaattatgtttctggcctcatgtgcaaagtgtaccgcttggatgctcttgagttaaagttgtgtcatataccatattatactcagagtcgacataccatcataaatgtgcagcatgctctttccagaatgagcgtttcagtaagcattcaagctgctcaacatatatctcgtag

SEQ ID NO: 6 - protein, 876 aa, artificial sequence
  YrSP_protein
MEPAGDSSVEAAIAWLVQTILATLLMDKMEEWIRQVGLADDVERLQSEVERVDTVVAAVKGRAAGNRPLSRALARVKELLYDADDLIDELDYYRLQQQVEGVTSDDPDGMRGAERVDEISRGHVDTLNCSVGKLRSPVWEHFTITETTIDGKRSKAKCNYCGNDFNCETKTNGTSSMKKHLEKEHSVTCTKKPGAHPPNPSSTGYATENVTLVEVGSSSNRKRKRTNKEPAQTTADNTRWDKAELSDTIKKITSQLQLQLQGILWAFSKVLEPHGSSSASSSNHHQPSTTSDQHAKTSSLAPRKVYGRVAEMNSIRNLIAEKKCDALTVLPIVGIAGVGKTTLAQSVYNDPDIKSQFHHKIWVCVSRKFDEVMLTREMLDFERHEGSPHENGRHEGISSLAKLQEILKDIIEYQSKSFLLILDDVWDSMDDHQWRKLVCPFVSSQAKGNLILVTTRNLSVAHMLGTREPIKLGALENDVMWLLLKSCAFRDVNYEGNQSLSIVGRQISEKLKGNPLAAETAGALLRKKFSIDYWKIILKNEDWKSMELGNGIMAALKLSYDQLPYHLQQCFSYCSIFPDGYQFLGEELVGFWMSQGFVKCNNSSQRLEQIGQCYLIDLVNLGFFEEVKREEPYLGCRVMYGICGLMHDFVIMVSRTDCASIDGLQRNKMPQTLRHLSIVTGSAYKKNQHGNIPRNNRFEENLRNTITSVSELRTLVLLGHYDFSFLLLFQDIFQKAHNLRVLQMSAPPADFLKHRFEEVDGSFPQILSKLYHLQVLDVGAYTDRTMPGCIDNLVSLRHLVVHKGVYSSIATIDNMLSFQEQHGFKFHISSGFEITRLQSTEHWMHVDTLEDVYEAGLVNNELSEKLHLSWKILLRT

SEQ ID NO: 7 - DNA, 4873 bp, Triticum aestivum
atggagccggcgggagactcttccgtggaggccgcgattgcatggctggtgcagaccatccttgcaacgctcctcatggacaagatggaggagtggattcggcaagtcgggcttgccgacgacgtcgagaggctccagtctgaggtcgagagagtcgacacggtggtggctgctgtgaaggggagggcagccgggaacaggcctctgtcccgggctctcgctcgtgtcaaggagcttctctacgacgccgacgacttgatcgacgagctagactactacaggctccaacaacaagtcgaaggaggtagtaagcataatcccattatatatcgaatatatgtaagctcaagatatttattttgggatggagggagtagtttgatcttaatttctggtccatatttttttcggcacagttacgagtgacgaccctgacggtatgcgtggagctgaaagagtggatgaaatatcaaggggccatgtcgatacactgaattgcagtgttggcaaattacgatccccggtatgggaacacttcacgatcacagaaacaactatcgacgggaagcgttcaaaagccaaatgtaactactgtggaaatgattttaattgcgaaacgaagacaaacgggacttcatctatgaaaaaacatttggagaaagagcattccgtgacttgtacgaagaaacctggagcccatccaccaaacccttcaaggtacccaaaggaaattatatgttgcatcagcgcatttatattcgtttatatatatctgcttgagagcccattgttgttctacatttcttctgataactgacccaccattttctctcttaatgcagcaccggctatgcaactgaaaatgtgacgcttgttgaagttggtagttcatccaacagaaaaagaaagagaacgaataaggagccagcacaaaccaccgcagataacacccgttgggacaaggctgagttatccgatacaataaaaaagattactagccagttacagttacagttacagggtatcctatgggctttcagtaaagttctcgagccacatgggtctagctctgcgtcgagttcaaatcatcaccaaccgagtacaacctcagatcagcacgcaaaaacatcaagtcttgctccaaggaaagtgtatggcagagtagcagaaatgaactccatcagaaatttaatagcagaaaagaaatgtgatgctctaactgttctgcctattgtgggcattgctggtgttggaaagacaactctcgctcaatctgtatacaatgatccagatataaaaagtcaatttcaccacaagatatgggtttgcgtgtcccgcaaatttgatgaagtgatgctcacaagggagatgttagactttgaaagacacgagggatctcctcatgaaaatggaaggcatgaaggaattagtagccttgctaagcttcaggagatcttgaaggacattatcgagtaccagtcaaagagttttctgcttattttagatgatgtatgggacagtatggatgatcatcaatggagaaaactggtgtgtccttttgtatcaagtcaagcaaagggtaatttaattctagtcacaaccagaaatttgtcagttgcacacatgttaggaacacgtgagccgataaagttgggtgctttggaaaatgatgttatgtggttgctgctcaagtcatgtgcatttcgtgatgtgaattatgaagggaaccaaagtctaagcattgtcgggaggcaaatatcagagaagttaaagggaaacccactagcagcagaaacagcgggggcactattaaggaagaaatttagcattgattattggaaaatcattttaaagaatgaagactggaaatccatggagctcggtaatggaatcatggctgctctaaagcttagctatgatcaacttccctaccatttacaacaatgtttctcatattgctccatattccccgacggttatcagtttcttggtgaggagttggtcggtttctggatgtcacagggatttgtaaagtgcaacaactctagtcagagattggagcagataggacagtgctatctgattgatttggttaacttaggcttctttgaagaagttaaaagagaagaaccatatctgggctgtcgagttatgtatggcatatgtggtctcatgcatgattttgtgattatggtgtcaaggactgactgtgcaagtatagatggtctgcagcgcaacaaaatgcctcaaactctacgacatttgtcaatagtaactggatccgcgtacaagaaaaatcagcacggaaacattcctcgtaataataggtttgaagaaaatctgagaaatacaattacatcagttagcgagttgaggacattggtgttacttgggcattatgacttttccttcttactattattccaagatatatttcaaaaggcacataacttacgtgtgctgcaaatgtctgcaccacctgctgattttctcaaacataggtttgaggaggtggatgggtctttccctcaaattttgagcaaattgtaccatctccaagtattagacgtcggtgcatacactgatcgtactatgcctggttgtattgataatcttgttagcctgcggcatcttgttgtacacaagggagtgtactcttccattgcaaccattgataatatgctatcatttcaggaacaacatggtttcaagtttcatatttctagtggctttgagataacacgactccaatccactgaacattggatgcatgttgatactctggaagatgtttatgaggcaggactggtaaacaatgaactctcagaaaagttgcacctgtcctggaagattctcctgaggacataggcatggaggttgaggattgggaaccacattgggacttaagggttctcgagatatctgggtataattttggttcgccaattgtggttgacatcattatcttggttacatcctcccagacggttgagatatccaattgtagtgaatggaaaatacttccatctttggaaagatttcagtttttgacaaatctggagttgagaaacctgcccaaagtaatagaaatactggttccttcactggaggagctagcattagttacaatgccaaagttgaagaaatgttcatgcactcccgtggaaggtatgagctctagactaagagcactgcggatcgaggattgtcaatcactgaaggagtttgatctgtttgagaacaatgataaattcgaaactgggcagaggtcatgggctcctagtcttagggaactaagtctggagaattgcccccatttgaaagtgttgaagcctcttccactctcactcatgtgttctgagttactcataagtggagtttcaacacttccgtacatgaaggggtcatctgatagaaagttatgtattgggtatgatgataagtatgactactatggttttgacgaatcttccgatgagttgaagatactggatgacaaaatttttatgttccataatctgaaaaacctcaaatcaatggtgatatatggttgccggaatctaagttccatttcgttaaaaggttttagttacctcatctctttaacgagcttggaaataagagactgtgaaaaactttttgcttcagatgagatgccagagcatacccttgaagatgtgacacctgcgaattgcaaggctttcccatctcttgaatgtctcagtattgattcatgtggtatagtggggaagtggctatctctgatgctgcaacatgcgccatgcctagaggagttgtatttgtcttcccgagaggaagaaaattcagaagaagaaaattcagaagaggaagaaaacagtatatcaaatcttagctcaaccagggagggcacatcatccggaaatccagatgacggattagctctagaccgactgttgcgcataccattaaatctcatctccattctaaagagtataactattgagagatgccctcatctaacatttaactggggcaaggaaggcgtctcgggatttacctcccttgagaagctaatcgttttggaccgccccgacatggtgcttacaaacggaagatggctcctcccaaactcacttggcgaacttgaaagcaatgactattcccgaggaacgctgcaaccctgctttcctagcgatatcactagccttaaaaagttaaaggtacgtcgcagcccaggtttgcaatctctacagctgcactcatgcatggcactggaagaattggatattcaagattgtcgaaggctcgctgcactgcagggtctgcaattccttggcagcctcacgcatttgaccatatacaactgccctggcttgccaccatttctggagagcttttcaaggcagggctatacgctgttacctcggctgaaaaggcttcacatccaagacccatctgtccttaccacgtcattctgcaggcaccttacctccctgcaacacctaaaacttacttggttggaagaagtgagactaacagatgagcaagagcaagcgcttgtgctcctcaagtccctgcaagagctccaatttcattattgttccaatctcgtagatcttcctgcggtgctgcacaaccttccttccctgaagactttgaaggtagatgggtgtaggggcatctcaaggctgccagaaacaggcctcccattttcgctggaagaactggaaatcgagtggtgcagcaaggagctcgctgatcaatgcaggctgctagcatcaaacaagctaaatatcaaaatttatttctgttcttgacttgactactggaggccatgttatgcaatatttcgacatatatagctgggtttcttttgaggcaatcttgtgcg

SEQ ID NO: 8 - protein, 48 aa, artificial sequence
  BED-II sequence
KAWDNFDVIEEENGQPIKARCKYCPTEIKCGPKSGTAGMLNHNKICKD

SEQ ID NO: 9 - DNA, 4761 bp, artificial sequence
  Yr7_CDS
atggagccggcgggagactcttccctggaggccgcgattgcatggctggtgcagaccatccttgcaacgctcctcatggacaagatggaggcctggattcagcaagtcgggcttgccgacgacgtcgagaggctccagtctgaggtcgagagagtcgacacggtggtggctgctgtgaaggggagggcagccgggaacatgcctctgtcccggtctctcgctcgtgtcaaggagcttctctatgacgccgacgacgtgatcgacgagctagactactacaggctccaacaccaagtcgaaggagttacaagtgacgagcctgacggtatgcgtggagctgaaagagtggatgaaatatcaaggggccatgtcgatacactgaatgtcagtgttggcaaattacggtccccggtatgggaacacttcaccatcacagaaacaactatcgacgggaagcgttcaaaagccaaatgtaagtactgtggaaatgattttaattgcgaaacgaagacaaacgggacttcatctatgaaaaaacatttggagaaggagcattccgtgacttgcacgaataaatctgcagtgcaccccccaaacacttcaagcaccggcgatgctacttgtaatgtgaggtcggttgaagttggtagttcgtccaacggaaaaagaaagagaacaaatgaggatccaacgcagaccaccgcagctaacatacacgcccaatgggacaaggctgagttatccaataggataattaaaattactgagaagttacagttacaggacatccagggggctttgagtaaagttctcgagccatatggatccagcgctacttcaagttcaaatcatcaccgcttgagtacagcatcagatcagcacccaacaacatcaagtcttgttccaatggaagtttatggcagagttgcagaaaagaataagatcaaaaagtcaataactgaaaaccaatctggtggtgtaaatgttctgcctattgtaggcattgcaggtgttggaaagacaactcttgctcaatttgtgtataatgatccagacgtgaaaagtcaatttcaccacaggatatgggtttgtgtgtcctgcaaatttgatgaagtgaagctcacaaaggagatgttagacttttttcctcgagaaaggcatgaaggaattaacaacttcgcgaagcttcaagagatcttgaaagaacatgtcgagtaccaagcaaagagttttctgctcattttagatgatgtctcggacagtatggattatcataaatggaacaaattgttgaaccctttgctatcaagtcaagcgaagaatataattctagtcacgaccagaaatttgtctgttgcacaaaggttaagcacacttgaaccgatcaagttaggtgctttagaaaacgatgatatgtggttattgctcaagtcatgtgcatttggttttgggaactatgaaggtacggaaaatctaagcactattggaagacaaatagcagagaagttaaagggcaatccgttagcagcagtaactgcaggggcactgttaagagataatcttagcattgatcattggagtaacattctcaagaatgagaagtggaaatcgctgggactcagtgggggcatcatgcctgctttgaagcttagttatgatgagttgacgtaccgtttacaacaatgtttctcgtattgctctatatttcctgacaaatataggtttctcgggaaggatttggtctatatttggatttctcagggatttgtgaattgcacccaaaataagagattggaggagacgggatgggaatatctgaatcaattggtaaatcttggattctttcaacaaattgaagaacaacaagaattggatggggaagaagaattctctctacgccgtcagatttggtactctatgtgtgatctcatgcatgatttcgcaaggatgatttcaaggactgaatgtgcgactatagatggtctacagtgcaataaaatattcccaactgtacagcatttgtcaatagtaaccggttctgcatacaacaaagatctgaaggggaacattcctcgtaatgagaagtttgaagaaaatatgagaaattcagttacatcagttaccaaattgagaacattggttgtgcttgggaactttgactctttctttgtacggttgttccaagatatattccagaaggcacaaaatttacgcctgctgctagtatctctagcatccacttatctgtctcaagtgcctgctgcattcaatgattttaattccttcctgtgcaatttggcaaatcctttgcatcttcgttacctaaaacttgagttggatgggattgtgccacaagttttgagtacgttttttcatcttcaagtattagatgttggatcaagcatggatacttctctacccaatggcttgttgcataatcttgttagcctgcgacatcttgttgcacacaagagagtccattcttccattactagcattggtaacatgacatctatccaggagctacatgattttgaagttcgaatttctagcggctttgagataacacgactccaatccatgaacgagcttgttcaacttgggttgtctcaacttgacagtgttaaaaccagggaggacgcttatggggcaggactaagaaacaaggaacacttagaagagcttcatttgtcctggaaggatgcatattcagagtatgagtatgccagtgacactgaatttgaatcttctgcaaacatggcaagagaagtgattgagggtcttgaaccacacatggatttaaaacatctacaaatatctcagtataatggtaccacttcaccagcttggcttgccaacaatatctcagttacctcattgcagacgcttcatcttgatgattgtggaggatggagaatacttccatctctgggaagtcttccattccttacaaaggtgaagttgagcagcatgctggaagtaattgaagtactgattccttcactggaggagctagttctaattaaaatgccgaagttagtgagatgctcaagcacttctgccgagggtctgagctctagcttaagggtactgcacattgaggattgtgaagcattgaaggagtttgatctgtttgagaacgattataattctgaaatcattcagggatcatggctgcctggtcttaggaatttgattctatattgttgccctcatttgaaagtgttgaagcctcttccaccttcaactaccttttctaaggtactcatcagagaaatttcaagatttccgtctatggaggtatcatctggtgagaagttacaaattgggaatattgatgtgtacataggcgatgattttgatgagtcttctgatgagttgagcatactggatgacaaaactttggcgttccataatcttagaaacctgaaatcgatggagatatatggttgcagaaatctaaggtctttttcgttcgaaggtttcagtcatcttgtctctttaacaagtttgaaaatagtaagctgtgaacaacttttcccttcagatgtgacggcagagtatacccttgaagatgtgacagctgtgaactgcaatgccttcccatatcttaaaagcctcagtatcgactcatgtggaatagcggggaagtggctatcgctgatgctgcagcatgcgccaggcctagaggaattgagtttaacaagttgcgcccatataacaagagtagtgttaccgatggaagaggaagaaaacaatctattaacaacagtactgtcatcaggaaatcaagatgaggcattgacatggttagttcgtgacggactcttgcacattccatcaaatctcgtctcctctctcaagaatatgagtattactcagtgccctcgcctaaagtttaactcaggcaaggactgcttctctggatttacctcgcttgagaagcttgaaatttggggatcgttggtggatgatgacggaagtgatgacctggagaatggaagttcttttgtgttcggagaggaggatcaacccctgggggcgaacggaagatggctcctcccgacatcacttcaggaacttcacatcgtgtcattgtattgccaagaaacgctgcaagtctgcttccctagagatatcaccagccttaaaaagttaagtgtacgttccggccaaggtttgcaatctctacagctgtactcatgcacggcactggaagaattggcaatttccggctctggatcggtcaccgtcactgtactagagggcacgcaacccgctggcagcctcgggcgtttgaatgtatcagactgtcctggcttgccatcacgtttggacagctttccaaggttgtgccctcggctggaaaggcttgacatcaatgacccatctgtccttaccacgccattctgcaagcacctcacctccctgcaacgcctaaaacttggcttcttgaaagtgacgagactaacagatgagcaagaacgagcgcttgtgctcctcaagtcactgaaagagctcgagattttttattgtactcatctcatagatcttcctgcggggctgcagacccttccttccctcaagagtttgaagatagaagagggtcgaggcatctcaaggctgccggaagcaggcctcccacattcgctggaagaactggaaatcaaaatttgcagcaagctagaagatgaatgcaggcggctagcaacatgcgaaggcaagctaaaagtcaaaattgatggtcgatatgtgaattaa

SEQ ID NO: 10 - DNA, 4569 bp, artificial sequence
  Yr5_CDS
atggagccggcgggagactcttccgtggaggccgcgattgcatggctggtgcagaccatccttgcaacgctcctcatggacaagatggaggagtggattcggcaagtcgggcttgccgacgacgtcgagaggctccagtctgaggtcgagagagtcgacacggtggtggctgctgtgaaggggagggcagccgggaacaggcctctgtcccgggctctcgctcgtgtcaaggagcttctctacgacgccgacgacttgatcgacgagctagactactacaggctccaacaacaagtcgaaggagttacgagtgacgaccctgacggtatgcgtggagctgaaagagtggatgaaatatcaaggggccatgtcgatacactgaattgcagtgttggcaaattacgatccccggtatgggaacacttcacgatcacagaaacaactatcgacgggaagcgttcaaaagccaaatgtaactactgtggaaatgattttaattgcgaaacgaagacaaacgggacttcatctatgaaaaaacatttggagaaagagcattccgtgacttgtacgaagaaacctggagcccatccaccaaacccttcaagcaccggctatgcaactgaaaatgtgacgcttgttgaagttggtagttcatccaacagaaaaagaaagagaacgaataaggagccagcacaaaccaccgcagataacacccgttgggacaaggctgagttatccgatacaataaaaaagattactagccagttacagttacagttacagggtatcctatgggctttcagtaaagttctcgagccacatgggtctagctctgcgtcgagttcaaatcatcaccaaccgagtacaacctcagatcagcacgcaaaaacatcaagtcttgctccaaggaaagtgtatggcagagtagcagaaatgaactccatcagaaatttaatagcagaaaagaaatgtgatgctctaactgttctgcctattgtgggcattgctggtgttggaaagacaactctcgctcaatctgtatacaatgatccagatataaaaagtcaatttcaccacaagatatgggtttgcgtgtcccgcaaatttgatgaagtgatgctcacaagggagatgttagactttgaaagacacgagggatctcctcatgaaaatggaaggcatgaaggaattagtagccttgctaagcttcaggagatcttgaaggacattatcgagtaccagtcaaagagttttctgcttattttagatgatgtatgggacagtatggatgatcatcaatggagaaaactggtgtgtccttttgtatcaagtcaagcaaagggtaatttaattctagtcacaaccagaaatttgtcagttgcacacatgttaggaacacgtgagccgataaagttgggtgctttggaaaatgatgttatgtggttgctgctcaagtcatgtgcatttcgtgatgtgaattatgaagggaaccaaagtctaagcattgtcgggaggcaaatatcagagaagttaaagggaaacccactagcagcagaaacagcgggggcactattaaggaagaaatttagcattgattattggaaaatcattttaaagaatgaagactggaaatccatggagctcggtaatggaatcatggctgctctaaagcttagctatgatcaacttccctaccatttacaacaatgtttctcatattgctccatattccccgacggttatcagtttcttggtgaggagttggtcggtttctggatgtcacagggatttgtaaagtgcaacaactctagtcagagattggagcagataggacagtgctatctgattgatttggttaacttaggcttctttgaagaagttaaaagagaagaaccatatctgggctgtcgagttatgtatggcatatgtggtctcatgcatgattttgtgattatggtgtcaaggactgactgtgcaagtatagatggtctgcagcgcaacaaaatgcctcaaactctacgacatttgtcaatagtaactggatccgcgtacaagaaaaatcagcacggaaacattcctcgtaataataggtttgaagaaaatctgagaaatacaattacatcagttagcgagttgaggacattggtgttacttgggcattatgacttttccttcttactattattccaagatatatttcaaaaggcacataacttacgtgtgctgcaaatgtctgcagcacctgctgattttctcaaacataggtttgaggaggtggatgggtctttccctcaaattttgagcaaattgtaccatctccaagtattagacgtcggtgcatacactgatcgtactatgcctggttgtattgataatcttgttagcctgcggcatcttgttgtacacaagggagtgtactcttccattgcaaccattgataatatgctatcatttcaggaacaacatggtttcaagtttcatatttctagtggctttgagataacacgactccaatccactgaacattggatgcatgttgatactctggaagatgtttatgaggcaggactggtaaacaatgaactctcagaaaagttgcacctgtcctggaaggattctcctgaggacataggcatggaggttgaggattgggaaccacattgggacttaagggttctcgagatatctgggtataattttggttcgccaattgtggttgacatcattatcttggttacatcctcccagacggttgagatatccaattgtagtgaatggaaaatacttccatctttggaaagatttcagtttttgacaaatctggagttgagaaacctgcccaaagtaatagaaatactggttccttcactggaggagctagcattagttacaatgccaaagttgaagaaatgttcatgcactcccgtggaaggtatgagctctagactaagagcactgcggatcgaggattgtcaatcactgaaggagtttgatctgtttgagaacaatgataaattcgaaactgggcagaggtcatgggctcctagtcttagggaactaagtctggagaattgcccccatttgaaagtgttgaagcctcttccactctcactcatgtgttctgagttactcataagtggagtttcaacacttccgtacatgaaggggtcatctgatagaaagttatgtattgggtatgatgataagtatgactactatggttttgacgaatcttccgatgagttgaagatactggatgacaaaatttttatgttccataatctgaaaaacctcaaatcaatggtgatatatggttgccggaatctaagttccatttcgttaaaaggttttagttacctcatctctttaacgagcttggaaataagagactgtgaaaaactttttgcttcagatgagatgccagagcatacccttgaagatgtgacacctgcgaattgcaaggctttcccatctcttgaatgtctcagtattgattcatgtggtatagtggggaagtggctatctctgatgctgcaacatgcgccatgcctagaggagttgtatttgtcttcccgagaggaagaaaattcagaagaagaaaattcagaagaggaagaaaacagtatatcaaatcttagctcaaccagggagggcacatcatccggaaatccagatgacggattagctctagaccgactgttgcgcataccattaaatctcatctccattctaaagagtataactattgagagatgccctcatctaacatttaactggggcaaggaaggcgtctcgggatttacctcccttgagaagctaatcgttttggaccgccccgacatggtgcttacaaacggaagatggctcctcccaaactcacttggcgaacttgaaagcaatgactattcccgaggaacgctgcaaccctgctttcctagcgatatcactagccttaaaaagttaaaggtacgtcgcagcccaggtttgcaatctctacagctgcactcatgcatggcactggaagaattggatattcaagattgtcgaaggctcgctgcactgcagggtctgcaattccttggcagcctcacgcatttgaccatatacaactgccctggcttgccaccatttctggagagcttttcaaggcagggctatacgctgttacctcggctgaaaaggcttcacatccaagacccatctgtccttaccacgtcattctgcaggcaccttacctccctgcaacacctaaaacttacttggttggaagaagtgagactaacagatgagcaagagcaagcgcttgtgctcctcaagtccctgcaagagctccaatttcattattgttccaatctcgtagatcttcctgcggtgctgcacaaccttccttccctgaagactttgaaggtagatgggtgtaggggcatctcaaggctgccagaaacaggcctcccattttcgctggaagaactggaaatcgagtggtgcagcaaggagctcgctgatcaatgcaggctgctagcatcaaacaagctaaatatcaaaattctcagtggaatctatgtatag

SEQ ID NO: 11 - DNA, 2631 bp, artificial sequence
  YrSP_CDS
atggagccggcgggagactcttccgtggaggccgcgattgcatggctggtgcagaccatccttgcaacgctcctcatggacaagatggaggagtggattcggcaagtcgggcttgccgacgacgtcgagaggctccagtctgaggtcgagagagtcgacacggtggtggctgctgtgaaggggagggcagccgggaacaggcctctgtcccgggctctcgctcgtgtcaaggagcttctctacgacgccgacgacttgatcgacgagctagactactacaggctccaacaacaagtcgaaggagttacgagtgacgaccctgacggtatgcgtggagctgaaagagtggatgaaatatcaaggggccatgtcgatacactgaattgcagtgttggcaaattacgatccccggtatgggaacacttcacgatcacagaaacaactatcgacgggaagcgttcaaaagccaaatgtaactactgtggaaatgattttaattgcgaaacgaagacaaacgggacttcatctatgaaaaaacatttggagaaagagcattccgtgacttgtacgaagaaacctggagcccatccaccaaacccttcaagcaccggctatgcaactgaaaatgtgacgcttgttgaagttggtagttcatccaacagaaaaagaaagagaacgaataaggagccagcacaaaccaccgcagataacacccgttgggacaaggctgagttatccgatacaataaaaaagattactagccagttacagttacagttacagggtatcctatgggctttcagtaaagttctcgagccacatgggtctagctctgcgtcgagttcaaatcatcaccaaccgagtacaacctcagatcagcacgcaaaaacatcaagtcttgctccaaggaaagtgtatggcagagtagcagaaatgaactccatcagaaatttaatagcagaaaagaaatgtgatgctctaactgttctgcctattgtgggcattgctggtgttggaaagacaactctcgctcaatctgtatacaatgatccagatataaaaagtcaatttcaccacaagatatgggtttgcgtgtcccgcaaatttgatgaagtgatgctcacaagggagatgttagactttgaaagacacgagggatctcctcatgaaaatggaaggcatgaaggaattagtagccttgctaagcttcaggagatcttgaaggacattatcgagtaccagtcaaagagttttctgcttattttagatgatgtatgggacagtatggatgatcatcaatggagaaaactggtgtgtccttttgtatcaagtcaagcaaagggtaatttaattctagtcacaaccagaaatttgtcagttgcacacatgttaggaacacgtgagccgataaagttgggtgctttggaaaatgatgttatgtggttgctgctcaagtcatgtgcatttcgtgatgtgaattatgaagggaaccaaagtctaagcattgtcgggaggcaaatatcagagaagttaaagggaaacccactagcagcagaaacagcgggggcactattaaggaagaaatttagcattgattattggaaaatcattttaaagaatgaagactggaaatccatggagctcggtaatggaatcatggctgctctaaagcttagctatgatcaacttccctaccatttacaacaatgtttctcatattgctccatattccccgacggttatcagtttcttggtgaggagttggtcggtttctggatgtcacagggatttgtaaagtgcaacaactctagtcagagattggagcagataggacagtgctatctgattgatttggttaacttaggcttctttgaagaagttaaaagagaagaaccatatctgggctgtcgagttatgtatggcatatgtggtctcatgcatgattttgtgattatggtgtcaaggactgactgtgcaagtatagatggtctgcagcgcaacaaaatgcctcaaactctacgacatttgtcaatagtaactggatccgcgtacaagaaaaatcagcacggaaacattcctcgtaataataggtttgaagaaaatctgagaaatacaattacatcagttagcgagttgaggacattggtgttacttgggcattatgacttttccttcttactattattccaagatatatttcaaaaggcacataacttacgtgtgctgcaaatgtctgcaccacctgctgattttctcaaacataggtttgaggaggtggatgggtctttccctcaaattttgagcaaattgtaccatctccaagtattagacgtcggtgcatacactgatcgtactatgcctggttgtattgataatcttgttagcctgcggcatcttgttgtacacaagggagtgtactcttccattgcaaccattgataatatgctatcatttcaggaacaacatggtttcaagtttcatatttctagtggctttgagataacacgactccaatccactgaacattggatgcatgttgatactctggaagatgtttatgaggcaggactggtaaacaatgaactctcagaaaagttgcacctgtcctggaagattctcctgaggacatag

SEQ ID NO: 12 - DNA, 5069 bp, artificial sequence
  Yr7_with_Ns
  misc_feature (908)..(908): n is a, c, g, or t
  misc_feature (1236)..(1236): n is a, c, g, or t
atggagccggcgggagactcttccctggaggccgcgattgcatggctggtgcagaccatccttgcaacgctcctcatggacaagatggaggcctggattcagcaagtcgggcttgccgacgacgtcgagaggctccagtctgaggtcgagagagtcgacacggtggtggctgctgtgaaggggagggcagccgggaacatgcctctgtcccggtctctcgctcgtgtcaaggagcttctctatgacgccgacgacgtgatcgacgagctagactactacaggctccaacaccaagtcgaaggaggtagtaagcataatcccattatatatcgaatctatgtgtgctactcaatagtttgatcttaatttctggtccatgtttcttttcggcacagttacaagtgacgagcctgacggtatgcgtggagctgaaagagtggatgaaatatcaaggggccatgtcgatacactgaatgtcagtgttggcaaattacggtccccggtatgggaacacttcaccatcacagaaacaactatcgacgggaagcgttcaaaagccaaatgtaagtactgtggaaatgattttaattgcgaaacgaagacaaacgggacttcatctatgaaaaaacatttggagaaggagcattccgtgacttgcacgaataaatctgcagtgcaccccccaaacacttcaaggtaccagcaggaatttataccttgcttcaacgaatttgttgtaattgtttatatacgtctgcttgagagcccattgttgttctgaatttcttctgataaccaacccaccatccttttcttactgcagcaccggcgatgctacttgtaatgtgaggtcggttgaagttggtagttcgtccaacggaaaaagaaagagaacaaatgaggatccnaaggctgagttatccaataggataattaaaattactgagaagttacagttacaggacatccagggggctttgagtaaagttctcgagccatatggatccagcgctacttcaagttcaaatcatcaccgcttgagtacagcatcagatcagcacccaacaacatcaagtcttgttccaatggaagtttatggcagagttgcagaaaagaataagatcaaaaagtcaataactgaaaaccaatctggtggtgtaaatgttctgcctattgtaggcattgcaggtgttggaaagacaactcttgctcaatttgtgtataatgatccagacncagaaaagaataagatcaaaaagtcaataactgaaaaccaatctggtggtgtaaatgttctgcctattgtaggcattgcaggtgttggaaagacaactcttgctcaatttgtgtataatgatccagacgtgaaaagtcaatttcaccacaggatatgggtttgtgtgtcctgcaaatttgatgaagtgaagctcacaaaggagatgttagacttttttcctcgagaaaggcatgaaggaattaacaacttcgcgaagcttcaagagatcttgaaagaacatgtcgagtaccaagcaaagagttttctgctcattttagatgatgtctcggacagtatggattatcataaatggaacaaattgttgaaccctttgctatcaagtcaagcgaagaatataattctagtcacgaccagaaatttgtctgttgcacaaaggttaagcacacttgaaccgatcaagttaggtgctttagaaaacgatgatatgtggttattgctcaagtcatgtgcatttggttttgggaactatgaaggtacggaaaatctaagcactattggaagacaaatagcagagaagttaaagggcaatccgttagcagcagtaactgcaggggcactgttaagagataatcttagcattgatcattggagtaacattctcaagaatgagaagtggaaatcgctgggactcagtgggggcatcatgcctgctttgaagcttagttatgatgagttgacgtaccgtttacaacaatgtttctcgtattgctctatatttcctgacaaatataggtttctcgggaaggatttggtctatatttggatttctcagggatttgtgaattgcacccaaaataagagattggaggagacgggatgggaatatctgaatcaattggtaaatcttggattctttcaacaaattgaagaacaacaagaattggatggggaagaagaattctctctacgccgtcagatttggtactctatgtgtgatctcatgcatgatttcgcaaggatgatttcaaggactgaatgtgcgactatagatggtctacagtgcaataaaatattcccaactgtacagcatttgtcaatagtaaccggttctgcatacaacaaagatctgaaggggaacattcctcgtaatgagaagtttgaagaaaatatgagaaattcagttacatcagttaccaaattgagaacattggttgtgcttgggaactttgactctttctttgtacggttgttccaagatatattccagaaggcacaaaatttacgcctgctgctagtatctctagcatccacttatctgtctcaagtgcctgctgcattcaatgattttaattccttcctgtgcaatttggcaaatcctttgcatcttcgttacctaaaacttgagttggatgggattgtgccacaagttttgagtacgttttttcatcttcaagtattagatgttggatcaagcatggatacttctctacccaatggcttgttgcataatcttgttagcctgcgacatcttgttgcacacaagagagtccattcttccattactagcattggtaacatgacatctatccaggagctacatgattttgaagttcgaatttctagcggctttgagataacacgactccaatccatgaacgagcttgttcaacttgggttgtctcaacttgacagtgttaaaaccagggaggacgcttatggggcaggactaagaaacaaggaacacttagaagagcttcatttgtcctggaaggatgcatattcagagtatgagtatgccagtgacactgaatttgaatcttctgcaaacatggcaagagaagtgattgagggtcttgaaccacacatggatttaaaacatctacaaatatctcagtataatggtaccacttcaccagcttggcttgccaacaatatctcagttacctcattgcagacgcttcatcttgatgattgtggaggatggagaatacttccatctctgggaagtcttccattccttacaaaggtgaagttgagcagcatgctggaagtaattgaagtactgattccttcactggaggagctagttctaattaaaatgccgaagttagtgagatgctcaagcacttctgccgagggtctgagctctagcttaagggtactgcacattgaggattgtgaagcattgaaggagtttgatctgtttgagaacgattataattctgaaatcattcagggatcatggctgcctggtcttaggaatttgattctatattgttgccctcatttgaaagtgttgaagcctcttccaccttcaactaccttttctaaggtactcatcagagaaatttcaagatttccgtctatggaggtatcatctggtgagaagttacaaattgggaatattgatgtgtacataggcgatgattttgatgagtcttctgatgagttgagcatactggatgacaaaactttggcgttccataatcttagaaacctgaaatcgatggagatatatggttgcagaaatctaaggtctttttcgttcgaaggtttcagtcatcttgtctctttaacaagtttgaaaatagtaagctgtgaacaacttttcccttcagatgtgacggcagagtatacccttgaagatgtgacagctgtgaactgcaatgccttcccatatcttaaaagcctcagtatcgactcatgtggaatagcggggaagtggctatcgctgatgctgcagcatgcgccaggcctagaggaattgagtttaacaagttgcgcccatataacaagagtagtgttaccgatggaagaggaagaaaacaatctattaacaacagtactgtcatcaggaaatcaagatgaggcattgacatggttagttcgtgacggactcttgcacattccatcaaatctcgtctcctctctcaagaatatgagtattactcagtgccctcgcctaaagtttaactcaggcaaggactgcttctctggatttacctcgcttgagaagcttgaaatttggggatcgttggtggatgatgacggaagtgatgacctggagaatggaagttcttttgtgttcggagaggaggatcaacccctgggggcgaacggaagatggctcctcccgacatcacttcaggaacttcacatcgtgtcattgtattgccaagaaacgctgcaagtctgcttccctagagatatcaccagccttaaaaagttaagtgtacgttccggccaaggtttgcaatctctacagctgtactcatgcacggcactggaagaattggcaatttccggctctggatcggtcaccgtcactgtactagagggcacgcaacccgctggcagcctcgggcgtttgaatgtatcagactgtcctggcttgccatcacgtttggacagctttccaaggttgtgccctcggctggaaaggcttgacatcaatgacccatctgtccttaccacgccattctgcaagcacctcacctccctgcaacgcctaaaacttggcttcttgaaagtgacgagactaacagatgagcaagaacgagcgcttgtgctcctcaagtcactgaaagagctcgagattttttattgtactcatctcatagatcttcctgcggggctgcagacccttccttccctcaagagtttgaagatagaagagggtcgaggcatctcaaggctgccggaagcaggcctcccacattcgctggaagaactggaaatcaaaatttgcagcaagctagaagatgaatgcaggcggctagcaacatgcgaaggcaagctaaaagtcaaaattgatggtcgatatgtgaattaa

SEQ ID NO: 13 - DNA, 4979 bp, artificial sequence
  curated_Yr7
atggagccggcgggagactcttccctggaggccgcgattgcatggctggtgcagaccatccttgcaacgctcctcatggacaagatggaggcctggattcagcaagtcgggcttgccgacgacgtcgagaggctccagtctgaggtcgagagagtcgacacggtggtggctgctgtgaaggggagggcagccgggaacatgcctctgtcccggtctctcgctcgtgtcaaggagcttctctatgacgccgacgacgtgatcgacgagctagactactacaggctccaacaccaagtcgaaggaggtagtaagcataatcccattatatatcgaatctatgtgtgctactcaatagtttgatcttaatttctggtccatgtttcttttcggcacagttacaagtgacgagcctgacggtatgcgtggagctgaaagagtggatgaaatatcaaggggccatgtcgatacactgaatgtcagtgttggcaaattacggtccccggtatgggaacacttcaccatcacagaaacaactatcgacgggaagcgttcaaaagccaaatgtaagtactgtggaaatgattttaattgcgaaacgaagacaaacgggacttcatctatgaaaaaacatttggagaaggagcattccgtgacttgcacgaataaatctgcagtgcaccccccaaacacttcaaggtaccagcaggaatttataccttgcttcaacgaatttgttgtaattgtttatatacgtctgcttgagagcccattgttgttctgaatttcttctgataaccaacccaccatccttttcttactgcagcaccggcgatgctacttgtaatgtgaggtcggttgaagttggtagttcgtccaacggaaaaagaaagagaacaaatgaggatccaacgcagaccaccgcagctaacatacacgcccaatgggacaaggctgagttatccaataggataattaaaattactgagaagttacagttacaggacatccagggggctttgagtaaagttctcgagccatatggatccagcgctacttcaagttcaaatcatcaccgcttgagtacagcatcagatcagcacccaacaacatcaagtcttgttccaatggaagtttatggcagagttgcagaaaagaataagatcaaaaagtcaataactgaaaaccaatctggtggtgtaaatgttctgcctattgtaggcattgcaggtgttggaaagacaactcttgctcaatttgtgtataatgatccagacgtgaaaagtcaatttcaccacaggatatgggtttgtgtgtcctgcaaatttgatgaagtgaagctcacaaaggagatgttagacttttttcctcgagaaaggcatgaaggaattaacaacttcgcgaagcttcaagagatcttgaaagaacatgtcgagtaccaagcaaagagttttctgctcattttagatgatgtctcggacagtatggattatcataaatggaacaaattgttgaaccctttgctatcaagtcaagcgaagaatataattctagtcacgaccagaaatttgtctgttgcacaaaggttaagcacacttgaaccgatcaagttaggtgctttagaaaacgatgatatgtggttattgctcaagtcatgtgcatttggttttgggaactatgaaggtacggaaaatctaagcactattggaagacaaatagcagagaagttaaagggcaatccgttagcagcagtaactgcaggggcactgttaagagataatcttagcattgatcattggagtaacattctcaagaatgagaagtggaaatcgctgggactcagtgggggcatcatgcctgctttgaagcttagttatgatgagttgacgtaccgtttacaacaatgtttctcgtattgctctatatttcctgacaaatataggtttctcgggaaggatttggtctatatttggatttctcagggatttgtgaattgcacccaaaataagagattggaggagacgggatgggaatatctgaatcaattggtaaatcttggattctttcaacaaattgaagaacaacaagaattggatggggaagaagaattctctctacgccgtcagatttggtactctatgtgtgatctcatgcatgatttcgcaaggatgatttcaaggactgaatgtgcgactatagatggtctacagtgcaataaaatattcccaactgtacagcatttgtcaatagtaaccggttctgcatacaacaaagatctgaaggggaacattcctcgtaatgagaagtttgaagaaaatatgagaaattcagttacatcagttaccaaattgagaacattggttgtgcttgggaactttgactctttctttgtacggttgttccaagatatattccagaaggcacaaaatttacgcctgctgctagtatctctagcatccacttatctgtctcaagtgcctgctgcattcaatgattttaattccttcctgtgcaatttggcaaatcctttgcatcttcgttacctaaaacttgagttggatgggattgtgccacaagttttgagtacgttttttcatcttcaagtattagatgttggatcaagcatggatacttctctacccaatggcttgttgcataatcttgttagcctgcgacatcttgttgcacacaagagagtccattcttccattactagcattggtaacatgacatctatccaggagctacatgattttgaagttcgaatttctagcggctttgagataacacgactccaatccatgaacgagcttgttcaacttgggttgtctcaacttgacagtgttaaaaccagggaggacgcttatggggcaggactaagaaacaaggaacacttagaagagcttcatttgtcctggaaggatgcatattcagagtatgagtatgccagtgacactgaatttgaatcttctgcaaacatggcaagagaagtgattgagggtcttgaaccacacatggatttaaaacatctacaaatatctcagtataatggtaccacttcaccagcttggcttgccaacaatatctcagttacctcattgcagacgcttcatcttgatgattgtggaggatggagaatacttccatctctgggaagtcttccattccttacaaaggtgaagttgagcagcatgctggaagtaattgaagtactgattccttcactggaggagctagttctaattaaaatgccgaagttagtgagatgctcaagcacttctgccgagggtctgagctctagcttaagggtactgcacattgaggattgtgaagcattgaaggagtttgatctgtttgagaacgattataattctgaaatcattcagggatcatggctgcctggtcttaggaatttgattctatattgttgccctcatttgaaagtgttgaagcctcttccaccttcaactaccttttctaaggtactcatcagagaaatttcaagatttccgtctatggaggtatcatctggtgagaagttacaaattgggaatattgatgtgtacataggcgatgattttgatgagtcttctgatgagttgagcatactggatgacaaaactttggcgttccataatcttagaaacctgaaatcgatggagatatatggttgcagaaatctaaggtctttttcgttcgaaggtttcagtcatcttgtctctttaacaagtttgaaaatagtaagctgtgaacaacttttcccttcagatgtgacggcagagtatacccttgaagatgtgacagctgtgaactgcaatgccttcccatatcttaaaagcctcagtatcgactcatgtggaatagcggggaagtggctatcgctgatgctgcagcatgcgccaggcctagaggaattgagtttaacaagttgcgcccatataacaagagtagtgttaccgatggaagaggaagaaaacaatctattaacaacagtactgtcatcaggaaatcaagatgaggcattgacatggttagttcgtgacggactcttgcacattccatcaaatctcgtctcctctctcaagaatatgagtattactcagtgccctcgcctaaagtttaactcaggcaaggactgcttctctggatttacctcgcttgagaagcttgaaatttggggatcgttggtggatgatgacggaagtgatgacctggagaatggaagttcttttgtgttcggagaggaggatcaacccctgggggcgaacggaagatggctcctcccgacatcacttcaggaacttcacatcgtgtcattgtattgccaagaaacgctgcaagtctgcttccctagagatatcaccagccttaaaaagttaagtgtacgttccggccaaggtttgcaatctctacagctgtactcatgcacggcactggaagaattggcaatttccggctctggatcggtcaccgtcactgtactagagggcacgcaacccgctggcagcctcgggcgtttgaatgtatcagactgtcctggcttgccatcacgtttggacagctttccaaggttgtgccctcggctggaaaggcttgacatcaatgacccatctgtccttaccacgccattctgcaagcacctcacctccctgcaacgcctaaaacttggcttcttgaaagtgacgagactaacagatgagcaagaacgagcgcttgtgctcctcaagtcactgaaagagctcgagattttttattgtactcatctcatagatcttcctgcggggctgcagacccttccttccctcaagagtttgaagatagaagagggtcgaggcatctcaaggctgccggaagcaggcctcccacattcgctggaagaactggaaatcaaaatttgcagcaagctagaagatgaatgcaggcggctagcaacatgcgaaggcaagctaaaagtcaaaattgatggtcgatatgtgaattaa

SEQ ID NO: 14 - DNA, 5069 bp, artificial sequence
  Yr7_Paragon_with_Ns
  misc_feature (837)..(837): n is a, c, g, or t
  misc_feature (1165)..(1165): n is a, c, g, or t
atggagccggcgggagactcttccctggaggccgcgattgcatggctggtgcagaccatccttgcaacgctcctcatggacaagatggaggcctggattcagcaagtcgggcttgccgacgacgtcgagaggctccagtctgaggtcgagagagtcgacacggtggtggctgctgtgaaggggagggcagccgggaacatgcctctgtcccggtctctcgctcgtgtcaaggagcttctctatgacgccgacgacgtgatcgacgagctagactactacaggctccaacaccaagtcgaaggaggtagtaagcataatcccattatatatcgaatctatgtgtgctactcaatagtttgatcttaatttctggtccatgtttcttttcggcacagttacaagtgacgagcctgacggtatgcgtggagctgaaagagtggatgaaatatcaaggggccatgtcgatacactgaatgtcagtgttggcaaattacggtccccggtatgggaacacttcaccatcacagaaacaactatcgacgggaagcgttcaaaagccaaatgtaagtactgtggaaatgattttaattgcgaaacgaagacaaacgggacttcatctatgaaaaaacatttggagaaggagcattccgtgacttgcacgaataaatctgcagtgcaccccccaaacacttcaaggtaccagcaggaatttataccttgcttcaacgaatttgttgtaattgtttatatacgtctgcttgagagcccattgttgttctgaatttcttctgataaccaacccaccatccttttcttactgcagcaccggcgatgctnacggaaaaagaaagagaacaaatgaggatccaacgcagaccaccgcagctaacatacacgcccaatgggacaaggctgagttatccaataggataattaaaattactgagaagttacagttacaggacatccagggggctttgagtaaagttctcgagccatatggatccagcgctacttcaagttcaaatcatcaccgcttgagtacagcatcagatcagcacccaacaacatcaagtcttgttccaatggaagtttatggcagagttgcagaaaagaataagatcaaaaagtcaataactgaaaaccaatctggtggtgtaaatgncttgagtacagcatcagatcagcacccaacaacatcaagtcttgttccaatggaagtttatggcagagttgcagaaaagaataagatcaaaaagtcaataactgaaaaccaatctggtggtgtaaatgttctgcctattgtaggcattgcaggtgttggaaagacaactcttgctcaatttgtgtataatgatccagacgtgaaaagtcaatttcaccacaggatatgggtttgtgtgtcctgcaaatttgatgaagtgaagctcacaaaggagatgttagacttttttcctcgagaaaggcatgaaggaattaacaacttcgcgaagcttcaagagatcttgaaagaacatgtcgagtaccaagcaaagagttttctgctcattttagatgatgtctcggacagtatggattatcataaatggaacaaattgttgaaccctttgctatcaagtcaagcgaagaatataattctagtcacgaccagaaatttgtctgttgcacaaaggttaagcacacttgaaccgatcaagttaggtgctttagaaaacgatgatatgtggttattgctcaagtcatgtgcatttggttttgggaactatgaaggtacggaaaatctaagcactattggaagacaaatagcagagaagttaaagggcaatccgttagcagcagtaactgcaggggcactgttaagagataatcttagcattgatcattggagtaacattctcaagaatgagaagtggaaatcgctgggactcagtgggggcatcatgcctgctttgaagcttagttatgatgagttgacgtaccgtttacaacaatgtttctcgtattgctctatatttcctgacaaatataggtttctcgggaaggatttggtctatatttggatttctcagggatttgtgaattgcacccaaaataagagattggaggagacgggatgggaatatctgaatcaattggtaaatcttggattctttcaacaaattgaagaacaacaagaattggatggggaagaagaattctctctacgccgtcagatttggtactctatgtgtgatctcatgcatgatttcgcaaggatgatttcaaggactgaatgtgcgactatagatggtctacagtgcaataaaatattcccaactgtacagcatttgtcaatagtaaccggttctgcatacaacaaagatctgaaggggaacattcctcgtaatgagaagtttgaagaaaatatgagaaattcagttacatcagttaccaaattgagaacattggttgtgcttgggaactttgactctttctttgtacggttgttccaagatatattccagaaggcacaaaatttacgcctgctgctagtatctctagcatccacttatctgtctcaagtgcctgctgcattcaatgattttaattccttcctgtgcaatttggcaaatcctttgcatcttcgttacctaaaacttgagttggatgggattgtgccacaagttttgagtacgttttttcatcttcaagtattagatgttggatcaagcatggatacttctctacccaatggcttgttgcataatcttgttagcctgcgacatcttgttgcacacaagagagtccattcttccattactagcattggtaacatgacatctatccaggagctacatgattttgaagttcgaatttctagcggctttgagataacacgactccaatccatgaacgagcttgttcaacttgggttgtctcaacttgacagtgttaaaaccagggaggacgcttatggggcaggactaagaaacaaggaacacttagaagagcttcatttgtcctggaaggatgcatattcagagtatgagtatgccagtgacactgaatttgaatcttctgcaaacatggcaagagaagtgattgagggtcttgaaccacacatggatttaaaacatctacaaatatctcagtataatggtaccacttcaccagcttggcttgccaacaatatctcagttacctcattgcagacgcttcatcttgatgattgtggaggatggagaatacttccatctctgggaagtcttccattccttacaaaggtgaagttgagcagcatgctggaagtaattgaagtactgattccttcactggaggagctagttctaattaaaatgccgaagttagtgagatgctcaagcacttctgccgagggtctgagctctagcttaagggtactgcacattgaggattgtgaagcattgaaggagtttgatctgtttgagaacgattataattctgaaatcattcagggatcatggctgcctggtcttaggaatttgattctatattgttgccctcatttgaaagtgttgaagcctcttccaccttcaactaccttttctaaggtactcatcagagaaatttcaagatttccgtctatggaggtatcatctggtgagaagttacaaattgggaatattgatgtgtacataggcgatgattttgatgagtcttctgatgagttgagcatactggatgacaaaactttggcgttccataatcttagaaacctgaaatcgatggagatatatggttgcagaaatctaaggtctttttcgttcgaaggtttcagtcatcttgtctctttaacaagtttgaaaatagtaagctgtgaacaacttttcccttcagatgtgacggcagagtatacccttgaagatgtgacagctgtgaactgcaatgccttcccatatcttaaaagcctcagtatcgactcatgtggaatagcggggaagtggctatcgctgatgctgcagcatgcgccaggcctagaggaattgagtttaacaagttgcgcccatataacaagagtagtgttaccgatggaagaggaagaaaacaatctattaacaacagtactgtcatcaggaaatcaagatgaggcattgacatggttagttcgtgacggactcttgcacattccatcaaatctcgtctcctctctcaagaatatgagtattactcagtgccctcgcctaaagtttaactcaggcaaggactgcttctctggatttacctcgcttgagaagcttgaaatttggggatcgttggtggatgatgacggaagtgatgacctggagaatggaagttcttttgtgttcggagaggaggatcaacccctgggggcgaacggaagatggctcctcccgacatcacttcaggaacttcacatcgtgtcattgtattgccaagaaacgctgcaagtctgcttccctagagatatcaccagccttaaaaagttaagtgtacgttccggccaaggtttgcaatctctacagctgtactcatgcacggcactggaagaattggcaatttccggctctggatcggtcaccgtcactgtactagagggcacgcaacccgctggcagcctcgggcgtttgaatgtatcagactgtcctggcttgccatcacgtttggacagctttccaaggttgtgccctcggctggaaaggcttgacatcaatgacccatctgtccttaccacgccattctgcaagcacctcacctccctgcaacgcctaaaacttggcttcttgaaagtgacgagactaacagatgagcaagaacgagcgcttgtgctcctcaagtcactgaaagagctcgagattttttattgtactcatctcatagatcttcctgcggggctgcagacccttccttccctcaagagtttgaagatagaagagggtcgaggcatctcaaggctgccggaagcaggcctcccacattcgctggaagaactggaaatcaaaatttgcagcaagctagaagatgaatgcaggcggctagcaacatgcgaaggcaagctaaaagtcaaaattgatggtcgatatgtgaattaa

SEQ ID NO: 15 - DNA, 5067 bp, artificial sequence
  curated-TraesCS2B01G48800_Ta_2B09
atgatggagccggcgggagactcttttgtggaggccgcgattgcatggctggtgcagaccatccttgcaacgctcctgatggacaagatggaggagtggattcggcaagtcggtcttgccgacgacgtcgagaggctccagcgcgaggtcgagagagtcgacatggtggtggctgctgtgaaggggagggcagccgggaacaggcctctgtcccgggctctcgctcgtgtcaaggagcttctctacgacgccgacgacgtggtcgacgaactggactactacaggctccaacagcaagtcgaaggaggtagtaagcataatcccattatatcgaaactattatgatacttaatactccctctgtttctaaatataagtatttttagaaatttccgtatgtagtccatattgaaatctctaaaaggaattatatttagtaacggagggcgtagtttgatcttaatttctggtccatatttcttttcggcacagttacgagtgacaagcctgacgatatgcgtggagctgaaagagtggatgaaatatcaaggggccatgtcgatacactgaatgtcagtgttgggaaattacggtcctcggtatgggaacactttaccatcacagaaactgtcgaccggaagcgttcaaaagccaaatgtaagtactgtagaaaggattttaattgcgaaacgaagacaaacgggacttcatctatgaaaaaacatttggagaaagagcattccgtaacttgtacgaagaaacgtggagcccatccaccaaacccttcaaggtacccaaaggaaattgtatgttgcaccagtgcatttgtattacaagtttatatatatctgcttgagagcccattgttgctctacatttcttctgataactgacccaccatccgtttcttgttgcagcaccggtgatgcgacttgtaatgtgaggtcggttgaagttggtagttcgtccaacggaaaaagaaagagaacaaatgaggatccaacacaaaccaccgcagctaacacacacacccaatgggacaaggctgagttttccaataggataattaaaattacaggccagttacagtcacaggacatccaaggggctttgagtaaagttcttgggccatatggacctagcgctacttcaagttcaagtcatcaccgcccgagtacaacctcagctcagcacccaacaacatcaagtcttgttccactggaagtttatggcagagttgcagaaaagaacaagatcaaaaagtcaataactgaaaaccaatctggtggtgtaaatgttctacctattgtaggcattgcaggtgttggaaagacaactctcgctcaatttgtgtataatgatccagacgtgaaaagtcaatttcaccacaggatatgggtttgtgtgtcccgtaaatttgatgaagtgaagctcacaaaggagatgttagacttttttcctcgagaaaggtatgaaggaattagcaattttgcgaagcttcaagagatcttgaaagaacatatcgagtaccagtcgaagagctttctgcttgtattagacgatgtctcggacaatgttgattatcataaatggaacaaattgttgtaccctttgatgtcaagtcaagcaaagggtaatataattctagtcacaaccaaaaatttgtctgttgcacaaaggttaagaacacttgaaccgatcaagttaggtgctttagaaaatgatgatatgtggttattgctcaagtcatgtgcatttggttttggggactacaaaggtccgggaaatctaagagctattggaatgcaaatagcagagaagttaaagggcaacccgttagcagcagtaactgcaggggcactgttaagagatcatcttagcgttgatcattggagtaacattctcaagaaagagaagtggaaatcgttgggactccatgggggcatcatgcctgctttgaagcttagctatgatgagctaccgtaccatttacaacaatgtttctcgtattgttctatattttctgaaaaatataggtttcttcggaaggaactggtctatatttggatttctcaaggatttttgaatcacactaagagattggaggagataggatgggaatgtctgaataatttggtgaacctgggattctttcagcagattggagagcaacaggaaggggatgaagatgaggaagaagatttttttctaggcagtaaaatttggtattgtatgtctggtctcatgcacgattttgcaaggatggtttcaaggactgagtgtgcaaccatggatggtcttcagtgtaataatatgttaccaactatacgtcacttgtcaattgtgaccaattctgcatatagcaaagaacagcatggaaccatacctcgcaatatcaagtttgaagagaacctgagaaatgcatttgcatcagtgaggaaattgaggacattagttttatttgggcactacgactctttcttcttcaaattgttccttgatatattccagaaggaccagaacttgcgtctgctgcaaatgtctgcaacatgtgctgattttgattccttcatgtgtagtttggtaaatcctgcacatcttcgctatctaaaacgtgaacctgatgaggtgaatggtgcttcccctcaaattttgagcaagttgtaccatcttcaaatattagatgttggctcatacactgatcctatacctgatggtaataataatctagttagcctgcggcatcttattccagaaaatggagtatactcttccattgctagcattggtagaatgacatcacttaaagagctacatcattttaaggttcggttttgttctagaggatttgagatatcacaactccaatgcatgaacgagcttgtacaacttggggtgtctcgagttgatagtgttaaaactcgggaggaggcttatggagcaggactgagaagcaaagaatacttgaaaaatctgcacttgtcctggaaggataccttgtcacagaaggaatgtgacactagctctgaatattctgcagacgaaaacgaggagctctcacaaatggatacagcaagagaggtgctcgagggacttgaacctcacatgaacttaaagcatctacatatatctgggtataatggtactacttcaccaacttggcttgccaacaatctctcagttacctccttgcagacgcttcaccttgatggttgtcgaagatggagaatacttccatctcttgaaagtcttccatttcttacaaagctgaagttgagcagcatgctggaagtaatagaagtattggttccttcactggaggagctagttttgatggacatgcctaagttagtgagatgctcaagcatttctgtgggggctctgaactctagcttacgagcactacggatcgaggattgtgaagcactaaaggagtttgatctgtttgagaacgatgataattctgaaatcattcaggggtcatggctgcctggtcttaggaatttgattgtgaaatgttgccctcatttgaaagtgttgaagcctctttcaccttcaactaccttttctaaggtagtcatcagagaagttccaagatttccgtatatggaggtatcatctggtgaaaagttagaaattgggaaatttgatgaggacggagatgattttgatgaatcttgtgatgagttgaggatactggatgacaaaattttggcattccacaatcttagaaacctcaaatcgatggagatatatggttgcagaaatctaaggtcttttctgttcgaaggtttcagtcatcttgtctctttattaagtttggatataacaaagtgtgaacaacttttctcttcggatatgtcgccagagtatacccttgaagatgtgagagctgtgaacttcaatgccttcccatttctcaaaaatctcagtattgactcatgcggaatagcggggaagtggctatcgctgatgctgcagcatgcgccaggcctagaggaattgcgtttaagatattgcgcacatataacaagagtagtgttaccgatggaagaggaagaaaacagtctcttaacaacagtagtgtcatcaggaaatcaagatgaggcattgacctggttagttcgtgacggactcttgcacattccatcaaatctcgtctcctctctcaagaagatgactattggtcagtgccctcgcctaaagtttaactcgggcaaggactgcttctctggatttacctcgcttgagaagcttgaaatttggggatcattggtggatgatgacggaagtgatgacctggagaatggaagtccttttgtgttcggagaggaggatcaacccctgggagcgaatggaagatggctcctcccgacatcacttcaggagcttaacatcgggtggttctgttaccaagaaacgctgcaaccctgctttcctagagatatcaccagccttaaagagttaagtgtacgttcaatccaaggtttgcaatctctacagctgcactcatgcacggcactggaaggattggagattagaggctgtgaatcgctcaccgtcactgtactagagggcatgcaacccattggcagcctcgtgcgtttgaatgtatcagacagtactggcttgccaccatgtttggagagcttttcaacgctgtgccctcggcttgaaaggctttgcaccgatgacccatctgtccttaccacgtcattctgcaagcacctcacctccctacaaagactagaacttagtttcttgaaagtgacgagactaacagatgagcaagagcaagcgcttgtgctgctcaaatccctgcaaaagctcgaattcatttggtgttctgctctagtagttcttcctgaggggctgcacacccttccttccctcaagagattggagataaaccagtgtggacgcatcacaaggctgccagaagcaggcctcccacattcgctggaagaactcgaaatccggtcttgcagccaggagctagatgatgaatgcaggcggctagcaacaagcaaactgaaagtcaagattgattggacgtatgtgaattaa

SEQ ID NO: 16 - protein, 1584 aa, artificial sequence
  curated_TraesCS2B01G48800_Ta_2B09
MMEPAGDSFVEAAIAWLVQTILATLLMDKMEEWIRQVGLADDVERLQREVERVDMVVAAVKGRAAGNRPLSRALARVKELLYDADDVVDELDYYRLQQQVEGVTSDKPDDMRGAERVDEISRGHVDTLNVSVGKLRSSVWEHFTITETVDRKRSKAKCKYCRKDFNCETKTNGTSSMKKHLEKEHSVTCTKKRGAHPPNPSSTGDATCNVRSVEVGSSSNGKRKRTNEDPTQTTAANTHTQWDKAEFSNRIIKITGQLQSQDIQGALSKVLGPYGPSATSSSSHHRPSTTSAQHPTTSSLVPLEVYGRVAEKNKIKKSITENQSGGVNVLPIVGIAGVGKTTLAQFVYNDPDVKSQFHHRIWVCVSRKFDEVKLTKEMLDFFPRERYEGISNFAKLQEILKEHIEYQSKSFLLVLDDVSDNVDYHKWNKLLYPLMSSQAKGNIILVTTKNLSVAQRLRTLEPIKLGALENDDMWLLLKSCAFGFGDYKGPGNLRAIGMQIAEKLKGNPLAAVTAGALLRDHLSVDHWSNILKKEKWKSLGLHGGIMPALKLSYDELPYHLQQCFSYCSIFSEKYRFLRKELVYIWISQGFLNHTKRLEEIGWECLNNLVNLGFFQQIGEQQEGDEDEEEDFFLGSKIWYCMSGLMHDFARMVSRTECATMDGLQCNNMLPTIRHLSIVTNSAYSKEQHGTIPRNIKFEENLRNAFASVRKLRTLVLFGHYDSFFFKLFLDIFQKDQNLRLLQMSATCADFDSFMCSLVNPAHLRYLKREPDEVNGASPQILSKLYHLQILDVGSYTDPIPDGNNNLVSLRHLIPENGVYSSIASIGRMTSLKELHHFKVRFCSRGFEISQLQCMNELVQLGVSRVDSVKTREEAYGAGLRSKEYLKNLHLSWKDTLSQKECDTSSEYSADENEELSQMDTAREVLEGLEPHMNLKHLHISGYNGTTSPTWLANNLSVTSLQTLHLDGCRRWRILPSLESLPFLTKLKLSSMLEVIEVLVPSLEELVLMDMPKLVRCSSISVGALNSSLRALRIEDCEALKEFDLFENDDNSEIIQGSWLPGLRNLIVKCCPHLKVLKPLSPSTTFSKVVIREVPRFPYMEVSSGEKLEIGKFDEDGDDFDESCDELRILDDKILAFHNLRNLKSMEIYGCRNLRSFLFEGFSHLVSLLSLDITKCEQLFSSDMSPEYTLEDVRAVNFNAFPFLKNLSIDSCGIAGKWLSLMLQHAPGLEELRLRYCAHITRVVLPMEEEENSLLTTVVSSGNQDEALTWLVRDGLLHIPSNLVSSLKKMTIGQCPRLKFNSGKDCFSGFTSLEKLEIWGSLVDDDGSDDLENGSPFVFGEEDQPLGANGRWLLPTSLQELNIGWFCYQETLQPCFPRDITSLKELSVRSIQGLQSLQLHSCTALEGLEIRGCESLTVTVLEGMQPIGSLVRLNVSDSTGLPPCLESFSTLCPRLERLCTDDPSVLTTSFCKHLTSLQRLELSFLKVTRLTDEQEQALVLLKSLQKLEFIWCSALVVLPEGLHTLPSLKRLEINQCGRITRLPEAGLPHSLEELEIRSCSQELDDECRRLATSKLKVKIDWTYVN

SEQ ID NO: 17 - DNA, 5343 bp, artificial sequence
  curated_TraesCS2B01G488400_Ta_2B10
atggcggccgcgattgggtggctggttgagaccatctctgcgaccctccaaatcgacaagctcgacgcctggattcggcaagtcggtcttgccgatgacatcgagaagctcaagtcggagatccggagagtcaacatagtggtcactgctgccaagggcaggggggtagggagcgagctgctggatggacctttcgctcttctggaggagcggctctatgaagccgacgacgtggtcgacgagctcgactactacaggctccaacaccaagtccaaggtctgccggcacctgcagatccaagcgagccagtcccactcccagtcccaggaggtaagcgtaaatctgtctagacccaagtaatccaagtctgctaattattagtttgatcttatgttgctccaaaaatgtaaattggtcgtatctgatcaaggacgaccgttctttaatttctggtccacgatttcttttggcacagttacaaggggtgagcccgaaggcgtgcttgtagctgagcaattcaatgagatatcgaggggcggtggtgatgtaccacagagcaatgttggcaaattacggtccgtggtatgggaacactttatgatcacagaaagagataacggaaaacccaacaaggcagtatgccgacactgtagcaatgagtttaagtgtgacaccaagacgaacggtacatcatctatgaaaaagcatttggagaatgagcattctgtgacttgtacaaagaaacctcctggagcacatctaccaaacccttcaaggtacttaaaagagaattgggtatagagagtagagtattctttctaatcttaagtgtacatttttaaaaagttgtttatatacatatgcttgaggcgattgtggtcctgattaataagcacatcccccgcaaaataaataaatacgcacctctttttttctcaccacagcaccggtgagcctactataattgccagctcatccagcaaaaaacgaaagagacgacggtccaaggcatgggaattttttgatgtcatagaagaagtaaacgaacagcctatgaaagcaagatgtaaatactgtcccgcagagatcaagtgcggcccaacaagtgggacagcaggtatgctcaaccataacaagatttgtaagaacaaacctggaccaaatgaccagttgccaaacctgtcaaggtaactaaagaatctatatgttgcgtcgaaaaacaattagaagtcattaagttaagagtctcattgtggttctaatagtcaattaacgttcttttttcttattgtagcaccggtgatgctaatgcggatgtgacgccaattctaataggtaactcgtccaccagaaaagggagaatggatgattccatacaaattgatgtgactaacacagtcaccccttgggacatggccgaattatccagcaggatacgaaaaatagctagtcagttgcaatacatccaagaggaaacgactgaaattctcaagctacatggatcggactctacttcaagttcagatcatcaccagagtacaacatcatatcagcacctcagaacatcaagtcttgttccaaggaatgtgtatggaagagttaaagaaaaggaacacatcatgaaattgatgatgacagaaggcagatctgacaaagtaattgttgtgcctattgtaggcattgcaggtattggaaagacaactctcactcaacttgtgtacaacgatccagaagtggaaaggcaatttgaacataggatatgggtttgggtgtctcgcaactttgatgaaatgaggctcacaagggatatgctgagctttgtttctcaagaaagtcatgaaggaataggctgctttgggaagcttcaggagatcctgagaagtcatgtcaaatcaaagagggttttacttattttagatgatgtatggtatgacaagaaagatgcccgatggaaccaactattggctccctttaagcctcatagtgccaatggcaatgtgattcttgtgacaactagaaaaatgaccgttgcaaaaatgattggaacagtggtgccaattaagttagctactattgaaaatgatgacttttggttattattcaaatcatgtgcttttgttgatggaaactatgaatgtcttggaaatcttagcactattggacggcaaatagcagaaaagttaaagggtaacccgttagcagcagtgactacaggggcactattaaggaaccaacttaccgttgatcattggagtaaaattctcaaggaagaaaattggaaatcattaggacttagtggaggcatcatgcctgctttgaagcttagttatgatgagttgacataccgtttacaacaatgtttcttgtattgttctatatttcctgacaaatataggtttcttggtaaggatttggtatatatgtggatttctcagggatttgtgaattgcacccaaaataagagattggaggagataggattggaatatctgaatcatttggtaaacctgggattctttcagcaaattgaagaacagcaagaattggatgaggaaaaagaattctctctacgcggtcagatttggtattctatgtgtgatctcatgcatgattttgcgaggatggtttcggtgactgaatatgcgaggatagatggtctgcagtgtaagaaaatcttaccgactatacactatttgtcaatagtaactggttctgcatacaacagagatctgcatgggaatattcctcgcaatgagaagtttgaagaaaatctgagaaattctgttacatcagttaccaaattgagaacactggttgtacttgggagctttgactatttctttgtacagttgttccaagatatatttcaaaaggcccaaaatttacgcctgctgcgagtatctccagaatccacttatctgtttcaagtgcctgcagcatccactgattttaattccttcctgtgcagtttggcaaatcctttgcatcttcgttatctaaaacttgatttagacgggattgtgccacaagttctcagtacttttcttcttcttcaagtattagatgttggctcaaacagggatacttctctacccaatagcttgcataatcttgttagcctgcgacatcttgttgcacacaagagagtccattcttccattgctagcattggcaacatgacatctatccaggagctacatgattttgaggttcgaatttctagcggctttgagattacacaactcaaatccatgaacaagcttgttcaacttggagtgtctcaacttgacagtgttaaaacccgggaggaggcttatggggcaggactaagaaacaaggaacacttagaagagcttcacttgtgttggaagcatgcattttcagtggataaggatgtcagtgacactagatttgaatcttctgcagacatggccagagaagtgattgagggtcttgaaccacacatggatctaaaacatctacaaatatctcggtataatggtaccacttcaccgacttggcttgccaataatatctcagttacctcactgcagacgcttcatcttgatgattgtggaggatggagaatacttccatctctgggaagtcttccatttcttacaaagttgaagttgagcaacatgtgggaagtaacagaagtattggttccttcactggaggagctaattttactcaacatgcccaagttagtgagatgctcaagtacttctgtgggggctctgaactttagtttacgagcactgcggatcgaggattgtgaagcactgaaggagttagatctgtttgagaacgatgataattctgaaatcattcaggggtcatggctgcctggtcttaggaatttgattgtgaaatattgccctcatttgaaagtgttgaagccacttccaccttcagctaccttttctaaggtactcatcaaagtggtttcaagatttccgtctatgaaggtatcatcgggtgaaaagttagaaatttgggatgctaattaccgcagaggcgatcgatcttgtgatgagttgatcatactggatgacaaaattttggtgttccataatcttagaaacctcaaatcgatggagatatttggttgcagaaatctaaggtctttctcgtttgaaggtttcagtcatctcgtctctttaacgagcttgaaaataagaggctgtgaaaaacttttctcttcacatgagatgccagccattgaacatgtgacagctgtgaactgcgattctttcccatctcttaaaagtctcagtattaagtcatgtggaatagcggggaagtggctatcgttgatgctgcagcatgcgccaggcctagagaaattgagtttaagatattgcgcacatataacaacagtactgttaccgatggaagaggaagaaaacaatctattaacaacagtactgtcatcaggaaatcaagatgaggcattgacctggttagctcgagagggactcttgcacattccatcaaatctcgtctcctctctcaagaatatgagtattagtgagtgccctcgtctaaaatttaactggggcacggactgcttctctggatttatctcgcttgagaagcttgaaatctggggatcgttggtggatgatgacggaagttatgaccccgagaatggaagttcttttgtgttcgaagaggaggatcaacccctgggggcgaacggaagatggctcctcccgacatcacttcaggaacttaacatcaggttcttgtgttaccaagaaacgctgcaaccctgctttactagagatatcaccagccttaaaaagttatatgtaagcttcagcccaggtttgcaatctctacagctgcactcatgcacggcactggaagaattggcaattgtcggctgtggatcagtcaccgtcactgtactagaagactctcctggcttgctgccatgtttggaaaggctttgcatcaatgacccatctgtccttaccacgtcattctgcaagcacctcacctccctgcaacgcctacgacttggtttcttgaaagtgaggagactaacagatgagcaagagcaagcgcttgtgctgctcaaatccctgaaagagttccaattctatttgtgtaatgatctcgtaaatcttcctgctgggctgcacacccttccttccctcaagaggttggagatagaacggtgtggacgcatctcaaggctgccagaagcaggcctcccacattcgctggaagaactgaaaatcgagtcttgcagccaggagctatatgatgaatgcaggcagctagcaacaagcaaactgaaagtcaaaattggtgggagatatgagaattaa

SEQ ID NO: 18 - protein, 1640 aa, artificial sequence
  curated_TraesCS2B01G488400_Ta_2B10
MAAAIGWLVETISATLQIDKLDAWIRQVGLADDIEKLKSEIRRVNIVVTAAKGRGVGSELLDGPFALLEERLYEADDVVDELDYYRLQHQVQGLPAPADPSEPVPLPVPGVTRGEPEGVLVAEQFNEISRGGGDVPQSNVGKLRSVVWEHFMITERDNGKPNKAVCRHCSNEFKCDTKTNGTSSMKKHLENEHSVTCTKKPPGAHLPNPSSTGEPTIIASSSSKKRKRRRSKAWEFFDVIEEVNEQPMKARCKYCPAEIKCGPTSGTAGMLNHNKICKNKPGPNDQLPNLSSTGDANADVTPILIGNSSTRKGRMDDSIQIDVTNTVTPWDMAELSSRIRKIASQLQYIQEETTEILKLHGSDSTSSSDHHQSTTSYQHLRTSSLVPRNVYGRVKEKEHIMKLMMTEGRSDKVIVVPIVGIAGIGKTTLTQLVYNDPEVERQFEHRIWVWVSRNFDEMRLTRDMLSFVSQESHEGIGCFGKLQEILRSHVKSKRVLLILDDVWYDKKDARWNQLLAPFKPHSANGNVILVTTRKMTVAKMIGTVVPIKLATIENDDFWLLFKSCAFVDGNYECLGNLSTIGRQIAEKLKGNPLAAVTTGALLRNQLTVDHWSKILKEENWKSLGLSGGIMPALKLSYDELTYRLQQCFLYCSIFPDKYRFLGKDLVYMWISQGFVNCTQNKRLEEIGLEYLNHLVNLGFFQQIEEQQELDEEKEFSLRGQIWYSMCDLMHDFARMVSVTEYARIDGLQCKKILPTIHYLSIVTGSAYNRDLHGNIPRNEKFEENLRNSVTSVTKLRTLVVLGSFDYFFVQLFQDIFQKAQNLRLLRVSPESTYLFQVPAASTDFNSFLCSLANPLHLRYLKLDLDGIVPQVLSTFLLLQVLDVGSNRDTSLPNSLHNLVSLRHLVAHKRVHSSIASIGNMTSIQELHDFEVRISSGFEITQLKSMNKLVQLGVSQLDSVKTREEAYGAGLRNKEHLEELHLCWKHAFSVDKDVSDTRFESSADMAREVIEGLEPHMDLKHLQISRYNGTTSPTWLANNISVTSLQTLHLDDCGGWRILPSLGSLPFLTKLKLSNMWEVTEVLVPSLEELILLNMPKLVRCSSTSVGALNFSLRALRIEDCEALKELDLFENDDNSEIIQGSWLPGLRNLIVKYCPHLKVLKPLPPSATFSKVLIKVVSRFPSMKVSSGEKLEIWDANYRRGDRSCDELIILDDKILVFHNLRNLKSMEIFGCRNLRSFSFEGFSHLVSLTSLKIRGCEKLFSSHEMPAIEHVTAVNCDSFPSLKSLSIKSCGIAGKWLSLMLQHAPGLEKLSLRYCAHITTVLLPMEEEENNLLTTVLSSGNQDEALTWLAREGLLHIPSNLVSSLKNMSISECPRLKFNWGTDCFSGFISLEKLEIWGSLVDDDGSYDPENGSSFVFEEEDQPLGANGRWLLPTSLQELNIRFLCYQETLQPCFTRDITSLKKLYVSFSPGLQSLQLHSCTALEELAIVGCGSVTVTVLEDSPGLLPCLERLCINDPSVLTTSFCKHLTSLQRLRLGFLKVRRLTDEQEQALVLLKSLKEFQFYLCNDLVNLPAGLHTLPSLKRLEIERCGRISRLPEAGLPHSLEELKIESCSQELYDECRQLATSKLKVKIGGRYEN

SEQ ID NO: 19 - DNA, 4532 bp, artificial sequence
  curated_TraesCS2B01G488600_TraesCS2B01G488700_ Ta_2B11
atggaggccgcgattgcatggctggtgcagaccatccttgcaaccctcctgatcgataagctcgatgcgtggattcggcaagtcgggcttgccgatgacgttgaaaagctcaagtcagagatcaggagagtcaagatggtggtctcggctgtgaaggagagagggatcaggaacgagtcgctggatgaatctctcgctcttctcgtggagcgactctacgaagccgacgacgtggtcgacgagctggattactacaggctccaagagctggttgaaggtgcccggccccggctgcctgcagatccaaccgtgctggttccttccaacctgcccatccaaggagaaggaggtacgcatacttcttcctgtagatccaacacaaagttctttcataggccgagtatccaagtgtgacaaactactagtaattgttagtctgatgatcctatcttacttaggacaaattaatgaaatttatattatctgatcaaggacgaccatgcttttctggtccatttttctgttggcacagctacaagaaacgagcccgaaggtaacagtgctggcaaatcacggtccgtggtctgggaaaactttacagtcacagaaactgttgacagaaagtccgccaaagcagtatgtagacactgtggcaatgagttcaagtgtgatacgaagatcaacggtacatcatctatgaagaaacatttagagaaggagcatcccgataagatgaaacctcctggagcgcatccaccaaacccttcaaggtacctaaagaagaattgagcatgagcccatttaattagaaatcgtttatatacctctttcttttttcttgaatggttatatacatcttcttgacagcgcactaattttggtcctaatagccaacccaccacttttttcttactgcagcactgctgagcctattgccattgccagctcatccaggggaaaaggaaagaaacagcggtccaaggcatgggataattttgatgttatagaaaatgacattggacagccaaccaaagcaatatgtaaatactgccacacagagatcaagtgcggaatgaagaccgggacagcgggtatgcttaaccataacaagatttgcaagaagaaacctgaaccaaatgaccagccaccaaacctgtcgaggtagctaccttgcatcagcaaatttttggatgttgttttataaacaatccccaccatggttctaatagccgtttgttcatgatctttttcttactgcaacattggtgatgctactgcaaatgcgacatatattgtggtttatgacgattcagctacaagaaaaagaaggagagtggatgaggagtcagcagaaatcactgcagctaatacacacacctgttgggacaaggctacattatccaatatgatacgaaaaattattagtcagttacaagagatccaagggcaagtgagggaggttatcgagttacatggatcagacttatcttccagttcaaatcaccatcaaaatacaaccttatatcagcgcctacggacatcaagtcttggtccaagaaaagtgtatggaagagttgcagaaaagaactccattgtaaggatgataacaggagaaaagtctggtggtttagttgttctgcctattgtaggcattgcaggtgttggcaaaacaactcttgctcaacttgtatacaatgatccatatttggatgatcattttgaccaaaggatatgggtttgggtgtctcgcaattttgatgaagtgagactaacaagggagattttgaactctgtttatcaagaaaggcatgaagatataaaatgttttgcgaagcttcaggagatcttgaagcatcaggccgactcacagcgacttttaatcattttagatgatgtctgggatgacatgaacgataatatccaacaccataaaatgttggctcctctggtatcaagtcatgtgaagggtaatgtgattctagtcacaaccagaagtatgtctgttgcacaaagcttaggcaccctcaagccagtcaagttaggtgctctggcaaatgatgacttttggttattgttcaaatcacacgcatttggttacgagaactgtcaggagcatcaaagtttaagtatcatcgggcggcaaatagccgagaagttaaagggcaacccattagcagttgtatctacagcagaactattacggaagaaacttaacaccgattattggagaatcgttctaaagaacgaagagtggaaatacatgcatcacaatagagggatcatggctgctctgaagcttagctatgatcaacttccgtaccatttacaacggtgtttctcatattgctccatattccctgacagttatcagtttcttagtgaggagttggtcggtttctggatatcacagggatttgtaaagtgcaacggctctagtcagagattggaggatatagggcggggatatctgattgatttggttaacctgggcttctttgaagaagctaaaagagaagaaccatatctaggcagtcaagttatgtatgccatatgcggtctcatgcatgattttgcgatgatggtttcaaggactgacagtgcaagtatagatggtcgaccctacaaaaaaatgcctcgaactctacgacatttgtcaatagtaaatggatccgcataccagaaagatcagcatgggaacatttatcatgatgagaagtttgaagaaaatctgaaaaatgcaattacatcagttagtgaactgaggacattagtgttacttgggcactatgacttttccttcttactattattccaatatatattccaaaaggcacataacttacgtgtgctacaaatgtctgcagcatctgctgattttctcaaacatgggattgaggaggtggatgggtctttccctcaaattttgagcaaattgtaccatctccaagtattagtcggttcatacaatgatcgtactatgcctggttgtattgataatcttgttagcctgcggcatcttgttgtacacaagggagtgtactcttccattgcaaccattgataatatgctatcatttcaggaacgacatggtttcaagtttcatatttctagtggctttgagataacacaactccaatccactgaacattggatgcatgttaatactctggaagatgtttatgaggcaggactggtaaacaatgaactctcagaaaagttgcacttgtcctggaaggattctcctgcggacatggtcatggaggttgagggttgggaaccacattgggacttaagggttctcgagatatctgggtataattttgcttggacaattatggttgacaacattatcttggttacctcctcccagacggttcacatatgcgattgcattgaatggaaaatacttccatctttggaaaggtttcggtttttgacaaagctggagttgagaaacctgcctaaagtaatacaaatactggttccttcactggaggagctagctttagttaaaatgccaaagttggagaaatgtacatgcacttccgtggaaggtatgagctctagactaagagcactgcagatcaaggattgtcaatcactgaaggagtttgatctgtttgagaacaacgataaattcgaaactgggcagaggtcataggctcctagtcttagggaactaagtctggagaattgcccccatttgaaagtgttgaagcctcttccacgctcaagcatgtgttctgagttactcatctgtgacgtttcaacacttccgtacatgaagggatcatctgatgaagagttatgtattgggtatgatggtgagtatggctatggttttgacgaatcttccgatgagttgaagatactggatgacaaaattttgctgttccataatctgaaaaacctcaaatcgatggtgatacatggttgccggaatctaagttccatttcattaaaaggttttagttacctcgtctctttaacgagcttgaaaataagaaattgtgaaaaactttttgcttcaaatgagatgccagagcataccctcgaagatgtgacacttgtgaattgcaaggctttcccatctctcgaatgtctcagtattgattcatgtggtatagtggggaagtggctatctttgatgctgcaacatgcgccatgcctagaggaattgtatttgtcttcccaagaggaagaaaaatcagaagaggaagaaaacagtatatcaaatcttagctcaaccagggagggcacatcatccggaaatccagatgacggattagctctagaccgactgttgtgcatcccattaaatctcatctccattctaaagaggataactattgagaggtgccctcatctaacatttaactggggcaaggaaggcgtctcgggatttacctcccttgagaagctagtcattttagaccgccctgacctgctctcgtcgttggtgcatacagacggaggatggctactcccgaactcacttggccaacttgaaatcgatggccattcccaagtaa

SEQ ID NO: 20 - DNA, 4532 bp, artificial sequence; identical to SEQ ID NO: 19
  curated_TraesCS2B01G488600_TraesCS2B01G488700_ Ta_2B11

SEQ ID NO: 21 - DNA, 7770 bp, artificial sequence
  curated_TraesCS2B01G734100LC_Ta_2B12
gtatattgtttctgctctgctcgcgtgctccccaccctcgagcctcgactccccccacactctccactgacaagaaaccatctccagcgaacatcttctgccggatctgatggcggcctcgattgggtggctggttgagaccatctctgcaaccctcaagatcgataagctcgatgcctggattcggcaagtcggacttgccgatgacatccagaagatcaagtcggagatctggaaagtccagacagtggtcactactctactgccaagagtacgggggtcgcaaacgagcttctggatgaagctttcgctcttctcgaagagcggctctatgaagccgacgatcttgtcgacgagctcgactactacaggctccaacaccaagtccaaggtctgcctgcccctgcagatccaagcgagctactccgaagaggtaagcgtaaatctctctacacccaattaatccaagtcagctaattattagtttgatcttatattgcgccaaaaatttaaattggtcgtatctgatcaaggacgccattgcttttctgctccacgatttcttttggcacagttacaaggggtgagcccgaaggtgtgcttgtagctgagcgactcaatgagataccgaggggtgatggtgatatagcacagagacagagcaatgttggcaaattacggtccgtggtatgggaacacttcacgatcacacaaagagataatggaaaacctgtcaaagcagtatgtgtacactgtagaaatgagtttaagtgcgatacgaagacgaacggtacatcatctatgaaaaagcatttggagaatgagcattctgtgacttgtgcaaagaaacctcctggagaacatccagcaaacccttcaaggtacttaaaagagaattgggtatagagtagagtattctttcaagctcagatgtacatacaccccttaccttgtactccctccgttccatattaatcgtcgctgattagtacaactaatatggaacggagggagtatgagggaggctatgagcacatttaagaaaaaagtgttcatatacatctgcttgaggccattatatgttcctaataaccccatctttttattactgcagcaccggtgagcctactgtaattggcagctcatccagcagaaaaggaaagagacgacggtccaaggcatgggaactttttgatgtcatacaagaagtaaacgaacagcctatgaaagcaagatgtaaatactgtcccacagagatcaagtgcggaccaacgagtgggacagcaggtatgctcaaccatagcaagatttgtatacctggactaaacaaccagccgccaaacccgtcaaggtaactaaagaatctatacattgcaccgaaaaatattagaagtcattaagttaagagtctcactgtggttctaatagccaattcacggtctttttcctattgcagcactagtgatgctaatgcaaatgtgacgccaattactgcggctaacacggtcaccccttgggacatggctgaattgtccaacaagattaaaaaaatagctggtcagttgcaatacatcggaagggaagtgggtgagattctaaagctacatggatccgactgtacttcaagttcagatcagcacctcagaacaccaagtcttgttccaaggaatgtgtatggaagagttaaggaaaaggaacacatcatgaaattgatgatgacagaaggcagatctgacaaattaattgttgtgcctattgtaggcattgcaggtgttggaaagacaactctcactcaacttgtatacaatgatgtagaagtggaaaggcaatttcaccatagaatatgggtttgggtgtctcgcaactttgatgaaatgaggctcacaagagagatgttgagctttgtttctcaagaaagacatgaaggaatagactgctttgtgaagcttcaggagatcttgaaaagttatgttaaatcaaagaggattttacttattttagatgatgtttgggatgacaagaacaattaccagtggaaccaactattggctccttttcggcacgacaatgctattggtaatgtgattcttgtgacaactagaaaattgtctgttgcaaaaatgattggaacaacaagaccaattaagttaggtgcattggaaaatgatgacttcgagttattgttcaaatcatgtgcattaggtgatggaaactatgaatttcctggaaattttagcacaattgggcagcacataatagagaagttaaagggcaaccccttagcagcaataactactgggtcgctattaagggatcatcttaccgctgatcattggagtaacattctcaagaaagaaagttggaagtcactgggagtcagtggaggcatcatgcctgctttgaagcttagttatgatgagctaccataccgtttacaacaatgtttctcttactgttctatatttcctaacaaatataggtttcttggtaaggatttagtctatatttggatttctcagggatttgtgaattgcacccaaaataagagattggaggatacagggtgggaatatctgaatcaattggtaaacctgggattctttcaacaaattgaagaacaacaagaattggatgaggaagaagaattctctctatgccgtcagatttggtactctatgtgtgatctcatgcatgattttgcgaggatggtttcaaggaccaaatgtgcgactatagatggtccacagtgcaataaaatattgccaactgtacagcatttgtcaatagtaaccggttctgcatacaacaaagatctgcacgggaacattcctcgtaatgagaagtttgaagaacatctgagaaattcagttacatcagttaccaagttgagaacattggttgtacttggaaaatttgactcttcctttgtacagttgttccaagatatattccaaaaggcacaaaatttacgcctgctacgagtatcttatccacttatctgtttcaagtgcctgaagcatccaccggttttaattccttcctgtgcagtttggcaaatcctttgcatcttcgttacctaaaacttgagttggatgggattgtgccacaagttttgagtacgtttttgcatcttcaagtattagatgttggatcaagcatggatacttctctacccaatggcttgttgcataatcttgttagcctgcgacatctagttgcacacaagagagtccattcttccattactagcattggtaacatgacatctatccaggagctacatgattttaaggttcgaatttctggtggctttgagataacacaactcaaatacatgaacgagcttgttcaacttggggtgtctcagcttgacagtgttaaaacccgggaggaggcttatggagcaggattaagaaacaaggaacacttagaagagcttcacttgtcctggaaggatgcatattcagagtatgagtttgtcagtgacactagatttgaatcttctgcaaacatggcaagagaagtgattgagggtcttgaaccatacatggatttaaaacatctacaaatatcttggtataatggtaccacttcaccagcttggcttgccaacaatatctcagttacctcattgcagtcgcttcatcttaattattgtggaacatggagaacacttccatctctgggaagtcttccatttcttacaaagctgaagttgagcaacatgtgggaagtaaaagaagtattgattccttcactggaggagctagttttgatcgacatgcctaagttagtgagatgctcaagcacttctgtcgagggtctgtgctccagcttaagggtactgcagatcaaatattgtaaagcattgaaggagtttgatctgtttgataacgatgataattctggaatcactcagggatcatggctgcccggtcttaggaatttgattctggattattaccctcatttggaagtgttgaagcctcttccaccttcaactacctgttgtaaggtactcatcagagaagttccaagatttccgtatatggaggtatcatctggagaaaagttagaaattgggaatacttatggctacagaggcgatggttttgatgaatcttctgatgaattgaggatactggatgacaaaactttggcattccataaccttggaaacctcaaattgatggagatatatggttgcagaaatctaaggtctttttcgttcgaaggttttagtcatcttgtctctttagcaagtttgacaatagtagactgcgaacaacttttcccttcagatgtgtcgccagagtatacccttgaggatgtgacagctatgaactgcaatgccttcccatctcttaaaagtctcagtattcagtcatgtggaatagcggggaagtggctatcgttgatgctgcaacatgcgccaggcctagagaaattggctttagcaaattgcgcccatataacaacagtactattaacaacagtattgtccgatggaagaggaagaaaacagactattaacaacagtactgtcatcaggaaatccagatgaggcattgacctggttagctcgagactgactcttgcacgttcagtcactcaagatgattgatatttgggactgcccccgcctaacatttaacggggccaaggaatgcttctctggatttacctcccttgagaagctagtcattcgaggctgccccgacctgttctcgtcattggtacataaagacgtaacagatgaccaggcaagcggaagatggctcctcccgaaatcacttcaggaacttgagatcgttgaatattcccaagaaaagctgcagctctgcttccctagagatatcacaagccttaaaaagttaaatgtatatcacagcccaggtttgcaatctctacggctgcactcatgcacggcactggaagaattggagattagatgctgtggatcgctcaccgtcactgaactagaaggcatacaaccccttggcagcctcgggcgtttgaatgtatcagactgtcctggcttgccaccatgtttggagagcttttcaacgctgtgccctcggctggaaaggcttgagatcgatgacccatctgtccttaccacgtcattctgcaagcacctcacctccctgcaaagactacatcttggtcccatgaaaatgacgagactcacagatgagcaagagcgggcgcttgtgctgctgaagtccctgcaagagctcgaattcaatcggtgtcgtgatctcgtagatcttcctgggggcctgcacaaccttccttccctcaagaggttaaagatatgggattgtctgggcatctcaaggctgccggaagcaggtctcccattttcactggaagaactggaaatcaatcattgcagcaaggaactagctgaccaatgcagtctgctagaaacaagcaagcgaaaagtgaaaattactttatgtactccaattgattactggctgctatgttaagcacatgtttctaagctgtctctgcttttgaggaaatcttccgccgtataccctcagagttgacagaccctcataaatgtgcagtgtgctcattccagaatgagctgtctctgcaggcattcaattaggctgctcaacatatactatcatgcaacaggtaaaccggcatgtttcgctgtttgctattcatcttgtcttgtcaactgaaaaatataattaattttcatttccttgactgcacagagaactactccctccgttcctaaatataagtctttgtagagattccactatagactacatacggagcaaaatgagtgaatctacgcttaaaatgcatttatatacattcgtatgtggttcatactaatatctctacaaagacttatatttaggaacggagggagtacacgagataaacctgcagatgttttatgttgtttgttgcacaagttgtgtccgaaatttccgccattcagatatgctctgcagctacaacaatgcaccttttcaaggaaaaaaaagctaaaacaaagcacttcagagacaggaatagtagctctcgtctgacacgagaaggaggatatgtggggttactcttaactaaattcatgtgttgatcagccagactcagaagtcaggatggcctcggcagacgcctaatgtgtgcaagaatgattaaagttggatatgcaagcctgtaacctggtgtgccgtcgccgattactagtttcctgttgtgatatcagcgacgcagtgtgtgtgtagtatactactatgctatcttggtacatcctaatgagctcatctcttcccattttcctttatctttgtgatgcttcaaactatctttgtgatgcagtgtgtctgtactatcctatcttggatcttcacagaattttgctactggtctggactcattctgtcagtggttgtttgctttgtggacttgtgctcgtggtctctgttttttcaagctgatcctgaagcttgctggagcctgtgaggcacgataaaaattctcatcaaagtgaggcacaataaagctcctcgtttcttgttgactgtacgagctcctttctccagtgtgtaactgaaaatgggacgagaatgccgaaggtttgctcataaggtcatatcaccatgcgaaaccccaacagtaacgtcggggaaacagagttgatatggcctcctgtaagaaaaaagagctggtacggcccgctccagtttcatcatttcattgccatccctcgcatgtgtagcgctgtatcggaggagctctcctcttttgcgtgatatattgcgttatcaataagaaaactattcatgtctttgcttcggatatttttatgtatctgaattttcttgatcagaagaaaactctttttactctgtttgtgatgctggacaagtcatgctgtcttcgaactgtgcatgaataattttgctcctgatctggagcacttacatcgagtggtagcttactttgatgtgtgcactaacaaaagattagaaaatgtacattatacctgatggcgtaatcaatcttttctgttgtgctcaagttgttgtcgatcatgcttatcgttttcagacttcctgagctggccggcctgtgaatgtggtaagcaaacaaattttctagtcaatgatatataggcacaagtaaagaacaggacaagttaactgaatccaaggcaacctgcacatctcagaaacaagtactcactcaaatcatactgttcaagtaagacgctacaggaagttaagctgcccatcgtcttaaaccagcataggatgctcccttaactcaaaataaagctgttaaaacaagctcctctgcaatgcaagaacttcatcagttcatggagaataaacagggagctcgacagtaccgcaggatgacgaggagccactgcccaccagagattggtaagttgcggttggatctggccacagcgcctccgcatcggcgcccagaggttggtcggatgggggatgttggcgagctcgcctgcgaggcgttccctgagcgcactgccatcacggcgggccagcccccgcttgcaggaacgtcgggcatcccgggcggcggcgtcttgcaactatcggcgcgtggcgtgggagggcaagcctgaagaagacaaactagctaaatgggccggacattggcacaggccattggcgcatatatttttatattttcccaaaaagtatacatattaaaaatatattcagtaatcactttatatttctcaaaaaaataatcaattta

SEQ ID NO: 22 - protein, 1474 aa, artificial sequence
  curated_TraesCS2B01G734100LC_Ta_2B12
MAASIGWLVETISATLKIDKLDAWIRQVGLADDIQKIKSEIWKVQTVVTTLLPRVRGSQTSFWMKLSLFSKSGSMKPTILSTSSTTTGSNTKSKVCLPLQIQASYSEEDAIAFLLHDFFWHSYKGTGEPTVIGSSSSRKGKRRRSKAWELFDVIQEVNEQPMKARCKYCPTEIKCGPTSGTAGMLNHSKICIPGLNNQPPNPSSTSDANANVTPITAANTVTPWDMAELSNKIKKIAGQLQYIGREVGEILKLHGSDCTSSSDQHLRTPSLVPRNVYGRVKEKEHIMKLMMTEGRSDKLIVVPIVGIAGVGKTTLTQLVYNDVEVERQFHHRIWVWVSRNFDEMRLTREMLSFVSQERHEGIDCFVKLQEILKSYVKSKRILLILDDVWDDKNNYQWNQLLAPFRHDNAIGNVILVTTRKLSVAKMIGTTRPIKLGALENDDFELLFKSCALGDGNYEFPGNFSTIGQHIIEKLKGNPLAAITTGSLLRDHLTADHWSNILKKESWKSLGVSGGIMPALKLSYDELPYRLQQCFSYCSIFPNKYRFLVLDVGSSMDTSLPNGLLHNLVSLRHLVAHKRVHSSITSIGNMTSIQELHDFKVRISGGFEITQLKYMNELVQLGVSQLDSVKTREEAYGAGLRNKEHLEELHLSWKDAYSEYEFVSDTRFESSANMAREVIEGLEPYMDLKHLQISWYNGTTSPAWLANNISVTSLQSLHLNYCGTWRTLPSLGSLPFLTKLKLSNMWEVKEVLIPSLEELVLIDMPKLVRCSSTSVEGLCSSLRVLQIKYCKALKEFDLFDNDDNSGITQGSWLPGLRNLILDYYPHLEVLKPLPPSTTCCKVLIREVPRFPYMEVSSGEKLEIGNTYGYRGDGFDESSDELRILDDKTLAFHNLGNLKLMEIYGCRNLRSFSFEGFSHLVSLASLTIVDCEQLFPSDVSPEYTLEDVTAMNCNAFPSLKSLSIQSCGIAGKWLSLMLQHAPGLEKLALANCAHITTSLKMIDIWDCPRLTFNGAKECFSGFTSLEKLVIRGCPDLFSSLVHKDVTDDQASGRWLLPKSLQELEIVEYSQEKLQLCFPRDITSLKKLNVYHSPGLQSLRLHSCTALEELEIRCCGSLTVTELEGIQPLGSLGRLNVSDCPGLPPCLESFSTLCPRLERLEIDDPSVLTTSFCKHLTSLQRLHLGPMKMTRLTDEQERALVLLKSLQELEFNRCRDLVDLPGGLHNLPSLKRLKIWDCLGISRLPEAGLPFSLEELEINHCSKELADQCSLLETSKRKCAHSRMSCLCRHSIRLLNIYYHATARLRSQDGLGRRLIVSVLSYLGSSQNFATGLDSFCQWLFALWTCARGLCFFKLILKLAGAYFLSWPACECAVKTSSSAMQELHQFMENKQGARQYRRMTRSHCPPEIGKLRLDLATAPPHRRPEVGRMGDVGELACEAFPERTAITAGQPPLAGTSGIPGGGVLQLSARGVGGQA

SEQ ID NO: 23 - DNA, 5419 bp, artificial sequence
  curated_TraesCS2B01G489400_Ta_2B13
atgttgctcggaatcttcgaaacagctgagcaggccgcgagaacctacgatgcggcggcgctgcgcttcaagggcgccaaggccaagctcaactaccccgagggtttccagggacgcaccgacctcggcttcaaagtcacccgcagcataccggacggattacaacaacatcgccactacccctccaccatggaggcgccagcaacgcagccgtcgccgcaacagcagccgaccgtcccagtcctcatgcggcacgaactgccgcctcagggcgccggcagctccaggggcgctgtcaacctgcccttcggcgccatgtcggccccgtccacgtcgtccacctcatcgccgcacatgctcgtccctccgcttgcgtccgaggaccatacaatgagaagaactgtaagtgtagaagaggaagctaacgacacacatgacggagtgacggcgcgcacacaatctagcaagtttgtgaacagtttttacggttttgcaagtgcgtgtgcattctttactttatctgactctggtcaaaggacgaccctttttctttttcttttggcagttgcaaggaacaacgccgaatgtatgcacggtgcagacagggtcgatgagatatcaaggggcgatgctgacacaccgagtaacattgttggcaaattgcggtccgtcgtatgggaacactttacgatcacagaaaaagataatggaaaaccgctcaaagcagtatgtagacactgtggcaatgagtttaagtgtgatacaaagaccaacggtacatcgtctatgaaaaaacatttggagaacgagcatgccgtgacctttaccaagaaacctcctagagggcgtccaccaaacccttcaaggtaccctcccaaaagagaattgggcatataccttgcatgagcatatttttagaaactcgttaatacacatctgcttcgggagcccgataattgtggtcctaatagccaacctaatgtctcattttcttactgcagcactagtgagcctatcttaatcggcaactcgtccaggacaaaaggaaagagacgatggtccaaggcatggcaactttttgatatcatagaagaagaaaacggagagcctatcaaagcaatatgtaaatattgtccaacaaagatcaagtgtggaccaatgtgtgggacagctggtatgctcaaccataacaagatttgtaagaacaaacctggaccatatgaccagtcaccaaacccatcaaggtagctaatgaatctataccttgcatcgacacatttttacaagtcatttaattaagaggtctcaccgtggttctagtagccaattcacggtttcttacattaattgctgcagcacgggtgatgctactgcacatgtgaagccttcatctagcagaaaaaggaggagacccgaatcaacacaaatgaccgcgcctaacaccgcgactggttgggacaaggtcgagatatccaataggatacaaaacataactagtgagctacaaggcatccaactggaagtgcctaaggctttctatccatgtggatcaagcttatcttcaaattcagatcaccaccagagtacaatctcagatcagcgcctaaagacatcaagtcttgttcaaaagaaagtgtatgggagagatgtagaaaagaactccatcgtgaagttggtgagggcaaaaaacaaatctcacggtgtaactattttgcctattgtagggattgcgggcgttggaaagacaactctcgctcaacttgtatacaatgatccatatagtgaaagtcaatttgatcacaagatatgggtttgggtgtctcacaactttgatggcatgaggctcacaagagaaatgttgacctctgtttctcaacaaaggcatgaaggaatagactgctttgtgaagcttcaggagatcttaaaaagtcatatcaaatcaaagagggttttactaattttagatgacgtctgggatgacaaggatgattgccgcttgaaccaactaatggctccttttaagaatgatagtgataatggcaatgtgattcttgtgacaactagaaaactttctgctgcaaaaatgattggaacaacggagccaattaagttaggtgctttagaaaaggatgacctctggttattgttcaaatcatgtgcatttggtgatgaaaactatgactgtcttggaaatattagcacaattggacgacaaatagcagagaagttagaaggcaacccgttggtagcagtaactacaggggcactattaagaggtcatcttaccgttgatcattggagtaacattctcaagaaagaaagttggaaatcactgggactcaatggaggcatcatgcctgctttgaagcttagttatgatgagttgccacaccatttacaacaatgtctctcacattgttctatatttcccaaaaaatataggtttcttggtaaggatttagtctatatttggatttctcagggattcgtggatcgcacccatttaagtgagagattggaggaggcaggattggaatatttgaatgatttgatgagcctgggattctttcagcaagttgaagaccagcaggatgaagatggggatgaggatgaggaagaagaatcctctctaggcagtcaaattcggtactctatgtgtggtctcatgcatgattttgccaagatggtttcaaggactgaatgtgcaactatagatggtctacactgcaaaatgctgccaaatatacgtcatttggcgatagtaactgattctgcatacaacaaagattggtatgggaacattcctcgtaatgagaattttgaagaaaatctgagaaacacggttacatcggtcagcaaattgaggacgctggttttagttgggcactatgactctttcttcatagaattgttccaaactatattccgaaaggcacataatttacgcctgctgcaagtgtctgcaacatccactggttttaactccttttgttgtgttttggcaaatcctttgcatctacgttatctaaaacttgagttgcacggggttgtgccacaagttttgagtaagtcctttcatcttcaagtattagatgttggctcagacatgaatacttctgtacccaatggcatgcataatcttgtcagcctgcgccatcttattgcacgcaacagagtgcgctcttcaattgctagcattggcatcatggcatctcttcaggagctacatgattttgaggttcgaaatgctagcggctttgagataacacaactccaatccatgaacgagcttgtacaacttggggtgtctcaacttgataatgttaaaactcgggatgacgcttatagggcaggactaagaaacaaagaacacttagaagagcttcatttgtcctggaagtatgcactgttagaaaatgaatatagcagtgaaaaggcaagagaagttcttgagggtcttgaaccacatatgggtttaaagcatctacaaatatctaagtataatggtactacttcaccaacttggcttgccaacaaaatctcggttacctccttgcagacacttcatcttgatgattgtcgtggatggagaatacttccatctctgggaagtcttccatttcttacaaagctgaagttgagcaccatgtgtgaagtaatagaagtattacttccttcactagaggacttggtactaattaacatgccaaagttagagagatgctcaagcacttctgtggagggtttgagctctaacttgagggtgctgcagatcgagcattgcaaagcactaacgtcatttgatctgcttgagaataatgataaattcaaaatcgagcagagctcgtgcttggctggtcttaggaaattaattttgtatgattgccctcgtttgaaagtgttgaaccctcttccaccttcaacaacatgttccgagttactcatcagtggagtttcaatacttccgagtatgaagggatcatcaagtgataatttacgtattgggctcattaatgagtctataatctatggcagtattgatggatacgctgatgagtcgaggataatggatgacaaaatttttgcgttccataatcttagaaacctcaaatcgatggtgatatttggttgccaaaatttaaggtcattttcatttgaagattttagtcatctcagctctttaaagaatttggaaatatcaatgtgcaaggaacttttctcttcagatgtgatgccagagcatacccttcaaaatgtggcaaccacgaaatgcagggccttcccatctcttgaaagtctcagtattaggtcatgtggaataacagggaagtgggtatctttgatgctccaacatgcgtggatccttgaggaattgagtttggaagattgcctacacacaacaataatacaattgccgacggaagaggaagaaaacagtctatcagatcttatctcagccagggaggactcatcatcaggagatcaagacacattgacctggttagctcgagatagactcttgcacattccatcaaatatcacctcctctctcaagtggttaaccatttggaagtgccgtggtgtaacatttaatgggagtgaaaaaggtttctccagatttacctcccttaaggagctacaaattaggggatgccccgagctagtcttgcatttggtggataaagatggaacttattactgcacgaacggaagatggttcctcccatcatcacttgaggtactgggcatcgacaactatttccaagaaaagcttcaaccctgctttctgaatgatctcaccagccttaaaaggttatccgtctcgtccaggccatggttgaaatctctacagctgcactcatgcacagcactagaagagttgaaagtcattcagtgtgaatcgctcacgacactagagggcttgcaattccttggcaccctcaggcatttgacagtatacgactgccctggcatgtctacctgtttgaagagcctttcatggcgctacgggctatgctctcggctggaaacgctcggaattggtgatccatcagtccttaccacatcattctgcaagctcctcacatcgctgcaatgcctaaaattatatcattttgggtgggaagtaacgaggctaaccgataaccaagagatagcccttgtgttcctcaagtccctgcaagagctccactttttgtgctgttatgatctagtagatcttcctgcggggctgcacaaccttccttccctcaagaagttgaaaatagacacttgtccgcgcgtctcaaggctgccgaaaacaggtctcccacttccgctggaagaactggaaatcgagttttgcagcaagaagctggctgatcaatgcaggctgctagaaacaagcaagctaaaagtcaaaattagtctatgctcttga

SEQ ID NO: 24 - protein, 1723 aa, artificial sequence
  curated_TraesCS2B01G489400
MLLGIFETAEQAARTYDAAALRFKGAKAKLNYPEGFQGRTDLGFKVTRSIPDGLQQHRHYPSTMEAPATQPSPQQQPTVPVLMRHELPPQGAGSSRGAVNLPFGAMSAPSTSSTSSPHMLVPPLASEDHTMRRTVSVEEEANDTHDGVTARTQSSKFVNSFYGFASACAFFTLSDSGQRTTLFLFLLAVARNNAECMHGADRVDEISRGDADTPSNIVGKLRSVVWEHFTITEKDNGKPLKAVCRHCGNEFKCDTKTNGTSSMKKHLENEHAVTFTKKPPRGRPPNPSSTSEPILIGNSSRTKGKRRWSKAWQLFDIIEEENGEPIKAICKYCPTKIKCGPMCGTAGMLNHNKICKNKPGPYDQSPNPSSTGDATAHVKPSSSRKRRRPESTQMTAPNTATGWDKVEISNRIQNITSELQGIQLEVPKAFYPCGSSLSSNSDHHQSTISDQRLKTSSLVQKKVYGRDVEKNSIVKLVRAKNKSHGVTILPIVGIAGVGKTTLAQLVYNDPYSESQFDHKIWVWVSHNFDGMRLTREMLTSVSQQRHEGIDCFVKLQEILKSHIKSKRVLLILDDVWDDKDDCRLNQLMAPFKNDSDNGNVILVTTRKLSAAKMIGTTEPIKLGALEKDDLWLLFKSCAFGDENYDCLGNISTIGRQIAEKLEGNPLVAVTTGALLRGHLTVDHWSNILKKESWKSLGLNGGIMPALKLSYDELPHHLQQCLSHCSIFPKKYRFLGKDLVYIWISQGFVDRTHLSERLEEAGLEYLNDLMSLGFFQQVEDQQDEDGDEDEEEESSLGSQIRYSMCGLMHDFAKMVSRTECATIDGLHCKMLPNIRHLAIVTDSAYNKDWYGNIPRNENFEENLRNTVTSVSKLRTLVLVGHYDSFFIELFQTIFRKAHNLRLLQVSATSTGFNSFCCVLANPLHLRYLKLELHGVVPQVLSKSFHLQVLDVGSDMNTSVPNGMHNLVSLRHLIARNRVRSSIASIGIMASLQELHDFEVRNASGFEITQLQSMNELVQLGVSQLDNVKTRDDAYRAGLRNKEHLEELHLSWKYALLENEYSSEKAREVLEGLEPHMGLKHLQISKYNGTTSPTWLANKISVTSLQTLHLDDCRGWRILPSLGSLPFLTKLKLSTMCEVIEVLLPSLEDLVLINMPKLERCSSTSVEGLSSNLRVLQIEHCKALTSFDLLENNDKFKIEQSSCLAGLRKLILYDCPRLKVLNPLPPSTTCSELLISGVSILPSMKGSSSDNLRIGLINESIIYGSIDGYADESRIMDDKIFAFHNLRNLKSMVIFGCQNLRSFSFEDFSHLSSLKNLEISMCKELFSSDVMPEHTLQNVATTKCRAFPSLESLSIRSCGITGKWVSLMLQHAWILEELSLEDCLHTTIIQLPTEEEENSLSDLISAREDSSSGDQDTLTWLARDRLLHIPSNITSSLKWLTIWKCRGVTFNGSEKGFSRFTSLKELQIRGCPELVLHLVDKDGTYYCTNGRWFLPSSLEVLGIDNYFQEKLQPCFLNDLTSLKRLSVSSRPWLKSLQLHSCTALEELKVIQCESLTTLEGLQFLGTLRHLTVYDCPGMSTCLKSLSWRYGLCSRLETLGIGDPSVLTTSFCKLLTSLQCLKLYHFGWEVTRLTDNQEIALVFLKSLQELHFLCCYDLVDLPAGLHNLPSLKKLKIDTCPRVSRLPKTGLPLPLEELEIEFCSKKLADQCRLLETSKLKVKISLCS

SEQ ID NO: 25 - DNA, 10172 bp, artificial sequence
  curated_TraesCS2D01G466600
tactgttgtacagttgtactttccccccatttgatggaggccgcgatcgcgtggctggtggagaccatccttgcaacactcctgatcgacaagcttgatgcttggattcgccaagccgggcttgccgatgacatcgagaagctcaagtcggagatcaggagaatcaagatggtgatctctgctctcaagggcagagggatccggaaagaggcactggctgaatctctcgcccttctggaggatcacctctacgtacgacgccggcgacgtggtggacgagctcgactactacaggctccaacagcaggtccggggacaagggggcactcccactgcctggccgcctgcagatccaagcgtgcatggtacgcgtactagtgctcgtagatccaaatcaaagtgtactaattattactagttcggtctaatatatcttgcttcaaaagacaaattgatcttatcttatcaagaatatgcatttctttcctgggcatgtgtttttgggcacagttgcaagcgacgagcggcaaggtgtggatggagccgagcgagtcaatgagataccgaggggcgatgctgctacacgtaatagcagtgttggcaaattacggtcgctcgtatgggagcacttcacgatcacacaaaaggatgacggaaagcctgtgaaagcaaaatgtacatactgtacagaagagttcagatgcgaaacaaagacgaatggcacgtcatctatgaggaaccatttggagaaagagcattccgtgatttgtacgaagagacctggagcgcatccaccaaatctttcaaggtaccttcaaaaggacttttgtttttcgaaaatgaggttgaatcttctgtctctgcattaagccatgcacacggccattttattatattattcaaaaatgccttatacaagatactaaaactttgatccttcagaatccatcttctagacgataaaagtcgcaccacctacaagcttgaggataatggtggtcatgatcagggccacatgccctgacctcacccctacacaaatcatccaaaaccggaacgccggtccagcggacccttagcgcatcacatgcgtacactccgaaagtcgccaccgccgccttttgcgaacccatcttcgatgtagggatcaatgaaaagaccttgtcaggtatgccgttgacgccaccgcgaagccagaccgcgtcaccgccctgcacgcgtccatcatcgagagtccgccgccgagacttgtcgtcttcgactcgtaagaccacacaactccacctcaggatcccttcggccagcacatgctccagaaaaacgatgcctcgggagggtaaacggctccgcgcgccgctatcatccgatccgggagacccggatctagggtttctcccagtgcggcctgggcgggaagacaacaactacatcaatgatgcctctaacaagaaaatgacgccgtcatcgtccgccatgacggaagtcggcgcatttttacgggtagcctcacctcctcgaacccatggctggcttccgatccacaaatcccggagggttgcggatctcccacatcaagcgtcgtagacgccggagaaaactccggccgccacacgcctccagcaacgaactcgggtatatgatcccttgatccaccgcccccgacacagccacgtgaagctgtctcctggcccgtcatccccgccagaggggccgctgccgccgccgtgtccggagccaccgctccagggcccctgcgccgtagattgctcactagaattaattgcattgtgagatttttgttagtatactttgtgttgttgtttgatcgcgattcttctgctctgtgttctcatctttgctagtagtatacacatacaaggaattgatttttgcgagaactataaagtgcaggttccgaaagcgttttcattgggatcgatctaaccacactggtaacaatgattgaccacagactgctcgggcttcatgccgggccttgggcttcgggctttcatgccgggccagactcgggcttgcatttagacaaaatgtcaggcttcatggtcaggctcgggcttgagatatgacggtcgggctttttaaagctgagcccaaaacccggcccggcccggcccaaggtatgcccaggtttgccgcccagtctcagtgtatagttgtaaaaaagagcctgaatcagatgtaacagcatggtctgtagtagtgatatatcttccaggggcccttttacaacacaaaaattgtgtgtgctgcctttaaatgcccactacttgggatcgtgcatatagctctgcttaccacactcattgcgtataatatgttagctcttgtgtgccacaaatagatgaatcgacctacaggctacaggacgctagtatggatctcctgatccagtgtggtgttgatagctctctctatcaacaggatctcctgatttatcacaactacagattttgctctactgaaactgaaacaacccgacacccaagcatatggtcttgctgaggggtcaaatgcataccctcatcgagagagaactgaacctttgggagatcttggaatcttaatgccaccaaaaaaatacttgagttgacccaaattcttaacctcaaatctgttgctaaacctcaccttcaggcgacttacctccacatttacatctcccatgataataatattgtccacattaataacaaggatgttaatttgtttcttaatgctgacataatatcgtatgatctccatttcattgtttgtggctcaccgaaacctgtcaaacctcgctctttgtaactgcttgtgacctcccgcaaaaaaaaaactgcttgtgacctcccgcaaaaaaaaaactgcttgtgaccatacaaagacttcttcaatttgcacacctttccattggttctggggtactaaaactagacggggtctccaaataacgctccatgcagatatgcattcttgacatccaagtatccaactgatccaatggccaaccaaagttagcggtgcaagaaataagtgatcttttttgcgagaaaattttcaatctattcattttcaatcatgcagtacaacgaataccagaaataatagaaattacatccagatctgtagaccacctagtgacgactaccaacactgacgcgagctgaaggcgcgccgctgtcatcgcccctccattggcggagttgggcacaacttgttgtagtagacagccgggaagtcgtcgtgctaagaccccgtaggaccagcgcaccagaacagcagtcgccgcagctgaagaataacgtagaccagaaggatccaatccgaagacacacgaacgtagacgaacaacgacgagatccgagcaaatccaccaaagatagatccgccggagacacacctccacacgcccaccaacggtgctagacgcactgccggaagggggctaggcggggagacctttattccatcttcaggaagccgatgccgtctcgtcttccttagcaggaacaaaccctagcaaaactgaaagaaacgactaaaaacggatccctcccgccggcccttgccgagatccaccgcgcccctagggccatcggagaggaggcggacctgcggcggcgtcggcgcgaggcagaaaccccaacttttttgtggaggaggaggaggcggctagaaaggcttccgtgtccgtaatagtcaatcccatagatttatggacttggaatgtgtttggttgacatctttgtttttgagcattttgcatacttttcccagttgagcctgtttgagctaatgcatgcaaaaaaccaacatctgcatgtagtttggttgcctacatttaggctacctgcatcagggaagcaatttttaccatggtatttggttgcttgcatcgcagttgttagacaaactacatgctgttaatttggttgcaaatggcataaggtctgatcacttctcactagtgatgaccttgccacacacgggttgaacattgcctcggtcctaacttggaaagatatggcaatttatcctagctactaacaaatagcatacaaattaagagccatatgcctgaataagggaaagttcatcgatgctaaatagggtgaagtccatcctcatcctttgttcttccaggcttcgctgtcaaatgcctccacaccatgactggagctgacaacatcatcaggcttcacatctttctcctccagcacaagttcatcacaacctcattgtaggatccagttatgaaggatgcaacatgcaagaacaagtttaccctgggtagggtaagggtgaaatgacttttgatccaggatcttaaacatattcttcatagctctaaatgccctctcaaccataactctaaggctggagtatcagagattaaaaagtttatgtggagtcgtaggatagtttctaccagagaactcgttcagatggtacctggttttcctgagaggtggaagagcacccggccgacatgcatagccaacatctcctaggtagaacttgccatcggggatattgatgccatcaggtctactcatgttgtcacttagaatgttagcatcagtgctgatccttcccaaccagctagcacatatgtgaacttcagatcgaagtcaacagcaccaagaacattctggcttatagaagaaattagtggtgttggtattacccttatagaagaaagaaaatgaaacaacaattaaaaacaaatgatgaaaaacttgcacacagtttgtactgaaattgcatatttttatgaatgcaaaaataggcagataataatgcaattttgcactacagtataatttatacacattgtataatacttttgtatatatttacacacgcacacctaatatttacacatacgcataaagaaaaagaaaaactgactagaaatacttgataaacaataataaatactaaaactagtacgaagctaaaagacaaaaactgaattttccctaaggtagaatgaattaggtgcattggtttcccctctaaaaaagaaataaagaaaacttgaaacagacgacaatagaaaattttgcacatgaaatgcgcggttgcacaatatgcaaaaacaagtataccgtaattttcagataacaaagacacatgcatgtgcatacatgcacatggctgcaatgcacgaagagcatacacaaagtcactcacaacaccagcaccagcacatgcaggtcccttgcaagcaggcaagacacacacatgcacgcacacaaaatctgacacataagaaaagaaaaaaacagacaaaatatttagtagaagaaaagagtgactgacccaaaagtaaatttcagaagacttaaatgtagcaaaactgatatacatcagcttgagagcccatggttttcctaatagccagcccaccatctttttctgactgcagcaccggcgagcctattgtaattggcagctcatccaagggaaaaggaaagaaacgacggtccaaggcatgggattcttttgatgtcataaaagaagtaaacggacagcctatcaaagcaagatgtaaatactgtcccacagagatcaagtgcggaaccgggaacgggacagcaggtatgctcaaccataacaagatttgtaagaagaaacctggactagatgaccagccaccaaactcgtcaaggtagctgatgaatctttgcaccgtgacatttttagggggttgtttaaataagagccccattgtggttctattttccaattgacggtctcttccttactgcagcaccaatgatactaccgcaaatgatgctaccacaaatgcaaggcctaatctaattggtgattcatctagcagaaaaagaaggagagttgatgaggaatccgcacaaaatatcgcagctaacacaagtaccccttggaacaaggctgaattatcaaacagaatacaacaaataattagtcggttacaggacatccgaggggaagtgagtgaggttttcaagctacatgaatcagactctgcttcaagtttagatcacaaccggagtacaacctcggatcagcatctgagaacatcaagtcttatttcaaggcaattgtatgggagagttgcagaaaagaaatccatcttgaagttgatgatgtcagatgacacatctaatagcataattgttctgcctattgtaggcgttgcaggtgttggaaagacagctctcactcaacttgtatacaatgaaccaaacgtggagagtcgatttcagcacagggtatggatttgggtgtctcgaaactttgatgaagtgaggataacaagggagatgttaaactttgtttctagagaaaaacatgaagaaataaactgctttgtgaagcttcaggagatcttgaaaattcatgtaaaatcaaagagggttttaataattttagatgatgtctgggatgacatgaacgactgccgatggaaccaattgttggctccttttaagtttaatagtgctaatggcaatgtgattcttgtgacaacaagaaaactatctgttgcaaaaatggttggaacaactgagccaattaagataggtgctttggaagaggacgatttctggttattgtttaaatcatgtgcacttggtgatagagcctctgaaaatcctggaaatctatgcactattggacgacaaatagcaggcaagttaaagggcaatccgttagcagcagtaactgcaggggcactattacgagatcatcttactgttgatcattggagtaacattctcaagaaagaagactggaaatcgttgggtctcagcggaggcatcatgcctgctttgaagcttagctatgatgaactgccataccatttacaaagatgcctatcatattgttctatatttcctaacaagcataagttctcgggtaaggatttggtttatatatggatttcccaaggatttgtgagttgcgccaatttaagtaagagcttggaggagataggatggcaatatttaattgatatgacgaacatgggcttatttcagcaagtcagaggagaagagtcgtcttcattctttcactcaaattgccaaacatggtatgttatgtgtggtcttatgcatgattttgcaaggatgatctcaagaactgagtgtgcaactatagatggtttacagtgcaatgggatgatgtcaactgtgcgacatttatcaatagtaactgactctgcatacaagaaagatcagcatgggaatattcttcgtaatgagaagttcgaagaatatctaaggagtacagttacatcagttggtaaattaaggacgttgattttacttgggcactatgactctttcttctcacagttgttcaaagatattttcaaagaggcacataatttacacctgctgcagatgtctgcaacatctgctgattttagttccttcctatgtggtttggcaagcgcggtgcatcttcgttatctaaaacttgagtcagatgggttggagggggattttccacaagttttggtcaatctttttcatcttcaggtattagatgttggctcaaacaccgatcctattttacctaatggcatgcataatcttgtgaacctgcggtatcttgttgcagaaaagggagtatactcttccattgctagcattggtagcatgacatcacttcaacaacttcataatattaaggttcaattttcttgtatcggctttgagataacacaactccagtctatgaacgagcttgtacaacttggtgtgtctgaacttgaaaatgtcaaaactagatatgaggctaatggagcaaaactgagagacaaaagacacttagaagagttgcgcttgttgtggacgcatactccgtcacgagatgaatatgccactgacacgagctttcaacatccagtggacaatgtagaaagagatgtagagctcttgccaatggttgaaagagggccaagttccgagccttgtctggacagagcaagagaggtgctagagggtcttgaaccacatcaagacttaaaacatcttcagatatctgggtactatggtgctacatccccaacttggcttgccaacaatatctcagttacctccctgcgaacccttcatctagacagttgtggagaatgggaaatacttccgtttatggaaaggtttccacttctgataaaactgaagttgaccaacctgcggaaagtaatcgaagtattggttccttcactggaggagctagttttagttgaaatgccaaagttgcaaagatgtttgtgcatttccgtggggggtctgagctctagcttaagggcattgcacatcgataagtgtcaagcactaaagacgtttgatctgtttatgaacgatcataaaatcaaactagagcagaggccatggttgtctggtcttaggaaattaattatgcgtgattgccctcatttaaaagtattgaaccctcttccaccttcagccaccttttctgagttactcatcagtggagtttcaacacttccaagtatgaaggggtcatctagtgaaacgttacatattggatctttcaattggtttattgatcactcttctggtgagttgacggtactggatgataaaatattggcattccacaacctgaggagaatcaaattgatgagaatatatggttgccggaatctaacttctatttcattcgaaggttttagtcatctcgtctctttagagaggttggaaatacactggtgcgaaaaattgttctcttcacatgtttttccagagcatatccttgaagatgtgccgactgcaaattgcaaggccttcccttctcttgaaagtctcactattgagttctgtggaatagcagggaagtggctatctctgatgctgcaacatgcgccaaacctagaagaattgattttagagaattgcccccgtataacaacgctgttatcgacagaagaggaagaaaacagtccatcaaatcttatcatggacagggggtactcgtcatcaggaaatctagatgacgcattggcagggttagctcaagacgaactcttgcacgttccatcaaatcccgtctcctctcttaggaagataactattcagggctgcccttgtctgacatttaatgggagcaagaacggcttctctagatttacctcccttgaggagataacgatctacaactgccccgagctgttctcgcctttggtgcataaagccggaaatgatgaccgcacaaacggaagatggctattcccaacatcacttggggaacttgacatcgacggctattcccaagagacgctgcagccgtgttttccaagtcctctcaccagccttaaaaagttggaggtactgagcagcccaggtttggaatctctgcagcttcagtcatgcacggcacttgaagagctgataattggaggctgtggatcactcaccgcactagagggcttgcaatccattggcaacctcaggcatttgaaagtatctgattgccctggcctgcctccatatttagagagcttgtcaaggcagggctatgagatctgccctcgactggaaggacttcacatcgatgacccatctgtccttagcaagtcattctgcaagcatctcacctccctccaacgcctagaactgggtcatttgagcatggaagcgacaacactgactgatgagcaagagagagcgcttctgctgcttaagtccctgcaagagctcgacatttgtggttgttatcatctcgtagatcttcctgcgaggctggacacccttacttccctcaataggttcaagatacattcctgctccatcatctcaaggctcccactagcattttagcagtacacatgtattcctgatgttttgtaatcaataatttgccacagacctgcatgcactaggctgcccagattctgtgaccactgtccctctgctctcctaaacttgggccatacattatgttatattcagaattgatataccctcataaatgtgcactatgctcaatgtaaaaaagaccgtctctctgcatatgattcggtcttcagacaattttcctaaagcccttctatcagttgtagcatgctttgccgtatgcgttaacaaaagattaacaaatgtacatgatagctgatggtctaatcaatctttctattgtgatcaggatgt

SEQ ID NO: 26 - protein, 1642 aa, artificial sequence
  curated_TraesCS2D01G466600
MEAAIAWLVETILATLLIDKLDAWIRQAGLADDIEKLKSEIRRIKMVISALKGRGIRKEALAESLALLEDHLYVRRRRRGGRARLLQAPTAGPGTRGHSHCLAACRSKLASDERQGVDGAERVNEIPRGDAATRNSSVGKLRSLVWEHFTITQKDDGKPVKAKCTYCTEEFRCETKTNGTSSMRNHLEKEHSVICTKRPGAHPPNLSSTGEPIVIGSSSKGKGKKRRSKAWDSFDVIKEVNGQPIKARCKYCPTEIKCGTGNGTAGMLNHNKICKKKPGLDDQPPNSSSTNDTTANDATTNARPNLIGDSSSRKRRRVDEESAQNIAANTSTPWNKAELSNRIQQIISRLQDIRGEVSEVFKLHESDSASSLDHNRSTTSDQHLRTSSLISRQLYGRVAEKKSILKLMMSDDTSNSIIVLPIVGVAGVGKTALTQLVYNEPNVESRFQHRVWIWVSRNFDEVRITREMLNFVSREKHEEINCFVKLQEILKIHVKSKRVLIILDDVWDDMNDCRWNQLLAPFKFNSANGNVILVTTRKLSVAKMVGTTEPIKIGALEEDDFWLLFKSCALGDRASENPGNLCTIGRQIAGKLKGNPLAAVTAGALLRDHLTVDHWSNILKKEDWKSLGLSGGIMPALKLSYDELPYHLQRCLSYCSIFPNKHKFSGKDLVYIWISQGFVSCANLSKSLEEIGWQYLIDMTNMGLFQQVRGEESSSFFHSNCQTWYVMCGLMHDFARMISRTECATIDGLQCNGMMSTVRHLSIVTDSAYKKDQHGNILRNEKFEEYLRSTVTSVGKLRTLILLGHYDSFFSQLFKDIFKEAHNLHLLQMSATSADFSSFLCGLASAVHLRYLKLESDGLEGDFPQVLVNLFHLQVLDVGSNTDPILPNGMHNLVNLRYLVAEKGVYSSIASIGSMTSLQQLHNIKVQFSCIGFEITQLQSMNELVQLGVSELENVKTRYEANGAKLRDKRHLEELRLLWTHTPSRDEYATDTSFQHPVDNVERDVELLPMVERGPSSEPCLDRAREVLEGLEPHQDLKHLQISGYYGATSPTWLANNISVTSLRTLHLDSCGEWEILPFMERFPLLIKLKLTNLRKVIEVLVPSLEELVLVEMPKLQRCLCISVGGLSSSLRALHIDKCQALKTFDLFMNDHKIKLEQRPWLSGLRKLIMRDCPHLKVLNPLPPSATFSELLISGVSTLPSMKGSSSETLHIGSFNWFIDHSSGELTVLDDKILAFHNLRRIKLMRIYGCRNLTSISFEGFSHLVSLERLEIHWCEKLFSSHVFPEHILEDVPTANCKAFPSLESLTIEFCGIAGKWLSLMLQHAPNLEELILENCPRITTLLSTEEEENSPSNLIMDRGYSSSGNLDDALAGLAQDELLHVPSNPVSSLRKITIQGCPCLTFNGSKNGFSRFTSLEEITIYNCPELFSPLVHKAGNDDRTNGRWLFPTSLGELDIDGYSQETLQPCFPSPLTSLKKLEVLSSPGLESLQLQSCTALEELIIGGCGSLTALEGLQSIGNLRHLKVSDCPGLPPYLESLSRQGYEICPRLEGLHIDDPSVLSKSFCKHLTSLQRLELGHLSMEATTLTDEQERALLLLKSLQELDICGCYHLVDLPARLDTLTSLNRFKIHSCSIISRLPLAF